SEQUENCE LISTING (70 sequences)

SEQ ID NO: 1 - protein, 9 aa, synthetic construct
  REGION 1..9: note = Exemplary initial peptide sequence
GMGQKDSYV

SEQ ID NO: 2 - protein, 9 aa, synthetic construct
  REGION 1..9: note = Exemplary initial peptide sequence
RMQKEITAL

SEQ ID NO: 3 - protein, 9 aa, synthetic construct
  REGION 1..9: note = Exemplary initial peptide sequence
QLAKLMATL

SEQ ID NO: 4 - protein, 9 aa, synthetic construct
  REGION 1..9: note = Exemplary initial peptide sequence
LMYAKRAFV

SEQ ID NO: 5 - protein, 9 aa, synthetic construct
  REGION 1..9: note = Synthetic peptide
SLYSYFQKV

SEQ ID NO: 6 - protein, 10 aa, synthetic construct
  REGION 1..10: note = Synthetic peptide
SLLPIMWQLY

SEQ ID NO: 7 - protein, 10 aa, synthetic construct
  REGION 1..10: note = Tumor associated antigen
ELAGIGILTV

SEQ ID NO: 8 - protein, 9 aa, synthetic construct
  REGION 1..9: note = Tumor associated antigen
SLLMWITQV

SEQ ID NO: 9 - protein, 5 aa, synthetic construct
  REGION 1..5: note = Elastin Like Peptide (ELP)
  VARIANT 4: note = Xaa is a variable amino acid which can modulate the effects of the motif
VPGXG

SEQ ID NO: 10 - skipped

SEQ ID NO: 11 - protein, 9 aa, synthetic construct
  REGION 1..9: note = Exemplary initial peptide sequence
IMALKQAGL

SEQ ID NO: 12 - protein, 9 aa, synthetic construct
  REGION 1..9: note = Exemplary initial peptide sequence
FLAIKLYVG

SEQ ID NO: 13 - protein, 9 aa, synthetic construct
  REGION 1..9: note = Exemplary initial peptide sequence
GAATKMAAV

SEQ ID NO: 14 - protein, 10 aa, synthetic construct
  REGION 1..10: note = Exemplary initial peptide sequence
QLALKKVEGV

SEQ ID NO: 15 - protein, 10 aa, synthetic construct
  REGION 1..10: note = Exemplary initial peptide sequence
IMALKKQAGL

SEQ ID NO: 16 - protein, 10 aa, synthetic construct
  REGION 1..10: note = Exemplary initial peptide sequence
FLAIKKLYVG

SEQ ID NO: 17 - protein, 10 aa, synthetic construct
  REGION 1..10: note = Exemplary initial peptide sequence
TEIGKDVIGL

SEQ ID NO: 18 - protein, 10 aa, synthetic construct
  REGION 1..10: note = Exemplary initial peptide sequence
ILKEKKVHVG

SEQ ID NO: 19 - protein, 9 aa, synthetic construct
  REGION 1..9: note = Labelled peptide structure
  SITE 5: note = FITC label
GMGQKDSYV

SEQ ID NO: 20 - protein, 9 aa, synthetic construct
  REGION 1..9: note = Labelled peptide structure
  SITE 4: note = FITC label
RMQKEITAL

SEQ ID NO: 21 - protein, 9 aa, synthetic construct
  REGION 1..9: note = Labelled peptide structure
  SITE 5: note = FITC label
IMALKQAGL

SEQ ID NO: 22 - protein, 9 aa, synthetic construct
  REGION 1..9: note = Labelled peptide structure
  SITE 5: note = FITC label
FLAIKLYVG

SEQ ID NO: 23 - protein, 9 aa, synthetic construct
  REGION 1..9: note = Labelled peptide structure
  SITE 5: note = FITC label
GAATKMAAV

SEQ ID NO: 24 - protein, 10 aa, synthetic construct
  REGION 1..10: note = Labelled peptide structure
  SITE 6: note = FITC label
QLALKKVEGV

SEQ ID NO: 25 - protein, 10 aa, synthetic construct
  REGION 1..10: note = Labelled peptide structure
  SITE 6: note = FITC label
IMALKKQAGL

SEQ ID NO: 26 - protein, 10 aa, synthetic construct
  REGION 1..10: note = Labelled peptide structure
  SITE 6: note = FITC label
FLAIKKLYVG

SEQ ID NO: 27 - protein, 10 aa, synthetic construct
  REGION 1..10: note = Labelled peptide structure
  SITE 5: note = FITC label
TEIGKDVIGL

SEQ ID NO: 28 - protein, 10 aa, synthetic construct
  REGION 1..10: note = Labelled peptide structure
  SITE 6: note = FITC label
ILKEKKVHVG

SEQ ID NO: 29-30 - skipped (2 entries)

SEQ ID NO: 31 - protein, 8 aa, synthetic construct
  REGION 1..8: note = Candidate peptide sequence
YTVKFALV

SEQ ID NO: 32 - protein, 8 aa, synthetic construct
  REGION 1..8: note = Candidate peptide sequence
MTYKFPVT

SEQ ID NO: 33 - protein, 8 aa, synthetic construct
  REGION 1..8: note = Candidate peptide sequence
QIPTKLAA

SEQ ID NO: 34 - protein, 9 aa, synthetic construct
  REGION 1..9: note = Candidate peptide sequence
FLQEKSPAV

SEQ ID NO: 35 - protein, 9 aa, synthetic construct
  REGION 1..9: note = Candidate peptide sequence
FLAIKLYGV

SEQ ID NO: 36 - protein, 9 aa, synthetic construct
  REGION 1..9: note = Candidate peptide sequence
FLLDKKIGV

SEQ ID NO: 37 - protein, 9 aa, synthetic construct
  REGION 1..9: note = Candidate peptide sequence
ALWAKSPGV

SEQ ID NO: 38 - protein, 9 aa, synthetic construct
  REGION 1..9: note = Candidate peptide sequence
FLLEKSRVV

SEQ ID NO: 39 - protein, 9 aa, synthetic construct
  REGION 1..9: note = Candidate peptide sequence
YLSEKVLAA

SEQ ID NO: 40 - protein, 9 aa, synthetic construct
  REGION 1..9: note = Candidate peptide sequence
FLAVKPDGV

SEQ ID NO: 41 - protein, 9 aa, synthetic construct
  REGION 1..9: note = Candidate peptide sequence
LLYFKVFGI

SEQ ID NO: 42 - protein, 9 aa, synthetic construct
  REGION 1..9: note = Candidate peptide sequence
ILISKLLGA

SEQ ID NO: 43 - protein, 9 aa, synthetic construct
  REGION 1..9: note = Candidate peptide sequence
YLLEKTRVA

SEQ ID NO: 44 - protein, 9 aa, synthetic construct
  REGION 1..9: note = Candidate peptide sequence
ALDFKLAAA

SEQ ID NO: 45 - protein, 9 aa, synthetic construct
  REGION 1..9: note = Candidate peptide sequence
QLALKVEGV

SEQ ID NO: 46 - protein, 9 aa, synthetic construct
  REGION 1..9: note = Candidate peptide sequence
FLKIKVTAA

SEQ ID NO: 47 - protein, 9 aa, synthetic construct
  REGION 1..9: note = Candidate peptide sequence
ALLAKRLGA

SEQ ID NO: 48 - protein, 9 aa, synthetic construct
  REGION 1..9: note = Candidate peptide sequence
GLTFKGPGA

SEQ ID NO: 49 - protein, 9 aa, synthetic construct
  REGION 1..9: note = Candidate peptide sequence
YAVTKTDGI

SEQ ID NO: 50 - protein, 9 aa, synthetic construct
  REGION 1..9: note = Candidate peptide sequence
IFINKLNGA

SEQ ID NO: 51 - protein, 9 aa, synthetic construct
  REGION 1..9: note = Candidate peptide sequence
ILKEKVHVG

SEQ ID NO: 52 - protein, 9 aa, synthetic construct
  REGION 1..9: note = Candidate peptide sequence
KVLGKGSFG

SEQ ID NO: 53 - protein, 9 aa, synthetic construct
  REGION 1..9: note = Candidate peptide sequence
RTSQKGTVA

SEQ ID NO: 54 - protein, 9 aa, synthetic construct
  REGION 1..9: note = Candidate peptide sequence
QSDPKPAVA

SEQ ID NO: 55 - protein, 9 aa, synthetic construct
  REGION 1..9: note = Candidate peptide sequence
LFLPKLVVG

SEQ ID NO: 56 - protein, 9 aa, synthetic construct
  REGION 1..9: note = Candidate peptide sequence
IDPYKEFGA

SEQ ID NO: 57 - protein, 9 aa, synthetic construct
  REGION 1..9: note = Candidate peptide sequence
IDTYKEFGA

SEQ ID NO: 58 - protein, 10 aa, synthetic construct
  REGION 1..10: note = Candidate peptide sequence
FLAIKKLYGV

SEQ ID NO: 59 - protein, 10 aa, synthetic construct
  REGION 1..10: note = Candidate peptide sequence
NLLEKDYFGL

SEQ ID NO: 60 - protein, 10 aa, synthetic construct
  REGION 1..10: note = Candidate peptide sequence
GLFGKTQVGV

SEQ ID NO: 61 - protein, 10 aa, synthetic construct
  REGION 1..10: note = Candidate peptide sequence
SLLFKTSVGV

SEQ ID NO: 62 - protein, 10 aa, synthetic construct
  REGION 1..10: note = Candidate peptide sequence
LLEGKVLPGV

SEQ ID NO: 63 - protein, 10 aa, synthetic construct
  REGION 1..10: note = Candidate peptide sequence
LLYFKKVFGI

SEQ ID NO: 64 - protein, 10 aa, synthetic construct
  REGION 1..10: note = Candidate peptide sequence
ALWAKSPGVL

SEQ ID NO: 65 - protein, 10 aa, synthetic construct
  REGION 1..10: note = Candidate peptide sequence
FLKIKKVTAA

SEQ ID NO: 66 - protein, 10 aa, synthetic construct
  REGION 1..10: note = Candidate peptide sequence
ALLAKKRLGA

SEQ ID NO: 67 - protein, 10 aa, synthetic construct
  REGION 1..10: note = Candidate peptide sequence
AALKKALAAA

SEQ ID NO: 68 - protein, 10 aa, synthetic construct
  REGION 1..10: note = Candidate peptide sequence
YAVTKKTDGI

SEQ ID NO: 69 - protein, 10 aa, synthetic construct
  REGION 1..10: note = Candidate peptide sequence
TTGLKPTVAL

SEQ ID NO: 70 - protein, 10 aa, synthetic construct
  REGION 1..10: note = Candidate peptide sequence
KVLGKGSFGK

(19) United States
(12) Patent Application Publication
(10) Pub. No.: US 2024/0272171 A1
(43) Pub. Date: Aug. 15, 2024

(54) Methods for Identification of Exchangeable MHC Binding Peptides and Methods of Use Thereof

(71) Applicant: PhenomeX Inc., Emeryville, CA (US)

(72) Inventor: Peter J. Beemiller, Emeryville, CA (US)

(73) Assignee: PhenomeX Inc., Emeryville, CA (US)

(21) Appl. No.: 18/332,207

(22) Filed: Jun. 9, 2023

Related U.S. Application Data
(63) Continuation of application No. PCT/US2021/062775, filed on Dec. 10, 2021.
(60) Provisional application No. 63/226,476, filed on Jul. 28, 2021.
(60) Provisional application No. 63/124,606, filed on Dec. 11, 2020.

(51) Int. Cl. G01N 33/68; G01N 33/543

(52) U.S. Cl. CPC G01N 33/6845; G01N 33/54306; G01N 2333/7051; G01N 2333/70539; G01N 2458/00

(57) ABSTRACT

Methods for identification of exchangeable MHC binding peptides are provided herein. Their use in proto-antigen-presenting surfaces and related kits, methods, and uses are also described. The proto-antigen-presenting surface can comprise a plurality of primary activating molecular ligands comprising a major histocompatibility complex (MHC) molecule configured to bind to a T cell receptor (TCR) of a T cell and a plurality of co-activating molecular ligands each including a TCR co-activating molecule or an adjunct TCR activating molecule, wherein an initial peptide is bound to the MHC molecules. Proto-antigen-presenting surfaces can be used to rapidly prepare antigen-presenting surfaces comprising one or more peptide antigens of interest by contacting the proto-antigen-presenting surface in the presence of an exchange factor and with one or more peptide antigens so as to displace the initial peptide. Methods of identifying suitable initial peptides having affinity for the MHC as well as being capable of displacement by an antigenic peptide are described.

This application is a continuation of International Patent Application No. PCT/US2021/062775, filed Dec. 10, 2021, which claims the benefit under 35 U.S.C. 119(e) of U.S. Provisional Application No. 63/124,606, filed on Dec. 11, 2020, and U.S. Provisional Application No. 63/226,476, filed on Jul. 28, 2021, each of which disclosures is herein incorporated by reference in its entirety.

The present application contains a Sequence Listing which has been submitted electronically in XML format. Said XML copy, created on Nov. 13, 2023, is named “01149-0020-00US-ST26.xml” and is 89,538 bytes in size. The information in the electronic format of the sequence listing is incorporated herein by reference in its entirety.

### INTRODUCTION AND SUMMARY

Immunotherapy offers a potentially powerful approach to treating cancers successfully. T lymphocyte activation is one aspect of preparing tumor-targeting cytotoxic T lymphocytes for use in immunotherapy. Identifying immunogenic antigen peptide sequences from tumor-associated antigens or other disease-associated antigens that can be used to activate T lymphocytes can facilitate such activation.

T lymphocytes become activated though exposure to an antigen presented by a major histocompatibility complex (MHC) together with one or more coactivating stimuli. The MHC generally binds tightly to a peptide antigen and does not fold properly without a peptide antigen, meaning that preparation of an MHC bound to a peptide antigen of interest for use in T cell activation has been non-trivial, including in situations where there are multiple possible antigens of interest that one desires to evaluate for immunogenicity. Heuristic models based on known antigens can be used to identify potential novel peptide antigens, but these models may suffer from a high false-positive rate. Accordingly, there is a need for rapid verification of the immunogenicity of peptide antigens. More generally, T cell activation may be improved by using more reproducible and better characterizable technologies.

The MHC may not retain a stable conformation in the absence of a peptide bound into the antigen-binding groove. When an unbound MHC molecule is presented on an antigen-presenting surface, it will not remain on the surface. In designing synthetic antigen presenting surfaces for activation of T cells, an initial peptide may be included to form a peptide-MHC complex to stably manufacture the ligands for attachment to the surfaces and subsequent use.

As discussed further herein, exchange factors, such as dipeptides (e.g., Glycine-Xaa where Xaa is Leu, Phe, Val, Arg, Met, Lys, Ile, homoleucine, cyclohexylalanine, or Norleucine) can react with an MHC, which is already or subsequently becomes surface-associated, to generate a proto-antigen-presenting surface. In some embodiments, Xaa of the dipeptide may be Leu, Phe, Val, Arg, Met, Ile, homoleucine, cyclohexylalanine, or Norleucine Such proto-antigen-presenting surfaces can then serve as substrates for generating antigen-presenting surfaces through displacement of the exchange factor with a peptide antigen. The antigen-presenting surfaces can then activate T cells if the peptide antigen is immunogenic. Thus, T cell activation provides a readout of peptide antigen immunogenicity.

The presently disclosed proto-antigen-presenting surfaces, initial, e.g., in-place, peptides for use therein, and related methods and uses can provide benefits such as more rapid evaluation of peptide antigen immunogenicity because the relatively laborious process of folding the MHC need not be performed with a peptide antigen of interest and need not be performed with each individual member of a set of peptide antigens being evaluated for immunogenicity. For example, an exemplary method comprises folding an MHC with an initial peptide, which may be any of the initial peptides described herein, and preparing a proto-antigen-presenting surface by associating the MHC with a suitable surface and contacting the MHC with an exchange factor to displace the initial peptide. The contacting step may occur before or after the associating step. An antigen-presenting surface can be prepared by contacting the proto-antigen-presenting surface with one or more peptide antigens of interest (e.g., one or more pools of peptide antigens) such that the one or more peptide antigens of interest displace the exchange factor and become associated with the MHC. The resulting surface can then be used to evaluate peptide antigen immunogenicity, e.g., by determining whether or to what extent it activates T lymphocytes. Additional embodiments include kits for preparing proto-antigen-presenting surfaces or antigen-presenting surfaces comprising an exchange factor and an MHC associated with a surface; methods of using antigen-presenting surfaces prepared as described herein to activate T lymphocytes; T lymphocytes prepared according to such methods; and methods of using such T lymphocytes, e.g., to treat diseases such as cancer.

In a first aspect, a kit for generating an antigen-presenting surface, is provided, the kit includes: (a) a covalently functionalized synthetic surface; (b) a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface; and (c) an initial peptide bound to the MHC molecule, wherein the initial peptide comprises IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

In another aspect, a kit for generating an antigen-presenting surface, is provided, the kit includes: (a) a covalently functionalized synthetic surface; (b) a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface; and (c) an initial peptide bound to the MHC molecule, wherein the initial peptide comprises GMGQKDSYV (SEQ ID NO: 1); RMQKEITAL (SEQ ID NO: 2); IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

In yet another aspect, a kit for generating an antigen-presenting surface, is provided, the kit includes: (a) a covalently functionalized synthetic surface; (b) a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface; and (c) an initial peptide bound to the MHC molecule, wherein the initial peptide has an affinity for binding a binding groove of the MHC molecule comprising a predicted Kd from about 1E+1 nM to about 1E+5 nM, from about 1E+1 nM to about 2E+5 nM, from about 1E+2 nM to about 1E+5 nM, from about 1E+2 nM to about 2E+4 nM, from about 1E+2 nM to about 1E+3 nM, from about 1E+2 nm to about 1E+4 nM, from about 1E+1 nm to about 2E+4 nM, from about 1E+2 nM to about 1E+3 nM, from about 1.5E+2 nM to about 1E+5 nM.

In some variations, the kit can further include one or more of: at least one co-activating molecule that comprises a second reactive moiety configured to react with or bind to the covalently functionalized surface, wherein each co-activating molecule is selected from a TCR co-activating molecule and an adjunct TCR activating molecule; a surface-blocking molecule capable of covalently binding to the covalently functionalized synthetic surface; a buffer suitable for performing an exchange reaction; and instructions for performing an exchange reaction wherein a peptide antigen displaces the initial peptide.

In some variations, the kit can further include an exchange factor, wherein the exchange factor is provided separately from the primary activating molecule and the initial peptide bound to the MHC molecule.

In a further aspect, a method of identifying an initial peptide sequence for stabilizing a major histocompatibility complex (MHC) molecule, is provided, the method includes: binding a first peptide sequence to a binding groove of the MHC molecule, wherein the first peptide sequence is a detectably labelled peptide sequence, thereby forming a detectably labelled peptide sequence:MHC molecule complex (LP:MHC complex) stabilizing the MHC molecule; performing an exchange reaction comprising contacting the LP:MHC complex with an exchange factor and a second peptide sequence for a first period of time, wherein the second peptide sequence is configured to stabilize the MHC molecule when bound to the binding groove; and detecting displacement of the detectably labelled peptide sequence from the binding groove of the MHC molecule.

In some variations, the detectably labelled peptide sequence includes a highly conserved self peptide sequence and minimal immunogenicity.

In some variations, an affinity of the detectably labelled peptide sequence for binding the binding groove includes a predicted Kd from about 1E+1 nM to about 1E+5 nM, from about 1E+1 nM to about 2E+5 nM, from about 1E+2 nM to about 1E+5 nM, from about 1E+2 nM to about 2E+4 nM, from about 1E+2 nM to about 1E+3 nM, from about 1E+2 nm to about 1E+4 nM, from about 1E+1 nm to about 2E+4 nM, from about 1E+2 nM to about 1E+3 nM, from about 1.5E+2 nM to about 1E+5 nM.

In another aspect, a method of analyzing stability of a complex comprising a major histocompatibility complex (MHC) molecule and a peptide antigen is provided, wherein the MHC molecule is: configured to bind to a T cell receptor (TCR); and stabilized by complexation with an initial peptide which is identified by the method above; wherein the method includes: contacting a plurality of the MHC molecules with the peptide antigen and an exchange factor, thereby forming peptide antigen-bound MHC molecules; and measuring total binding and/or an extent of dissociation of the peptide antigen from the MHC molecule.

In some variations, measuring total binding and/or the extent of dissociation includes measuring binding of an agent to the MHC molecule, wherein the agent specifically binds to (i) the initial peptide, and/or (ii) a peptide-bound conformation of the MHC molecule.

In another aspect, a method of analyzing stability of a plurality of complexes each including a histocompatibility complex (MHC) molecule and a peptide antigen is provided, including performing any of the methods of analyzing stability of a complex comprising a major histocompatibility complex (MHC) molecule and a peptide antigen, as described herein, with each of a plurality of different peptide antigens

In a further aspect, a kit for generating an antigen-presenting surface is provided, the kit including: (a) a covalently functionalized synthetic surface; (b) a primary activating molecule that includes a major histocompatibility complex (MHC) molecule configured to bind to a T cell receptor (TCR), and a first reactive moiety configured to react with or bind to the covalently functionalized surface; and (c) an initial peptide bound to the MHC molecule, wherein the initial peptide is a peptide sequence configured to stabilize the MHC molecule identified by any of the methods of identifying an initial peptide sequence for stabilizing a major histocompatibility complex (MHC) molecule as described herein.

In yet another aspect, a method of forming a proto-antigen-presenting surface is provided, the method including: synthesizing a plurality of primary activating molecules, wherein synthesizing each of the plurality of primary activating molecules includes reacting a major histocompatibility complex (MHC) molecule with an initial peptide (e.g. in the presence of an initial peptide), thereby forming a primary activating molecule, including the MHC molecule complexed with the initial peptide; wherein the initial peptide is the peptide sequence configured to stabilize the MHC molecule identified by any of the methods of identifying an initial peptide sequence for stabilizing a major histocompatibility complex (MHC) molecule as described herein; and reacting the plurality of primary activating molecules with a first plurality of binding moieties disposed on a covalently functionalized synthetic surface, thereby forming the proto-antigen-presenting surface.

In a further aspect, a proto-antigen-presenting surface is provided, the surface including: a plurality of primary activating molecular ligands, wherein each primary activating molecular ligand includes a major histocompatibility complex (MHC) molecule configured to bind to a T cell receptor (TCR) of a T cell; and wherein an initial peptide is bound to the MHC molecule, wherein the initial peptide is the peptide sequence configured to stabilize the MHC molecule identified by any of the methods of identifying an initial peptide sequence for stabilizing a major histocompatibility complex (MHC) molecule as described herein; and a plurality of co-activating molecular ligands each including a TCR co-activating molecule or an adjunct TCR activating molecule.

In another aspect, a method of preparing an antigen-presenting surface including a peptide antigen is provided, the method including: reacting the peptide antigen with any of the proto-antigen-presenting surfaces as described herein, wherein the initial peptide is substantially displaced and the peptide antigen becomes associated with the MHC molecules.

In yet another aspect, a method of screening a plurality of peptide antigens for T-cell activation is provided, the method including: reacting a plurality of different peptide antigens with a plurality of proto-antigen-presenting surfaces, which may be any of the proto-antigen-presenting surfaces as described herein, thereby substantially displacing exchange factors or initial peptides and forming a plurality of antigen-presenting surfaces; contacting a plurality of T cells with the antigen-presenting surfaces; and monitoring the T cells for activation, wherein activation of a T cell indicates that a peptide antigen associated with the surface with which the T cell was contacted is able to contribute to T cell activation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates a microfluidic device and a system with associated control equipment according to some embodiments of the disclosure.

FIG. 1B illustrates a microfluidic device with sequestration pens according to an embodiment of the disclosure.

FIGS. 2A to 2B illustrate a microfluidic device having sequestration pens according to some embodiments of the disclosure.

FIG. 2C illustrates a sequestration pen of a microfluidic device according to some embodiments of the disclosure.

FIG. 3 illustrates a sequestration pen of a microfluidic device according to some embodiments of the disclosure.

FIGS. 4A to 4B illustrate electrokinetic features of a microfluidic device according to some embodiments of the disclosure.

FIG. 5A illustrates a system for use with a microfluidic device and associated control equipment according to some embodiments of the disclosure.

FIG. 5B illustrates an imaging device according to some embodiments of the disclosure.

FIG. 6 is a graphical representation of T cell activation pathways according to an embodiment of the disclosure.

FIGS. 7A and 7B are schematic representations of preparation of antigen-presenting surfaces according to various embodiments of the disclosure.

FIG. 8 is a schematic representation of the process of preparing an antigen presenting surface according to an embodiment of the disclosure

FIG. 9 is a graphical representation of Fourier Transform Infrared spectra of a covalently functionalized polystyrene bead at selected steps of the functionalization.

FIGS. 10A-10D are graphical representations of various characterization parameters for activation of T cells, according to an embodiment of the disclosure.

FIGS. 11A-11E are graphical representations of cell product characterization according to an embodiment of the disclosure.

FIG. 12 is a graphical representation of cell product characterization according to an embodiment of the disclosure.

FIG. 13 is a graphical representation of cytotoxicity experiments according to one embodiment of the disclosure.

FIGS. 14A-14C are graphical representations of cell product characterization according to an embodiment of the disclosure.

FIGS. 15A-15I are graphical representations of the characterization of activation using an antigen-presenting surface according to some embodiments of the disclosure.

FIGS. 16A-16F are graphical representations of characterization of activation using antigen-presenting surfaces according to some embodiments of the disclosure.

FIGS. 17A-17E are graphical representations of the characterization of the cellular product obtained using an antigen-presenting surface according to some embodiments of the disclosure.

FIG. 18 shows a quantitation of the peptide switching for the indicated peptides (SEQ ID NOs: 5 and 6 from left to right).

FIGS. 19A-19B show a time course of median fluorescence intensity versus time for binding of a conformationally sensitive antibody which only recognizes pHLAs in the folded, complex conformation to pHLA beads loaded with either SLYSYFQKV (SEQ ID NO: 5) (FIG. 27A) or SLLPIMWQLY (SEQ ID NO: 6) (FIG. 27B) peptides.

FIGS. 20A-20D show levels of surface markers for cultured cells following culture with standard pHLA or switched pHLA.

FIGS. 21A-21C show frequencies of types of T cells following culture with standard pHLA or switched pHLA.

FIG. 22 is a graphical representation of Class 1 HLA-peptide binding interactions according to an embodiment of the present disclosure. HLA molecules are not structurally stable without peptide binding to the HLA, and if unbound, HLA molecules do not remain at the surface of Antigen-Presenting Cells (APCs). Class 1 HLA-peptide interactions are mediated by six pockets in the binding groove. Pocket A and Pocket F anchor the peptide in the groove. Exogenous peptides can be loaded onto APCs, which suggests a mechanism for direct peptide exchange or reloading of peptide after loss of an initial binding peptide.

FIG. 23 is a graphical representation of peptide exchange with a HLA according to some embodiments of the present disclosure. Peptide exchange reaction steps show that both the in-place peptide as well as the exchange catalyst contribute to the overall reaction kinetics.

FIGS. 24A and 24B show the P9 and P8 residue preferences for binding peptides for HLA-A0101, and demonstrate a high preference for Tyr at P9 in this group, while distribution of amino acid type found at P8 for these binding peptides show a more varied distribution.

FIGS. 25A-25J show the P9 residue preferences for binding peptide for a variety of HLA alleles. It is shown that each HLA allele has a different pattern of amino acid preferences at P9.

FIG. 26 is a graphical representation of stability (y-axis) vs affinity (x-axis) of a set of peptide sequences assessed as effective in-place peptides according to some embodiments of the present disclosure.

FIGS. 27A and 27B are graphical representations relating the predicted affinity (x-axis) of the peptides shown in Table 5 for HLA*A0201 against the predicted stability (y-axis) of the peptide within the complex. FIG. 27A plots the y-axis as a log, while FIG. 27B plots the y-axis in an arithmetic relationship.

FIG. 28 shows the same graphical representation as FIG. 27A, while distinguishing the 10 candidate peptides of Table 5 for HLA*A0201 (see also Table 6), selected for testing in the methods described herein, shown as solid black data points while the remainder of the peptides of Table 5, are shown in gray data points. As can be seen, the ten candidate peptides are generally not the peptides with the highest affinity and highest stability. The oval encloses the region within the graph where suitable in-place peptides may be found.

FIG. 29 shows the ten peptides selected for testing as in-place peptides for HLA*A0201. The first peptide in the list (LMYAKRAFV (SEQ ID NO: 4)) has higher affinity/stability and is selected for use as the displacing peptide in an embodiment of the methods. As noted, GMGQKDSYV (SEQ ID NO: 1) and GAATKMAAV (SEQ ID NO: 13) were found to have affinity and exchangeability lending each of them to be the most suitable in-place peptides, permitting stoichiometric loading of antigenic peptides and/or candidate antigenic peptides to be most efficiently loaded into the HLA. The remainder of the ten peptides are RMQKEITAL (SEQ ID NO: 2); IMALKQAGL (SEQ ID NO: 11); FLAIKLYVG (SEQ ID NO: 12); QLALKKVEGV (SEQ ID NO: 14); IMALKKQAGL (SEQ ID NO: 15); FLAIKKLYVG (SEQ ID NO: 16); and TEIGKDVIGL (SEQ ID NO: 17).

FIGS. 30A-30C show the results of an embodiment of the methods described herein. FIGS. 30A and 30B show the flow cytometry results showing counts of exchanged, unexchanged, and unstained HLA complexes with GMGQKDSYV (SEQ ID NO: 1) and FLAIKKLYVG (SEQ ID NO: 16), respectively, as the in-place peptide. FIG. 30C shows the exchangeability for three peptides. GMGQKDSYV (SEQ ID NO: 1) demonstrated exchange efficiency of about 1.0, indicating nearly complete exchange, while FLAIKKLYVG (SEQ ID NO: 16) had an exchange efficiency of about 0.65. LMYAKRAFV (SEQ ID NO: 4), having a higher affinity and stability, had an exchange efficiency of about 0.30.

### DETAILED DESCRIPTION OF CERTAIN EMBODIMENTS

This specification describes exemplary embodiments and applications of the disclosure. The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. Also, unless the context dictates otherwise, directions (e.g., above, below, top, bottom, side, up, down, under, over, upper, lower, horizontal, vertical, “x,” “y,” “z,” etc.), if provided, are relative and provided solely byway of example and for ease of illustration and discussion and not byway of limitation. In addition, where reference is made to a list of elements (e.g., elements a, b, c), such reference is intended to include any one of the listed elements by itself, any combination of less than all of the listed elements, and/or a combination of all of the listed elements. The term “or” is used in an inclusive sense, i.e., equivalent to “and/or,” unless the context dictates otherwise. It is noted that, as used in this specification and the appended claims, the singular forms “a,” “an,” and “the,” and any singular use of any word, include plural referents unless expressly and unequivocally limited to one referent. As used herein, the terms “comprise,” “include,” and grammatical variants thereof are intended to be non-limiting, such that recitation of items in a list is not to the exclusion of other like items that can be substituted or added to the listed items. Section divisions in the specification are for ease of review only and do not limit any combination of elements discussed. In case of any contradiction or conflict between material incorporated by reference and the expressly described content provided herein, the expressly described content controls.

Where dimensions of microfluidic features are described as having a width or an area, the dimension typically is described relative to an x-axial and/or y-axial dimension, both of which lie within a plane that is parallel to the substrate and/or cover of the microfluidic device. The height of a microfluidic feature may be described relative to a z-axial direction, which is perpendicular to a plane that is parallel to the substrate and/or cover of the microfluidic device. In some instances, a cross sectional area of a microfluidic feature, such as a channel or a passageway, may be in reference to a x-axial/z-axial, a y-axial/z-axial, or an x-axial/y-axial area.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques.

### Definitions

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one. As used herein, the term “plurality” can be 2, 3, 4, 5, 6, 7, 8, 9, 10, or more.

As used herein, “alkyl” refers to a straight or branched hydrocarbon chain radical consisting solely of carbon and hydrogen atoms, containing no unsaturation, having from one to six carbon atoms (e.g., C₁-C₆ alkyl). Whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated. In some embodiments, it is a C₁-C₃ alkyl group. Typical alkyl groups include, but are in no way limited to, methyl, ethyl, propyl, isopropyl, n-butyl, iso-butyl, sec-butyl isobutyl, tertiary butyl, pentyl, isopentyl, neopentyl, hexyl, and the like. The alkyl is attached to the rest of the molecule by a single bond, for example, methyl (Me), ethyl (Et), n-propyl, 1-methylethyl (iso-propyl), n-butyl, n-pentyl, 1,1-dimethylethyl (t-butyl), hexyl, and the like.

Unless stated otherwise specifically in the specification, an alkyl group may be optionally substituted by one or more substituents which independently are: aryl, arylalkyl, heteroaryl, heteroarylalkyl, hydroxy, halo, cyano, trifluoromethyl, trifluoromethoxy, nitro, trimethylsilanyl, —OR′, —SR′, —OC(O)—R′, —N(R′)₂, —C(O)R′, —C(O)OR′, —OC(O)N(R′)₂, —C(O)N(R′)₂, —N(R′)C(O)OR′, —N(R′)C(O)R′, —N(R′)C(O)N(R′)₂, N(R′)C(NR′)N(R′)₂, —N(R′)S(O)ₜR′ (where t is 1 or 2), —S(O)ₜOR′ (where t is 1 or 2), —S(O)ₜN(R′)₂ (where t is 1 or 2), or PO₃(R′)₂ where each R′ is independently hydrogen, alkyl, fluoroalkyl, aryl, aralkyl, heterocycloalkyl, or heteroaryl.

As referred to herein, a fluorinated alkyl moiety is an alkyl moiety having one or more hydrogens of the alkyl moiety replaced by a fluoro substituent. A perfluorinated alkyl moiety has all hydrogens attached to the alkyl moiety replaced by fluoro substituents.

As referred to herein, a “halo” moiety is a bromo, chloro, or fluoro moiety.

As referred to herein, an “olefinic” compound is an organic molecule which contains an “alkene” moiety. An alkene moiety refers to a group consisting of at least two carbon atoms and at least one carbon-carbon double bond. The non-alkene portion of the molecule may be any class of organic molecule, and in some embodiments, may include alkyl or fluorinated (including but not limited to perfluorinated) alkyl moieties, any of which may be further substituted.

As used herein, “air” refers to the composition of gases predominating in the atmosphere of the earth. The four most plentiful gases are nitrogen (typically present at a concentration of about 78% by volume, e.g., in a range from about 70-80%), oxygen (typically present at about 20.95% by volume at sea level, e.g. in a range from about 10% to about 25%), argon (typically present at about 1.0% by volume, e.g. in a range from about 0.1% to about 3%), and carbon dioxide (typically present at about 0.04%, e.g., in a range from about 0.01% to about 0.07%). Air may have other trace gases such as methane, nitrous oxide or ozone, trace pollutants and organic materials such as pollen, diesel particulates and the like. Air may include water vapor (typically present at about 0.25%, or may be present in a range from about 10 ppm to about 5% by volume). Air may be provided for use in culturing experiments as a filtered, controlled composition and may be conditioned as described herein.

As used herein, the term “plurality” can be 2, 3, 4, 5, 6, 7, 8, 9, 10, or more.

As used herein, the term “disposed” encompasses within its meaning “located.”

As used herein, a “microfluidic device” or “microfluidic apparatus” is a device that includes one or more discrete microfluidic circuits configured to hold a fluid, each microfluidic circuit comprised of fluidically interconnected circuit elements, including but not limited to region(s), flow path(s), channel(s), chamber(s), and/or pen(s), and at least one port configured to allow the fluid (and, optionally, micro-objects suspended in the fluid) to flow into and/or out of the microfluidic device. Typically, a microfluidic circuit of a microfluidic device will include a flow region, which may include a microfluidic channel, and at least one chamber, and will hold a volume of fluid of less than about 1 mL, e.g., less than about 750, 500, 250, 200, 150, 100, 75, 50, 25, 20, 15, 10, 9, 8, 7, 6, 5, 4, 3, or 2 μL. In certain embodiments, the microfluidic circuit holds about 1-2, 1-3, 1-4, 1-5, 2-5, 2-8, 2-10, 2-12, 2-15, 2-20, 5-20, 5-30, 5-40, 5-50, 10-50, 10-75, 10-100, 20-100, 20-150, 20-200, 50-200, 50-250, or 50-300 μL. The microfluidic circuit may be configured to have a first end fluidically connected with a first port (e.g., an inlet) in the microfluidic device and a second end fluidically connected with a second port (e.g., an outlet) in the microfluidic device.

As used herein, a “nanofluidic device” or “nanofluidic apparatus” is a type of microfluidic device having a microfluidic circuit that contains at least one circuit element configured to hold a volume of fluid of less than about 1 μL, e.g., less than about 750, 500, 250, 200, 150, 100, 75, 50, 25, 20, 15, 10, 9, 8, 7, 6, 5, 4, 3, 2, 1 nL or less. A nanofluidic device may comprise a plurality of circuit elements (e.g., at least 2, 3, 4, 5, 6, 7, 8, 9, 10, 15, 20, 25, 50, 75, 100, 150, 200, 250, 300, 400, 500, 600, 700, 800, 900, 1000, 1500, 2000, 2500, 3000, 3500, 4000, 4500, 5000, 6000, 7000, 8000, 9000, 10,000, or more). In certain embodiments, one or more (e.g., all) of the at least one circuit elements is configured to hold a volume of fluid of about 100 pL to 1 nL, 100 pL to 2 nL, 100 pL to 5 nL, 250 pL to 2 nL, 250 pL to 5 nL, 250 pL to 10 nL, 500 pL to 5 nL, 500 pL to 10 nL, 500 pL to 15 nL, 750 pL to 10 nL, 750 pL to 15 nL, 750 pL to 20 nL, 1 to 10 nL, 1 to 15 nL, 1 to 20 nL, 1 to 25 nL, or 1 to 50 nL. In other embodiments, one or more (e.g., all) of the at least one circuit elements are configured to hold a volume of fluid of about 20 nL to 200 nL, 100 to 200 nL, 100 to 300 nL, 100 to 400 nL, 100 to 500 nL, 200 to 300 nL, 200 to 400 nL, 200 to 500 nL, 200 to 600 nL, 200 to 700 nL, 250 to 400 nL, 250 to 500 nL, 250 to 600 nL, or 250 to 750 nL.

A microfluidic device or a nanofluidic device may be referred to herein as a “microfluidic chip” or a “chip”; or “nanofluidic chip” or “chip”.

A “microfluidic channel” or “flow channel” as used herein refers to flow region of a microfluidic device having a length that is significantly longer than both the horizontal and vertical dimensions. For example, the flow channel can be at least 5 times the length of either the horizontal or vertical dimension, e.g., at least 10 times the length, at least 25 times the length, at least 100 times the length, at least 200 times the length, at least 500 times the length, at least 1,000 times the length, at least 5,000 times the length, or longer. In some embodiments, the length of a flow channel is about 100,000 microns to about 500,000 microns, including any value therebetween. In some embodiments, the horizontal dimension is about 100 microns to about 1000 microns (e.g., about 150 to about 500 microns) and the vertical dimension is about 25 microns to about 200 microns, (e.g., from about 40 to about 150 microns). It is noted that a flow channel may have a variety of different spatial configurations in a microfluidic device, and thus is not restricted to a perfectly linear element. For example, a flow channel may be, or include one or more sections having, the following configurations: curve, bend, spiral, incline, decline, fork (e.g., multiple different flow paths), and any combination thereof. In addition, a flow channel may have different cross-sectional areas along its path, widening and constricting to provide a desired fluid flow therein. The flow channel may include valves, and the valves may be of any type known in the art of microfluidics. Examples of microfluidic channels that include valves are disclosed in U.S. Pat. Nos. 6,408,878 and 9,227,200, each of which is herein incorporated by reference in its entirety.

As used herein, the term “obstruction” refers generally to a bump or similar type of structure that is sufficiently large so as to partially (but not completely) impede movement of target micro-objects between two different regions or circuit elements in a microfluidic device. The two different regions/circuit elements can be, for example, a microfluidic sequestration pen and a microfluidic channel, or a connection region and an isolation region of a microfluidic sequestration pen.

As used herein, the term “constriction” refers generally to a narrowing of a width of a circuit element (or an interface between two circuit elements) in a microfluidic device. The constriction can be located, for example, at the interface between a microfluidic sequestration pen and a microfluidic channel, or at the interface between an isolation region and a connection region of a microfluidic sequestration pen.

As used herein, the term “transparent” refers to a material which allows visible light to pass through without substantially altering the light as is passes through.

As used herein, the term “micro-object” refers generally to any microscopic object that may be isolated and/or manipulated in accordance with the present disclosure. Non-limiting examples of micro-objects include: inanimate micro-objects such as microparticles; microbeads (e.g., polystyrene beads, Luminex™ beads, or the like); magnetic beads; microrods; microwires; quantum dots, and the like; biological micro-objects such as cells; biological organelles; vesicles, or complexes; synthetic vesicles; liposomes (e.g., synthetic or derived from membrane preparations); lipid nanorafts, and the like; or a combination of inanimate micro-objects and biological micro-objects (e.g., microbeads attached to cells, liposome-coated micro-beads, liposome-coated magnetic beads, or the like). Beads may include moieties/molecules covalently or non-covalently attached, such as fluorescent labels, proteins, carbohydrates, antigens, small molecule signaling moieties, or other chemical/biological species capable of use in an assay. Lipid nanorafts have been described, for example, in Ritchie et al. (2009) “Reconstitution of Membrane Proteins in Phospholipid Bilayer Nanodiscs,” Methods Enzymol., 464:211-231.

As used herein, the term “cell” is used interchangeably with the term “biological cell.” Non-limiting examples of biological cells include eukaryotic cells, plant cells, animal cells, such as mammalian cells, reptilian cells, avian cells, fish cells, or the like, prokaryotic cells, bacterial cells, fungal cells, protozoan cells, or the like, cells dissociated from a tissue, such as muscle, cartilage, fat, skin, liver, lung, neural tissue, and the like, immunological cells, such as T cells, B cells, natural killer cells, macrophages, and the like, embryos (e.g., zygotes), oocytes, ova, sperm cells, hybridomas, cultured cells, cells from a cell line, cancer cells, infected cells, transfected and/or transformed cells, reporter cells, and the like. A mammalian cell can be, for example, from a human, a mouse, a rat, a horse, a goat, a sheep, a cow, a primate, or the like.

A colony of biological cells is “clonal” if all of the living cells in the colony that are capable of reproducing are daughter cells derived from a single parent cell. In certain embodiments, all the daughter cells in a clonal colony are derived from the single parent cell by no more than 10 divisions. In other embodiments, all the daughter cells in a clonal colony are derived from the single parent cell by no more than 14 divisions. In other embodiments, all the daughter cells in a clonal colony are derived from the single parent cell by no more than 17 divisions. In other embodiments, all the daughter cells in a clonal colony are derived from the single parent cell by no more than 20 divisions. The term “clonal cells” refers to cells of the same clonal colony.

As used herein, a “colony” of biological cells refers to 2 or more cells (e.g. about 2 to about 20, about 4 to about 40, about 6 to about 60, about 8 to about 80, about 10 to about 100, about 20 to about 200, about 40 to about 400, about 60 to about 600, about 80 to about 800, about 100 to about 1000, or greater than 1000 cells).

As used herein, the term “maintaining (a) cell(s)” refers to providing an environment comprising both fluidic and gaseous components and, optionally a surface, that provides the conditions necessary to keep the cells viable and/or expanding.

As used herein, the term “expanding” when referring to cells, refers to increasing in cell number.

As referred to herein, “gas permeable” means that the material or structure is permeable to at least one of oxygen, carbon dioxide, or nitrogen. In some embodiments, the gas permeable material or structure is permeable to more than one of oxygen, carbon dioxide and nitrogen and may further be permeable to all three of these gases.

A “component” of a fluidic medium is any chemical or biochemical molecule present in the medium, including solvent molecules, ions, small molecules, antibiotics, nucleotides and nucleosides, nucleic acids, amino acids, peptides, proteins, sugars, carbohydrates, lipids, fatty acids, cholesterol, metabolites, or the like.

As used herein in reference to a fluidic medium, “diffuse” and “diffusion” refer to thermodynamic movement of a component of the fluidic medium down a concentration gradient.

The phrase “flow of a medium” means bulk movement of a fluidic medium primarily due to any mechanism other than diffusion. For example, flow of a medium can involve movement of the fluidic medium from one point to another point due to a pressure differential between the points. Such flow can include a continuous, pulsed, periodic, random, intermittent, or reciprocating flow of the liquid, or any combination thereof. When one fluidic medium flows into another fluidic medium, turbulence and mixing of the media can result.

The phrase “substantially no flow” refers to a rate of flow of a fluidic medium that, when averaged over time, is less than the rate of diffusion of components of a material (e.g., an analyte of interest) into or within the fluidic medium. The ratio of a rate of flow of a component in a fluidic medium (i.e., advection) divided by the rate of diffusion of such component can be expressed by a dimensionless Peclet number. Thus, a region within a microfluidic device that experiences substantially no flow in one in which the Peclet number is less than 1. The Peclet number associated with a particular region within the microfluidic device can vary with the component or components of the fluidic medium being considered (e.g., the analyte of interest), as the rate of diffusion of a component or components in a fluidic medium can depend on, for example, temperature, the size, mass, and/or shape of the component(s), and the strength of interactions between the component(s) and the fluidic medium. In certain embodiments, the Peclet number associated with a particular region of the microfluidic device and a component located therein can be 0.95 or less, 0.9 or less, 0.85 or less, 0.8 or less, 0.75 or less, 0.7 or less, 0.65 or less, 0.6 or less, 0.55 or less, 0.5 or less, 0.4 or less, 0.3 or less, 0.2 or less, 0.1 or less, 0.05 or less, 0.01 or less, 0.005 or less, or 0.001 or less.

As used herein in reference to different regions within a microfluidic device, the phrase “fluidically connected” means that, when the different regions are substantially filled with fluid, such as fluidic media, the fluid in each of the regions is connected so as to form a single body of fluid. This does not mean that the fluids (or fluidic media) in the different regions are necessarily identical in composition. Rather, the fluids in different fluidically connected regions of a microfluidic device can have different compositions (e.g., different concentrations of solutes, such as proteins, carbohydrates, ions, or other molecules) which are in flux as solutes move down their respective concentration gradients and/or fluids flow through the device.

As used herein, a “flow path” refers to one or more fluidically connected circuit elements (e.g., channel(s), region(s), chamber(s) and the like) that define, and are subject to, the trajectory of a flow of medium. A flow path is thus an example of a swept region of a microfluidic device. Other circuit elements (e.g., unswept regions) may be fluidically connected with the circuit elements that comprise the flow path without being subject to the flow of medium in the flow path.

As used herein, “isolating a micro-object” confines a micro-object to a defined area within the microfluidic device.

A microfluidic (or nanofluidic) device can comprise “swept” regions and “unswept” regions. As used herein, a “swept” region is comprised of one or more fluidically interconnected circuit elements of a microfluidic circuit, each of which experiences a flow of medium when fluid is flowing through the microfluidic circuit. The circuit elements of a swept region can include, for example, regions, channels, and all or parts of chambers. As used herein, an “unswept” region is comprised of one or more fluidically interconnected circuit element of a microfluidic circuit, each of which experiences substantially no flux of fluid when fluid is flowing through the microfluidic circuit. An unswept region can be fluidically connected to a swept region, provided the fluidic connections are structured to enable diffusion but substantially no flow of media between the swept region and the unswept region. The microfluidic device can thus be structured to substantially isolate an unswept region from a flow of medium in a swept region, while enabling substantially only diffusive fluidic communication between the swept region and the unswept region. For example, a flow channel of a micro-fluidic device is an example of a swept region while an isolation region (described in further detail below) of a microfluidic device is an example of an unswept region.

As used herein, a “non-sweeping” rate of fluidic medium flow means a rate of flow in a flow region, such as a microfluidic channel, which is sufficient to permit components of a second fluidic medium in an isolation region of the sequestration pen to diffuse into the first fluidic medium in the flow region and/or components of the first fluidic medium to diffuse into the second fluidic medium in the isolation region; and further wherein the first medium does not substantially flow into the isolation region.

As used herein, “synthetic surface” refers to an interface between a support structure and a gaseous/liquid medium, where the synthetic surface is prepared by non-biological processes. The synthetic surface may have biologically derived materials connected to it, e.g., primary and co-activating molecules as described herein, to provide an antigen-presenting synthetic surface, provided that the synthetic surface is not expressed by a biological organism. Typically, the support structure is solid, such as the non-surface exposed portions of a bead, a wafer, or a substrate, cover or circuit material of a microfluidic device and does not enclose a biological nucleus or organelle.

As used herein, “co-activating” refers to a binding interaction between a biological macromolecule, fragment thereof, or synthetic or modified version thereof and a T cell, other than the primary T cell receptor/antigen:MHC binding interaction, that enhances a productive immune response to produce activation of the T cell. Co-activating interactions are antigen-nonspecific interactions, e.g., between a T-cell surface protein able to engage in intracellular signaling such as CD28, CD2, ICOS, etc., and an agonist thereof. “Co-activation” and “co-activating” as used herein is equivalent to the terms “co-stimulation” and “co-stimulating”, respectively.

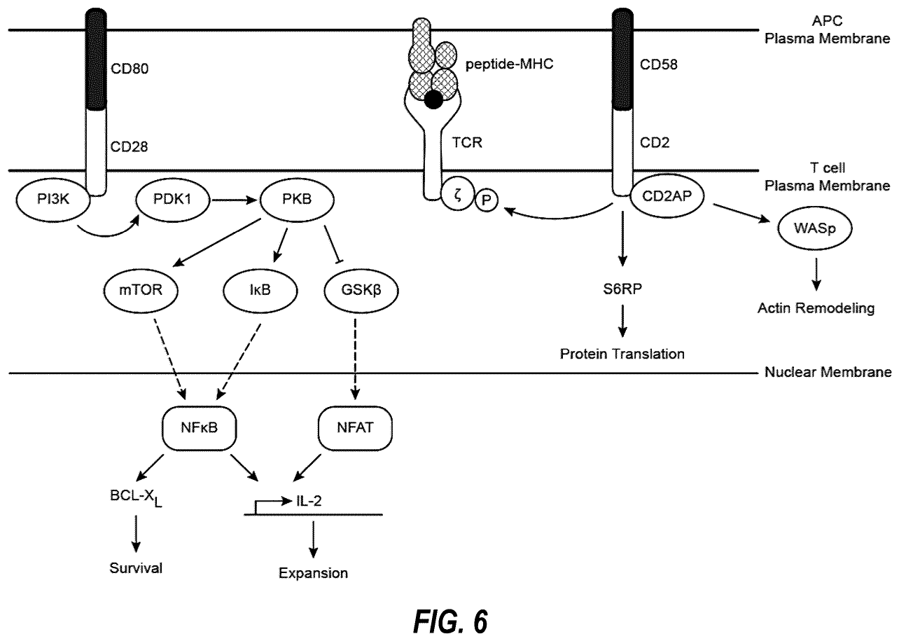

As used herein, a “TCR co-activating molecule” is a biological macromolecule, fragment thereof, or synthetic or modified version thereof that binds to one or more co-receptors on a T Cell that activate distal signaling molecules which amplify and/or complete the response instigated by antigen specific binding of the TCR. In one example, signaling molecules such as transcription factors Nuclear Factor kappa B (NF kB) and Nuclear factor of activated T cells (NFAT) are activated by the TCR co-activating molecule. The TCR co-activating molecule can be, for example, an agonist of the CD28 receptor, which signals through the phosphoinositide 3 kinase (PI3K)/Akt pathway. See FIG. 6.

As used herein, “CD28high” refers to a phenotype of high CD28 surface expression in a T cell. Those skilled in the art are familiar with the CD28high phenotype and appropriate ways of identifying CD28high T cells. Unless otherwise indicated, CD28high T cells include T cells that meet any of the following criteria. In some embodiments, a CD28high T cell is a T cell that expresses higher levels of CD28 than a resting CD8+ T cell. A CD28high T cell may also express higher levels of CD28 than an irrelevant non-antigen specific T cell. In some embodiments, CD28high T cells are a population in which the level of surface CD28 which can be measured by FACS is equal to or greater than the level of surface CD28 present on circulating memory T cells which can be measured by FACS. In some embodiments, a CD28high T cell has a level of surface CD28 equal to or greater than the level of surface CD28 present on circulating memory T cells from the same sample or individual. Expression of surface CD28 can be determined by FACS and the mean (e.g., geometric mean) or median level of surface CD28 present on circulating memory T cells can be used for determining whether a given T cell is CD28high. In some embodiments, a CD28high T cell is a T cell that expresses CD28 at a significantly higher level than expression typical of naïve CD8 T cells from the same sample or individual, e.g., higher than 75%, 80%, 85%, 87.5%, 90%, 92.5%, or 95% of the naïve T cells. Naïve CD8 T cells can be identified and characterized by known methods, e.g., flow cytometrically, as CD8+ cells expressing detectable CD28 and minimal or no CD45RO.

As used herein, a “TCR adjunct activating molecule” stimulates classes of signaling molecules which amplify the antigen-specific TCR interaction and are distinct from the TCR co-activating molecules. For example, TCR proximal signaling by phosphorylation of the TCR proximal signaling complex is one route by which TCR adjunct activating molecules can act. The TCR adjunct activating molecule may be, for example, an agonist of the CD2 receptor. See FIG. 6.

As used herein, an “activated T cell” is a T cell that has experienced antigen (or a descendant thereof) and is capable of mounting an antigen-specific response to that antigen. Activated T cells are generally positive for at least one of CD28, CD45RO, CD127, and CD197.

As used herein, a “biotin functionality” refers to a moiety of a larger molecule or ligand wherein the moiety comprises a covalently bound form of biotin (and may further comprise a linker). In general, a molecule that is biotinylated comprises a biotin functionality.

As used herein, a “molecular ligand” refers to a surface-associated form of a molecule. The surface-association may be a covalent or noncovalent association.

As used herein, an “exchange factor” refers to a compound of the general formula A-B, wherein A comprises one or more amino acid residues and B comprises a C-terminal amino acid residue, wherein the side chain of the C-terminal amino acid residue comprises at least three non-hydrogen atoms (e.g., carbon, nitrogen, oxygen, and/or sulfur). A and B may be but are not necessarily linked by a peptide bond formed between the carboxyl of the first amino acid residue and the amine of the second amino acid residue. The amino acid residues may be but are not necessarily members of the set of 20 canonical naturally occurring amino acids. For example, nonstandard amino acids such as homoleucine, norleucine, cyclohexylalanine, and the like are encompassed. Modified amino acid residues, e.g., wherein the residues comprise an alternative linkage such as a lactam or piperazinone in place of a simple peptide bond are also encompassed, as are peptide-like compounds as described in US2014/0370524. Exchange factors can bind in the antigen-binding pocket of a major histocompatibility complex, e.g. for instance, a F binding pocket of the MHC binding groove when the MHC is an HLA molecule, but have sufficiently low affinity to be displaced by peptide antigens suitable for binding to and presentation by the MHC. When present in excess relative to a peptide antigen already bound to the MHC, exchange factors can displace the already bound peptide and then be displaced in turn by a new peptide antigen, thereby catalyzing a peptide exchange reaction.

As used herein, a “peptide antigen” (also sometimes referred to as an antigenic peptide) refers to a peptide that can bind in the antigen-binding pocket (also known as the antigen-binding groove or peptide-binding groove) of a major histocompatibility complex (MHC). In some embodiments, a peptide antigen is able to contribute to activation of a T lymphocyte, such as a cytotoxic T lymphocyte (e.g., which can be a naïve T cell, a central memory T cell, or the like), when the peptide antigen is bound in the antigen-binding pocket of a major histocompatibility complex (MHC), e.g., a class I MHC. In some embodiments, the peptide antigen is a candidate peptide that may or may not be able to contribute to activation of a T lymphocyte, such as a cytotoxic T lymphocyte, when the peptide antigen is bound in the antigen-binding pocket of a major histocompatibility complex (MHC), e.g., a class I MHC.

As used herein, an “initial peptide” refers to a peptide that can bind to an MHC molecule and then undergo displacement from the MHC molecule in an exchange reaction in the presence of an exchange factor and an incoming peptide antigen. “In-place peptide” is used throughout the specification and claims equivalently with “initial peptide”.

As used herein, a peptide is “non-immunogenic” when it is not capable of generating an adaptive immune response in the in the organism from which it originated, which may be a mammal, such as a human. Non-immunogenic peptides include peptides against which the organism's immune system has been tolerized.

As used herein, a “non-antigen-presenting surface” refers to a surface or region of a larger surface substantially free of primary activating molecular ligands.

### Overview

Immunotherapy for cancer is a promising development, but often requires specifically activated T lymphocytes which are compatible with the subject of the therapy. However, current approaches for activating T lymphocytes present several disadvantageous aspects. These include the need to identify suitable peptide antigens for use in activation using laborious approaches involving generating dendritic cells or otherwise preparing MHCs comprising candidate peptide antigens so that one can evaluate their immunogenicity. Dendritic cells must be obtained from donor sources, limiting throughput. Dendritic cells must be matured for each sequence of T lymphocyte activation, which requires a lead time of about 7 days. Irradiation of dendritic cells is also required, which limits where such processing can be performed. On the other hand, synthetic antigen-presentation approaches have used folding reactions to prepare MHCs comprising candidate peptide antigens, which require considerable time and effort.

Replacing the use of autologous antigen presenting dendritic cells and folding reactions with proto-antigen presenting synthetic surfaces for generating MHCs complexed with peptide antigens for evaluating immunogenicity and activating T lymphocytes may afford more rapid or cost-effective results or may enable greater reliability in stimulating and expanding T lymphocytes for a therapeutically relevant population by facilitating identification of more immunogenic peptide antigens. Proto-antigen presenting synthetic surfaces may be engineered for antigen-specific activation of T lymphocytes upon reaction with a peptide antigen, providing more controllable, characterizable, reproducible and/or more rapid development of populations of activated T lymphocytes having desirable phenotypes for treatment of cancer. Antigen-presenting synthetic surfaces generated from such proto-antigen presenting synthetic surfaces can also allow for more control and selectivity over T cell activation, including more precise targeting of desired T cell phenotypes following activation, e.g., enrichment of particular forms of memory T cells. Furthermore, proto-antigen-presenting synthetic surfaces can also exploit economies of scale and/or provide reproducibility to a greater degree than using autologous antigen presenting dendritic cells or folding reactions to prepare MHCs comprising a peptide antigen. As such, this technology can make cellular therapies available to patients in need thereof in greater numbers and/or in less time. Providing T cells useful for cellular therapies more rapidly can be especially important for patients with advanced disease. The structure of such proto-antigen-presenting synthetic surfaces and their methods of preparation and use are described herein. In some embodiments, the proto-antigen-presenting synthetic surfaces comprise primary activating molecular ligands in combination with TCR co-activating molecules and/or adjunct TCR activating molecules, which serve to activate T cells together with the MHC upon formation of a complex with a peptide antigen. In some embodiments, the proto-antigen-presenting synthetic surfaces and their methods of preparation and use provide one or more of the foregoing advantages, or at least provide the public with a useful choice.

### Proto-Antigen-Presenting Synthetic Surfaces.

A proto-antigen-presenting synthetic surface provided herein for activating a T lymphocyte (T cell) comprises a plurality of primary activating molecular ligands, wherein each primary activating molecular ligand includes a major histocompatibility complex (MHC) molecule configured to bind to a T cell receptor (TCR) of a T cell and wherein an exchange factor or an initial peptide is bound to the MHC molecules. The initial peptide can be a peptide sequence configured to stabilize the MHC molecule and can be identified by any of the methods of identifying an initial peptide sequence for stabilizing an MHC molecule described herein. In some embodiments, the proto-antigen-presenting synthetic surface further comprises an exchange factor separately from the primary activating molecular ligands. The exchange factor or initial peptide may have any of the features described herein for exchange factors or initial peptides, respectively. In some embodiments, the exchange factor or initial peptide is bound in the antigen-binding groove of the MHC. In some embodiments, the MHC molecule is an MHC Class I molecule. In some other embodiments, the MHC molecule is an MHC Class II molecule.

Primary activating molecular ligand (or Primary activating molecule). As used herein, the term “primary activating molecule” is interchangeably used with the term “primary activating molecular ligand,” with the distinction that when referring to a primary activating moiety that is not coupled to a surface, the term “primary activating molecule” is typically used, while when the primary activating moiety is coupled to a surface, covalently or non-covalently, directly or indirectly, the term “primary activating molecular ligand” is typically used. In various embodiments, the proto-antigen-presenting synthetic surface is configured to generate an antigen-presenting synthetic surface that can activate a T lymphocyte in vitro. The primary activating molecular ligand may comprise a MHC molecule having an amino acid sequence and may be connected covalently to the proto-antigen-presenting synthetic surface via a C-terminal connection. The MHC molecule may present a N-terminal portion of the MHC molecule oriented away from the surface, thereby facilitating specific binding of the MHC molecule with the TCR of a T lymphocyte disposed upon the surface. The MHC molecule may include a MHC peptide. Clusters of at least four of the MHC molecules may be disposed at locations upon the proto-antigen-presenting synthetic surface such that when the surface is exposed to an aqueous environment, an MHC tetramer may be formed.

In some embodiments, each of the plurality of primary activating molecular ligands may be covalently connected to the antigen presenting synthetic surface via a linker. In some embodiments, an MHC molecule of a primary activating molecular ligand may be connected to the proto-antigen-presenting synthetic surface through a covalent linkage. Covalent linkages can be formed, for example, using Click chemistry and an appropriate Click reagent pair. Likewise, other ligands described herein, such as co-activating molecular ligands (comprising TCR co-activating molecules and/or adjunct TCR activating molecules), growth stimulatory molecular ligands, and additional stimulatory molecular ligands may be covalently connected to the surface of the antigen presenting synthetic surface via a linker, and the linkage can be formed using Click chemistry and an appropriate Click reagent pair.

In other embodiments, the MHC molecule may be connected to the antigen presenting synthetic surface noncovalently through a coupling group (CG), such as a biotin/streptavidin binding pair interaction. Further examples of coupling groups include, but are not limited to biotin/avidin, biotin/NeutrAvidin, and digoxygenin/anti-digoxygenin. Streptavidin, avidin, and NeutrAvidin represent examples of biotin-binding agents. Further, since some binding pair members such as streptavidin have multiple binding sites (e.g., four in streptavidin), a primary activating molecular ligand may be coupled to the antigen presenting synthetic surface by a biotin/streptavidin/biotin linkage. Again, this may also be true for CG binding pair members involved in associating other ligands described herein (such as co-activating molecular ligands, TCR co-activating molecules, adjunct TCR activating molecules, growth stimulatory molecules, and additional stimulatory molecules) with the surface.

In some embodiments, one member (e.g., streptavidin) of the CG binding pair may itself be covalently bound to the surface, e.g., through one or more linkers. The covalent linkage to the surface can be through a series of about 15, 20, 25, 30, 35, 40, 45, 50, 55, 60, 65, 70, 75, 80, 85, 95, 100, 200 bond lengths, or any number of bond lengths therebetween. In some embodiments, the member of the CG binding pair covalently bound to the surface is bound through a Click reagent pair. This may also be true for CG binding pair members involved in associating other ligands described herein (such as co-activating molecular ligands, TCR co-activating molecules, adjunct TCR activating molecules, growth stimulatory molecules, and additional stimulatory molecules) with the surface.

In some embodiments, a first member of the CG binding pair is covalently associated with the primary activating molecular ligand and a second member of the CG binding pair is non-covalently associated with the surface. For example, the first member of the CG binding pair can be a biotin covalently associated with the primary activating molecular ligand; and the second member of the CG binding pair can be a streptavidin non-covalently associated with the surface (e.g., through an additional biotin, wherein the additional biotin is covalently associated with the surface forming a biotin/streptavidin/biotin linkage). In some embodiments, the biotin covalently associated with the surface is linked to the surface through a series of about 15, 20, 25, 30, 35, 40, 45, 50, 55, 60, 65, 70, 75, 80, 85, 95, 100, 200 bond lengths, or any number of bond lengths therebetween. For example, the biotin covalently associated with the surface may be linked to the surface through a series of one or more linkers having a total length as described. Again, this may also be true for CG binding pair members involved in associating other ligands described herein (such as co-activating molecular ligands, TCR co-activating molecules, adjunct TCR activating molecules, growth stimulatory molecules, and additional stimulatory molecules) with the surface. Noncovalently associating the second member of the CG binding pair, such as streptavidin, with the surface may facilitate loading ligands such as primary activating molecular ligands, co-activating molecular ligands, TCR co-activating molecules, and adjunct TCR activating molecules at greater densities than if the second member of the CG binding pair is covalently associated with the surface.

In some embodiments, the primary activating molecular ligand (e.g., comprising a MHC molecule) can include an exchange factor as described herein, e.g., in the section concerning exchange factors. Any suitable exchange factor may be used.

Other ligands described herein, such as co-activating molecular ligands (comprising TCR co-activating molecules and/or adjunct TCR activating molecules), growth stimulatory molecular ligands, additional stimulatory molecular ligands, and adhesion stimulatory molecular ligands may be noncovalently coupled to the antigen presenting synthetic surface as described therein for the primary activating molecular ligand.

In some embodiments, the density of the plurality of primary activating molecular ligands on the proto-antigen-presenting synthetic surface (or in each portion or sub-region where it is attached) may be about 50, 400, 500, 600, 1×10³, 1.5×10³, 2×10³, 2.5×10³, 5×10³, 7.5×10³, 1×10⁴, 1.25×10⁴, 1.75×10⁴, 2×10⁴, 3×10⁴ molecules per square micron, or any range therebetween. For example, about 50 to about 500 molecules per square micron; about 4×10² to about 2×10³ molecules per square micron; about 1×10³ to about 2×10⁴ molecules per square micron; about 5×10³ to about 3×10⁴ molecules per square micron; about 4×10² to about 3×10⁴ molecules per square micron; about 4×10² to about 2×10³ molecules per square micron; about 2×10³ to about 5×10³ molecules per square micron; about 5×10³ to about 2×10⁴ molecules per square micron; about 1×10⁴ to about 2×10⁴ molecules per square micron; or about 1.25×10⁴ to about 1.75×10⁴ molecules per square micron. In some embodiment, the plurality of primary activating molecular ligands is disposed upon substantially all of the antigen-presenting surface at the stated density.

Co-activating and adjunct activating molecular ligands. As used herein, the term “co-activating molecule” is interchangeably used with the term “co-activating molecular ligand” and the term “adjunct activating molecule” is interchangeably used with the term “adjunct activating molecular ligand,” with the distinction that when referring to an adjunct activating moiety that is not coupled to a surface, the term “adjunct activating molecule” is typically used, while when the adjunct activating moiety is coupled to a surface, covalently or non-covalently, directly or indirectly, the term “adjunct activating molecular ligand” is typically used. In addition to the plurality of primary activating molecular ligand, the antigen presenting synthetic surface can further include a plurality of co-activating molecular ligands, each comprising a TCR co-activating molecule or an adjunct TCR activating molecule. In some embodiments, each of the plurality of primary activating molecular ligands and the plurality of co-activating molecular ligands may be specifically bound to the antigen presenting synthetic surface. In some embodiments, the plurality of co-activating molecular ligands include a plurality of TCR co-activating molecules. In some embodiments, the plurality of co-activating molecular ligands include a plurality of adjunct TCE activating molecules. In other embodiments, the plurality of co-activating molecular ligands may include TCR co-activating molecules and adjunct TCR activating molecules. The TCR co-activating molecules and the adjunct TCR activating molecules can be present in a ratio of one to the other such as about 100:1 to about 1:100 mol:mol (or about 100:1 to about 90:1, about 90:1 to about 80:1, about 80:1 to about 70:1, about 70:1 to about 60:1, about 60:1 to about 50:1, about 50:1 to about 40:1, about 40:1 to about 30:1, about 30:1 to about 20:1, about 20:1 to about 10:1, about 10:1 to about 1:1, about 1:1 to about 1:10, about 1:10 to about 1:20, about 1:20 to about 1:30, about 1:30 to about 1:40, about 1:40 to about 1:50, about 1:50 to about 1:60, about 1:60 to about 1:70, about 1:70 to about 1:80, about 1:80 to about 1:90, or about 1:90 to about 1:100; or from about 3:1 to about 1:3; or from about 1:2 to about 2:1; or about 1:1, or any ratio selected to be between these values). In some embodiments, the plurality of co-activating molecular ligands may include TCR co-activating molecules and adjunct TCR activating molecules in a ratio ranging from about 20:1 to about 1:20 mol:mol or about 3:1 to about 1:3 mol:mol. In some embodiments, a ratio of the primary activating molecular ligands to the co-activating molecular ligands present on the proto-antigen-presenting surface is about 1:10 to about 2:1 mol:mol, about 1:5 to about 2:1 mol:mol, about 1:2 to about 2:1 mol:mol, about 1:10 to about 1:1 mol:mol, about 1:5 to about 1:1 mol:mol, about 1:1 to about 2:1, or about 1:2 to about 1:1 mol:mol, or any ratio selected to be between these values.

The TCR co-activating molecule may include a protein, e.g., an antibody or a fragment thereof. In some embodiments, the TCR co-activating molecule can activate signaling molecules such as transcription factors Nuclear Factor kappa B (NF kB) and Nuclear factor of activated T cells (NFAT). In some embodiments, the TCR co-activating molecule is an agonist of the CD28 receptor, which signals through the phosphoinositide 3 kinase (PI3K)/Akt pathway. In some embodiments, the TCR co-activating molecule may be a CD28 binding molecule (e.g., including a CD80 molecule) or a fragment thereof which retains binding ability to CD28 at a certain level of specificity. The level of specificity needed for a fragment can be determined or pre-selected to ensure that the fragment of the CD28 binding molecule binds to CD28 with a specificity that is at least 10%, 25%, 40%, 50%, 60%, 75%, 80% or more of the level of specificity that the parent CD28 binding molecule has for CD28. In some embodiments, the TCR co-activating molecule may include an anti-CD28 antibody or a fragment thereof (e.g., a CD28-binding fragment) retaining binding ability to CD28.

The adjunct TCR activating molecule may include a protein, e.g., an antibody or a fragment thereof, configured to provide adhesion stimulation. In some embodiments, the TCR adjunct activating molecule which activates TCR proximal signaling, e.g., by phosphorylation of the TCR proximal signaling complex. In some embodiments, the adjunct TCR activating molecules (or additional co-activating molecular ligands) comprise one or more of a CD2 agonist, a CD27 agonist, or a CD137 agonist. The adjunct TCR activating molecules or additional co-activating molecular ligands may alternatively each be a fragment of an antibody to CD2, CD27, or CD137, or any combination thereof. For example, the adjunct TCR activating molecule may be a CD2 binding protein (e.g., CD58) or a fragment thereof, where the fragment retains binding ability with CD2. The fragment may retain at least 10%, 25%, 40%, 50%, 60%, 75%, 80% or more of the level of activity of the parent CD2 binding protein. Varlilumab (CDX-1127) is an exemplary anti-CD27 antibody. Utomilumab (PF-05082566) is an exemplary anti-CD137 antibody. CD70 or an extracellular portion thereof may also be used as a CD27 agonist. TNFSF9, also known as CD137L, or an extracellular portion thereof may also be used as a CD137 agonist. In some embodiments, there may be more than one kinds of the adjunct TCR activating molecules described herein being coupled to the antigen presenting synthetic surface.

In some embodiments, the TCR co-activating molecule is a CD28-binding protein and the adjunct TCR activating molecule is a CD 2-binding protein. Exemplary pathways that can be activated through the CD28 and CD2 receptors (and additional details) are shown in FIG. 8.

In some embodiments, each of the plurality of co-activating molecular ligands may be covalently connected to the proto-antigen-presenting synthetic surface via a linker similarly to that described above for the primary activating molecular ligand. For example, the TCR co-activating molecule or adjunct TCR activating molecule may further comprise a site-specific C-terminal biotin moiety that interacts with a streptavidin, which may be associated covalently or noncovalently with the surface as described herein. A site-specific C-terminal biotin moiety can be added to a TCR co-activating molecule or adjunct TCR activating molecule using known methods, e.g., using a biotin ligase such as the BirA enzyme. See, e.g., Fairhead et al., Methods Mol Biol 1266:171-184, 2015. Further examples of coupling groups include biotin/avidin, biotin/NeutrAvidin, and digoxygenin/anti-digoxygenin. In some embodiments, one of the CG binding pair may itself be covalently bound to the surface, e.g., through a linker, as described above. See the examples for exemplary TCR co-activating molecules or adjunct TCR activating molecules.

In some embodiments, the density of the plurality of co-activating molecular ligands on the proto-antigen-presenting synthetic surface (or in each portion or sub-region where it is attached) is about 20, 200, 250, 500, 600, 1×10³, 1.5×10³, 2×10³, 5×10³, 7.5×10³, 1×10⁴, 1.25×10⁴, 1.5×10⁴, 1.75×10⁴, 2×10⁴, 3×10⁴, 2×10⁵ molecules per square micron, or any range therebetween. For example, from about 20 to about 2×10⁵ molecules per square micron; from about 20 to about 250 molecules per square micron; from about 2×10² to about 1×10³ molecules per square micron; from about 2×10² to about 2×10³ molecules per square micron; about 5×10² to about 5×10³ molecules per square micron; about 5×10² to about 1×10⁴ molecules per square micron; about 1×10³ to about 1×10⁴ molecules per square micron; about 5×10² to about 2×10⁴ molecules per square micron; about 5×10² to about 1.5×10⁴ molecules per square micron; from about 1×10³ to about 2×10⁴ molecules per square micron; from about 5×10³ to about 1×10⁴ molecules per square micron; about 5×10³ to about 2×10⁴ molecules per square micron, about 5×10³ to about 1.5×10⁴ molecules per square micron, about 1×10⁴ to about 2×10⁴ per square micron, about 1×10⁴ to about 1.5×10⁴ molecules per square micron, from about 1.25×10⁴ to about 1.75×10⁴ molecules per square micron, from about 1.25×10⁴ to about 1.5×10⁴ molecules per square micron; or from about 1.25×10⁴ to about 2×10⁵ molecules per square micron. In some embodiments, the plurality of co-activating molecular ligands is disposed upon substantially all of the antigen-presenting surface at the stated density.

Growth stimulatory molecular ligand (or Growth stimulatory molecule). As used herein, the term “growth stimulatory molecular ligand” is interchangeably used with the term “growth stimulatory molecule”,” with the distinction that when referring to a growth stimulatory moiety that is not coupled to a surface, the term “growth stimulatory molecule” is typically used, while when the growth stimulatory moiety is coupled to a surface, covalently or non-covalently, directly or indirectly, the term “growth stimulatory molecular ligand” is typically used. The proto-antigen-presenting synthetic surface may further include at least one growth stimulatory molecular ligand. The growth stimulatory molecular ligand may be a protein or peptide. The growth stimulatory protein or peptide may be a cytokine or fragment thereof which retains activity of the parent molecule. The fragment may retain at least 10%, 25%, 40%, 50%, 60%, 75%, 80% or more of the level of activity of the parent growth stimulatory protein or peptide, and has a level of activity sufficient to act as a growth stimulatory molecular ligand. The growth stimulatory protein or peptide may be a growth factor receptor ligand. The growth stimulatory molecular ligand may comprise IL-21 or a fragment thereof. In some embodiments, the growth stimulatory molecular ligand may be attached to a surface either covalently or via a biotin/streptavidin binding interaction, where the surface is not the same surface as the proto-antigen-presenting synthetic surface having MHC molecules connected thereto. For example, the surface to which the growth stimulatory molecular ligand is attached can be a second surface of a microfluidic device also comprising a first, proto-antigen-presenting synthetic surface.

In yet other embodiments, there may be additional growth stimulatory molecular ligands, which may be one or more cytokines, or fragments thereof, which retains activity of the parent cytokine. The fragment may retain at least 10%, 25%, 40%, 50%, 60%, 75%, 80% or more of the level of activity of the parent cytokine. In some embodiments, additional stimulatory molecular ligands including, but not limited to IL-2 or IL-7 may be connected to the proto-antigen-presenting synthetic surface or to another surface that is not the proto-antigen-presenting synthetic surface, as discussed above with respect to growth stimulatory molecular ligands.

Adhesion stimulatory molecular ligand (or Adhesion stimulatory molecule). As used herein, the term “adhesion stimulatory molecular ligand” is interchangeably used with the term “adhesion stimulatory molecule,” with the distinction that when referring to an adhesion stimulatory moiety that is not coupled to a surface, the term “adhesion stimulatory molecule” is typically used, while when the adhesion stimulatory moiety is coupled to a surface, covalently or non-covalently, directly or indirectly, the term “adhesion stimulatory molecular ligand” is typically used. In some embodiments, the proto-antigen-presenting synthetic surface comprises an adhesion stimulatory molecular ligand, which is a ligand for a cell adhesion receptor including an ICAM protein sequence or fragment thereof which retains activity of the parent molecule. The fragment may retain at least 10%, 25%, 40%, 50%, 60%, 75%, 80% or more of the level of activity of the parent adhesion receptor, and has a level of activity sufficient to act as an adhesion stimulatory molecular ligand. In some embodiments the adhesion stimulatory molecular ligands may be connected to the proto-antigen-presenting synthetic surface or to another surface that is not the proto-antigen-presenting synthetic surface, as discussed above with respect to growth stimulatory molecular ligands. Exemplary adherent motifs that may be used also include poly-L-lysine, amine and the like, and the tripeptide sequence RGD, which is available as a biotinylated reagent and is easily adaptable to the methods described herein. Other larger biomolecules that may be used include fibronectin, laminin or collagen, amongst others. A surface modification having a structure of Formula XXVI as defined in WO2017/205830, including a polyglutamic acid surface contact moiety, can induce adherent cells to attach and grow viably. Another motif that may assist in providing an adherent site is an Elastin Like Peptide (ELP), which includes a repeat sequence of VPGXG (SEQ ID NO: 9), where X is a variable amino acid which can modulate the effects of the motif.

Surface-blocking molecular ligands (or surface-blocking molecule). As used herein, the term “surface-blocking stimulatory molecular ligand” is interchangeably used with the term “surface-blocking stimulatory molecule,” with the distinction that when referring to a surface-blocking moiety that is not coupled to a surface, the term “surface-blocking molecule” is typically used, while when the surface-blocking moiety is coupled to a surface, covalently or non-covalently, directly or indirectly, the term “surface-blocking molecular ligand” is typically used. In some embodiments, the proto-antigen-presenting surface comprises a plurality of surface-blocking molecular ligands, which may include a linker and a terminal surface-blocking group. In some embodiments, the surface (e.g., the proto-antigen-presenting surface or the covalently functionalized surface) comprises a plurality of binding moieties configured for binding the surface-blocking molecule. The binding moieties configured for binding the surface-blocking molecule may be additional to the binding moieties configured for binding the primary activating molecular ligands. The terminal surface-blocking group may be a hydrophilic moiety, an amphiphilic moiety, a zwitterionic moiety, or a negatively charged moiety. In some embodiments, the terminal blocking group comprises a terminal hydroxyl group. In some embodiments, the terminal blocking group comprises a terminal carboxyl group. In some embodiments, the terminal blocking group comprises a terminal zwitterionic group. The plurality of surface-blocking molecular ligands may have all the same terminal surface-blocking group or may have a mixture of terminal surface-blocking groups. Without being bound by theory, the terminal surface-blocking group as well as a hydrophilic linker of the surface-blocking molecular ligand may interact with water molecules in the aqueous media surrounding the proto-antigen-presenting synthetic surface to create a more hydrophilic surface overall. This enhanced hydrophilic nature may render the contact between the proto-antigen-presenting synthetic surface and a cell more compatible and more similar to natural intercellular interactions and/or cell-extracellular fluidic environment in-vivo.

The linker can include a linear chain of 6 or more atoms (e.g., 7, 8, 9, 10, 11, 12, 13, 14, 15, 20, 25, 30, 35, 40, 45, 50, 75, 100, or more atoms) covalently linked together. In some embodiments, the linker may have a linear structure. The linker can comprise, for example, a polymer. The polymer may include a polymer including alkylene ether moieties. A wide variety of alkylene ether containing polymers may be suitable for use on the surfaces described herein. One class of alkylene ether containing polymers is polyethylene glycol (PEG M_{w}<100,000 Da), which are known in the art to be biocompatible. In some embodiments, a PEG may have an M_{w} of about 88 Da, 100 Da, 132 Da, 176 Da, 200 Da, 220 Da, 264 Da, 308 Da, 352 Da, 396 Da, 440 Da, 500 Da, 600 Da, 700 Da, 800 Da, 900 Da, 1000 Da, 1500 Da, 2000 Da, 5000 Da, 10,000 Da or 20,000 Da, or may have a M_{w} that falls within a range defined by any two of the foregoing values. In some embodiments, the PEG polymer has a polyethylene moiety repeat of about 3, 4, 5, 10, 15, 25 units, or any value therebetween. In some embodiments, the PEG is a carboxyl substituted PEG moiety. In some embodiments, the PEG is a hydroxyl substituted PEG moiety. In some embodiments, the PEG moiety can have a backbone linear chain length of about 10 atoms to about 100 atoms.

In some embodiments, each of the plurality of surface-blocking molecular ligands may have a linker having the same length as the linkers of the other ligands of the plurality. In other embodiments, the linkers of the plurality of surface-blocking molecular ligands may have varied lengths. In some embodiments, the surface-blocking group and the length of the linker may be same for each of the plurality of surface-blocking molecular ligands. Alternatively, the surface blocking group and the length of the linker may vary within the plurality of the surface-blocking molecular ligands and may include 2, 3, or 4 different surface-blocking groups and/or 2, 3, 4, or more different lengths, chosen in any combination. In general, the surface-blocking molecular ligands have a length and/or structure that is sufficiently short so as not to sterically hinder the binding and/or function of the primary activating molecular ligands and the co-activating molecular ligands. For example, in some embodiments, the length of the surface-blocking molecular ligands is equal to or less than the length of the other linkers bound to the surface (e.g., linkers that connect coupling groups, primary activating molecular ligands, co-stimulating molecular ligands, or other ligands). In some embodiments, the length of the surface-blocking molecular ligands is about 1 or more angstroms (e.g., about 2, 3, 4, 5, 10, 15, 20, 25, 30, 35, 40, 45, 50, 75, 100, or more angstroms) less than the length of the other linkers bound to the surface (e.g., linkers that connect coupling groups, primary activating molecular ligands, co-stimulating molecular ligands, or other ligands). In some embodiments, the length of the surface-blocking molecular ligands is about 1 to about 100 angstroms (e.g., about 2 to about 75, about 3 to about 50, about 4 to about 40, or about 5 to about 30 angstroms) less than the length of the other linkers bound to the surface. When the surface-blocking molecular ligands have a length that is the same or somewhat less than the length of the other linkers bound to the surface, the resulting surface effectively presents the ligands attached to the other linkers in a manner that is readily available for coupling and/or interacting with cells. With respect to proto-antigen-presenting beads, including a surface-blocking molecular ligand such as a hydrophilic polymer, e.g., a PEG or PEO polymer and/or ligands comprising terminal hydroxyl or carboxyl groups, may beneficially reduce aggregation of the beads through hydrophobic interactions. The surface-blocking molecular ligands can be attached to the surface after the primary and other (e.g., coactivating, adjunct, etc.) ligands discussed above or may be introduced before any of the activating or co-activating species are attached to the surface, as set forth in any embodiments disclosed herein.

Surface. The proto-antigen-presenting synthetic surface may comprise metal, glass, ceramic, polymer, metal oxide, or a combination thereof. In some embodiments, the proto-antigen-presenting synthetic surface is a surface of a wafer having any kind of configuration, a surface of a bead (can include a magnetic material), at least one inner surface of a fluidic circuit containing device (e.g., microfluidic device) configured to contain a plurality of cells, or an inner surface of a tube (e.g., glass or polymer tube). The proto-antigen-presenting microfluidic device may be any microfluidic device as described herein, and may have any combination of features described herein. In some embodiments, the wafer having a proto-antigen-presenting synthetic surface configured to activate T lymphocytes may be sized to fit within a well of a standard 48, 96 or 384 wellplate. In various embodiments, beads having a proto-antigen-presenting synthetic surface configured to activate T lymphocytes may be disposed for use within a wellplate or within a fluidic circuit containing device.

Surface-area to volume ratios of beads. Without wishing to be bound by any particular theory, certain experiments have indicated that it may be advantageous to provide and use beads for T cell activation that have relatively defined surface-area to volume ratios. Such beads may present the relevant ligands in a more accessible way so that they interact more efficiently with T cells during activation. Such beads may provide a desired degree of T cell activation with fewer ligands needed than beads with higher surface-area to volume ratios and/or may provide a higher degree of T cell activation or more T cells with desired features (e.g., antigen specificity and/or marker phenotypes described herein) than beads with higher surface-area to volume ratios. An ideally spherical solid has the lowest possible surface-area to volume ratio. Accordingly, in some embodiments, the bead surface-area is within 10% of the surface-area of a sphere of an equal size (volume or diameter), and is referred to herein as “substantially spherical.” For example, for a bead with a 2.8 μm diameter (1.4 μm radius), the corresponding ideal sphere would have a surface area of 4πr²=24.63 μm². A substantially spherical 2.8 μm diameter bead with a surface-area within 10% of the surface-area of an ideal sphere of an equal volume or diameter would therefore have a surface-area less than or equal to 27.093 μm² (24.63×110%=27.093). It is noted that certain commercially available beads are reported as having higher surface areas; for example, Dynabeads M-270 Epoxy are described in their product literature as having a specific surface area of 2-5 m²/g and a 2.8 μm diameter, and the literature also indicates that 1 mg of the product comprises 6 to 7×10⁷ beads. Multiplying the specific surface area by 1 mg/6-7×10⁷ beads gives a surface area per bead of 28 to 83 μm² per bead, which is more than 10% greater than the surface-area of an ideal sphere with a 2.8 μm diameter. Polymer beads having a surface area more than 10% greater than the surface-area of an ideal sphere are referred to herein as a “convoluted bead.” In some embodiments, a polymer bead may be either substantially spherical or convoluted. In some embodiments, the polymer bead is not convoluted, but is substantially spherical.

Unpatterned surface. In various embodiments, the proto-antigen-presenting synthetic surface may be a unpatterned surface having a plurality of primary activating molecular ligands distributed evenly thereon. As used herein, a surface having a ligand “distributed evenly” thereon is characterized in that no portion of the surface having a size of 10% the total surface area, or greater, has a statistically significant higher concentration of ligand as compared to the average ligand concentration of the total surface area of the surface. The primary activating molecular ligands can comprise MHC molecules, each of which may include an initial peptide or a tumor associated antigen. The unpatterned surface may further include a plurality of co-activating molecular ligands (e.g., TCR co-activating molecules and/or adjunct TCR activating molecules) distributed evenly thereon. The co-activating molecular ligands may be as described above for proto-antigen-presenting surfaces, in any combination. The density of the primary activating molecular ligands and the co-activating molecular ligands may the same ranges as described above for proto-antigen-presenting surfaces. The unpatterned proto-antigen-presenting synthetic surface may further include additional growth stimulatory, adhesive, and/or surface-blocking molecular ligands, as described above for proto-antigen-presenting surfaces, each of which (if present) can be evenly distributed on the unpatterned surface. For example, the unpatterned surface can include an adjunct stimulatory molecule such as IL-21 connected to the surface. The primary activating molecular ligands, co-activating molecular ligands, and/or additional ligands may be linked to the surface as described above for the proto-antigen-presenting surfaces.

Patterned surface. In various embodiments, the proto-antigen-presenting synthetic surface may be patterned and may have a plurality of regions, each region including a plurality of the primary activating molecular ligands comprising MHC molecules, where the plurality of regions is separated by a region configured to substantially exclude the primary activating molecular ligands. The proto-antigen-presenting synthetic surface may be a planar surface. In some embodiments, each of the plurality of regions including the at least a plurality of the primary activating molecular ligands may further include a plurality of the co-activating molecular ligands, e.g., a TCR co-activating molecule and/or an adjunct TCR activating molecule. The co-activating molecular ligands may be any of the co-activating molecular ligands as described above and in any combination. The primary activating molecular ligands and/or co-activating molecular ligands may be linked to the surface as described above for the proto-antigen-presenting surfaces. The density of the primary activating molecular ligands and/or the co-activating molecular ligands in each of the regions containing the primary activating molecular ligands and/or the co-activating molecular ligands may be in the same range as the densities described above for proto-antigen-presenting surfaces. In some embodiments, each of the plurality of regions comprising at least the plurality of the primary activating molecular ligands has an area of about 0.10 square microns to about 4.0 square microns. In some embodiments, each of the plurality of regions comprising at least the plurality of the primary activating molecular ligands has an area of about 0.8 square microns to about 4.0 square microns. In other embodiments, the area of each of the plurality of regions may be about 0.20 square microns to about 0.8 square microns. The plurality of regions may be separated from each other by about 2 microns, about 3 microns, about 4 microns, or about 5 microns. The pitch between each region of the plurality and its neighbor may be about 2 microns, about 3 microns, about 4 microns, about 5 microns, or about 6 microns.

In various embodiments, the region configured to substantially exclude the primary activating molecular ligands comprising MHC molecules may also be configured to substantially exclude TCR co-activating molecules and/or adjunct TCR activating molecules.

In some embodiments, the region configured to substantially exclude the primary activating molecular ligands and optionally the TCR co-activating molecules and/or adjunct TCR activating molecules may be configured to include one or more of surface-blocking molecular ligands, growth stimulatory molecular ligands, additional stimulatory molecular ligands, and adhesion stimulatory molecular ligands. In some embodiments, the growth stimulatory molecular ligands and/or additional stimulatory molecular ligands include a cytokine or fragment thereof, and may further include IL-21 or fragment thereof. In some embodiments, the region configured to substantially exclude the primary activating molecular ligands and optionally the TCR co-activating molecules and/or adjunct TCR activating molecules may further be configured to include one or more supportive moieties. The supportive moieties may provide adhesive motifs to support T lymphocyte growth or may provide hydrophilic moieties providing a generally supportive environment for cell growth. The moiety providing adhesive support may include a peptide sequence including a RGD motif. In other embodiments, the moiety providing adhesive support may be an ICAM sequence. A moiety providing hydrophilicity may be a moiety such as a PEG moiety or carboxylic acid substituted PEG moiety.

Microfluidic device. In some embodiments, a microfluidic device comprises a patterned proto-antigen-presenting synthetic surface having a plurality of regions according to any of the foregoing embodiments. While the proto-antigen-presenting surface of microfluidic device may be any microfluidic (or nanofluidic) device as described herein, the disclosure is not so limited. Other classes of microfluidic devices, including but not limited to microfluidic devices including microwells or microchambers such as described in WO2014/153651 (filed on Mar. 28, 2014), WO2016/115537 (filed on Jan. 15, 2016), or WO2017/124101 (filed on Jan. 17, 2017), may be modified to either incorporate an antigen presenting surface as described in this section, or may be used in combination with the proto-antigen-presenting beads or proto-antigen-presenting wafers as described herein in the methods described in this disclosure.

In some embodiments, the proto-antigen-presenting synthetic surface is an inner surface of a microfluidic device comprising one or more sequestration pens and a channel. At least part of a surface within one or more such sequestration pens may comprise a plurality of primary activating molecular ligands and a plurality of co-activating molecular ligands, e.g., comprising TCR co-activating molecules and/or adjunct TCR activating molecules. The primary activating molecular ligands and the co-activating molecular ligands may be any described above for proto-antigen-presenting surfaces, and may be present in any concentration or combination as described above. The nature of the ligands attachment to the surface of the microfluidic device may be any described above as for proto-antigen-presenting surfaces. In some embodiments, this surface within the one or more such sequestration pens can further comprise one or more of surface-blocking molecular ligands, growth stimulatory molecular ligands, additional stimulatory molecular ligands, and adhesion stimulatory molecular ligands. At least part of a surface of the channel may comprise surface-blocking molecular ligands, e.g., any of the regions configured to substantially exclude the primary activating molecular ligands described herein. In some embodiments, the surface of the channel comprises surface-blocking molecular ligands and optionally other non-stimulatory ligands, but is substantially free of other ligands present on the surface of the sequestration pen, e.g., primary activating molecular ligands and co-activating molecular ligands.

Modulation of cell-to-surface adhesion. In some embodiments, it can be useful to modulate the capacity for cells to adhere to surfaces within the microfluidic device. A surface that has substantially hydrophilic character may not provide anchoring points for cells requiring mechanical stress of adherence to grow and expand appropriately. A surface that presents an excess of such anchoring moieties may prevent successfully growing adherent cells from being exported from within a sequestration pen and out of the microfluidic device. In some embodiments, a covalently bound surface modification comprises surface contact moieties to help anchor adherent cells, e.g. adhesion stimulatory molecular ligands as described above. The structures of the surfaces described herein and the methods of preparing them provide the ability to select the amount of anchoring moieties that may be desirable for a particular use. A very small percentage of adherent type motifs may be needed to provide a sufficiently adhesion enhancing environment. In some embodiments, the adhesion stimulatory molecular ligands may be present at a ratio of about 0.00001:1.0; 0.00005:1.0; 0.0001:1.0; 0.0005:1; 0.001:1.0; 0.005:1.0; 0.01:1.0; 0.05:1.0; or 0.1:1.0 w/w % of the surface-blocking molecular ligands the covalently bound surface modifications. In some embodiments, the adhesion stimulatory molecular ligands are introduced to the surface before cells are introduced to the microfluidic device. Alternatively, an adhesion enhancing modified surface may be provided before introducing cells, and a further addition of another adhesion enhancing moiety may be made, which is designed to attach to the first modified surface either covalently or non-covalently (e.g., as in the base of biotin/streptavidin binding).

In some embodiments, adhesion enhancing surface modifications may modify the surface in a random pattern of individual molecules of a surface modifying ligand. In some other embodiments, a more concentrated pattern of adhesion enhancing surface modifications may be introduced by using polymers containing multiple adhesion enhancing motifs such as positively charged lysine side chains, which can create small regions of surface modification surrounded by the remainder of the surface, which may have hydrophilic surface modifications to modulate the adhesion enhancement. This may be further elaborated by use of dendritic polymers, having multiple adhesion enhancing ligands. A dendritic polymer type surface modifying compound or reagent having multiple adhesion enhancing ligands may be present in a very small proportion relative to surface-blocking molecular ligands, while still providing adhesion enhancement. Further a dendritic polymer type surface modifying compound or reagent may itself have a mixed set of end functionalities which can additionally modulate the behavior of the overall surface.

In some embodiments, it may be desirable to provide regioselective introduction of surfaces. For example, in the context of a microfluidic device comprising a microfluidic channel and sequestration pens, it may be desirable to provide a first type of surface within the microfluidic channel while providing a second type of surface within the sequestration pens opening off of the channel that provides the ability to both culture adherent-type cells successfully as well as easily export them (e.g., using dielectrophoretic or other forces) when desired. In some embodiments, the adhesion enhancing modifications may include cleavable moieties. The cleavable moieties may be cleavable under conditions compatible with the cells being cultured within, such that at any desired timepoint, the cleavable moiety may be cleaved and the nature of the surface may alter to be less enhancing for adhesion. The underlying cleaved surface may be usefully non-fouling such that export is enhanced at that time. While the examples discussed herein focus on modulating adhesion and motility, the use of these regioselectivity modified surfaces are not so limited. Different surface modifications for any kind of benefit for cells being cultured therein may be incorporated into the surface having a first and a second surface modification according to the disclosure.

In some embodiments, in the context of a microfluidic device comprising a microfluidic channel and sequestration pens, a surface of the flow region (e.g., microfluidic channel) may be modified with a first covalently bound surface modification and a surface of the at least one sequestration pen may be modified with a second covalently bound surface modification, wherein the first and the second covalently bound surface modification have different surface contact moieties, different reactive moieties, or a combination thereof. The first and the second covalently bound surface modifications may be selected from any of Formula XXX, Formula V, Formula VII, Formula XXXI, Formula VIII, and/or Formula IX, all of which are as defined in WO2017/205830. When the first and the second covalently bound surface modifications both include functionalized surface of Formula XXX, Formula V, or Formula VII as defined in WO2017/205830, then orthogonal reaction chemistries are selected for the choice of the first reactive moiety and the second reactive moiety. In various embodiments, all the surfaces of the flow region may be modified with the first covalent surface modification and all the surfaces of the at least one sequestration pen may be modified with the second covalent modification.

The proto-antigen-presenting surfaces described herein can be used to prepare an antigen-presenting surface that presents a peptide antigen, e.g., by reacting the peptide antigen with the proto-antigen-presenting surface, wherein the exchange factor or initial peptide is substantially displaced and the peptide antigen becomes associated with the MHC molecules.

### Exchange Factors.

Exchange factors are provided in various kits and surfaces described herein and are used in various methods and uses described herein. The following description is provided with respect to all disclosed embodiments herein involving exchange factors.

An exchange factor is a compound including the general formula A-B, wherein A comprises one or more amino acid residues and B comprises a C-terminal amino acid residue, wherein the side chain of the C-terminal amino acid residue comprises at least three non-hydrogen atoms (e.g., carbon, nitrogen, oxygen, and/or sulfur). In some embodiments, A and B are linked by a peptide bond. In some embodiments, A and B are linked through an alternative linkage, such as a lactam or piperazinone. In some embodiments, the exchange factor is 2, 3, 4, or 5 amino acid residues in length. In some embodiments, one or more amino acid residues of the exchange factor are nonstandard amino acid residues (i.e., different from the 20 canonical amino acid residues that are specified by the standard genetic code). Exemplary nonstandard amino acid residues include norleucine, homoleucine, and cyclohexylalanine (in which a proton of the methyl side chain of alanine is substituted with a cyclohexyl). In some embodiments, the penultimate residue from the C-terminus of the exchange factor (e.g., the N-terminal residue of a dipeptide) has a side chain comprising 0, 1, or 2 non-hydrogen atoms (e.g., G, A, S, or C). The penultimate residue from the C-terminus is the residue immediately adjacent to the C-terminal residue. In some embodiments, the N-terminal residue of the exchange factor (e.g., the N-terminal residue of a dipeptide) has a free N-terminal amine. In some embodiments, the C-terminal residue of the exchange factor is Leu, Phe, Val, Arg, Met, Lys, or Ile. In some embodiments, the C-terminal residue of the exchange factor is homoleucine, norleucine, or cyclohexylalanine. In some embodiments, the penultimate residue from the C-terminus of the exchange factor is Gly, Ala, Ser, or Cys. In some embodiments, the penultimate residue from the C-terminus of the exchange factor is Gly. In some embodiments, the penultimate residue from the C-terminus of the exchange factor is Ala. In some embodiments, the exchange factor is a dipeptide, such as GL, GF, GV, GR, GM, G (homoleucine), G (cyclohexylalanine), G (Norleucine), GK, GI, AL, AF, AV, AR, AM, A (homoleucine), A (cyclohexylalanine), A (Norleucine), AK, or AI. The A or G in any of the foregoing may alternatively be substituted with S or C.

See Saini et al., Proc Nat'l Acad Sci USA (2013) 110, 15383-88, and Saini et al., Proc Nat'l Acad Sci USA (2015) 112, 202-07, for discussion of exemplary exchange factors and their use to displace an initial peptide from an MHC and then undergo displacement by a subsequent peptide.

### Major Histocompatibility Complexes (MHC).

The following description is provided with respect to any embodiment (e.g., surface, kit, use, or method) described herein involving an MHC. In some embodiments, the MHC molecule is an MHC Class I molecule. In some embodiments, the MHC molecule is an MHC Class II molecule.

Many different MHC Class I alleles are known and have been sequenced. MHC Class I sequences for the HLA-A, HLA-B, and HLA-C heavy chains are available, e.g., through the hla.alleles.org website (see hla.alleles.org/data/hla-a.html, hla.alleles.org/data/hla-b.html, and hla.alleles.org/data/hla-c.html for links to HLA nucleotide and amino acid sequences). In some embodiments, the MHC comprises an HLA-A. In some embodiments, the MHC comprises an HLA-B. In some embodiments, the MHC comprises an HLA-C.

In some embodiments, the HLA-A is an HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*43, HLA-A*66, HLA-A*68, HLA-A*69, HLA-A*74, or HLA-A*80.

In some embodiments, the HLA-B is an HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*81, HLA-B*82, or HLA-B*83.

In some embodiments, the HLA-C is an HLA-C*01, HLA-C*02, HLA-C*03, HLA-C*04, HLA-C*05, HLA-C*06, HLA-C*07, HLA-C*08, HLA-C*12, HLA-C*14, HLA-C*15, HLA-C*16, HLA-C*17, or HLA-C*18.

In some embodiments, an initial peptide is bound to an MHC molecule, e.g., in a kit described herein or during or at the beginning of a method described herein (e.g., before an exchange reaction). The initial peptide may be any of the initial peptides described herein.

### Initial Peptides.

An initial peptide is provided in various kits and surfaces described herein and is used in various methods and uses described herein. The following description is provided with respect to all disclosed embodiments herein involving initial peptides.

In some embodiments, the initial peptide comprises at least 4 or 5 amino acid residues. In some embodiments, the initial peptide has a length of about 4, 5, 6, 7, 8, 9, 10, 11, 12, 13, 14, 15, 16, 17, or 18 amino acid residues. In some embodiments, the initial peptide has a length that ranges from 8 to 10 amino acid residues, 13 to 15 amino acid residues, or 13 to 18 amino acid.

In some embodiments, the initial peptide comprises a lysine as the fourth or fifth amino acid residue (counting from the N-terminus, i.e., wherein the N-terminal residue is the first residue). In some embodiments, the initial peptide comprises a label. In some embodiments, the fourth or fifth amino acid residue (e.g., lysine) is labeled. In some embodiments, the label is a fluorescent label. In some embodiments, the label is radioactive. In some embodiments, the label is a chemical moiety (e.g., dinitrophenyl (DNP)) which can be specifically bound by an detection agent (e.g., a labeled antibody) when the initial peptide is bound to MHC. Where an initial peptide is labeled, it can facilitate monitoring of the progress of an exchange reaction to exchange an initial peptide initially bound to an MHC molecule for a peptide antigen in the presence of an exchange factor, wherein the extent of exchange of the initial peptide for the peptide antigen can be determined by detecting the extent to which the label is associated with the MHC molecule.

In some embodiments, the initial peptide comprises a sequence from a naturally occurring (e.g., mammalian or human) polypeptide. In some embodiments, the sequence of the initial peptide consists of sequence from a naturally occurring (e.g., mammalian or human) polypeptide (e.g., a sequence that appears in a wild-type (e.g., mammalian or human) polypeptide). In some embodiments, the initial peptide is non-immunogenic or minimally immunogenic in the organism from which it originated, e.g., in a mammal or in humans. In some embodiments, the initial peptide may or may not be an antigenic peptide, as long as the antigenic peptide has a suitable affinity for MHC molecule, e.g., moderate or reduced affinity relative to another antigenic peptide, such that it is displaceable, as described herein. In some embodiments, the sequence of the initial peptide comprises or consists of sequence from a highly conserved protein (e.g., a protein with a below average mutation rate; in some embodiments the mutation rate is at least one or two standard deviations below the average amino acid mutation rate in the organism). In some embodiments, the sequence of the initial peptide comprises or consists of sequence from a cytoskeletal polypeptide, e.g., an actin or tubulin polypeptide. In some embodiments, the sequence of the initial peptide comprises or consists of sequence from a ribosomal polypeptide, e.g., the RPSA, RPS2, RPL3, RPL4, RPL5, RPL6, RPL7A, or RPP0 polypeptides. Ribosomal and cytoskeletal polypeptides are examples of highly conserved polypeptides, which should be non-immunogenic because of tolerization. It can be beneficial to use such polypeptides because, in the event that a residual amount of the initial polypeptide remains bound to the MHC molecule following an exchange reaction, the MHC molecules comprising the initial polypeptide will not result in stimulation of antigen-specific T cells because T cells specific for tolerized polypeptides generally do not exist.

Exemplary initial peptide sequences are shown in Table 1 below.

TABLE 1

| Protein Source | UniProt ID | Peptide Position | Sequence | SEQ ID NO |
|---|---|---|---|---|
| ACTB | P60709 | 46-54 | GMGQKDSYV | SEQ ID NO: 1 |
| ACTB | P60709 | 312-320 | RMQKEITAL | SEQ ID NO: 2 |
| MYH9 | P35579 | 653-661 | QLAKLMATL | SEQ ID NO: 3 |
| TBA1A | Q71U36 | 397-405 | LMYAKRAFV | SEQ ID NO: 4 |

An initial peptide may be used that binds the MHC molecule with high affinity and/or a low off-rate or long half-life. In some embodiments, the binding of the initial peptide to the MHC molecule has a half-life of at least about 0.1, 0.2, 0.4, 1.5, 2, 4, 6, 8, 10, 12, 14, 16, 18, 20, 24, 28, 32, 36, 48, or 72 hours. In some embodiments, the binding of the initial peptide to the MHC molecule has a half-life in the range of about 4-10, 4-12, 8-16, 10-15, 12-20, 15-20, 20-25, 20-28, 24-32, 25-30, 28-36, 30-35, 32-40, 35-40, 36-48, or 48-72 hours. Half-lives may be determined in the absence of an exchange factor and/or using the conditions described for the stability assay of Example 22. It can be beneficial to the stability of the MHC to be bound to an initial peptide with high affinity and/or a low off-rate or long half-life. For example, a MHC molecule including a beta macroglobulin (e.g., a beta-2-microglobulin) may lose the beta microglobulin subunit if the initial peptide dissociates prior to an exchange reaction, which may adversely impact the function of the MHC molecule. The high affinity and/or a low off-rate or long half-life does not pose a substantial obstacle to the exchange reaction because the exchange reaction can be driven by stoichiometry (i.e., an excess of the exchange factor and peptide antigen). In some embodiments, the affinity of the initial peptide to a binding groove of the MHC includes a predicted Kd from about 1E+1 nM to about 1E+5 nM, from about 1E+1 nM to about 2E+5 nM, from about 1E+2 nM to about 1E+5 nM, from about 1E+2 nM to about 2E+4 nM, from about 1E+2 nM to about 1E+3 nM, from about 1E+2 nm to about 1E+4 nM, from about 1E+1 nm to about 2E+4 nM, from about 1E+2 nM to about 1E+3 nM, from about 1.5E+2 nM to about 1E+5 nM.

### Methods of Identification of an Initial Peptide for MHC Stabilization.

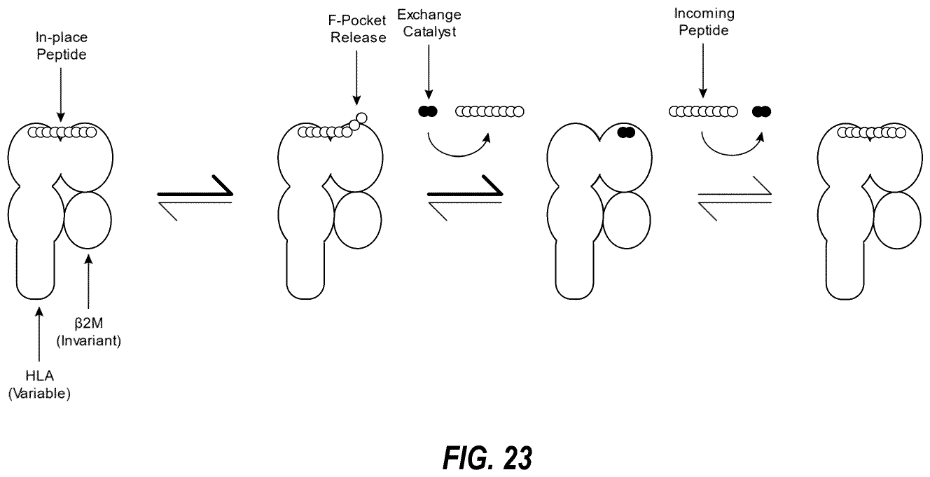

An initial peptide (alternatively, an in-place peptide) may be used for MHC stabilization. The MHC molecule generally does not retain a stable conformation in the absence of a peptide bound into the antigen-binding groove. When an unbound MHC molecule is presented on a surface of an Antigen Presenting Cell (APC), it will not remain on the surface but will be recycled. In designing synthetic antigen presenting surfaces for activation of T cells, an initial peptide needs to be included in a peptide:MHC (e.g., LP:MHC complex or pHLA complex) in order to stably manufacture the ligands for attachment to the surfaces and subsequent use. The subsequent use typically includes an exchange reaction (e.g., in FIG. 23) for displacing the initial peptide with a selected peptide of interest, e.g., an antigenic peptide of interest. The process of displacing the initial peptide has been thought to depend significantly or solely on the use of an exchange factor (or, an exchange catalyst), and not on the nature of the binding of the initial peptide. It has been discovered that, in contrast to this previous conception of the binding/displacement process, the specific affinity and release kinetics of the initial peptide itself also contributes significantly to the overall displacement process.

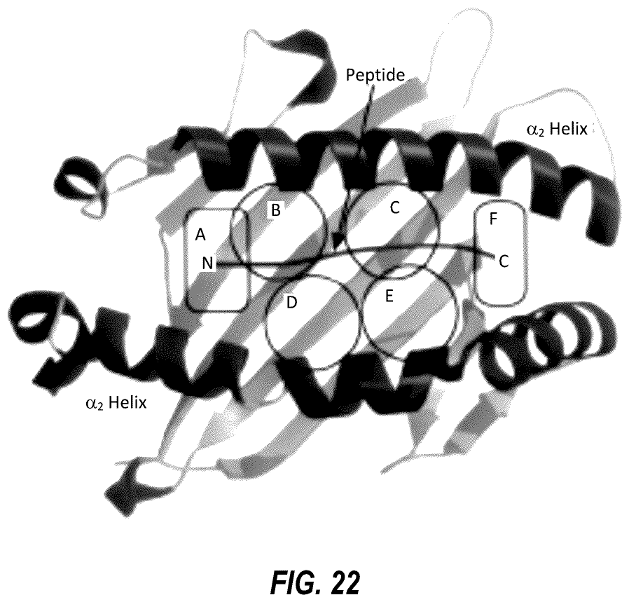

In the examples discussed here (HLA-A*01 and HLA-A*02) but generalizable to other MHC molecules, binding to the binding groove is mediated by a set of six binding pockets, A-F (FIG. 22). Without being bound by theory, if the initial peptide has a high affinity for the binding groove, in particular, binding pocket F of the binding groove, the kinetics of release from the anchoring binding pocket F may limit the ability of the exchange factor to displace the initial peptide, and subsequently permit displacement by the selected peptide of interest. Methods are presented here to design candidate peptide sequences which modulate the anchoring kinetics at binding pocket F (stability of binding) while still binding the binding groove with high enough affinity to permit effective assembly of the pHLA complex (e.g., efficient manufacturability) while also providing the additional kinetic release from the binding pocket to boost efficiency of displacement by the exchange factor. This contribution to the kinetics of the overall exchange process permits the subsequent displacement by the selected peptide of interest to occur more efficiently.

For example, HLA molecules are not structurally stable without peptide binding to the HLA, and if unbound, HLA molecules does not remain at the surface of Antigen-Presenting Cells (APCs). As shown in FIG. 22, Class 1 HLA-peptide interactions are mediated by six binding pockets (A-F) in the binding groove. Pocket A and Pocket F anchor the peptide in the groove. Exogenous peptides can be loaded onto APCs, which suggests a mechanism for direct peptide exchange or reloading of peptide after loss of an initial binding peptide.

The mechanism of peptide exchange reaction in heavy chain (HC)-β2m complexes (FIG. 23) show that both the in-place peptide as well as the exchange catalyst contribute to the overall reaction kinetics. There are certain properties of exchange catalysts that contribute to successful peptide exchange with HLA molecules. The exchange catalyst may bind to the F-Pocket with rapid on-rate and allow for rapid displacement of the in-place peptide once the peptide exchange reaction is started. The exchange catalyst may unbind the F-Pocket with sufficiently high kinetics to allow an incoming peptide of interest to bind. The exchange catalyst facilitates stoichiometric control of loading of incoming peptides with different affinities. However, the exchange catalyst, while necessary, may not be sufficient to allow successful peptide exchange with HLA. The properties of the initial peptide (or in-place peptide) also contribute to successful peptide exchange. The in-place peptide may have both affinity for the HLA of interest, which permits the peptide:HLA complex (pHLA) to be produced (e.g., manufactured) in a reasonable yield, as well as significant F-pocket release kinetics which permits a potential antigenic incoming peptide to displace the in-place peptide in suitable yield.

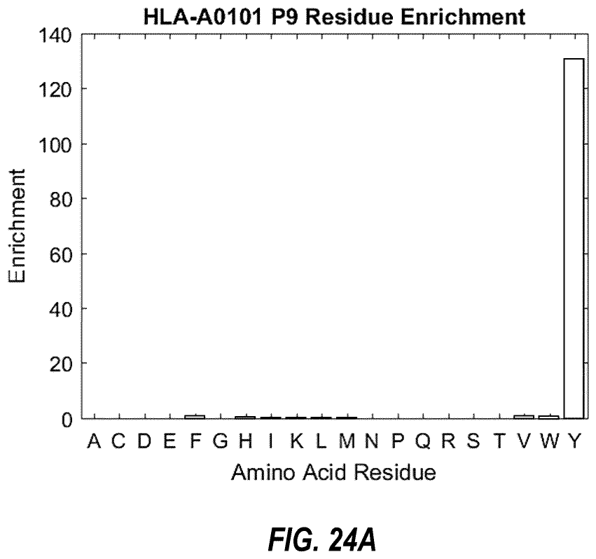

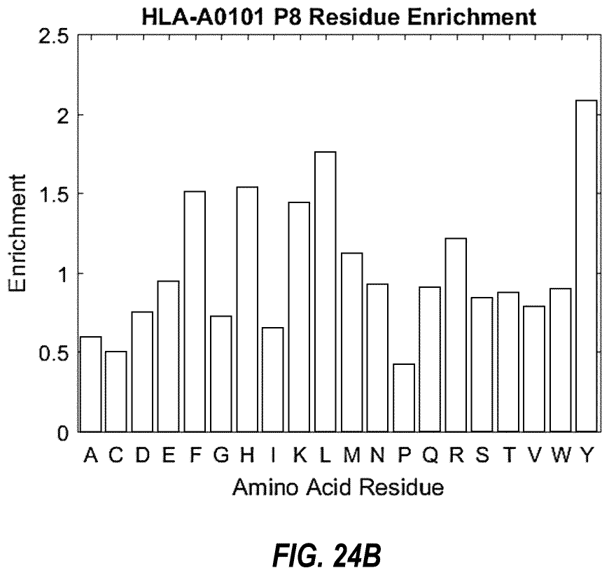

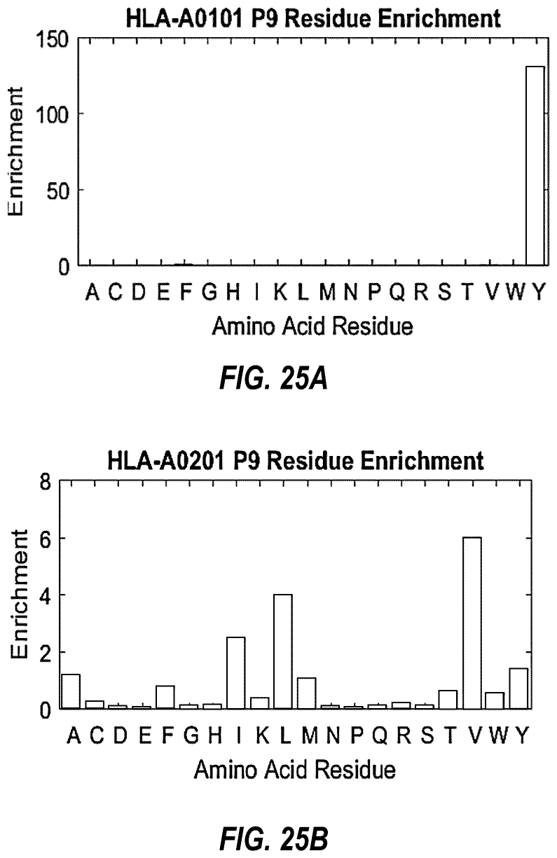

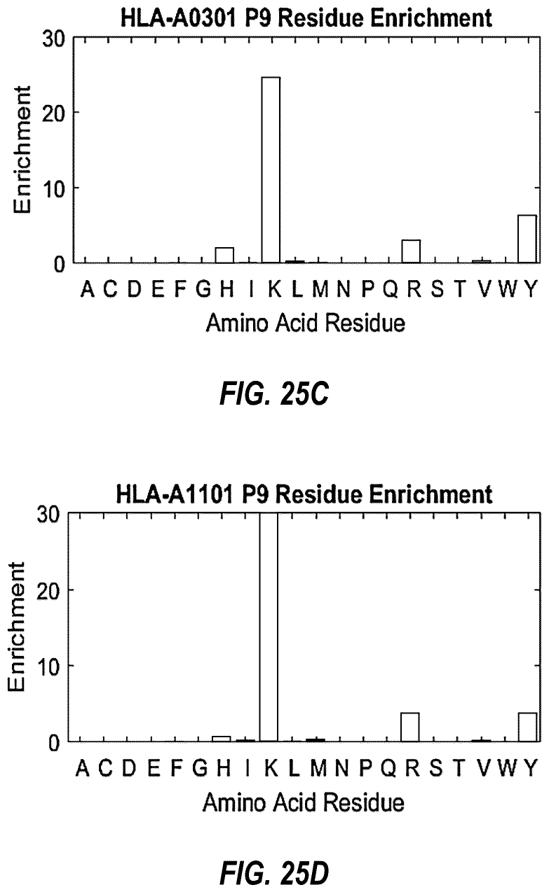

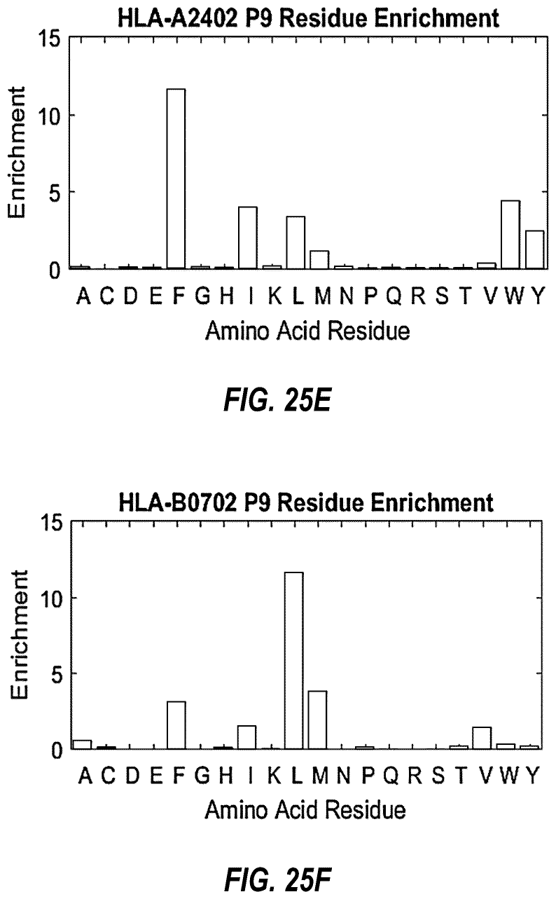

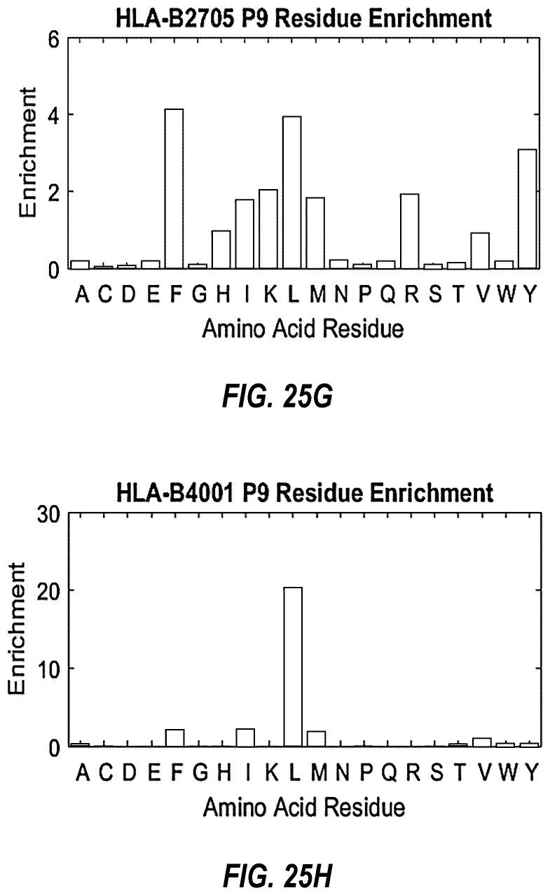

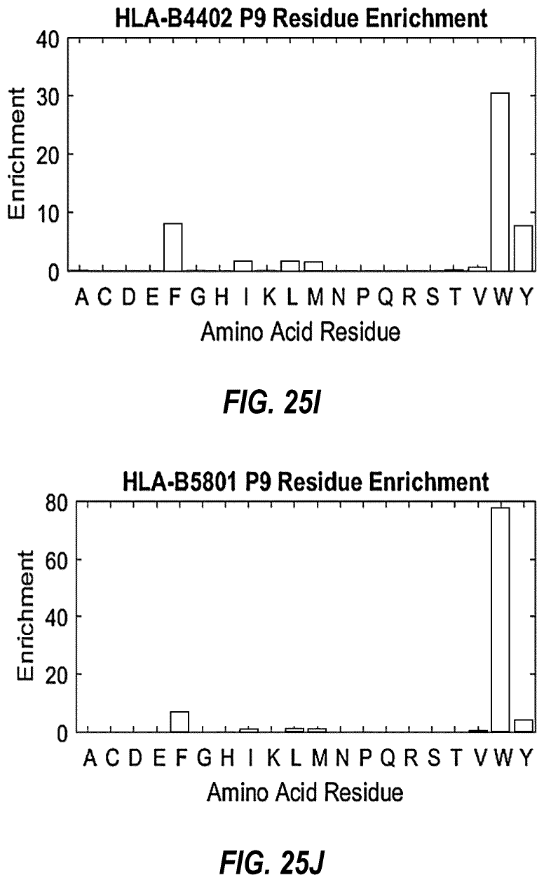

Accordingly, in some embodiments, the in-place peptide may be engineered to alter the balance between affinity and stability. For example, in some embodiments, the naturally occurring amino acid residues at the P8/P9 position of the in-place peptide may be replaced with sub-optimal anchoring or non-anchoring amino acid residues to alter the balance between affinity and stability. Selection of such amino acid residue may depend on the specific HLA and specific amino acid residue position therein. For example, the P9 and P8 residue preferences for binding peptides for HLA-A*0101 (FIGS. 24A and 24B) demonstrate a high preference for Tyr at P9 in this group, while distribution of amino acid type found at P8 for these binding peptides show a more varied distribution. For the same P9 position, while HLA-A*0101 has the preference for Tyr, each of a variety of HLA-A and HLA-B alleles has a different pattern of amino acid preferences (FIGS. 25A-25J).

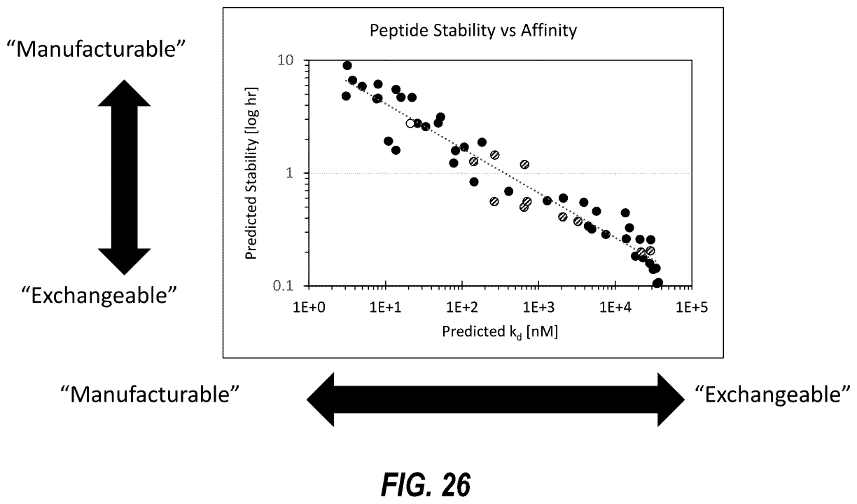

As shown in FIG. 26, there is a balance between manufacturability and exchangeability. In this context, manufacturability refers to the ability to form the pHLA complex, e.g., for the peptide sequence to effectively bind and stabilize the HLA, where peptides having predicted high stability within the pHLA and high affinity for binding with the HLA will readily form the pHLA. Exchangeability in this context refers to the ability of the in-place peptide to be displaced by contacting the pHLA complex with the exchange factor and the potential antigenic peptide. If manufacturability is too high, the in-place peptide may not be displaced effectively, thus not affording the desired functionality of an in-place peptide.

Additionally, design considerations are given to provide a location for labelling the candidate peptide sequence such that a detectable label may be introduced into the candidate initial peptide without affecting the binding to the binding groove of the MHC. Again, in the examples discussed here for HLA-A*01 and HLA-A*02, an amino acid at position 4 or 5 of the candidate peptide is not involved in binding to the binding pockets of the binding groove, and faces out of the groove. Thus, detectable labelling may be performed when that amino acid residue is selected to be lysine or another easily labelled amino acid.

Additionally, methods for evaluating the ease of displacement of the detectably labelled candidate peptide are described here, permitting evaluation of a series of candidate peptides sequences for their suitability as initial peptides (i.e., in-place peptide) in providing pHLA, and generalizable to pMHC, for providing antigen-specific activating surfaces. Rapid evaluation of many antigenic peptides may then be accomplished.

Accordingly, in some embodiments, methods of identifying an initial peptide sequence for stabilizing an MHC molecule are provided. The methods may comprise: binding a detectably labelled peptide sequence to a binding groove of the MHC molecule, forming a detectably labelled peptide sequence:MHC molecule complex (LP:MHC complex) thereby stabilizing the MHC molecule; performing an exchange reaction including contacting the LP:MHC complex with an exchange factor and a second peptide sequence for a first period of time, wherein the second peptide sequence is configured to stabilize the MHC molecule when bound to the binding groove; and detecting displacement of the detectably labelled peptide sequence from the binding groove of the MHC molecule.

In some embodiments, the detectably labelled peptide sequence may include a highly conserved self peptide sequence (e.g., a peptide sequence with a below average mutation rate; optionally wherein the mutation rate is at least one or two standard deviations below the average amino acid mutation rate in the organism) and has minimal immunogenicity. Further, the detectably labelled peptide sequence may be labelled at an amino acid residue that does not interfere with forming the LP:MHC complex.

In some embodiments, an affinity of the detectably labelled peptide sequence for binding the binding groove may include a predicted K_{d} from about 1E+1 nM to about 1E+5 nM, from about 1E+2 nM to about 2E+4 nM, or from about 1E+2 nM to about 1E+3 nM. In some embodiments, the predicted K_{d} may be from about 1E+2 nm to about 1E+4 nM. In some embodiments, the predicted K_{d} may be from about 1E+1 nm to about 2E+4 nM. In some embodiments, the predicted K_{d} may be from about 1E+2 nM to about 1E+3 nM. In some embodiments, the predicted K_{d} may be from about 1.5E+2 nM to about 1E+5 nM.

In some embodiments, a rate of release of the detectably labelled peptide sequence from the binding groove may include a predicted peptide stability of the LP:MHC complex. The predicted peptide stability can be presented as half-life of the detectably labelled peptide binding to the LP:MHC complex, which can be at least about 0.1, 0.2, 0.4, 1.5, 2, 4, 6, 8, 10, 12, 14, 16, 18, 20, 24, 28, 32, 36, 48, or 72 hours, or any number therebetween. In some embodiments, the half-life can be from about 0.1 to about 1.5 hr, from about 0.2 to about 2.0 hr, from about 0.3 to about 2.0 hr, or from about 0.4 to about 2.0 hr. In some embodiments, the half-life can be from about 0.1 to about 1.5 hr. In some embodiments, the half-life can be from about 0.2 to about 2 hr. In some embodiments, the half-life can be from about 0.3 to about 2 hr. In some embodiments, the half-life can be from about 0.4 to about 2.0 hr.

In some embodiments, the first period of time is about 2 hr to about 6 hr or 1 hr to about 10 hr. In some embodiments, the first period of time is about 1 hour, about 2 hr, about 3 hr, about 4 hr, about 5 hr, about 6 hr, about 7 hr, about 8 hr, about 9 hr, or about 10 hr. In some embodiments, the first period of time is about 4 hr.

In some embodiments, when the MHC molecule includes an HLA-A molecule, the second peptide sequence is LMYAKRAFV (SEQ ID NO: 4).

In some embodiments, detecting displacement of the detectably labelled peptide may include determining loss of fluorescence from complexes including the MHC molecule. Determining the loss of fluorescence from complexes including the MHC molecule may include capturing MHC complexes after the first period of time to capture objects described elsewhere herein (e.g., micro-objects or beads); and determining fluorescence of the captured MHC complexes, thereby determining a proportion of displacement of the detectably labelled peptide from the LP:MHC complexes.

In some embodiments, the detectably labelled peptide may include at least 4 or 5 amino acid residues; or has a length of about 4, 5, 6, 7, 8, 9, 10, 11, 12, 13, 14, 15, 16, 17, or 18 amino acid residues (e.g., ranging from 8 to 10 amino acid residues, or 13 to 18 amino acid residues). In some variations, the detectably labelled peptide may include 9 or 10 amino acid residues. In some variations, the detectably labelled peptide sequence may include a lysine as the fourth or fifth amino acid residue of the detectably labelled peptide sequence. In some embodiments, the detectably labelled peptide may be labelled at the lysine residue.

In some embodiments, the detectably labelled peptide may have an amino acid sequence selected from: GMGQKDSYV (SEQ ID NO: 1); RMQKEITAL (SEQ ID NO: 2); IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18). In other embodiments, the detectably labelled peptide may have an amino acid sequence selected from: IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

In some embodiments, the detectably labelled peptide may have an amino acid sequence of GMGQKDSYV (SEQ ID NO: 1) or GAATKMAAV (SEQ ID NO: 13).

In some embodiments, the second peptide sequence displaces at least 60% (65%, 70%, 75%, 80%, 85%, 90%, 95%, 99%) of the detectably labelled peptide bound to the MHC molecules, thereby identifying the second peptide as a suitable initial peptide sequence.

In some embodiments, the MHC molecule may include any one of HLA-A*01, 02, 03, 24, 26, 30; HLA-B*15, 35, 40, 44, 51, 52; and DRB1*03, 04, 09, 11, 13, 07. In some embodiments, the MHC may include any one of HLA-A*01, HLA-A*02, HLA-A*03, HLA-A*11, or HLA-A*24. In some embodiments, the MHC may include any one of HLA-B*07, HLA-B*27, HLA-B*40, HLA-B*44, or HLA-B*58.

In some embodiments, the detectably labelled peptide sequence may include C-terminal amino acid residues selected to modulate release kinetics from a binding pocket of the binding groove of the MHC molecule. In some variations, the binding pocket may be a F binding pocket of the binding groove of the MHC molecule.

### Peptide Antigens.

Peptide antigens are provided in various kits and surfaces described herein and are used in various methods and uses described herein. The following description is provided with respect to all disclosed embodiments herein involving peptide antigens. As noted herein, peptide antigens include candidate peptide antigens that may or may not be immunogenic when presented by an MHC, in addition to peptide antigens with known or verifiable immunogenicity, where immunogenicity refers to the ability of a peptide antigen to contribute to activation of a T lymphocyte, such as a cytotoxic T lymphocyte, when the peptide antigen is bound in the antigen-binding groove of a major histocompatibility complex (MHC), e.g., a class I MHC.

In some embodiments, a peptide antigen is 7-11 amino acid residues in length, e.g., 7, 8, 9, 10, or 11 amino acid residues in length. In some embodiments, a peptide antigen is 8, 9, or 10 amino acid residues in length. In some embodiments, a peptide antigen comprises a tumor associated antigen. The peptide can be from an extracellular domain of the tumor associated antigen. An antigen is considered tumor associated if it is expressed at a higher level on a tumor cell than on a healthy cell of the type from which the tumor cell was derived. The T cell which recognizes this tumor associated antigen is an antigen specific T cell. Any tumor associated antigen may be utilized in the antigen presenting surface described herein. Some non-limiting examples of tumor associated antigens include MART1 (peptide sequence ELAGIGILTV (SEQ ID NO: 7)), for melanoma, NYESO1 (peptide sequence SLLMWITQV (SEQ ID NO: 8)), involved in melanoma and some carcinomas, SLC45A2, TCL1, and VCX3A, but the disclosure is not so limited. Additional examples of tumor associated antigens include peptides comprising a segment of amino acid sequence from a protein expressed on the surface of a tumor cell such as CD19, CD20, CLL-1, TRP-2, LAGE-1, HER2, EphA2, FOLR1, MAGE-A1, mesothelin, SOX2, PSM, CA125, T antigen, etc. In some embodiments, the tumor associated antigen is a neoantigenic peptide, e.g., encoded by a mutant gene in a tumor cell. For detailed discussion of neoantigenic peptides, see, e.g., US 2011/0293637.

Upon reaction with a proto-antigen-presenting surface, the peptide antigen (e.g., tumor associated antigen) may become noncovalently associated with the primary activating molecular ligand (e.g., MHC molecule), e.g., through binding in the antigen-binding groove of the MHC molecule. Such binding may involve displacement of the previous occupant of the pocket (an exchange factor, or an initial peptide whose displacement is catalyzed by an exchange factor). The peptide antigen may be presented by the primary activating molecular ligand (e.g., MHC molecule) in an orientation which can initiate activation of a T lymphocyte.

In some embodiments, a population of peptide antigens is provided, e.g., in one or more pools. For example, such a population can be prepared from material from a tumor sample, and may be enriched for tumor associated antigens and/or neoantigenic peptides. The one or more pools can be used together with one or more proto-antigen-presenting surfaces described herein to generate a population of antigen-presenting surfaces, e.g., for use in screening the members of the population of peptide antigens for immunogenicity.

### Methods of Forming a Proto-Antigen-Presenting Synthetic Surface

A method of forming a proto-antigen-presenting synthetic surface for activating a T lymphocyte (T cell), is provided, comprising: synthesizing a plurality of primary activating molecules, comprising reacting major histocompatibility complex (MHC) molecules with initial peptide, thereby forming a plurality of primary activating molecules each comprising an MHC molecule complexed with an initial peptide; wherein:

, and the method further comprises reacting the plurality of primary activating molecules with a first plurality of binding moieties disposed on a covalently functionalized synthetic surface (forming a plurality of primary activating molecular ligands), thereby forming the proto-antigen-presenting surface. The initial peptide may be any of the embodiments of initial peptide described elsewhere herein.

In some embodiments, reacting the plurality of primary activating molecules with the first plurality of binding moieties disposed on the covalently functionalized synthetic surface comprises reacting a first reactive moiety of each of the primary activating molecules with a corresponding one of the plurality of first binding moieties. In some embodiments, reacting the plurality of primary activating molecules with the first plurality of binding moieties disposed on the covalently functionalized synthetic surface further comprises adding a first reactive moiety to the MHC molecule of each of the plurality of primary activating molecules prior to reacting the plurality of primary activating molecules with the first plurality of binding moieties.

In some embodiments, the method further comprises reacting the plurality of primary activating molecules with exchange factor, optionally in the presence of a peptide antigen, thereby forming an antigen-presenting surface.

In some embodiments, before reacting a plurality of MHC molecules with the exchange factor, an initial peptide is bound to the MHC molecule.

In some embodiments, a method of preparing a proto-antigen-presenting surface further comprises reacting a plurality of co-activating molecular ligands with a second plurality of binding moieties of the covalently functionalized surface. In some embodiments, each of the plurality of co-activating molecular ligands comprises a TCR co-activating molecule or an adjunct TCR activating molecule. In some embodiments, each of the plurality of co-activating molecular ligands comprises a second reactive moiety and the TCR co-activating molecule or the adjunct TCR activating molecule. In some embodiments, a plurality of co-activating molecular ligands are present on the covalently functionalized synthetic surface or are added to the covalently functionalized synthetic surface by reacting a plurality of co-activating molecules with the second plurality of binding moieties of the covalently functionalized synthetic surface configured for binding the second reactive moieties.

In some embodiments, the covalently functionalized synthetic surface presents a plurality of azido groups. In such embodiments, the first reactive moieties can be configured to react with the azido groups of the covalently functionalized synthetic surface so as to form covalent bonds. Where present, the second reactive moieties can also be configured to react with the azido groups of the covalently functionalized synthetic surface so as to form covalent bonds.

In some embodiments, the covalently functionalized synthetic surface presents a plurality of biotin-binding agents, and wherein the first reactive moieties are configured to specifically bind to the biotin-binding agent. In some such embodiments, the first reactive moieties comprise or consist essentially of biotin. Where present, the second reactive moieties can also comprise or consist essentially of biotin. The biotin-binding agent may be covalently attached to the covalently functionalized synthetic surface or noncovalently attached to the covalently functionalized synthetic surface, e.g., through biotin functionalities.

The covalently functionalized synthetic surface used to prepare a proto-antigen-presenting surface may be any of the surface types described herein, e.g., a wafer, an inner surface of a tube (e.g., glass or polymer tube), an inner surface of a microfluidic device, or a bead. The surface material may comprise, e.g., metal, glass, ceramic, polymer, a metal oxide, or a combination thereof. The microfluidic device may be any microfluidic device as described herein, and may have any combination of features. The bead can be a bead with a surface-area that is within 10% of the surface-area of a sphere of an equal volume or diameter, as discussed herein in the section regarding proto-antigen-presenting synthetic surfaces. In some embodiments, the bead may be a bead having a surface area that exceeds 10% of the surface area of a sphere of an equal volume or diameter, as discussed herein for antigen presenting surfaces. In some embodiments, the bead is not a bead that has a surface area that exceeds 10% of the surface area of a sphere of an equal volume or diameter, as discussed herein for antigen presenting surfaces.

The primary activating molecules and co-activating molecules may each be any such molecule described herein, and any combination thereof may be used. Thus, a primary activating molecule can comprise an MHC molecule and, optionally, an initial peptide or exchange factor; and a co-activating molecule can comprise any of the TCR co-activating molecules described herein or any of the adjunct TCR activating molecules described herein. As noted above, the MHC molecules may be synthesized in the presence of the initial peptide, e.g., so that they bind the initial peptide in the antigen-binding groove upon folding. In some embodiments, the MHC molecules may be synthesized in the presence of the exchange factor, e.g., so that they bind the exchange factor in the antigen-binding groove upon folding. Alternatively, the MHC molecules may be reacted with the exchange factor after synthesis. This approach can displace an initially bound peptide (or initial peptide) from the antigen-binding groove, catalyzing the introduction of an incoming peptide, which may be an antigenic peptide, or candidate antigenic peptide. Such displacement can then provide an antigen-presenting surface for activation of T cells.

Where the MHC molecules are synthesized in the presence of the initial peptide, they are subsequently incorporated into primary activating molecules through a process comprising adding a first reactive moiety (e.g., biotin or moieties configured to react with azido groups), as discussed in detail elsewhere herein. Such a method further comprises reacting the first reactive moieties of the plurality of primary activating molecules with a first plurality of binding moieties disposed on a covalently functionalized synthetic surface, thereby forming the proto-antigen-presenting surface.

Where the MHC molecules are synthesized in the presence of the exchange factor, they are subsequently incorporated into primary activating molecules through a process comprising adding a first reactive moiety (e.g., biotin or moieties configured to react with azido groups), as discussed in detail elsewhere herein. Such a method further comprises reacting the first reactive moieties of the plurality of primary activating molecules with a first plurality of binding moieties disposed on a covalently functionalized synthetic surface, thereby forming the proto-antigen-presenting surface.

Where the MHC molecules are reacted with the exchange factor after synthesis, such a reaction may occur before or after being incorporated into primary activating molecules through a process comprising adding a first reactive moiety (e.g., biotin), as discussed in detail elsewhere herein. Such a reaction may also occur before or after reacting the first reactive moieties of the plurality of primary activating molecules with a first plurality of binding moieties disposed on a covalently functionalized synthetic surface. That is, the exchange factor can be reacted with the MHC molecules in solution or when they are already associated with a surface.

In some embodiments, reacting a plurality of primary activating molecules with a first plurality of binding moieties of a covalently functionalized synthetic surface comprising binding moieties comprises forming a noncovalent association between the primary activating molecules and the binding moieties. For example, the primary activating molecules can comprise biotin and the binding moieties can comprise a biotin-binding agent such as streptavidin (e.g., which may be covalently bound to the surface or which may be non-covalently bound to a second biotin which itself is covalently bound to the surface). In some embodiments, the biotin-binding agent such as streptavidin is linked to the surface through a series of about 15, 20, 25, 30, 35, 40, 45, 50, 55, 60, 65, 70, 75, 80, 85, 95, 100, 200 bond lengths, or any number of bond lengths therebetween. For example, the biotin-binding agent may be linked to the surface through a series of one or more linkers having a selected length as described. In some embodiment, the linker linking the plurality of biotin-binding agents can have a length of about 20 Angstroms to about 100 Angstroms. In another example, both the binding moieties and the primary activating molecules can comprise biotin and a free, multivalent biotin-binding agent, such as streptavidin, can be used as a noncovalent linking agent. Any other suitable noncovalent binding pair, such as those described elsewhere herein, can also be used. In some embodiment, the linker of biotin or biotin-binding agent functionality may include a polyethylene glycol (PEG) moiety, which can include a (PEG)₁₃ repeating sequence and/or a (PEG)₄ repeating sequence.

Alternatively, reacting a plurality of primary activating molecules with a first plurality of binding moieties of a covalently functionalized synthetic surface comprising binding moieties can comprise forming a covalent bond. For example, an azide-alkyne reaction (such as any of those described elsewhere herein) can be used to form the covalent bond, where the primary activating molecules and the binding moieties comprise, respectively, an azide and an alkyne, or an alkyne and an azide. Other reaction pairs may be used, as is known in the art, including but not limited to maleimide and sulfides. More generally, exemplary functionalities useful for forming covalent bonds include azide, carboxylic acid and active esters thereof, succinimide ester, maleimide, keto, sulfonyl halides, sulfonic acid, dibenzocyclooctyne, alkene, alkyne, and the like. Skilled artisans are familiar with appropriate combinations and reaction conditions for forming covalent bonds using such moieties.

Where the covalently functionalized synthetic surface comprises a covalently associated biotin, the surface can further comprise noncovalently associated biotin-binding agent (e.g., streptavidin), such that the surface can be reacted with primary activating molecules and co-activating molecules that comprise biotin moieties. In some embodiments, the method of preparing a proto-antigen-presenting synthetic surface comprises reacting a covalently functionalized synthetic surface comprising a covalently associated biotin with a biotin-binding agent (e.g., streptavidin), and then with primary activating molecules and co-activating molecules comprising biotin moieties. In some embodiments, the biotin of the covalently functionalized surface is linked to the surface through a series of about 15, 20, 25, 30, 35, 40, 45, 50, 55, 60, 65, 70, 75, 80, 85, 95, 100, 200 bond lengths, or any number of bond lengths therebetween. In some embodiments, the streptavidin moiety is disposed upon the covalently functionalized synthetic surface or the proto-antigen-presenting surface in a density from about 4×10² to about 3×104⁴, 5×10³ to about 3×10⁴, 6×10² to about 5×10³, about 5×10³ to about 2×10⁴, about 1×10⁴ to about 2×10⁴, or about 1.25×10⁴ to about 1.75×10⁴ molecules per square micron molecules per square micron, in each portion or sub-region where it is attached. In some embodiments, the biotin-binding agent is disposed upon substantially all of the covalently functionalized synthetic surface or the proto-antigen-presenting surface.

In some embodiments, the reaction provides any of the densities described herein of primary activating molecular ligands on the surface, such as about 50, 400, 500, 1×10³, 2×10³, 5×10³, 1×10⁴, 1.25×10⁴, 1.75×10⁴, 2×10⁴, 3×10⁴ molecules per square micron, or any range therebetween.

In some embodiments, reacting a plurality of co-activating molecules, each comprising: a T cell receptor (TCR) co-activating molecule; or an adjunct TCR activating molecule, with a second plurality of binding moieties of the covalently functionalized synthetic surface comprises forming a noncovalent association between the co-activating molecules and the binding moieties. Any of the embodiments described above or set forth in any embodiments disclosed herein with respect to primary activating molecules involving noncovalent binding pairs such as biotin and a biotin-binding agent such as streptavidin may be used.

Alternatively, reacting a plurality of co-activating molecules with a second plurality of binding moieties of the covalently functionalized synthetic surface can comprise forming a covalent bond. For example, an azide-alkyne reaction (such as any of those described elsewhere herein) can be used to form the covalent bond, where the primary activating molecules and the binding moieties comprise, respectively, an azide and an alkyne, or an alkyne and an azide.

In some embodiments, the reaction provides any of the densities described herein of co-activating molecular ligands on the surface, such as about 20, 200, 250, 500, 1×10³, 2×10³, 5×10³, 1×10⁴, 1.25×10⁴, 1.5×10⁴, 1.75×10⁴, 2×10⁴, 2×10⁵, molecules per square micron, or any range therebetween.

In some embodiments, the reaction provides TCR co-activating molecules and adjunct TCR activating molecules on the surface in any of the ratios described herein, such as 100:1 to 1:100, 20:1 to 1:20, 10:1 to 1:10, 5:1 to 1:5, or 3:1 to 1:3, wherein each of the foregoing values can be modified by “about.”

In some embodiments, the reactions described above or set forth in any embodiments disclosed herein provide primary activating molecular ligands and co-activating molecular ligands on the surface in any of the ratios described herein, such as about 1:1 to about 2:1; about 1:1; or about 3:1 to about 1:3.

In some embodiments, a method of preparing a proto-antigen-presenting surface further comprises reacting a plurality of surface-blocking molecules with a third plurality of binding moieties of the covalently functionalized surface, wherein each of the binding moieties of the third plurality is configured for binding the surface-blocking molecule. Any surface-blocking molecule described elsewhere herein may be used. Any of the reaction approaches described herein for forming noncovalent associations or a covalent bond may be used.

In some embodiments, a method of preparing a proto-antigen-presenting surface further comprises reacting a plurality of adhesion stimulatory molecular ligands, wherein each adhesion stimulatory molecular ligand includes a ligand for a cell adhesion receptor including an ICAM protein sequence, with a fourth plurality of binding moieties of the covalently functionalized bead, wherein each of the binding moieties of the fourth plurality is configured for binding with the cell adhesion receptor ligand molecule. Any of the reaction approaches described herein for forming noncovalent associations or a covalent bond may be used.

In some embodiments, the covalently functionalized synthetic surface or the proto-antigen-presenting surface further includes a first portion and a second portion, wherein the distribution of the at least one plurality of biotin-binding agent or biotin functionalities is located in the first portion of the covalently modified synthetic surface, and the distribution of the at least one plurality of the surface-blocking molecular ligands is located in the second portion. In some embodiments, a second plurality of surface-blocking molecular ligands is disposed in the first portion of the covalently functionalized synthetic surface or the proto-antigen-presenting surface. The first portion of the covalently functionalized synthetic surface or the proto-antigen-presenting surface can further include a plurality of first regions, each first region including at least a subset of the plurality of the biotin-binding agent or biotin functionalities, wherein each of the plurality of first regions is separated from another of the plurality of first regions by the second region configured to substantially exclude the streptavidin or biotin functionalities. Each of the plurality of first regions including at least the subset of the plurality of the streptavidin or biotin functionalities can have an area of about 0.10 square microns to about 4.0 square microns. The area of each of the plurality of first regions including at least the subset of the plurality of the primary activating molecular ligands can be about 0.8 square microns to about 4.0 square microns.

In some embodiments, a method of preparing a proto-antigen-presenting surface further comprises producing the intermediate reactive surface. This can include, e.g., reacting at least a first portion of surface-exposed moieties disposed at a surface of a synthetic reactive surface with a plurality of intermediate preparation molecules including reactive moieties, thereby producing the intermediate reactive surface. Methods of preparing a covalently functionalized surface, which can be used as the intermediate reactive surface, are described in detail elsewhere herein. Producing the intermediate reactive surface can comprise any of the features described herein with respect to methods of preparing a covalently functionalized surface.

In some embodiments, the methods further comprise modulating the capacity for cells to adhere to surfaces within the microfluidic device, e.g., by providing anchoring points for cells requiring mechanical stress of adherence to grow and expand appropriately. This can be accomplished by introducing a covalently bound surface modification comprising surface contact moieties to help anchor adherent cells. Any of the surface contact moieties described elsewhere herein can be used.

The covalently functionalized synthetic surface can comprise moieties suitable for use in any of the reactions described herein.

### Methods of Preparing a Covalently Functionalized Surface.

In some embodiments, preparation of a proto-antigen-presenting surface from a covalently functionalized surface further comprises preparing a covalently functionalized surface including a plurality of streptavidin or biotin functionalities and at least a first plurality of surface-blocking molecular ligands. In some embodiments, preparing the covalently functionalized surface comprises reacting at least a first subset of reactive moieties of an intermediate reactive synthetic surface with a plurality of linking reagents, each linking reagent including streptavidin or biotin; and reacting at least a second subset of reactive moieties of the intermediate reactive synthetic surface with a plurality of surface-blocking molecules, thereby providing the covalently functionalized synthetic surface including at the least one plurality of streptavidin or biotin functionalities and at the least first plurality of surface-blocking molecular ligands. Generally only one or the other of a linking reagent including streptavidin or a linking reagent including biotin is used. The intermediate reactive synthetic surface may be any of the surface types described herein, e.g., a bead, wafer, inner surface of a microfluidic device, or tube (e.g., glass or polymer tube). The surface material may comprise, e.g., metal, glass, ceramic, polymer, a metal oxide, or a combination thereof. The microfluidic device may be any microfluidic device as described herein, and may have any combination of features. The bead can be a bead with a surface-area is within 10% of the surface-area of a sphere of an equal volume or diameter, as discussed herein in the section regarding proto-antigen-presenting synthetic surfaces.

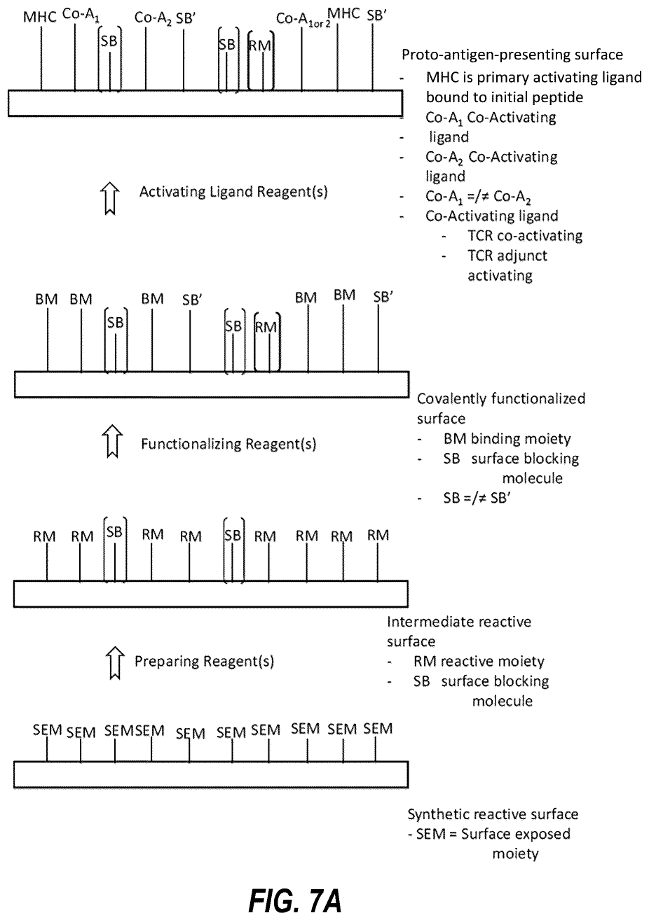

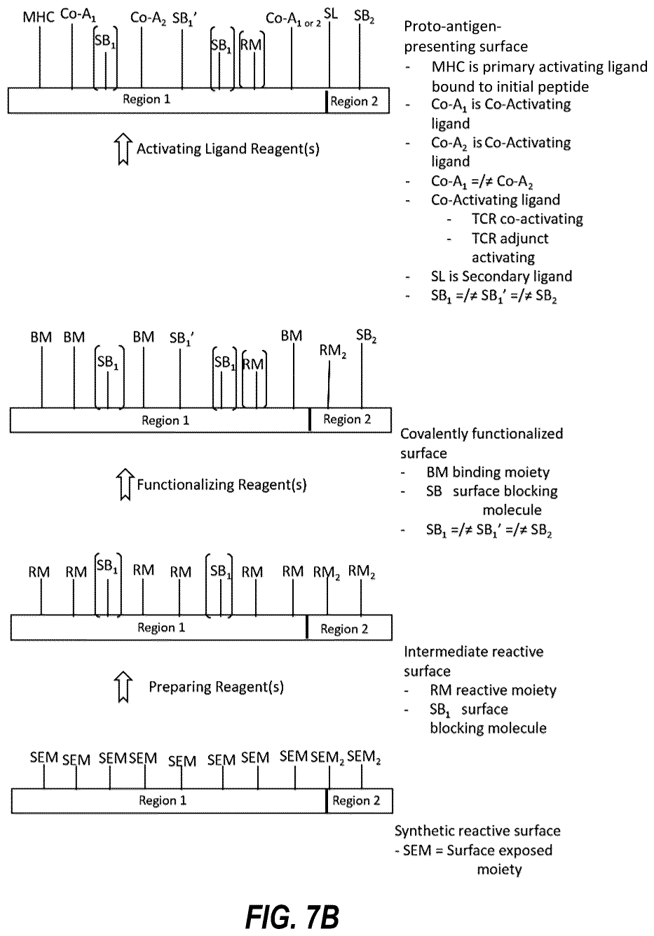

FIGS. 7A and 7B show the structure of a proto-antigen-presenting synthetic surface as it is constructed from an unmodified surface according to certain exemplary methods, comprising adding the activating, co-activating and surface-blocking molecular ligands in one or more steps. FIG. 7A shows the process and structure for a proto-antigen-presenting synthetic surface having a single region, while FIG. 7B shows the process and structure of each intermediate and final product for a proto-antigen-presenting synthetic surface having two regions.

Turning to FIG. 7A, the schematic representation illustrates an exemplary procedure for preparing a proto-antigen-presenting surface starting with a synthetic reactive surface comprising a plurality of surface-exposed moieties (SEM). Reactive moieties RM and surface-blocking molecular ligands SB, if added at this point in the preparation, are introduced by reacting the SEMs with appropriate preparing reagent(s), providing an intermediate reactive surface. The reactive moieties RM introduced to the intermediate reactive surface may be any reactive moiety described herein and may be linked to the intermediate reactive surface by any linker described herein. The intermediate reactive surface includes at least reactive moieties RM, and, in some embodiments, may include surface-blocking molecular ligands SB, which may be any surface-blocking molecular ligand as described herein.

The intermediate reactive surface is then treated with functionalizing reagents including binding moieties BM, where the functionalizing reagents react with the reactive moieties RM to introduce binding moiety BM ligands. The binding moieties so introduced may be any binding moiety BM described herein. The binding moiety BM may be streptavidin or biotin. In some embodiments, the binding moiety BM is streptavidin which is covalently attached via a linker to the covalently functionalized surface, through a reaction with a reactive moiety RM. In some other embodiments, the covalently functionalized surface may introduce a streptavidin binding moiety non-covalently, in a two part structure. This two part structure is introduced by contacting the intermediate reactive surface with a first functionalizing reagent to introduce a biotin moiety covalently attached via a linker through reaction with the reactive moieties RM. Subsequent introduction of streptavidin, as a second functionalizing reagent, provides the covalently functionalized surface wherein the binding moiety BM, streptavidin, is non-covalently attached to a biotin moiety which itself is covalently attached to the surface.

Surface-blocking molecular ligands SB′ may be introduced at the same time as the introduction of the binding moieties or may be introduced to the covalently functionalized surface subsequent to the introduction of the binding moieties. The surface-blocking molecular ligands SB′ may be any surface-blocking molecular ligand as described herein and may be the same as or different from surface-blocking molecular ligands SB, if surface-blocking molecular ligands SB are present. In some embodiments, surface-blocking molecular ligands SB may be present and there may be no surface-blocking molecular ligands SB′. Alternatively, there may be surface-blocking molecular ligands SB′ but no surface-blocking molecular ligands SB. In some embodiments, both surface-blocking molecular ligands SB and SB′ are present.

Without being bound by theory, there may be some reactive moieties RM left unreacted upon the covalently functionalized surface but there are insufficient numbers of reactive moieties RM present to prevent the product antigen presenting synthetic surface from functioning.

Primary activating molecular ligands MHC and Co-activating molecular ligands Co-A₁ and Co-A₂ are introduced by reacting the binding moieties BM of the covalently functionalized surface with appropriate activating ligand reagents, providing the proto-antigen-presenting synthetic surface in the case where the MHC at the time of reaction with the binding moieties comprises an exchange factor. Alternatively, the MHC at the time of reaction with the binding moieties may comprise an initial peptide and the proto-antigen-presenting surface can be provided by contacting the MHC (already associated with the surface) with an exchange factor, e.g., at molar excess under conditions suitable for displacement of the initial peptide by the exchange factor. Co-A₁ and Co-A₂ may be the same or different co-activating ligands. For example, Co-A₁ and Co-A₂ can comprise one, the other, or collectively both of a TCR co-activating molecule and an adjunct TCR activating molecule. Co-A₁ and/or Co-A₂, may be any combination of TCR co-activating molecule and an adjunct TCR activating molecule as described herein.

In some embodiments, the primary activating molecular ligand MHC may be introduced to the covalently functionalized surface, before the covalently functionalized surface is contacted with the co-activating molecular ligands Co-A₁ and/or Co-A₂. In other embodiments, the primary activating molecular ligand MHC may be introduced to the covalently functionalized surface concurrently with or subsequently to the introduction of the Co-Activating molecular ligands Co-A₁ and Co-A₂. In some embodiments, not shown in FIG. 7A, after introduction of the primary activating molecular ligand MHC and co-activating molecular ligands Co-A₁ and/or Co-A₂, surface-blocking molecular ligands SB may be introduced to the antigen presenting synthetic surface by reacting surface-blocking molecules with remaining reactive moieties RM still present on the proto-antigen-presenting synthetic surface. Also included but not illustrated in FIG. 7A, is the introduction of Secondary Ligands SL, which may be one or more growth stimulatory molecular ligands and/or adhesion stimulatory molecular ligands. Secondary Ligands SL may be any of these classes of ligands.

FIG. 7B provides a schematic illustration of an exemplary procedure for preparing a proto-antigen-presenting surface comprising first and second regions starting with a synthetic reactive surface comprising a plurality of surface-exposed moieties (SEM). The surface exposed moieties SEM in Region 1 may be different from the surface exposed moieties SEM₂ in Region 2, as shown in FIG. 8, where different materials may be present at the surface of the synthetic reactive surface. Reactive moieties RM are introduced in region 1 and substantially not in region 2, while reactive moieties RM₂ are introduced in region 2, and substantially not in region 1, due to the use of orthogonal chemistries for each of SEM and SEM₂. For example, as shown in FIG. 6, the SEM of region 1 may be reacted with an alkoxysiloxane reagent comprising an azide RM, while the SEM2 of region 2 may be reacted with a phosphonic acid reagent comprising an alkynyl RM. Surface-blocking molecular ligands SB₁ are introduced in region 1, and substantially not in region 2, by reacting the SEMs with appropriate preparing reagent(s) (e.g., for a surface like region 1 of FIG. 8, the reagent would be an alkoxysiloxane reagent including a surface-blocking group SB). An intermediate reactive surface having differentiated reactive moieties result from this process.

Based on the differentiated reactive moieties RM and RM₂, further orthogonal chemistries can introduce binding moieties BM and surface-blocking molecular ligands SB₁′ in region 1 and not substantially in region 2, and surface-blocking molecular ligands SB₂ are introduced in region 2, and not substantially in region 1. Thus, a covalently functionalized surface having two different regions is provided. The SB₁′ may be the same as or different from SB₁; SB₁′ may be the same as or different from SB₂; and SB₂ may be the same as or different from SB₁.

Primary activating molecular ligands MHC and Co-Activating molecular Ligands Co-A1 and Co-A2 are introduced in region 1 by reacting binding moieties BM with appropriate activating ligand reagents, and secondary ligands SL are formed in region 2 by reacting RMs with appropriate reagent(s), providing the proto-antigen-presenting synthetic surface in the case where the MHC at the time of reaction with the binding moieties comprises an exchange factor. Alternatively, the MHC at the time of reaction with the binding moieties may comprise an initial peptide and the proto-antigen-presenting surface can be provided by contacting the MHC (already associated with the surface) with an exchange factor, e.g., at molar excess under conditions suitable for displacement of the initial peptide by the exchange factor. Secondary Ligands SL may be any of the classes of molecular ligands as described for FIG. 7A. The primary activating molecular ligand MHC may be introduced before introducing the Co-Activating molecular Ligands, similarly to the process described for FIG. 7A. Co-A₁ and Co-A₂ may be the same or different co-activating molecular ligands. For example, Co-A₁ and Co-A₂ can comprise one, the other, or collectively both of a TCR co-activating molecule and an adjunct TCR activating molecule. Each of SEM, RM, SB, primary activating molecular ligand MHC, Co-activating molecular Ligands Co-A₁ and Co-A₂, and secondary ligands SL may be any SEM, RM, SB, primary activating molecular ligand MHC, Co-activating molecular Ligands Co-A₁ and Co-A₂, and secondary ligands SL described herein.

In embodiments in which the linking reagents include biotin, the method can further comprise noncovalently associating streptavidin with the biotin. In such embodiments, with reference to FIG. 7A, the conversion of a reactive moiety RM to a binding moiety BM can comprise covalently attaching a biotin (corresponding to the additional biotin in the above description) through reaction with the RM and then associating a streptavidin noncovalently with the covalently attached biotin.

In some embodiments, the reactive moieties of an intermediate reactive synthetic surface are linked to the surface through a series of 5, 10, 11, 12, 13, 14, 15, 16, 17, 18, 19, 20, or, in some embodiments, greater numbers of bonds. For example, the reactive moieties can be linked through a series of 15 bonds, e.g., using (11-(X)undecyl)trimethoxy silane, where X is the reactive moiety (e.g., X can be azido). With respect to linking reagents including biotin, biotin can then be covalently associated using a linking reagent such as one having the general structure DBCO-PEG₄-biotin (commercially available from BroadPharm). In some embodiments, the biotin is linked to the surface through a series of about 15, 20, 25, 30, 35, 40, 45, 50, 55, 60, 65, 70, 75, 80, 85, 95, 100, 200 bond lengths, or any number of bond lengths therebetween. With respect to linking reagents including streptavidin, streptavidin can then be covalently associated using a linking reagent such as one having the general structure DBCO-PEG₁₃-succinimide, followed by reaction of streptavidin with the succinimide. In some embodiments, the streptavidin is linked to the surface through a series of about 15, 20, 25, 30, 35, 40, 45, 50, 55, 60, 65, 70, 75, 80, 85, 95, 100, 200 bond lengths, or any number of bond lengths therebetween. The number of bonds through which a moiety is linked to a surface can be varied, e.g., by using reagents similar to those mentioned above but with alkylene and/or PEG chains of different lengths.

In some embodiments, the reactive moieties of at least first region of the intermediate reactive synthetic surface include azide moieties. In some embodiments, covalent bonds are formed through an azide-alkyne reaction, such as any azide-alkyne reaction described elsewhere herein.

In some embodiments, the covalently functionalized synthetic surface includes a second region wherein the plurality of streptavidin functionalities is excluded. In some embodiments, the at least first plurality of surface-blocking molecular ligands are disposed in the second region of the covalently functionalized synthetic surface.

In some embodiments, a method further includes reacting a second plurality of surface-blocking molecules with a second subset of reactive moieties in the at least first region of the intermediate reactive synthetic surface.

In some embodiments, the reacting of the plurality of streptavidin functionalities and the reacting of the at least first plurality of surface-blocking molecules is performed at a plurality of sub-regions of the at least first region of the covalently prepared synthetic surface including reactive moieties.

In some embodiments, the second portion of the reactive synthetic surface includes surface exposed moieties configured to substantially not react with the pluralities of the primary activating molecules and the co-activating molecules.

In some embodiments, a method further includes preparing the intermediate reactive synthetic surface, including: reacting at least a first surface preparing reagent including azide reactive moieties with surface-exposed moieties disposed at at least a first region of a reactive synthetic surface.

In some embodiments, the surface-exposed moieties are nucleophilic moieties. In some embodiments, the nucleophilic moiety of the surface is a hydroxide, amino or thiol. In some other embodiments, the nucleophilic moiety of the surface may be a hydroxide.

In some embodiments, the surface-exposed moieties are displaceable moieties.

In some embodiments, where two modifying reagents are used, the reaction of the first modifying reagent and the reaction of the second modifying reagent with the surface may occur at random locations upon the surface. In other embodiments, the reaction of the first modifying reagent may occurs within a first region of the surface and reaction of the second modifying reagent may occur within a second region of the surface abutting the first region. For example, the surfaces within the channel of a microfluidic device may be selectively modified with a first surface modification and the surfaces within the sequestration pen, which abut the surfaces within the channel, may be selectively modified with a second, different surface modification.

In yet other embodiments, the reaction of the first modifying reagent may occurs within a plurality of first regions separated from each other on the at least one surface, and the reaction of the second modifying reaction may occur at a second region surrounding the plurality of first regions separated from each other.

In various embodiments, modification of one or more surfaces of a microfluidic device to introduce a combination of a first surface modification and a second surface modification may be performed after the microfluidic device has been assembled. For one nonlimiting example, the first and second surface modification may be introduced by chemical vapor deposition after assembly of the microfluidic device. In another nonlimiting example, a functionalized surface having a first surface modification having a first reactive moiety and a second surface modification having a second, orthogonal reactive moiety may be introduced. Differential conversion to two different surface modifying ligands having two different surface contact moieties can follow.

In some embodiments, at least one of the combination of first and second surface modification may be performed before assembly of the microfluidic device. In some embodiments, modifying the at least one surface may be performed after assembly of the microfluidic device.

In some embodiments, a covalently functionalized surface is prepared comprising a binding agent. In some embodiments, the binding agent can be a multivalent binding agent, for example, a tetravalent binding agent (e.g., streptavidin functionalities, which may be covalently associated or noncovalently associated with a covalently bound biotin), a trivalent binding agent, a divalent binding agent, and/or a monovalent binding agent. In some embodiments, the distribution of the plurality of binding agent on the covalently functionalized synthetic surface is: for a tetravalent binding agent, from about 6×10² to about 5×10³ molecules per square micron; for a trivalent binding agent, from about 1.5×10³ to about 1×10⁴, about 1.5×10³ to about 7.5×10³, or about 3×10³ to about 7.5×10³ molecules per square micron; for a divalent binding agent, from about 2.5×10³ to about 1.5×10⁴, about 2.5×10³ to about 1×10⁴, or about 5×10³ to about 1×10⁴ molecules per square micron; or for a monovalent binding agent, from about 5×10³ to about 3×10⁴, about 5×10³ to about 2×10⁴, or about 1×10⁴ to about 2×10⁴ molecules per square micron, in each region where it is attached.

In some embodiments, a covalently functionalized surface is prepared comprising a binding agent, in which the distribution of the plurality of binding agent (e.g., streptavidin functionalities, which may be covalently associated or noncovalently associated with a covalently bound biotin) on the covalently functionalized synthetic surface is from about 1×10⁴ to about 1×10⁶ molecules per square micron, in each region where it is attached.

In some embodiments, a combined method comprising preparing a covalently functionalized surface and then preparing a proto-antigen-presenting synthetic surface is provided. As such, any suitable combination of steps for preparing the covalently functionalized surface and steps for preparing the proto-antigen-presenting synthetic surface may be used.

### Additional Aspects of Surface Preparation and Covalently Functionalized Surfaces.

Any method of preparing a surface described herein, including methods of preparing an proto-antigen-presenting synthetic surface, may further comprise one or more of the following aspects. A covalently functionalized surface may further comprise one or more of the following aspects applicable to such surfaces, such as reactive groups.

Azide-alkyne reactions. In some embodiments, covalent bonds are formed by reacting an alkyne, such as an acyclic alkyne, with an azide. For example, a “Click” cyclization reaction may be performed, which is catalyzed by a copper (I) salt. When a copper (I) salt is used to catalyze the reaction, the reaction mixture may optionally include other reagents which can enhance the rate or extent of reaction. When an alkyne, e.g., of a surface modifying reagent or a functionalized surface is a cyclooctyne, the “Click” cyclization reaction with an azide of the corresponding functionalized surface or the surface modifying reagent may be copper-free. A “Click” cyclization reaction can thereby be used to couple a surface modifying ligand to a functionalized surface to form a covalently modified surface.

Copper catalysts. Any suitable copper (I) catalyst may be used. In some embodiments, copper (I) iodide, copper (I) chloride, copper (I) bromide or another copper (I) salt. In other embodiments, a copper (II) salt may be used in combination with a reducing agent such as ascorbate to generate a copper (I) species in situ. Copper sulfate or copper acetate are non-limiting examples of a suitable copper (II) salt. In other embodiments, a reducing agent such as ascorbate may be present in combination with a copper (I) salt to ensure sufficient copper (I) species during the course of the reaction. Copper metal may be used to provide Cu(I) species in a redox reaction also producing Cu(II) species. Coordination complexes of copper such as [CuBr(PPh₃)₃], silicotungstate complexes of copper, [Cu(CH₃CN)₄]PF₆, or (Eto)₃P CuI may be used. In yet other embodiments, silica supported copper catalyst, copper nanoclusters or copper/cuprous oxide nanoparticles may be employed as the catalyst.

Other reaction enhancers. As described above, reducing agents such as sodium ascorbate may be used to permit copper (I) species to be maintained throughout the reaction, even if oxygen is not rigorously excluded from the reaction. Other auxiliary ligands may be included in the reaction mixture, to stabilize the copper (I) species. Triazolyl containing ligands can be used, including but not limited to tris(benzyl-1H-1,2,3-triazol-4-yl) methylamine (TBTA) or 3 [tris(3-hydroxypropyltriazolylmethyl)amine (THPTA). Another class of auxiliary ligand that can be used to facilitate reaction is a sulfonated bathophenanthroline, which is water soluble, as well, and can be used when oxygen can be excluded. Other chemical couplings as are known in the art may be used to couple a surface modifying reagent to a functionalized surface.

Cleaning the surface. The surface to be modified may be cleaned before modification to ensure that the nucleophilic moieties on the surface are freely available for reaction, e.g., not covered by oils or adhesives. Cleaning may be accomplished by any suitable method including treatment with solvents including alcohols or acetone, sonication, steam cleaning and the like. Alternatively, or in addition, such pre-cleaning can include cleaning (e.g., of the cover, the microfluidic circuit material, and/or the substrate in the context of components of a microfluidic device) in an oxygen plasma cleaner, which can remove various impurities, while at the same time introducing an oxidized surface (e.g. oxides at the surface, which may be covalently modified as described herein). Alternatively, liquid-phase treatments, such as a mixture of hydrochloric acid and hydrogen peroxide or a mixture of sulfuric acid and hydrogen peroxide (e.g., piranha solution, which may have a ratio of sulfuric acid to hydrogen peroxide from about 3:1 to about 7:1) may be used in place of an oxygen plasma cleaner. This can advantageously provide more sites for modification on the surface, thereby providing a more closely packed modified surface layer.

Components of microfluidic devices. A surface of a material that may be used as a component of a microfluidic device may be modified before assembly thereof. Alternatively, a partially or completely constructed microfluidic device may be modified such that all surfaces that will contact biomaterials including biomolecules and/or micro-objects (which may include biological micro-objects) are modified at the same time. In some embodiments, the entire interior of a device and/or apparatus may be modified, even if there are differing materials at different surfaces within the device and/or apparatus. This discussion also applies to the methods of preparing an proto-antigen-presenting synthetic surface described herein.

When an interior surface of a microfluidic device reacted with a surface modifying reagent, the reaction may be performed by flowing a solution of the surface modifying reagent into and through the microfluidic device.

Surface modifying reagent solutions and reaction conditions. In various embodiments, the surface modifying reagent may be used in a liquid phase surface modification reaction, e.g., wherein the surface modifying reagent is provided in solution, such as an aqueous solution. Other useful solvents include aqueous dimethyl sulfoxide (DMSO), DMF, acetonitrile, or an alcohol may be used. For example, surfaces activated with tosyl groups or labeled with epoxy groups can be modified in liquid phase reactions. Reactions to couple biotin or proteins such as antibodies, MHCs, or streptavidin to a binding moiety can also be performed as liquid phase reactions.

The reaction may be performed at room temperature or at elevated temperatures. In some embodiments, the reaction is performed at a temperature in a range from about 15° C. to about 60° C.; about 15° C. to about 55° C.; about 15° C. to about 50° C.; about 20° C. to about 45° C. In some embodiments, the reaction to convert a functionalized surface of a microfluidic device to a covalently modified surface is performed at a temperature of about 15° C., 20° C., 25° C., 30° C., 35° C., 40° C., 45° C., 50° C., 55° C., or about 60° C.

Alternatively, a surface modifying reagent may be used in a vapor phase surface modification reaction. For example, silica surfaces and other surfaces comprising hydroxyl groups can be modified in a vapor phase reaction. In some embodiments, a surface (e.g., a silicon surface) is treated with plasma (e.g., using an oxygen plasma cleaner; see the Examples for exemplary treatment conditions). In some embodiments, a surface, such as a plasma treated and/or silicon surface, is reacted under vacuum with a preparing reagent, e.g., comprising a methoxysilane and an azide, such as (11-azidoundecyl) trimethoxy silane. The preparing reagent can be provided initially in liquid form in a vessel separate from the surface and can be vaporized to render it available for reaction with the surface. A water source such as a hydrated salt, e.g., magnesium sulfate heptahydrate can also be provided, e.g., in a further separate vessel. For example, foil boat(s) in the bottom of a vacuum reactor can be used as the separate vessel(s). Exemplary reaction conditions and procedures include pumping the chamber to about 750 mTorr using a vacuum pump and then sealing the chamber. The vacuum reactor can then be incubated at a higher-than ambient temperature for an appropriate length of time, e.g., by placing it within an oven heated at 110° C. for 24-48 h. Following the reaction period, the chamber can be allowed to cool and an inert gas such as argon can be introduced to the evacuated chamber. The surface can be rinsed with one or more appropriate liquids such as acetone and/or isopropanol, and then dried under a stream of inert gas such as nitrogen. Confirmation of introduction of the modified surface can be obtained using techniques such as ellipsometry and contact angle goniometry.

Additional modified surfaces, surface-modifying reagents, and related methods that can be employed in accordance with this disclosure are described in WO2017/205830, published Nov. 30, 2017, which is incorporated herein by reference for all purposes.

### Methods of Activating a T Lymphocyte.

A method of activating T lymphocytes is provided, comprising: preparing an antigen-presenting surface as described herein; contacting a plurality of T lymphocytes with the antigen-presenting synthetic surface; and, culturing the plurality of T lymphocytes in contact with the proto-antigen-presenting synthetic surface, thereby converting at least a portion of the plurality of T Lymphocytes to activated T lymphocytes. Any proto-antigen-presenting surface described herein may be used to generate the antigen-presenting surface. In some embodiments, the MHC molecule is an MHC Class 1 molecule. In various embodiments, the plurality of MHC molecules may each include an amino acid sequence, and further may be connected to the surface via a C-terminal connection of the amino acid sequence. Alternatively, the MHC molecule can be connected to the surface through a noncovalent association. Any noncovalent association can be used, e.g., biotinylation of the MHC and binding thereof to streptavidin on the surface. In various embodiments, the MHC molecule may further include a peptide antigen following displacement of an exchange factor, such as any of the exchange factors described herein. In some embodiments, the peptide antigen is a tumor associated antigen, e.g., any of the tumor associated antigens described herein.

In some embodiments, the co-activating molecules may be connected to the proto-antigen-presenting synthetic surface, as described herein. The T cell receptor (TCR) co-activating molecule or an adjunct TCR activating molecule of the plurality of co-activating molecules may be any TCR co-activating molecule or any adjunct TCR activating molecule as described herein and may be provided in any ratio described herein.

In various embodiments the method may further include contacting the plurality of T lymphocytes with a plurality of growth stimulatory molecular ligands. In some embodiments, each of the growth stimulatory molecular ligands may include a growth factor receptor ligand. In some embodiments, contacting the plurality of T lymphocytes with the plurality of growth stimulatory molecular ligands may be performed after a first period of culturing of at least one day. In some embodiments, the plurality of growth stimulatory molecular ligands may include IL-21 or a fragment thereof. In various embodiments, the plurality of growth stimulatory molecular ligands may be connected to the antigen-presenting synthetic surface. In some embodiments, the plurality of growth stimulatory molecular ligands may be connected to a surface (e.g., of a bead) that is a different surface than the antigen-presenting synthetic surface including the biomolecules including MHC molecules. In some embodiments, the plurality of growth stimulatory molecular ligands may be connected to the antigen-presenting synthetic surface including MHC molecules.

In various embodiments, the method may include using antigen presenting surfaces on beads. When beads having antigen presenting surfaces are used, the ratio of beads to T lymphocytes may be about 1:1; about 3:1; about 5:1; about 7:1; about 10:1; or any ratio selected to be between these values. The beads may have antigen presenting MHC molecules and anti-CD28 antibodies attached thereto in any method as described herein. In some embodiments, IL-21 may also be attached to the antigen presenting surface of the bead. In other embodiments, IL-21 may be attached to a second bead that has IL-21 as the only biomolecule contributing to activation.

In other embodiments, the method may be performed using a planar surface which may be patterned or unpatterned.

In various embodiments, a first period of culturing may be performed for 4, 5, 6, 7, or 8 days. During the first period of culturing, growth stimulatory molecules such as IL-21, IL-2, and/or IL-7 may be added in solution or may be added on bead to feed the T lymphocytes.

At the end of a first period of culture, the population of cells may include a mixture of unactivated and activated T lymphocytes. Flow cytometry using multiple cell surface markers can be performed to determine the extent of activation and the phenotype of the cells analyzed.

A second period of culture can be performed. If the antigen presenting surfaces are beads, a second aliquot of beads containing the primary activating molecular ligand including the MHC molecule, which includes the tumor associated antigen and co-activating molecules (e.g., TCR co-activating molecules and/or adjunct TCR activating molecules, such as anti-CD28 antibodies and/or anti-CD2 antibodies, respectively) may be provided to the T lymphocytes, e.g., by addition to the wellplate, chamber of the fluidic circuit containing device, or microfluidic device having sequestration pens as described herein. The antigen presenting beads may further include additional growth stimulatory molecules, e.g., IL-21, connected thereto. The antigen presenting beads may be added to the cells being cultured in a ratio of about 1:1; about 3:1; about 5:1; about 7:1; about 10:1; or any ratio selected to be between these values to the cells. In some embodiments, a second aliquot of IL-21 may be added as a second set of beads having IL-21 connected thereto, or further, may be added as a solution. IL-2 and IL-7 may also be added during the second period of culturing to activate additional numbers of T lymphocytes.

When a patterned or unpatterned wafer, inner surface of a fluidic circuit containing device, inner surface of a tube, or inner surface of a microfluidic device having sequestration pens is used, a second period of culturing may be accomplished by continuing to culture in contact with same antigen presenting surface. Alternatively, a new antigen presenting surface may be brought into contact with the T lymphocytes resultant from the first period of culturing. In other embodiments, antigen presenting beads, like any described above or set forth in any embodiments disclosed herein, may be added to the wells or interior chamber of a fluidic circuit containing device or the sequestration pens of a microfluidic device. Growth stimulatory molecules such as IL-21, IL-2, IL-7, or a combination thereof may be added in solution or on beads. In some embodiments, IL-2 and IL-7 are added.

At the conclusion of the second culturing period, flow cytometry analysis can be performed to determine the extent of activation and to determine the phenotype of the further activated T lymphocytes present at that time.

In some embodiments, a third period of culturing may be included. The third period may have any of the features described herein with respect to the second period. In some embodiments, the third period is performed in the same way as the second period. For example, and all of the actions employed in the second period of culturing may be repeated to further activate T lymphocytes in the wells of the wellplate, in a tube, or in the chamber of a fluidic circuit containing device or a microfluidic device having sequestration pens.

In some embodiments, the T lymphocytes being activated comprise CD8+ T lymphocytes, such as naïve CD8+ T lymphocytes. In some embodiments, the T lymphocytes being activated are enriched for CD8+ T lymphocytes, such as naïve CD8+ T lymphocytes. Alternatively, in some embodiments, the T lymphocytes being activated comprise CD4+ T lymphocytes, such as naïve CD4+ T lymphocytes. In some embodiments, the T lymphocytes being activated are enriched for CD4+T lymphocytes, such as naïve CD4+ T lymphocytes. CD4+ T lymphocytes can be used, e.g., if T cells specific for a Class II-restricted antigen are desired.

In some embodiments, the method produces activated T lymphocytes that are CD45RO+. In some embodiments, the method produces activated T lymphocytes that are CD28+. In some embodiments, the method produces activated T lymphocytes that are CD28+CD45RO+. In some embodiments, the method produces activated T lymphocytes that are CD197+. In some embodiments, the method produces activated T lymphocytes that are CD127+. In some embodiments, the method produces activated T lymphocytes that are positive for CD28, CD45RO, CD127 and CD197, or at least any combination of three of the foregoing markers, or at least any combination of two of the foregoing markers. The activated T lymphocytes with any of the foregoing phenotypes can further be CD8+. In some embodiments, any of the foregoing phenotypes that is CD28+ comprises a CD28high phenotype.

In some embodiments, the method produces a population of T cells comprising antigen-specific T cells, wherein at least 65%, 70%, 75%, 80%, 85%, 86%, 87%, 88%, 89%, 90%, 91%, 92%, 93%, 94%, 95%, 96%, 97%, or 98% of the antigen-specific T cells are CD45RO+/CD28High cells, wherein each of the foregoing values can be modified by “about.” Alternatively or in addition, in some embodiments, the method produces a population of T cells wherein at least 1%, 1.5%, 2%, 3,%, 4%, 5%, 6%, 7%, 8%, 9%, 10%, or 11%, of the T cells are antigen-specific T cells; or wherein 1%-2%, 2%-3%, 3%-4%, 4%-5%, 5%-6%, 6%-7%, 7%-8%, 8%-9%, 9%-10%, 10%-11%, or 11%-12% of the T cells are antigen-specific T cells, wherein each of the foregoing values can be modified by “about.” The content of the population of T cells can be determined on the “crude” product of the method following contact with the antigen-presenting surface and optionally further expansion steps, i.e., before/without enriching or separating product T cells having a particular phenotype. The determination of antigen-specificity and/or T cell marker phenotype can exclude dead cells.

In some embodiments, the method provides a population of T cells in which the fraction of T cells that are antigen-specific is increased relative to the starting population.

### Cells and Compositions.

An activated T lymphocyte produced by any method described herein is provided.

In some embodiments, the activated T lymphocytes are CD45RO+. In some embodiments, the activated T lymphocytes are CD28+. In some embodiments, the activated T lymphocytes are CD28+CD45RO+. In some embodiments, the activated T lymphocytes are CD197+. In some embodiments, the activated T lymphocytes are CD127+. In some embodiments, the activated T lymphocytes are positive for CD28, CD45RO, CD127 and CD197, at least any combination of three of the foregoing markers, or at least any combination of two of the foregoing markers. The activated T lymphocytes with any of the foregoing phenotypes can further be CD8+. In some embodiments, any of the foregoing phenotypes that is CD28+ comprises a CD28high phenotype.

In some embodiments, a population of T cells comprising activated T cells produced by any method described herein is provided. The population can have any of the features described above for T cell populations.

In some embodiments, a microfluidic device is provided comprising a population of T cells provided herein. The microfluidic device can be any of the antigen-presenting microfluidic devices or other microfluidic devices described herein.

In some embodiments, a pharmaceutical composition is provided comprising a population of T cells provided herein. The pharmaceutical composition can further comprise, e.g., saline, glucose, and/or Human Serum Albumin. The composition may be an aqueous composition and can be provided in frozen or liquid form. A pharmaceutical composition can be provided as a single dose, e.g., within a syringe, and can comprise 10 million, 100 million, 1 billion, or 10 billion cells. The number of cells administered is indication specific, patient specific (e.g., size of patient), and will also vary with the purity and phenotype of the administered cells.

### Methods of Treatment.

Provided herein is a method of treating a subject in need of treating a cancer; including: obtaining a sample comprising T lymphocytes from the subject; separating the T lymphocytes from other cells in the sample; contacting the T lymphocytes with an antigen-presenting synthetic surface including MHC molecules, wherein the antigen-presenting synthetic surface is prepared according to any method described herein, where the MHC molecules include an antigen specific for the cancer of the subject; producing a plurality of T lymphocytes activated to be specific against the cancer of the subject; separating the plurality of specific activated T lymphocytes from non-activated T lymphocytes; and, introducing the plurality of specific activated T lymphocytes into the subject. Also provided herein is a plurality of specific activated T lymphocytes for use in treating a cancer, wherein the plurality is prepared by a method including: obtaining a sample comprising T lymphocytes from the subject; separating the T lymphocytes from other cells in the sample; contacting the T lymphocytes with an antigen-presenting synthetic surface including MHC molecules according to any method described herein, where the MHC molecules include an antigen specific for the cancer of the subject; producing a plurality of T lymphocytes activated to be specific against the cancer of the subject; and separating the plurality of specific activated T lymphocytes from non-activated T lymphocytes. Also provided herein is the use of a plurality of specific activated T lymphocytes for the manufacture of a medicament for treating a cancer, wherein the plurality is prepared by a method including: obtaining a sample comprising T lymphocytes from the subject; separating the T lymphocytes from other cells in the sample; contacting the T lymphocytes with an antigen-presenting synthetic surface including MHC molecules according to any method described herein, where the MHC molecules include an antigen specific for the cancer of the subject; producing a plurality of T lymphocytes activated to be specific against the cancer of the subject; and separating the plurality of specific activated T lymphocytes from non-activated T lymphocytes. The antigen-presenting synthetic surface may be produced by any proto-antigen-presenting synthetic surface as described herein. In some embodiments, the proto-antigen-presenting surface may include an initial peptide, e.g., in-place peptide, as described herein, or may be identified by any method of initial peptide identification as described herein.

Also provided is a method of treating a subject in need of treating a cancer; including introducing a plurality of specific activated T lymphocytes into the subject, wherein the plurality of specific activated T lymphocytes were produced by a method described herein. Also provided is a method of treating a subject in need of treating a cancer, including introducing a population of specific activated T lymphocytes described herein into the subject. Such methods can further comprise separating activated T lymphocytes from non-activated T lymphocytes. Also provided is a plurality of specific activated T lymphocytes for use in treating a subject in need of treating a cancer, wherein the plurality of specific activated T lymphocytes were produced by a method described herein. Also provided is a population of specific activated T lymphocytes described herein for use in treating a subject in need of treating a cancer. Also provided is a use of a plurality of specific activated T lymphocytes for the manufacture of a medicament for treating a subject in need of treating a cancer, wherein the plurality of specific activated T lymphocytes were produced by a method described herein. Also provided is a use of a population of specific activated T lymphocytes described herein for the manufacture of a medicament for treating a subject in need of treating a cancer. Such a plurality or population of specific activated T lymphocytes can be further prepared by separating activated T lymphocytes from non-activated T lymphocytes.

In some embodiments, separating the plurality of specific activated T lymphocytes may further include detecting surface biomarkers of the specific activated T lymphocytes. In some other embodiments, separating the plurality of specific activated T lymphocytes may further include detecting surface biomarkers of the non-activated T lymphocytes.

In some embodiments, the specific activated T lymphocytes are autologous (i.e., derived from the subject to which they are to be administered).

In various embodiments, the methods or the preparation of the plurality or population of specific activated T lymphocytes may further include rapidly expanding the activated T lymphocytes to provide an expanded population of activated T lymphocytes. In some embodiments, the rapid expansion may be performed after separating the specific activated T lymphocytes from the non-activated T lymphocytes. The generation of sufficient levels of T lymphocytes may be achieved using rapid expansion methods described herein or known in the art. See, e.g., the Examples below; Riddell, U.S. Pat. No. 5,827,642; Riddell et al., U.S. Pat. No. 6,040,177, and Yee and Li, PCT Patent App. Pub. No. WO2009/045308 A2.

Uses of T cells in treatment of human subjects (e.g., for adoptive cell therapy) are known in the art. T cells prepared according to the methods described herein can be used in such methods. For example, adoptive cell therapy using tumor-infiltrating lymphocytes including MART-1 antigen specific T cells have been tested in the clinic (Powell et al., Blood 105:241-250, 2005). Also, administration of T cells coactivated with anti-CD3 monoclonal antibody and IL-2 was described in Chang et al., J. Clinical Oncology 21:884-890, 2003. Additional examples and/or discussion of T cell administration for the treatment of cancer are provided in Dudley et al., Science 298:850-854, 2002; Roszkowski et al., Cancer Res 65(4): 1570-76, 2005; Cooper et al., Blood 101: 1637-44, 2003; Yee, US Patent App. Pub. No. 2006/0269973; Yee and Li, PCT Patent App. Pub. No. WO2009/045308 A2; Gruenberg et al., US Patent App. Pub. No. 2003/0170238; Rosenberg, U.S. Pat. No. 4,690,915; and Alajez et al., Blood 105:4583-89, 2005.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

In some embodiments, the number of cells in the composition is at least 10⁹, or at least 10¹⁰ cells. In some embodiments, a single dose can comprise at least 10 million, 100 million, 1 billion, or 10 billion cells. The number of cells administered is indication specific, patient specific (e.g., size of patient), and will also vary with the purity and phenotype of the administered cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the composition comprising the cells is generally in a volume of a liter or less, can be 500 mls or less, even 250 mls or 100 mls or less. Hence the density (e.g. concentration) of the desired cells may be greater than 10⁶ cells/ml, greater than 10⁷ cells/ml, or 10⁸ cells/ml or greater. The clinically relevant number of immune cells can be apportioned into multiple infusions that cumulatively equal or exceed 10⁹, 10¹⁰ or 10¹¹ cells.

In some embodiments, T lymphocytes described herein or prepared according to a method described herein may be used to confer immunity to individuals against a tumor or cancer cells. By “immunity” is meant a lessening of one or more physical symptoms associated with cancer cells or a tumor against an antigen of which the lymphocytes have been activated. The cells may be administered by infusion, with each infusion greater than 10⁶ cells/ml, greater than 10⁷ cells/ml, or 10⁸ cells/ml or greater or in a range of at least 10⁶ to 10¹⁰ cells/ml, e.g., in the range of at least 10⁷ to 10⁹ cells/ml. The clones may be administered by a single infusion, or by multiple infusions over a range of time. However, since different individuals are expected to vary in responsiveness, the type and amount of cells infused, as well as the number of infusions and the time range over which multiple infusions are given are determined by the attending physician, and can be determined by examination.

Following the transfer of cells back into patients, methods may be employed to maintain their viability by treating patients with cytokines that could include IL-21 and IL-2 (Bear et al., Cancer Immunol. Immunother. 50:269-74, 2001; and Schultze et al., Br. J. Haematol. 113:455-60, 2001). In another embodiment, cells are cultured in the presence of IL-21 before administration to the patient. See, e.g., Yee, US Patent App. Pub. No. 2006/0269973. IL-21 can increase T cell frequency in a population comprising activated T cells to levels that are high enough for expansion and adoptive transfer without further antigen-specific T cell enrichment. Accordingly, such a step can further decrease the time to therapy and/or obviate a need for further selection and/or cloning.

### Kits for Generating an Antigen-Presenting Synthetic Surface.

A kit is provided for generating an antigen-presenting synthetic surface for activating a T lymphocyte (T cell), including: a covalently functionalized surface, such as any covalently functionalized synthetic surface described herein; a primary activating molecule that includes a major histocompatibility complex (MHC) molecule configured to bind to a T cell receptor (TCR), and a first reactive moiety configured to react with or bind to the covalently functionalized surface; and at least one of: an exchange factor (e.g., provided separately from the primary activating molecule); an exchange factor bound to the MHC molecule; or an initial peptide bound to the MHC molecule, wherein the initial peptide is the peptide sequence configured to stabilize the MHC molecule identified by any of the methods of identifying an initial peptide sequence for stabilizing an MHC molecule described herein.

In some embodiments, the exchange factor is provided separately from the primary activating molecule. For example, where an initial peptide (e.g., any of the initial peptides described herein) is bound to the MHC molecule, the exchange factor may be provided as a separate reagent. Alternatively, the exchange factor may be bound to the MHC molecule, e.g., bound in the antigen-binding groove of the MHC molecule. In some embodiments, the covalently functionalized surface comprises a plurality of first coupling agents. The first coupling agent may be a biotin-binding agent. The primary activating molecular ligand may be configured to bind a first subset of the plurality of first coupling agents. The biotin-binding agent may be streptavidin. In some embodiments, each of the plurality of MHC molecules may further include at least one biotin functionality. Other coupling chemistries may be used, as is known in the art, wherein other site specific protein tags may be attached to the MHC protein, which are configured to covalently attach to recognition protein based species attached to the covalently functionalized synthetic surface. These coupling strategies can provide the equivalent site specific and specifically orienting attachment of the MHC molecule as provided by C-terminal biotinylation of the MHC molecule. The covalently functionalized synthetic surface may be a wafer, a bead, at least one inner surface of a microfluidic device, or a tube.

Such a kit may be intended for use with one or more peptide antigens supplied by the user. In some embodiments, the kit further includes a buffer suitable for performing an exchange reaction wherein a peptide antigen displaces the initial peptide or exchange factor and/or instructions for performing an exchange reaction wherein a peptide antigen displaces the initial peptide or exchange factor. Exemplary conditions for performing an exchange reaction wherein a peptide antigen displaces the initial peptide upon catalysis by an exchange factor include those described, e.g., in Saini et al., Proc Nat'l Acad Sci USA (2013) 110, 15383-88, and Saini et al., Proc Nat'l Acad Sci USA (2015) 112, 202-07.

In some embodiments, the kit further comprises a surface-blocking molecule capable of covalently binding to the covalently functionalized synthetic surface. For example, the surface-blocking molecule can be a PEG acid such as (PEG)₄-COOH. Other surface-blocking molecules, such as those described elsewhere herein, may also be provided.

The kit may further comprise at least one co-activating molecule that includes a second reactive moiety configured to react with or bind to the covalently functionalized surface. The kit may further include a reagent including a plurality of co-activating molecules, each configured to bind one of a second subset of the plurality of first coupling agents, e.g., noncovalently or covalently associated biotin-binding agents of the covalently functionalized synthetic surface. In some embodiments, each of the plurality of co-activating molecules may include a biotin functionality. Each of the co-activating molecules may include a T cell receptor (TCR) co-activating molecule, an adjunct TCR activating molecule, or any combination thereof.

In some embodiments, the reagent is provided in individual containers containing the T cell receptor (TCR) co-activating molecule and/or an adjunct TCR activating molecule. Alternatively, the reagent including the plurality of co-activating molecules may be provided in one container containing the TCR co-activating molecules and/or the adjunct TCR activating molecules of the plurality of co-activating molecular ligands in a ratio from about 100:1 to 1:100 mol:mol (e.g. molar ratios). In some embodiments the reagent including the plurality of co-activating molecules includes a mixture of TCR co-activating molecules and adjunct TCR activating molecules wherein the ratio of the TCR co-activating molecules to the adjunct TCR activating molecules of the plurality of co-activating molecular ligands is 100:1 to 90:1, 90:1 to 80:1, 80:1 to 70:1, 70:1 to 60:1, 60:1 to 50:1, 50:1 to 40:1, 40:1 to 30:1, 30:1 to 20:1, 20:1 to 10:1, 10:1 to 1:1, 1:1 to 1:10, 1:10 to 1:20, 1:20 to 1:30, 1:30 to 1:40, 1:40 to 1:50, 1:50 to 1:60, 1:60 to 1:70, 1:70 to 1:80, 1:80 to 1:90, or 1:90 to 1:100 mol:mol, or any ratio selected to be between these values, wherein each of the foregoing values is modified by “about”. In some embodiments, the reagent including a plurality of co-activating molecules contains the TCR co-activating molecules and the adjunct TCR activating molecules of the plurality of co-activating molecular ligands in a ratio from about 20:1 to about 1:20.

In some embodiments, the kit for preparing an antigen presenting synthetic surface may further include a reagent including adhesion stimulatory molecules, wherein each adhesion stimulatory molecule includes a ligand for a cell adhesion receptor including an ICAM protein sequence configured to react with a third subset of the plurality of noncovalently or covalently associated biotin-binding agent functionalities of the covalently functionalized synthetic surface. In some embodiments, the adhesion stimulatory molecule may include a biotin functionality.

In some embodiments, the kit for preparing an antigen presenting synthetic surface may further include a reagent including growth stimulatory molecules, wherein each growth stimulatory molecule may include a growth factor receptor ligand. In some embodiments, the growth factor receptor ligand may include a cytokine or a fragment thereof. In some embodiments, the cytokine may include IL-21 or a fragment thereof. In some embodiments, the growth stimulatory molecule may be attached to a covalently modified bead.

In some embodiments, the kit for preparing an antigen presenting synthetic surface may further include a reagent including one or more additional growth-stimulatory molecules. In some embodiments, the one or more additional growth-stimulatory molecules include IL2 and/or IL7, or fragments thereof. In some embodiments, the growth stimulatory molecule may be attached to a covalently modified bead.

### Kits for Activating T Lymphocytes.

Also provided is a kit for activating T lymphocytes, including a proto-antigen-presenting surface as described herein. The proto-antigen-presenting surface may include an MHC complexed with an initial peptide, wherein the initial peptide is any initial peptide as described herein. The kit can further comprise instructions for performing an exchange reaction wherein a peptide antigen displaces the initial peptide or exchange factor bound to the primary activating ligand of the proto-antigen-presenting synthetic surface and/or a buffer suitable for performing an exchange reaction wherein a peptide antigen displaces the initial peptide or exchange factor. Such a kit may be intended for use with one or more peptide antigens supplied by the user.

The kit can further includes a plurality of co-activating molecules. Each of the plurality of co-activating molecules is selected from a TCR co-activating molecule and an adjunct TCR activating molecule as described herein. Each of the plurality of co-activating molecules can also include a second reactive moiety which is configured to react with or bind to the proto-antigen-presenting synthetic surface.

In some embodiments, the proto-antigen-presenting synthetic surface presents a plurality of co-activating molecular ligand, each is selected from a TCR co-activating molecule and an adjunct TCR activating molecule as described herein. The ratio of the TCR co-activating molecule and an adjunct TCR activating molecule can be as described herein, for example, about 1:10 to about 2:1, about 1:5 to about 2:1, about 1:2 to about 2:1, about 1:10 to about 1:1, about 1:5 to about 1:1, about 1:1 to about 2:1, or about 1:2 to about 1:1, or any ratio selected to be between these values.

In some embodiments, the kit can further include a plurality of adhesion stimulatory molecule or the proto-antigen-presenting synthetic surface presents a plurality of adhesion stimulatory molecular ligands. Such adhesion stimulatory molecule or ligand can be a ligand for a cell adhesion receptor including an ICAM protein sequence or fragment thereof which retains activity of the parent molecule. The fragment may retain at least 10%, 25%, 40%, 50%, 60%, 75%, 80% or more of the level of activity of the parent adhesion receptor, and has a level of activity sufficient to act as an adhesion stimulatory molecular ligand. Other types of adhesion stimulatory molecular ligands can be as described herein.

In some embodiments, the kit can further include a plurality surface-blocking molecules or the proto-antigen-presenting surface presents a plurality surface-blocking molecular ligands. The surface-blocking molecules or surface-blocking molecular ligands can be as described herein. In some embodiment, each of the plurality of surface-blocking molecular ligands includes a polyethylene glycol (PEG) moiety, a carboxylic acid moiety, or a combination thereof.

The kit can further comprise growth stimulatory molecules, wherein each growth stimulatory molecule may include a growth factor receptor ligand. The growth stimulatory molecules can be provided as free molecules, attached to the antigen presenting synthetic surface (in the same or a different region than the primary activating molecular ligand), or attached to a different covalently modified synthetic surface. For example, the kit can further comprise a plurality of covalently modified beads comprising a growth stimulatory molecule. In some embodiments, the growth factor receptor ligand molecule may include a cytokine or a fragment thereof. In some embodiments, the growth factor receptor ligand may include IL-21. In other embodiments, the kit may include one or more additional (e.g., a second or second and third) growth stimulatory molecules. In some embodiments, the one or more additional growth stimulatory molecules may include IL-2 and/or IL-7, or fragments thereof. Additional growth stimulatory molecules can be provided as a free molecule, attached to the antigen presenting synthetic surface (in the same or a different region than the primary activating molecular ligand), or attached to a different covalently modified synthetic surface, such as a bead.

### Methods of Screening a Plurality of Peptide Antigens for T Cell Activation.

Also provided herein are methods of screening a plurality of peptide antigens for T cell activation. The proto-antigen-presenting surfaces can be used to rapidly generate antigen-presenting surfaces comprising various peptide antigens of interest, e.g., which may be immunogenic in the context of T cell activation. Such methods can comprise reacting a plurality of different peptide antigens with a plurality of proto-antigen-presenting surfaces, such as any proto-antigen-presenting surfaces described herein, thereby substantially displacing the exchange factors or initial peptides and forming a plurality of antigen-presenting surfaces; contacting a plurality of T cells with the antigen-presenting surfaces; and monitoring the T cells for activation, wherein activation of a T cell indicates that a peptide antigen associated with the surface with which the T cell was contacted is able to contribute to T cell activation.

The proto-antigen-presenting surfaces can be any of the surface types described herein, such as beads, surfaces of a microfluidic device, well plate, etc. To be clear, where the surfaces are surfaces of a larger article such as a microfluidic device or well plate, the plurality of surfaces may be surfaces at different locations on a single article (e.g., well plate or microfluidic device) or surfaces of different articles. For example, a plurality of proto-antigen-presenting surfaces of a microfluidic device can be separated by regions of non-antigen-presenting surface. In some embodiments, the proto-antigen-presenting surfaces can be in different sequestration pens of the microfluidic device while the non-antigen-presenting surface can be in a channel or region connecting the openings of the sequestration pens.

In some embodiments, the proto-antigen-presenting surfaces are reacted separately with the plurality of different peptide antigens, thereby generating a plurality of different antigen-presenting surfaces. In this approach, individual surfaces comprise an individual peptide antigen and thus the extent of T cell activation attributable to that surface provides a readout of the immunogenicity of that particular peptide antigen.

In some embodiments, the proto-antigen-presenting surfaces are reacted separately with pools of members of the plurality of different peptide antigens, thereby generating a plurality of different antigen-presenting surfaces. In this approach, the individual antigen-presenting surfaces comprise more than one peptide antigen, and thus the extent of T cell activation attributable to that surface provides a readout of the immunogenicity of one or more of the peptide antigens associated with the surface. The particular peptide antigen or antigens responsible for the T cell activation can be identified by further analysis, e.g., using the approach of preparing individual surfaces comprising individual antigens described above.

The pools can be overlapping or non-overlapping pools. Overlapping pools provide more information about the individual peptide antigens being tested, in that when activation occurs with a subset of tested surfaces, it can be possible to identify a subset of peptide antigens most likely to be responsible based on which antigens were present on the surfaces that exhibited activation. Non-overlapping pools provide more bandwidth, in that a greater total number of peptide antigens can be tested using a given number of pools, pool sizes, and surfaces when the pools are non-overlapping. A possible workflow for identifying immunogenic peptide antigens from an initial candidate set is to first perform screening using non-overlapping pools, then generate overlapping sub-pools from members of the initial pool sets that showed activation, and then screen individual peptide antigens that the overlapping sub-pool results indicate are potentially immunogenic.

In some embodiments, the method can further include (i) determining that T cells contacted with a pool of antigen-presenting beads (or one or more of the antigen-presenting surfaces of the microfluidic device, or one or more of the antigen-presenting surfaces one or more well plates) underwent activation and (ii) contacting additional T cells with a member or subset of members of the pool, or with one or more additional antigen-presenting surfaces including the same peptide antigen or peptide antigens as a member or subset of members of the pool.

Where beads are used as the surface, T cells may be contacted separately with members of the plurality of different antigen-presenting beads. For example, an individual bead can be contacted with one or more T cells, e.g., in a chamber, such as a sequestration pen or well, while other individual beads are contacted with other T cells in other chambers. Alternatively, T cells can be contacted with a pool of the different antigen-presenting beads. In another alternative, T cells can be contacted with a plurality of pools of the different antigen-presenting beads. For example, T cells in a first chamber (such as a sequestration pen or well) can be contacted with a first pool and T cells in a second chamber (such as a sequestration pen or well) can be contacted with a second pool. The first and second pools may be overlapping or non-overlapping.

In some embodiments, the plurality of proto-antigen-presenting surfaces is a plurality of proto-antigen-presenting surfaces in wells of one or more well plates. In such embodiments, the wells may also comprise non-antigen-presenting regions. This can be beneficial through reducing the amount of reagents needed to prepare the antigen-presenting surfaces within the wells and/or through avoiding overstimulation of the T cells.

Monitoring the T cells for activation in any screening method described herein may comprise detecting one or more of various markers consistent with activation (e.g., in combination with being antigen-specific). For example, T cells that are CD45RO+, CD28+, CD28^{High}, CD127+, and/or CD197+ may be detected. In some embodiments, the T cells are or include CD8+ T cells.

### Methods of Analyzing Stability of a Complex Comprising a Major Histocompatibility Complex (MHC) Molecule and a Peptide Antigen

Also provided herein are methods of analyzing stability of a complex comprising a major histocompatibility complex (MHC) molecule configured to bind to a T cell receptor (TCR) and a peptide antigen. In some embodiments, the method comprises contacting a plurality of the MHC molecules with an exchange factor and the peptide antigen, thereby forming peptide antigen-bound MHC molecules. An initial peptide (e.g., as described elsewhere herein) may be bound to the MHC molecule before contact with the peptide antigen and exchange factor. The contacting step may be performed over a period of time sufficient for the peptide antigen to substantially displace the initial peptide from the MHC molecules and/or become for the MHC molecules to become bound to the peptide antigen, e.g., at room temperature for about 4 hours or more, or under refrigeration (e.g., about 4° C.) overnight or for about 10, 12, or 15 hours or more.

In some embodiments, a plurality of primary activating molecular ligands comprise the MHC molecules and the plurality of primary activating molecular ligands are specifically bound to a covalently functionalized synthetic surface. In other embodiments, (1) a plurality of primary activating molecules comprise the MHC molecules and first reactive moieties or (2) a plurality of primary activating molecules is prepared by adding first reactive moieties to the MHC molecules; and the method further comprises reacting the first reactive moieties of the plurality of primary activating molecules with a first plurality of binding moieties disposed on a covalently functionalized synthetic surface. The method further comprises measuring total binding and/or an extent of dissociation of the peptide antigen from the MHC molecule. The covalently functionalized surface may be any such surface described herein. In some embodiments, the covalently functionalized surface is the surface of a bead.

Measuring the total binding and/or extent of dissociation can comprise, e.g., measuring binding of an agent (e.g., antibody, such as that produced by Biolegend Clone W6/32) to the MHC molecule, wherein the agent specifically binds to (i) the initial peptide, and/or (ii) a peptide-bound conformation of the MHC molecule. The peptide-bound conformation is the conformation that exists when a peptide (e.g., an antigenic peptide or an initial peptide) is bound in the peptide binding cleft formed by the alpha chain of the MHC molecule. Typically, for a MHC Class I molecule, the peptide binding cleft binds to peptides having a length of 8-10 amino acid residues, whereas for an MHC Class II molecule, the peptide binding cleft binds to peptides having a length of 13-18 amino acid residues. In some embodiments, a beta microglobulin (e.g., beta-2-microglobulin) is part of the MHC molecule in its peptide-bound conformation. The beta microglobulin may dissociate from the MHC molecule as part of a transition to a peptide-unbound conformation, e.g., simultaneous with or upon dissociation of the peptide antigen from the MHC molecule. Thus, the agent can be used to discriminate between MHC molecules that retain the peptide antigen and those that do not. The agent may be labeled directly (e.g., by conjugation to a label) or indirectly (e.g., by binding of a secondary antibody comprising a label). The label may be a fluorescent label.

Various approaches for measuring label (e.g., fluorescence) levels associated with a surface may be employed. In some embodiments, measuring the total binding and/or extent of dissociation comprises performing flow cytometry. Flow cytometry can rapidly and accurately quantify the amount of a labeled agent as discussed above that is bound to an MHC molecule associated with an appropriate solid support, such as a bead. Observing changes in such binding over time can permit analysis of stability, e.g., in terms of an appropriate kinetic parameter, such as a half-life or off-rate.

Such methods can be useful to evaluate the suitability of a peptide antigen for preparing and using an antigen-presenting surface as described herein. Peptide antigens that form more stable complexes with MHC molecules can provide more effective stimulation of T cells because the complexes are longer lived and therefore have more time to interact with the T cells. For example, in some embodiments, a peptide antigen is identified as being capable of forming a complex with an MHC molecule that has a half-life of at least about 0.1, 0.2, 0.4, 1.5, 2, 4, 6, 8, 10, 12, 14, 16, 18, 20, 24, 28, 32, 36, 48, or 72 hours, or in the range of about 4-10, 4-12, 8-16, 10-15, 12-20, 15-20, 20-25, 20-28, 24-32, 25-30, 28-36, 30-35, 32-40, 35-40, 36-48, or 48-72 hours.

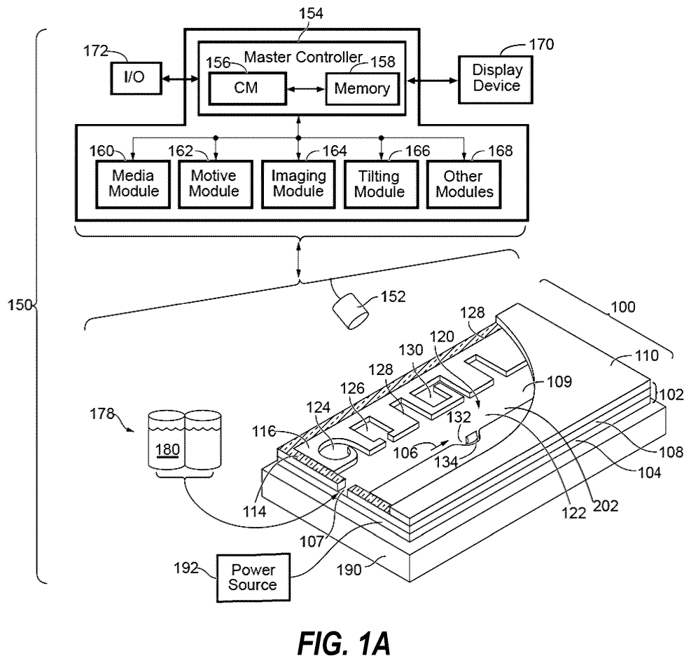

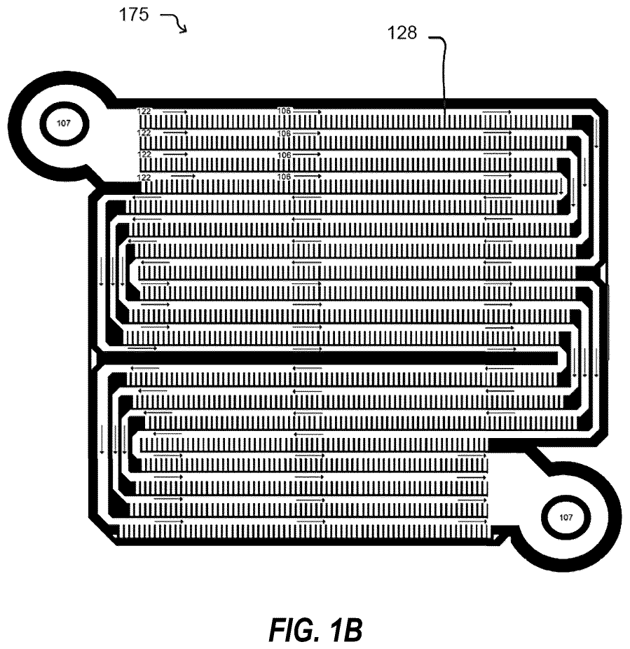

Microfluidic device/system feature cross-applicability. It should be appreciated that various features of microfluidic devices, systems, and motive technologies described herein may be combinable or interchangeable. For example, features described herein with reference to the microfluidic device 100, 175, 200, 300, 320, 400, 450, 520 and system attributes as described in FIGS. 1A-5B may be combinable or interchangeable.

Microfluidic devices. FIG. 1A illustrates an example of a microfluidic device 100. A perspective view of the microfluidic device 100 is shown having a partial cut-away of its cover 110 to provide a partial view into the microfluidic device 100. The microfluidic device 100 generally comprises a microfluidic circuit 120 comprising a flow path 106 through which a fluidic medium 180 can flow, optionally carrying one or more micro-objects (not shown) into and/or through the microfluidic circuit 120.

As generally illustrated in FIG. 1A, the microfluidic circuit 120 is defined by an enclosure 102. Although the enclosure 102 can be physically structured in different configurations, in the example shown in FIG. 1A the enclosure 102 is depicted as comprising a support structure 104 (e.g., a base), a microfluidic circuit structure 108, and a cover 110. The support structure 104, microfluidic circuit structure 108, and cover 110 can be attached to each other. For example, the microfluidic circuit structure 108 can be disposed on an inner surface 109 of the support structure 104, and the cover 110 can be disposed over the microfluidic circuit structure 108. Together with the support structure 104 and cover 110, the microfluidic circuit structure 108 can define the elements of the microfluidic circuit 120, forming a three-layer structure.

The support structure 104 can be at the bottom and the cover 110 at the top of the microfluidic circuit 120 as illustrated in FIG. 1A. Alternatively, the support structure 104 and the cover 110 can be configured in other orientations. For example, the support structure 104 can be at the top and the cover 110 at the bottom of the microfluidic circuit 120. Regardless, there can be one or more ports 107 each comprising a passage into or out of the enclosure 102. Examples of a passage include a valve, a gate, a pass-through hole, or the like. As illustrated, port 107 is a pass-through hole created by a gap in the microfluidic circuit structure 108. However, the port 107 can be situated in other components of the enclosure 102, such as the cover 110. Only one port 107 is illustrated in FIG. 1A but the microfluidic circuit 120 can have two or more ports 107. For example, there can be a first port 107 that functions as an inlet for fluid entering the microfluidic circuit 120, and there can be a second port 107 that functions as an outlet for fluid exiting the microfluidic circuit 120. Whether a port 107 function as an inlet or an outlet can depend upon the direction that fluid flows through flow path 106.

The support structure 104 can comprise one or more electrodes (not shown) and a substrate or a plurality of interconnected substrates. For example, the support structure 104 can comprise one or more semiconductor substrates, each of which is electrically connected to an electrode (e.g., all or a subset of the semiconductor substrates can be electrically connected to a single electrode). The support structure 104 can further comprise a printed circuit board assembly (“PCBA”). For example, the semiconductor substrate(s) can be mounted on a PCBA.

The microfluidic circuit structure 108 can define circuit elements of the microfluidic circuit 120. Such circuit elements can comprise spaces or regions that can be fluidly interconnected when microfluidic circuit 120 is filled with fluid, such as flow regions (which may include or be one or more flow channels), chambers (which class of circuit elements may also include sub-classes including sequestration pens), traps, and the like. Circuit elements can also include barriers, and the like. The barrier defining the microfluidic sequestration pen can extend from a surface of the base of the microfluidic device to a surface of the cover of the microfluidic device. In the microfluidic circuit 120 illustrated in FIG. 1A, the microfluidic circuit structure 108 comprises a frame 114 and a microfluidic circuit material 116. The frame 114 can partially or completely enclose the microfluidic circuit material 116. The frame 114 can be, for example, a relatively rigid structure substantially surrounding the microfluidic circuit material 116. For example, the frame 114 can comprise a metal material. However, the microfluidic circuit structure need not include a frame 114. For example, the microfluidic circuit structure can consist of (or consist essentially of) the microfluidic circuit material 116.

The microfluidic circuit material 116 can be patterned with cavities or the like to define the circuit elements and interconnections of the microfluidic circuit 120, such as chambers, pens and microfluidic channels. The microfluidic circuit material 116 can comprise a flexible material, such as a flexible polymer (e.g., rubber, plastic, elastomer, silicone, polydimethylsiloxane (“PDMS”), or the like), which can be gas permeable. Other examples of materials that can form the microfluidic circuit material 116 include molded glass, an etchable material such as silicone (e.g., photo-patternable silicone or “PPS”), photo-resist (e.g., SU8), or the like. In some embodiments, such materials—and thus the microfluidic circuit material 116—can be rigid and/or substantially impermeable to gas. Regardless, microfluidic circuit material 116 can be disposed on the support structure 104 and inside the frame 114.

The microfluidic circuit 120 can include a flow region in which one or more chambers can be disposed and/or fluidically connected thereto. A chamber can have one or more openings fluidically connecting the chamber with one or more flow regions. In some embodiments, a flow region comprises or corresponds to a microfluidic channel 122. Although a single microfluidic circuit 120 is illustrated in FIG. 1A, suitable microfluidic devices can include a plurality (e.g., 2 or 3) of such microfluidic circuits. In some embodiments, the microfluidic device 100 can be configured to be a nanofluidic device. As illustrated in FIG. 1A, the microfluidic circuit 120 may include a plurality of microfluidic sequestration pens 124, 126, 128, and 130, where each sequestration pens may have one or more openings. In some embodiments of sequestration pens, a sequestration pen may have only a single opening in fluidic communication with the flow path 106. In some other embodiments, a sequestration pen may have more than one opening in fluidic communication with the flow path 106, e.g., n number of openings, but with n−1 openings that are valved, such that all but one opening is closable. When all the valved openings are closed, the sequestration pen limits exchange of materials from the flow region into the sequestration pen to occur only by diffusion. In some embodiments, the sequestration pens comprise various features and structures (e.g., isolation regions) that have been optimized for retaining micro-objects within the sequestration pen (and therefore within a microfluidic device such as microfluidic device 100) even when a medium 180 is flowing through the flow path 106.

The cover 110 can be an integral part of the frame 114 and/or the microfluidic circuit material 116. Alternatively, the cover 110 can be a structurally distinct element, as illustrated in FIG. 1A. The cover 110 can comprise the same or different materials than the frame 114 and/or the microfluidic circuit material 116. In some embodiments, the cover 110 can be an integral part of the microfluidic circuit material 116. Similarly, the support structure 104 can be a separate structure from the frame 114 or microfluidic circuit material 116 as illustrated, or an integral part of the frame 114 or microfluidic circuit material 116. Likewise, the frame 114 and microfluidic circuit material 116 can be separate structures as shown in FIG. 1A or integral portions of the same structure. Regardless of the various possible integrations, the microfluidic device can retain a three-layer structure that includes a base layer and a cover layer that sandwich a middle layer in which the microfluidic circuit 120 is located.

In some embodiments, the cover 110 can comprise a rigid material. The rigid material may be glass or a material with similar properties. In some embodiments, the cover 110 can comprise a deformable material. The deformable material can be a polymer, such as PDMS. In some embodiments, the cover 110 can comprise both rigid and deformable materials. For example, one or more portions of cover 110 (e.g., one or more portions positioned over sequestration pens 124, 126, 128, 130) can comprise a deformable material that interfaces with rigid materials of the cover 110. Microfluidic devices having covers that include both rigid and deformable materials have been described, for example, in U.S. Pat. No. 10,058,865 (Breinlinger et al.), the contents of which are incorporated herein by reference. In some embodiments, the cover 110 can further include one or more electrodes. The one or more electrodes can comprise a conductive oxide, such as indium-tin-oxide (ITO), which may be coated on glass or a similarly insulating material. Alternatively, the one or more electrodes can be flexible electrodes, such as single-walled nanotubes, multi-walled nanotubes, nanowires, clusters of electrically conductive nanoparticles, or combinations thereof, embedded in a deformable material, such as a polymer (e.g., PDMS). Flexible electrodes that can be used in microfluidic devices have been described, for example, in U.S. Pat. No. 9,227,200 (Chiou et al.), the contents of which are incorporated herein by reference. In some embodiments, the cover 110 and/or the support structure 104 can be transparent to light. The cover 110 may also include at least one material that is gas permeable (e.g., PDMS or PPS).

In the example shown in FIG. 1A, the microfluidic circuit 120 is illustrated as comprising a microfluidic channel 122 and sequestration pens 124, 126, 128, 130. Each pen comprises an opening to channel 122, but otherwise is enclosed such that the pens can substantially isolate micro-objects inside the pen from fluidic medium 180 and/or micro-objects in the flow path 106 of channel 122 or in other pens. The walls of the sequestration pen extend from the inner surface 109 of the base to the inside surface of the cover 110 to provide enclosure. The opening of the sequestration pen to the microfluidic channel 122 is oriented at an angle to the flow 106 of fluidic medium 180 such that flow 106 is not directed into the pens. The vector of bulk fluid flow in channel 122 may be tangential or parallel to the plane of the opening of the sequestration pen, and is not directed into the opening of the pen. In some instances, pens 124, 126, 128, 130 are configured to physically isolate one or more micro-objects within the microfluidic circuit 120. Sequestration pens in accordance with the present disclosure can comprise various shapes, surfaces and features that are optimized for use with DEP, OET, OEW, fluid flow, magnetic forces, centripetal, and/or gravitational forces, as will be discussed and shown in detail below.

The microfluidic circuit 120 may comprise any number of microfluidic sequestration pens. Although five sequestration pens are shown, microfluidic circuit 120 may have fewer or more sequestration pens. As shown, microfluidic sequestration pens 124, 126, 128, and 130 of microfluidic circuit 120 each comprise differing features and shapes which may provide one or more benefits useful for maintaining, isolating, assaying or culturing biological micro-objects. In some embodiments, the microfluidic circuit 120 comprises a plurality of identical microfluidic sequestration pens.

In the embodiment illustrated in FIG. 1A, a single flow path 106 containing a single channel 122 is shown. However, other embodiments may contain multiple channels 122 within a single flow path 106, as shown in FIG. 1B. The microfluidic circuit 120 further comprises an inlet valve or port 107 in fluid communication with the flow path 106, whereby fluidic medium 180 can access the flow path 106 (and channel 122). In some instances, the flow path 106 comprises a substantially straight path. In other instances, the flow path 106 is arranged in a non-linear or winding manner, such as a zigzag pattern, whereby the flow path 106 travels across the microfluidic device 100 two or more times, e.g., in alternating directions. The flow in the flow path 106 may proceed from inlet to outlet or may be reversed and proceed from outlet to inlet.

One example of a multi-channel device, microfluidic device 175, is shown in FIG. 1B, which may be like microfluidic device 100 in other respects. Microfluidic device 175 and its constituent circuit elements (e.g., channels 122 and sequestration pens 128) may have any of the dimensions discussed herein. The microfluidic circuit illustrated in FIG. 1B has two inlet/outlet ports 107 and a flow path 106 containing four distinct channels 122. The number of channels into which the microfluidic circuit is sub-divided may be chosen to reduce fluidic resistance. For example, the microfluidic circuit may include 1, 2, 3, 4, 5, 6, 7, 8, 9, 10, or more channels to provide a selected range of fluidic resistance. Microfluidic device 175 further comprises a plurality of sequestration pens opening off of each channel 122, where each of the sequestration pens is similar to sequestration pen 128 of FIG. 1A, and may have any of the dimensions or functions of any sequestration pen as described herein. However, the sequestration pens of microfluidic device 175 can have different shapes, such as any of the shapes of sequestration pens 124, 126, or 130 of FIG. 1A or as described anywhere else herein. Moreover, microfluidic device 175 can include sequestration pens having a mixture of different shapes. In some instances, a plurality of sequestration pens is configured (e.g., relative to a channel 122) such that the sequestration pens can be loaded with target micro-objects in parallel.

Returning to FIG. 1A, microfluidic circuit 120 further may include one or more optional micro-object traps 132. The optional traps 132 may be formed in a wall forming the boundary of a channel 122, and may be positioned opposite an opening of one or more of the microfluidic sequestration pens 124, 126, 128, 130. The optional traps 132 may be configured to receive or capture a single micro-object from the flow path 106, or may be configured to receive or capture a plurality of micro-objects from the flow path 106. In some instances, the optional traps 132 comprise a volume approximately equal to the volume of a single target micro-object. In some instances, the trap 132 comprises a side passage 134 that is smaller than the target micro-object in order to facilitate flow through the trap 132.

Sequestration pens. The microfluidic devices described herein may include one or more sequestration pens, where each sequestration pen is suitable for holding one or more micro-objects (e.g., biological cells, or groups of cells that are associated together). The sequestration pens may be disposed within and open to a flow region, which in some embodiments is a microfluidic channel. Each of the sequestration pens can have one or more openings for fluidic communication to one or more microfluidic channels. In some embodiments, a sequestration pen may have only one opening to a microfluidic channel.

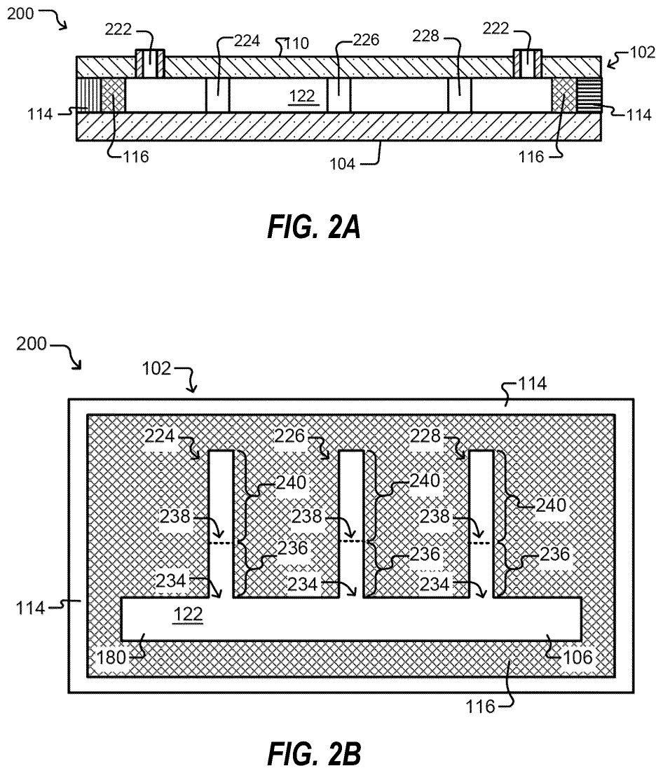

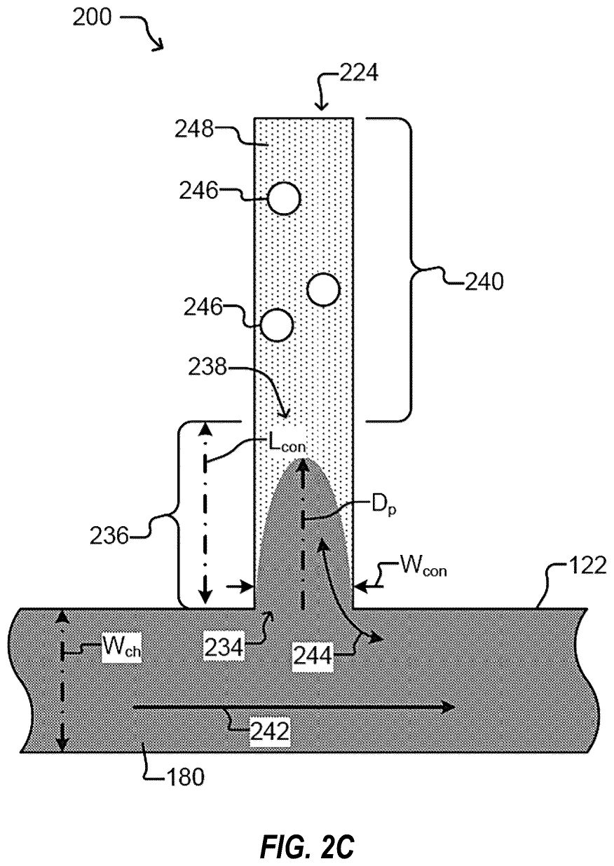

FIGS. 2A-2C show sequestration pens 224, 226, and 228 of a microfluidic device 200, which may be like sequestration pen 128 of FIG. 1A. Each sequestration pen 224, 226, and 228 can comprise an isolation region 240 and a connection region 236 fluidically connecting the isolation region 240 to a flow region, which may, in some embodiments include a microfluidic channel, such as channel 122. The connection region 236 can comprise a proximal opening 234 to the flow region (e.g., microfluidic channel 122) and a distal opening 238 to the isolation region 240. The connection region 236 can be configured so that the maximum penetration depth of a flow of a fluidic medium (not shown) flowing in the microfluidic channel 122 past the sequestration pen 224, 226, and 228 does not extend into the isolation region 240, as discussed below for FIG. 2C. In some embodiments, streamlines from the flow in the microfluidic channel do not enter the isolation region. Thus, due to the connection region 236, a micro-object (not shown) or other material (not shown) disposed in the isolation region 240 of a sequestration pen 224, 226, and 228 can be isolated from, and not substantially affected by, a flow of fluidic medium 180 in the microfluidic channel 122.

The sequestration pens 224, 226, and 228 of FIGS. 2A-2C each have a single opening which opens directly to the microfluidic channel 122. The opening of the sequestration pen may open laterally from the microfluidic channel 122, as shown in FIG. 2A, which depicts a vertical cross-section of microfluidic device 200. FIG. 2B shows a horizontal cross-section of microfluidic device 200. An electrode activation substrate 206 can underlie both the microfluidic channel 122 and the sequestration pens 224, 226, and 228. The upper surface of the electrode activation substrate 206 within an enclosure of a sequestration pen, forming the floor of the sequestration pen, can be disposed at the same level or substantially the same level of the upper surface the of electrode activation substrate 206 within the microfluidic channel 122 (or flow region if a channel is not present), forming the floor of the flow channel (or flow region, respectively) of the microfluidic device. The electrode activation substrate 206 may be featureless or may have an irregular or patterned surface that varies from its highest elevation to its lowest depression by less than about 3 micrometers (microns), 2.5 microns, 2 microns, 1.5 microns, 1 micron, 0.9 microns, 0.5 microns, 0.4 microns, 0.2 microns, 0.1 microns or less. The variation of elevation in the upper surface of the substrate across both the microfluidic channel 122 (or flow region) and sequestration pens may be equal to or less than about 10%, 7%, 5%, 3%, 2%, 1%. 0.9%, 0.8%, 0.5%, 0.3% or 0.1% of the height of the walls of the sequestration pen. Alternatively, the variation of elevation in the upper surface of the substrate across both the microfluidic channel 122 (or flow region) and sequestration pens may be equal to or less than about 2%, 1%. 0.9%, 0.8%, 0.5%, 0.3%, 0.2%, or 0.1% of the height of the substrate. While described in detail for the microfluidic device 200, this may also apply to any of the microfluidic devices described herein.

The microfluidic channel 122 and connection region 236 can be examples of swept regions, and the isolation regions 240 of the sequestration pens 224, 226, and 228 can be examples of unswept regions. Sequestration pens like 224, 226, 228 have isolation regions wherein each isolation region has only one opening, which opens to the connection region of the sequestration pen. Fluidic media exchange in and out of the isolation region so configured can be limited to occurring substantially only by diffusion. As noted, the microfluidic channel 122 and sequestration pens 224, 226, and 228 can be configured to contain one or more fluidic media 180. In the example shown in FIGS. 2A-2B, ports 222 are connected to the microfluidic channel 122 and allow the fluidic medium 180 to be introduced into or removed from the microfluidic device 200. Prior to introduction of the fluidic medium 180, the microfluidic device may be primed with a gas such as carbon dioxide gas. Once the microfluidic device 200 contains the fluidic medium 180, the flow 242 (see FIG. 2C) of fluidic medium 180 in the microfluidic channel 122 can be selectively generated and stopped. For example, as shown, the ports 222 can be disposed at different locations (e.g., opposite ends) of the flow region (microfluidic channel 122), and a flow 242 of the fluidic medium can be created from one port 222 functioning as an inlet to another port 222 functioning as an outlet.

FIG. 2C illustrates a detailed view of an example of a sequestration pen 224, which may contain one or more micro-objects 246, according to some embodiments. The flow 242 of fluidic medium 180 in the microfluidic channel 122 past the proximal opening 234 of the connection region 236 of sequestration pen 224 can cause a secondary flow 244 of the fluidic medium 180 into and out of the sequestration pen 224. To sequester the micro-objects 246 in the isolation region 240 of the sequestration pen 224 from the secondary flow 244, the length L_{con} of the connection region 236 of the sequestration pen 224 (i.e., from the proximal opening 234 to the distal opening 238) should be greater than the penetration depth Dₚ of the secondary flow 244 into the connection region 236. The penetration depth Dₚ depends upon a number of factors, including the shape of the microfluidic channel 122, which may be defined by a width W_{con} of the connection region 236 at the proximal opening 234; a width W_{ch} of the microfluidic channel 122 at the proximal opening 234; a height H_{ch} of the channel 122 at the proximal opening 234; and the width of the distal opening 238 of the connection region 236. Of these factors, the width W_{con} of the connection region 236 at the proximal opening 234 and the height H_{ch} of the channel 122 at the proximal opening 234 tend to be the most significant. In addition, the penetration depth Dₚ can be influenced by the velocity of the fluidic medium 180 in the channel 122 and the viscosity of fluidic medium 180. However, these factors (i.e., velocity and viscosity) can vary widely without dramatic changes in penetration depth Dₚ. For example, for a microfluidic chip 200 having a width W_{con} of the connection region 236 at the proximal opening 234 of about 50 microns, a height H_{ch} of the channel 122 at the proximal opening 122 of about 40 microns, and a width W_{ch} of the microfluidic channel 122 at the proximal opening 122 of about 100 microns to about 150 microns, the penetration depth Dₚ of the secondary flow 244 ranges from less than 1.0 times W_{con} (i.e., less than 50 microns) at a flow rate of 0.1 microliters/sec to about 2.0 times W_{con} (i.e., about 100 microns) at a flow rate of 20 microliters/sec, which represents an increase in Dₚ of only about 2.5-fold over a 200-fold increase in the velocity of the fluidic medium 180.

In some embodiments, the walls of the microfluidic channel 122 and sequestration pen 224, 226, or 228 can be oriented as follows with respect to the vector of the flow 242 of fluidic medium 180 in the microfluidic channel 122: the microfluidic channel width W_{ch} (or cross-sectional area of the microfluidic channel 122) can be substantially perpendicular to the flow 242 of medium 180; the width W_{con} (or cross-sectional area) of the connection region 236 at opening 234 can be substantially parallel to the flow 242 of medium 180 in the microfluidic channel 122; and/or the length L_{con} of the connection region can be substantially perpendicular to the flow 242 of medium 180 in the microfluidic channel 122. The foregoing are examples only, and the relative position of the microfluidic channel 122 and sequestration pens 224, 226 and 228 can be in other orientations with respect to each other.

In some embodiments, for a given microfluidic device, the configurations of the microfluidic channel 122 and the opening 234 may be fixed, whereas the rate of flow 242 of fluidic medium 180 in the microfluidic channel 122 may be variable. Accordingly, for each sequestration pen 224, a maximal velocity Vₘₐₓ for the flow 242 of fluidic medium 180 in channel 122 may be identified that ensures that the penetration depth Dₚ of the secondary flow 244 does not exceed the length L_{con} of the connection region 236. When Vₘₐₓ is not exceeded, the resulting secondary flow 244 can be wholly contained within the connection region 236 and does not enter the isolation region 240. Thus, the flow 242 of fluidic medium 180 in the microfluidic channel 122 (swept region) is prevented from drawing micro-objects 246 out of the isolation region 240, which is an unswept region of the microfluidic circuit, resulting in the micro-objects 246 being retained within the isolation region 240. Accordingly, selection of microfluidic circuit element dimensions and further selection of the operating parameters (e.g., velocity of fluidic medium 180) can prevent contamination of the isolation region 240 of sequestration pen 224 by materials from the microfluidic channel 122 or another sequestration pen 226 or 228. It should be noted, however, that for many microfluidic chip configurations, there is no need to worry about Vₘₐₓ per se, because the chip will break from the pressure associated with flowing fluidic medium 180 at high velocity through the chip before Vₘₐₓ can be achieved.

Components (not shown) in the first fluidic medium 180 in the microfluidic channel 122 can mix with the second fluidic medium 248 in the isolation region 240 substantially only by diffusion of components of the first medium 180 from the microfluidic channel 122 through the connection region 236 and into the second fluidic medium 248 in the isolation region 240. Similarly, components (not shown) of the second medium 248 in the isolation region 240 can mix with the first medium 180 in the microfluidic channel 122 substantially only by diffusion of components of the second medium 248 from the isolation region 240 through the connection region 236 and into the first medium 180 in the microfluidic channel 122. In some embodiments, the extent of fluidic medium exchange between the isolation region of a sequestration pen and the flow region by diffusion is greater than about 90%, 91%, 92%, 93%, 94% 95%, 96%, 97%, 98%, or greater than about 99% of fluidic exchange.

In some embodiments, the first medium 180 can be the same medium or a different medium than the second medium 248. In some embodiments, the first medium 180 and the second medium 248 can start out being the same, then become different (e.g., through conditioning of the second medium 248 by one or more cells in the isolation region 240, or by changing the medium 180 flowing through the microfluidic channel 122).

As illustrated in FIG. 2C, the width W_{con} of the connection region 236 can be uniform from the proximal opening 234 to the distal opening 238. The width W_{con} of the connection region 236 at the distal opening 238 can be any of the values identified herein for the width W_{con} of the connection region 236 at the proximal opening 234. In some embodiments, the width of the isolation region 240 at the distal opening 238 can be substantially the same as the width W_{con} of the connection region 236 at the proximal opening 234. Alternatively, the width W_{con} of the connection region 236 at the distal opening 238 can be different (e.g., larger or smaller) than the width W_{con} of the connection region 236 at the proximal opening 234. In some embodiments, the width W_{con} of the connection region 236 may be narrowed or widened between the proximal opening 234 and distal opening 238. For example, the connection region 236 may be narrowed or widened between the proximal opening and the distal opening, using a variety of different geometries (e.g., chamfering the connection region, beveling the connection region). Further, any part or subpart of the connection region 236 may be narrowed or widened (e.g., a portion of the connection region adjacent to the proximal opening 234).

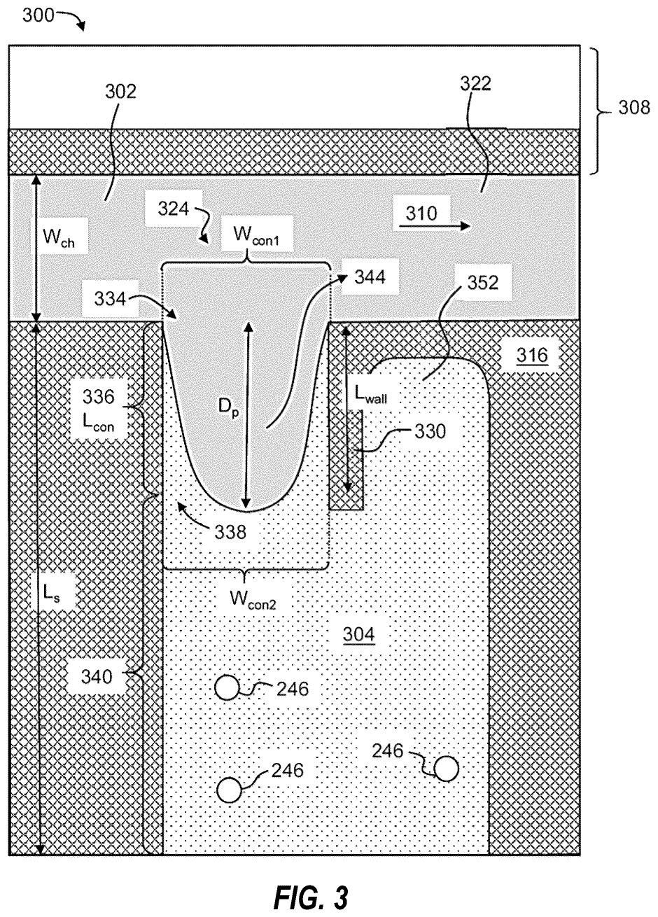

FIG. 3 depicts another exemplary embodiment of a microfluidic device 300 containing microfluidic circuit structure 308, which includes a channel 322 and sequestration pen 324, which has features and properties like any of the sequestration pens described herein for microfluidic devices 100, 175, 200, 400, 520 and any other microfluidic devices described herein.

The exemplary microfluidic devices of FIG. 3 include a microfluidic channel 322, having a width W_{ch}, as described herein, and containing a flow 310 of first fluidic medium 302 and one or more sequestration pens 324 (only one illustrated in FIG. 3). The sequestration pens 324 each have a length Ls, a connection region 336, and an isolation region 340, where the isolation region 340 contains a second fluidic medium 304. The connection region 336 has a proximal opening 334, having a width W_{con1}, which opens to the microfluidic channel 322, and a distal opening 338, having a width W_{con2}, which opens to the isolation region 340. The width W_{con1} may or may not be the same as W_{con2}, as described herein. The walls of each sequestration pen 324 may be formed of microfluidic circuit material 316, which may further form the connection region walls 330. A connection region wall 330 can correspond to a structure that is laterally positioned with respect to the proximal opening 334 and at least partially extends into the enclosed portion of the sequestration pen 324. In some embodiments, the length L_{con} of the connection region 336 is at least partially defined by length L_{wall} of the connection region wall 330. The connection region wall 330 may have a length L_{wall}, selected to be more than the penetration depth Dₚ of the secondary flow 344. Thus, the secondary flow 344 can be wholly contained within the connection region without extending into the isolation region 340.

The connection region wall 330 may define a hook region 352, which is a sub-region of the isolation region 340 of the sequestration pen 324. Since the connection region wall 330 extends into the inner cavity of the sequestration pen, the connection region wall 330 can act as a physical barrier to shield hook region 352 from secondary flow 344, with selection of the length of L_{wall}, contributing to the extent of the hook region. In some embodiments, the longer the length L_{wall} of the connection region wall 330, the more sheltered the hook region 352.

In sequestration pens configured like those of FIGS. 2A-2C and 3, the isolation region may have a shape and size of any type, and may be selected to regulate diffusion of nutrients, reagents, and/or media into the sequestration pen to reach to a far wall of the sequestration pen, e.g., opposite the proximal opening of the connection region to the flow region (or microfluidic channel). The size and shape of the isolation region may further be selected to regulate diffusion of waste products and/or secreted products of a biological micro-object out from the isolation region to the flow region via the proximal opening of the connection region of the sequestration pen. In general, the shape of the isolation region is not critical to the ability of the sequestration pen to isolate micro-objects from direct flow in the flow region.

In some other embodiments of sequestration pens, the isolation region may have more than one opening fluidically connecting the isolation region with the flow region of the microfluidic device. However, for an isolation region having a number of n openings fluidically connecting the isolation region to the flow region (or two or more flow regions), n−1 openings can be valved. When the n−1 valved openings are closed, the isolation region has only one effective opening, and exchange of materials into/out of the isolation region occurs only by diffusion.

Examples of microfluidic devices having pens in which biological micro-objects can be placed, cultured, and/or monitored have been described, for example, in U.S. Pat. No. 9,857,333 (Chapman, et al.), U.S. Pat. No. 10,010,882 (White, et al.), and U.S. Pat. No. 9,889,445 (Chapman, et al.), each of which is incorporated herein by reference in its entirety.

Microfluidic circuit element dimensions. Various dimensions and/or features of the sequestration pens and the microfluidic channels to which the sequestration pens open, as described herein, may be selected to limit introduction of contaminants or unwanted micro-objects into the isolation region of a sequestration pen from the flow region/microfluidic channel; limit the exchange of components in the fluidic medium from the channel or from the isolation region to substantially only diffusive exchange; facilitate the transfer of micro-objects into and/or out of the sequestration pens; and/or facilitate growth or expansion of the biological cells. Microfluidic channels and sequestration pens, for any of the embodiments described herein, may have any suitable combination of dimensions, may be selected by one of skill from the teachings of this disclosure.

For any of the microfluidic devices described herein, a microfluidic channel may have a uniform cross sectional height along its length that is a substantially uniform cross sectional height, and may be any cross sectional height as described herein. At any point along the microfluidic channel, the substantially uniform cross sectional height of the channel, the upper surface of which is defined by the inner surface of the cover and the lower surface of which is defined by the inner surface of the base, may be substantially the same as the cross sectional height at any other point along the channel, e.g., having a cross sectional height that is no more than about 10%, about 9%, about 8%, about 7%, about 6%, about 5%, about 4%, about 3%, about 2% or about 1% or less, different from the cross-sectional height of any other location within the channel.

Additionally, the chamber(s), e.g., sequestration pen(s), of the microfluidic devices described herein, may be disposed substantially in a coplanar orientation relative to the microfluidic channel into which the chamber(s) open. That is, the enclosed volume of the chamber(s) is formed by an upper surface that is defined by the inner surface of the cover, a lower surface defined by the inner surface of the base, and walls defined by the microfluidic circuit material. Therefore, the lower surface of the chamber(s) may be coplanar to the lower surface of the microfluidic channel, e.g., substantially coplanar. The upper surface of the chamber may be coplanar to the upper surface of the microfluidic channel, e.g., substantially coplanar. Accordingly, the chamber(s) may have a cross-sectional height, which may have any values as described herein, that is the same as the channel, e.g., substantially the same, and the chamber(s) and microfluidic channel(s) within the microfluidic device may have a substantially uniform cross sectional height throughout the flow region of the microfluidic device, and may be substantially coplanar throughout the microfluidic device.

Coplanarity of the lower surfaces of the chamber(s) and the microfluidic channel(s) can offer distinct advantage with repositioning micro-objects within the microfluidic device using DEP or magnetic force. Penning and unpenning of micro-objects, and in particular selective penning/selective unpenning, can be greatly facilitated when the lower surfaces of the chamber(s) and the microfluidic channel to which the chamber(s) open have a coplanar orientation.

The proximal opening of the connection region of a sequestration pen may have a width (e.g., W_{con} or W_{con1}) that is at least as large as the largest dimension of a micro-object (e.g., a biological cell, which may be a plant cell, such as a plant protoplast) for which the sequestration pen is intended. In some embodiments, the proximal opening has a width (e.g., W_{con} or W_{con1}) of about 20 microns, about 40 microns, about 50 microns, about 60 microns, about 75 microns, about 100 microns, about 150 microns, about 200 microns, or about 300 microns. The foregoing are examples only, and the width (e.g., W_{con} or W_{con1}) of a proximal opening can be selected to be a value between any of the values listed above (e.g., about 20-200 microns, about 20-150 microns, about 20-100 microns, about 20-75 microns, about 20-60 microns, about 50-300 microns, about 50-200 microns, about 50-150 microns, about 50-100 microns, about 50-75 microns, about 75-150 microns, about 75-100 microns, about 100-300 microns, about 100-200 microns, or about 200-300 microns).

In some embodiments, the connection region of the sequestration pen may have a length (e.g., L_{con}) from the proximal opening to the distal opening to the isolation region of the sequestration pen that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25. times, at least 2.5 times, at least 2.75 times, at least 3.0 times, at least 3.5 times, at least 4.0 times, at least 4.5 times, at least 5.0 times, at least 6.0 times, at least 7.0 times, at least 8.0 times, at least 9.0 times, or at least 10.0 times the width (e.g., W_{con} or W_{con1}) of the proximal opening. Thus, for example, the proximal opening of the connection region of a sequestration pen may have a width (e.g., W_{con} or W_{con1}) from about 20 microns to about 200 microns (e.g., about 50 microns to about 150 microns), and the connection region may have a length L_{con} that is at least 1.0 times (e.g., at least 1.5 times, or at least 2.0 times) the width of the proximal opening. As another example, the proximal opening of the connection region of a sequestration pen may have a width (e.g., W_{con} or W_{con1}) from about 20 microns to about 100 microns (e.g., about 20 microns to about 60 microns), and the connection region may have a length L_{con} that is at least 1.0 times (e.g., at least 1.5 times, or at least 2.0 times) the width of the proximal opening.

The microfluidic channel of a microfluidic device to which a sequestration pen opens may have specified size (e.g., width or height). In some embodiments, the height (e.g., H_{ch}) of the microfluidic channel at a proximal opening to the connection region of a sequestration pen can be within any of the following ranges: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height (e.g., H_{ch}) of the microfluidic channel (e.g., 122) can be selected to be between any of the values listed above. Moreover, the height (e.g., H_{ch}) of the microfluidic channel 122 can be selected to be any of these heights in regions of the microfluidic channel other than at a proximal opening of a sequestration pen.

The width (e.g., W_{ch}) of the microfluidic channel at the proximal opening to the connection region of a sequestration pen can be within any of the following ranges: about 20-500 microns, 20-400 microns, 20-300 microns, 20-200 microns, 20-150 microns, 20-100 microns, 20-80 microns, 20-60 microns, 30-400 microns, 30-300 microns, 30-200 microns, 30-150 microns, 30-100 microns, 30-80 microns, 30-60 microns, 40-300 microns, 40-200 microns, 40-150 microns, 40-100 microns, 40-80 microns, 40-60 microns, 50-1000 microns, 50-500 microns, 50-400 microns, 50-300 microns, 50-250 microns, 50-200 microns, 50-150 microns, 50-100 microns, 50-80 microns, 60-300 microns, 60-200 microns, 60-150 microns, 60-100 microns, 60-80 microns, 70-500 microns, 70-400 microns, 70-300 microns, 70-250 microns, 70-200 microns, 70-150 microns, 70-100 microns, 80-100 microns, 90-400 microns, 90-300 microns, 90-250 microns, 90-200 microns, 90-150 microns, 100-300 microns, 100-250 microns, 100-200 microns, 100-150 microns, 100-120 microns, 200-800 microns, 200-700 microns, or 200-600 microns. The foregoing are examples only, and the width (e.g., W_{ch}) of the microfluidic channel can be a value selected to be between any of the values listed above. Moreover, the width (e.g., W_{ch}) of the microfluidic channel can be selected to be in any of these widths in regions of the microfluidic channel other than at a proximal opening of a sequestration pen. In some embodiments, the width W_{ch} of the microfluidic channel at the proximal opening to the connection region of the sequestration pen (e.g., taken transverse to the direction of bulk flow of fluid through the channel) can be substantially perpendicular to a width (e.g., W_{con} or W_{con1}) of the proximal opening.

A cross-sectional area of the microfluidic channel at a proximal opening to the connection region of a sequestration pen can be about 500-50,000 square microns, 500-40,000 square microns, 500-30,000 square microns, 500-25,000 square microns, 500-20,000 square microns, 500-15,000 square microns, 500-10,000 square microns, 500-7,500 square microns, 500-5,000 square microns, 1,000-25,000 square microns, 1,000-20,000 square microns, 1,000-15,000 square microns, 1,000-10,000 square microns, 1,000-7,500 square microns, 1,000-5,000 square microns, 2,000-20,000 square microns, 2,000-15,000 square microns, 2,000-10,000 square microns, 2,000-7,500 square microns, 2,000-6,000 square microns, 3,000-20,000 square microns, 3,000-15,000 square microns, 3,000-10,000 square microns, 3,000-7,500 square microns, or 3,000 to 6,000 square microns. The foregoing are examples only, and the cross-sectional area of the microfluidic channel at the proximal opening can be selected to be between any of the values listed above. In various embodiments, and the cross-sectional area of the microfluidic channel at regions of the microfluidic channel other than at the proximal opening can also be selected to be between any of the values listed above. In some embodiments, the cross-sectional area is selected to be a substantially uniform value for the entire length of the microfluidic channel.

In some embodiments, the microfluidic chip is configured such that the proximal opening (e.g., 234 or 334) of the connection region of a sequestration pen may have a width (e.g., W_{con} or W_{con1}) from about 20 microns to about 200 microns (e.g., about 50 microns to about 150 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. As another example, the proximal opening (e.g., 234 or 334) of the connection region of a sequestration pen may have a width (e.g., W_{con} or W_{con1}) from about 20 microns to about 100 microns (e.g., about 20 microns to about 60 microns), the connection region may have a length L_{con} (e.g., 236 or 336) that is at least 1.0 times (e.g., at least 1.5 times, or at least 2.0 times) the width of the proximal opening, and the microfluidic channel may have a height (e.g., H_{ch}) at the proximal opening of about 30 microns to about 60 microns. The foregoing are examples only, and the width (e.g., W_{con} or W_{con1}) of the proximal opening (e.g., 234 or 274), the length (e.g., L_{con}) of the connection region, and/or the width (e.g., W_{ch}) of the microfluidic channel (e.g., 122 or 322), can be a value selected to be between any of the values listed above. Generally, however, the width (W_{con} or W_{con1}) of the proximal opening of the connection region of a sequestration pen is less than the width (W_{ch}) of the microfluidic channel. In some embodiments, the width (W_{con} or W_{con1}) of the proximal opening is about 8%, 9%, 10%, 11%, 12%, 13%, 14%, 15%, 16%, 17%, 18%, 19%, 20%, 22%, 24%, 25%, or 30% of the width (W_{ch}) of the microfluidic channel. That is, the width (W_{ch}) of the microfluidic channel may be at least 2.5 times, 3.0 times, 3.5 times, 4.0 times, 4.5 times, 5.0 times, 6.0 times, 7.0 times, 8.0 times, 9.0 times or at least 10.0 times the width (W_{con} or W_{con1}) of the proximal opening of the connection region of the sequestration pen.

In some embodiments, the size We (e.g., cross-sectional width W_{ch}, diameter, area, or the like) of the channel 122, 322, 618, 718 can be about one and a quarter (1.25), about one and a half (1.5), about two, about two and a half (2.5), about three (3), or more times the size Wo (e.g., cross-sectional width W_{con}, diameter, area, or the like) of a chamber opening, e.g., sequestration pen opening 234, 334, and the like. This can reduce the extent of secondary flow and the rate of diffusion (or diffusion flux) through the opening 234, 334 for materials diffusing from a selected chamber (e.g., like sequestration pens 224, 226 of FIG. 2B) into channel 122, 322, 618, 718 and subsequently re-entering a downstream or adjacent chamber (e.g., like sequestration pen 228). The rate of diffusion of a molecule (e.g., an analyte of interest, such as an antibody) is dependent on a number of factors, including (without limitation) temperature, viscosity of the medium, and the coefficient of diffusion Do of the molecule. For example, the Do for an IgG antibody in aqueous solution at about 20° C. is about 4.4×10⁻⁷ cm²/sec, while the kinematic viscosity of cell culture medium is about 9×10⁻⁴ m²/sec. Thus, an antibody in cell culture medium at about 20° C. can have a rate of diffusion of about 0.5 microns/sec. Accordingly, in some embodiments, a time period for diffusion from a biological micro-object located within a sequestration pen such as 224, 226, 228, 324 into the channel 122, 322, 618, 718 can be about 10 minutes or less (e.g., about 9, 8, 7, 6, 5 minutes, or less). The time period for diffusion can be manipulated by changing parameters that influence the rate of diffusion. For example, the temperature of the media can be increased (e.g., to a physiological temperature such as about 37° C.) or decreased (e.g., to about 15° C., 10° C., or 4° C.) thereby increasing or decreasing the rate of diffusion, respectively. Alternatively, or in addition, the concentrations of solutes in the medium can be increased or decreased as discussed herein to isolate a selected pen from solutes from other upstream pens.

Accordingly, in some variations, the width (e.g., W_{ch}) of the microfluidic channel at the proximal opening to the connection region of a sequestration pen may be about 50 to 500 microns, about 50 to 300 microns, about 50 to 200 microns, about 70 to 500 microns, about to 70-300 microns, about 70 to 250 microns, about 70 to 200 microns, about 70 to 150 microns, about 70 to 100 microns, about 80 to 500 microns, about 80 to 300 microns, about 80 to 250 microns, about 80 to 200 microns, about 80 to 150 microns, about 90 to 500 microns, about 90 to 300 microns, about 90 to 250 microns, about 90 to 200 microns, about 90 to 150 microns, about 100 to 500 microns, about 100 to 300 microns, about 100 to 250 microns, about 100 to 200 microns, or about 100 to 150 microns. In some embodiments, the width W_{ch} of the microfluidic channel at the proximal opening to the connection region of a sequestration pen may be about 70 to 250 microns, about 80 to 200 microns, or about 90 to 150 microns. The width W_{con} of the opening of the chamber (e.g., sequestration pen) may be about 20 to 100 microns; about 30 to 90 microns; or about 20 to 60 microns. In some embodiments, W_{ch} is about 70-250 microns and W_{con} is about 20 to 100 microns; W_{ch} is about 80 to 200 microns and W_{con} is about 30 to 90 microns; W_{ch} is about 90 to 150 microns, and W_{con} is about 20 to 60 microns; or any combination of the widths of W_{ch} and W_{con} thereof.

In some embodiments, the proximal opening (e.g., 234 or 334) of the connection region of a sequestration pen has a width (e.g., W_{con} or W_{con1}) that is 2.0 times or less (e.g., 2.0, 1.9, 1.8, 1.5, 1.3, 1.0, 0.8, 0.5, or 0.1 times) the height (e.g., H_{ch}) of the flow region/microfluidic channel at the proximal opening, or has a value that lies within a range defined by any two of the foregoing values.

In some embodiments, the width W_{con1} of a proximal opening (e.g., 234 or 334) of a connection region of a sequestration pen may be the same as a width W_{con2} of the distal opening (e.g., 238 or 338) to the isolation region thereof. In some embodiments, the width W_{con1} of the proximal opening may be different than a width W_{con2} of the distal opening, and W_{con1} and/or W_{con2} may be selected from any of the values described for W_{con} or W_{con1}. In some embodiments, the walls (including a connection region wall) that define the proximal opening and distal opening may be substantially parallel with respect to each other. In some embodiments, the walls that define the proximal opening and distal opening may be selected to not be parallel with respect to each other.

The length (e.g., L_{con}) of the connection region can be about 1-600 microns, 5-550 microns, 10-500 microns, 15-400 microns, 20-300 microns, 20-500 microns, 40-400 microns, 60-300 microns, 80-200 microns, about 100-150 microns, about 20-300 microns, about 20-250 microns, about 20-200 microns, about 20-150 microns, about 20-100 microns, about 30-250 microns, about 30-200 microns, about 30-150 microns, about 30-100 microns, about 30-80 microns, about 30-50 microns, about 45-250 microns, about 45-200 microns, about 45-100 microns, about 45-80 microns, about 45-60 microns, about 60-200 microns, about 60-150 microns, about 60-100 microns or about 60-80 microns. The foregoing are examples only, and length (e.g., L_{con}) of a connection region can be selected to be a value that is between any of the values listed above.

The connection region wall of a sequestration pen may have a length (e.g., L_{wall}) that is at least 0.5 times, at least 0.6 times, at least 0.7 times, at least 0.8 times, at least 0.9 times, at least 1.0 times, at least 1.1 times, at least 1.2 times, at least 1.3 times, at least 1.4 times, at least 1.5 times, at least 1.75 times, at least 2.0 times, at least 2.25 times, at least 2.5 times, at least 2.75 times, at least 3.0 times, or at least 3.5 times the width (e.g., W_{con} or W_{con1}) of the proximal opening of the connection region of the sequestration pen. In some embodiments, the connection region wall may have a length L_{wall} of about 20-200 microns, about 20-150 microns, about 20-100 microns, about 20-80 microns, or about 20-50 microns. The foregoing are examples only, and a connection region wall may have a length L_{wall} selected to be between any of the values listed above.

A sequestration pen may have a length Ls of about 40-600 microns, about 40-500 microns, about 40-400 microns, about 40-300 microns, about 40-200 microns, about 40-100 microns or about 40-80 microns. The foregoing are examples only, and a sequestration pen may have a length Ls selected to be between any of the values listed above.

According to some embodiments, a sequestration pen may have a specified height (e.g., Hs). In some embodiments, a sequestration pen has a height Hs of about 20 microns to about 200 microns (e.g., about 20 microns to about 150 microns, about 20 microns to about 100 microns, about 20 microns to about 60 microns, about 30 microns to about 150 microns, about 30 microns to about 100 microns, about 30 microns to about 60 microns, about 40 microns to about 150 microns, about 40 microns to about 100 microns, or about 40 microns to about 60 microns). The foregoing are examples only, and a sequestration pen can have a height Hs selected to be between any of the values listed above.

The height H_{con} of a connection region at a proximal opening of a sequestration pen can be a height within any of the following heights: 20-100 microns, 20-90 microns, 20-80 microns, 20-70 microns, 20-60 microns, 20-50 microns, 30-100 microns, 30-90 microns, 30-80 microns, 30-70 microns, 30-60 microns, 30-50 microns, 40-100 microns, 40-90 microns, 40-80 microns, 40-70 microns, 40-60 microns, or 40-50 microns. The foregoing are examples only, and the height H_{con} of the connection region can be selected to be between any of the values listed above. Typically, the height H_{con} of the connection region is selected to be the same as the height H_{ch} of the microfluidic channel at the proximal opening of the connection region. Additionally, the height Hs of the sequestration pen is typically selected to be the same as the height H_{con} of a connection region and/or the height H_{ch} of the microfluidic channel. In some embodiments, Hs, H_{con}, and H_{ch} may be selected to be the same value of any of the values listed above for a selected microfluidic device.

The isolation region can be configured to contain only one, two, three, four, five, or a similar relatively small number of micro-objects. In other embodiments, the isolation region may contain more than 10, more than 50 or more than 100 micro-objects. Accordingly, the volume of an isolation region can be, for example, at least 1×10⁴, 1×10⁵, 5×10⁵, 8×10⁵, 1×10⁶, 2×10⁶, 4×10⁶, 6×10⁶, 1×10⁷, 3×10⁷, 5×10⁷ 1×10⁸, 5×10⁸, or 8×10⁸ cubic microns, or more. The foregoing are examples only, and the isolation region can be configured to contain numbers of micro-objects and volumes selected to be between any of the values listed above (e.g., a volume between 1×10⁵ cubic microns and 5×10⁵ cubic microns, between 5×10⁵ cubic microns and 1×10⁶ cubic microns, between 1×10⁶ cubic microns and 2×10⁶ cubic microns, or between 2×10⁶ cubic microns and 1×10⁷ cubic microns).

According to some embodiments, a sequestration pen of a microfluidic device may have a specified volume. The specified volume of the sequestration pen (or the isolation region of the sequestration pen) may be selected such that a single cell or a small number of cells (e.g., 2-10 or 2-5) can rapidly condition the medium and thereby attain favorable (or optimal) growth conditions. In some embodiments, the sequestration pen has a volume of about 5×10⁵, 6×10⁵, 8×10⁵, 1×10⁶, 2×10⁶, 4×10⁶, 8×10⁶, 1×10⁷, 3×10⁷, 5×10⁷, or about 8×10⁷ cubic microns, or more. In some embodiments, the sequestration pen has a volume of about 1 nanoliter to about 50 nanoliters, 2 nanoliters to about 25 nanoliters, 2 nanoliters to about 20 nanoliters, about 2 nanoliters to about 15 nanoliters, or about 2 nanoliters to about 10 nanoliters. The foregoing are examples only, and a sequestration pen can have a volume selected to be any value that is between any of the values listed above.

According to some embodiments, the flow of fluidic medium within the microfluidic channel (e.g., 122 or 322) may have a specified maximum velocity (e.g., Vₘₐₓ). In some embodiments, the maximum velocity (e.g., Vₘₐₓ) may be set at around 0.2, 0.5, 0.7, 1.0, 1.3, 1.5, 2.0, 2.5, 3.0, 3.5, 4.0, 4.5, 5.0, 5.5, 6.0, 6.7, 7.0, 7.5, 8.0, 8.5, 9.0, 10, 11, 12, 13, 14, 15, 16, 17, 18, 19, 20, 22, 24, or 25 microliters/sec. The foregoing are examples only, and the flow of fluidic medium within the microfluidic channel can have a maximum velocity (e.g., Vₘₐₓ) selected to be a value between any of the values listed above. The flow of fluidic medium within the microfluidic channel typically may be flowed at a rate less than the Vₘₐₓ. While the Vₘₐₓ may vary depending on the specific size and numbers of channel and sequestration pens opening thereto, a fluidic medium may be flowed at about 0.1 microliters/sec to about 20 microliters/sec; about 0.1 microliters/sec to about 15 microliters/sec; about 0.1 microliters/sec to about 12 microliters/sec, about 0.1 microliters/sec to about 10 microliters/sec; about 0.1 microliter/sec to about 7 microliters/sec without exceeding the Vₘₐₓ. In some portions of a typical workflow, a flow rate of a fluidic medium may be about 0.1 microliters/sec; about 0.5 microliters/sec; about 1.0 microliters/sec; about 2.0 microliters/sec; about 3.0 microliters/sec; about 4.0 microliters/sec; about 5.0 microliters/sec; about 6.0 microliters/sec; about 7.0 microliters/sec; about 8.0 microliters/sec; about 9.0 microliters/sec; about 10.0 microliters/sec; about 11.0 microliters/sec; or any range defined by two of the foregoing values, e.g., 1-5 microliters/sec or 5-10 microliters/sec. The flow rate of a fluidic medium in the microfluidic channel may be equal to or less than about 12 microliters/sec; about 10 microliters/sec; about 8 microliters/sec, or about 6 microliters/sec.

In various embodiment, the microfluidic device has sequestration pens configured as in any of the embodiments discussed herein where the microfluidic device has about 5 to about 10 sequestration pens, about 10 to about 50 sequestration pens, about 25 to about 200 sequestration pens, about 100 to about 500 sequestration pens, about 200 to about 1000 sequestration pens, about 500 to about 1500 sequestration pens, about 1000 to about 2500 sequestration pens, about 2000 to about 5000 sequestration pens, about 3500 to about 7000 sequestration pens, about 5000 to about 10,000 sequestration pens, about 7,500 to about 15,000 sequestration pens, about 12,500 to about 20,000 sequestration pens, about 15,000 to about 25,000 sequestration pens, about 20,000 to about 30,000 sequestration pens, about 25,000 to about 35,000 sequestration pens, about 30,000 to about 40,000 sequestration pens, about 35,000 to about 45,000 sequestration pens, or about 40,000 to about 50,000 sequestration pens. The sequestration pens need not all be the same size and may include a variety of configurations (e.g., different widths, different features within the sequestration pen).

Coating solutions and coating agents. In some embodiments, at least one inner surface of the microfluidic device includes a coating material that provides a layer of organic and/or hydrophilic molecules suitable for maintenance, expansion and/or movement of biological micro-object(s) (i.e., the biological micro-object exhibits increased viability, greater expansion and/or greater portability within the microfluidic device). The conditioned surface may reduce surface fouling, participate in providing a layer of hydration, and/or otherwise shield the biological micro-objects from contact with the non-organic materials of the microfluidic device interior.

In some embodiments, substantially all the inner surfaces of the microfluidic device include the coating material. The coated inner surface(s) may include the surface of a flow region (e.g., channel), chamber, or sequestration pen, or a combination thereof. In some embodiments, each of a plurality of sequestration pens has at least one inner surface coated with coating materials. In other embodiments, each of a plurality of flow regions or channels has at least one inner surface coated with coating materials. In some embodiments, at least one inner surface of each of a plurality of sequestration pens and each of a plurality of channels is coated with coating materials. The coating may be applied before or after introduction of biological micro-object(s), or may be introduced concurrently with the biological micro-object(s). In some embodiments, the biological micro-object(s) may be imported into the microfluidic device in a fluidic medium that includes one or more coating agents. In other embodiments, the inner surface(s) of the microfluidic device (e.g., a microfluidic device having an electrode activation substrate such as, but not limited to, a device including dielectrophoresis (DEP) electrodes) may be treated or “primed” with a coating solution comprising a coating agent prior to introduction of the biological micro-object(s) into the microfluidic device. Any convenient coating agent/coating solution can be used, including but not limited to: serum or serum factors, bovine serum albumin (BSA), polymers, detergents, enzymes, and any combination thereof.

Synthetic polymer-based coating materials. The at least one inner surface may include a coating material that comprises a polymer. The polymer may be non-covalently bound (e.g., it may be non-specifically adhered) to the at least one surface. The polymer may have a variety of structural motifs, such as found in block polymers (and copolymers), star polymers (star copolymers), and graft or comb polymers (graft copolymers), all of which may be suitable for the methods disclosed herein. A wide variety of alkylene ether containing polymers may be suitable for use in the microfluidic devices described herein, including but not limited to Pluronic® polymers such as Pluronic® L44, L64, P85, and F127 (including F127NF). Other examples of suitable coating materials are described in US2016/0312165, the contents of which are herein incorporated by reference in their entirety.

Covalently linked coating materials. In some embodiments, the at least one inner surface includes covalently linked molecules that provide a layer of organic and/or hydrophilic molecules suitable for maintenance/expansion of biological micro-object(s) within the microfluidic device, providing a conditioned surface for such cells. The covalently linked molecules include a linking group, wherein the linking group is covalently linked to one or more surfaces of the microfluidic device, as described below. The linking group is also covalently linked to a surface modifying moiety configured to provide a layer of organic and/or hydrophilic molecules suitable for maintenance/expansion/movement of biological micro-object(s).

In some embodiments, the covalently linked moiety configured to provide a layer of organic and/or hydrophilic molecules suitable for maintenance/expansion of biological micro-object(s) may include alkyl or fluoroalkyl (which includes perfluoroalkyl) moieties; mono- or polysaccharides (which may include but is not limited to dextran); alcohols (including but not limited to propargyl alcohol); polyalcohols, including but not limited to polyvinyl alcohol; alkylene ethers, including but not limited to polyethylene glycol; polyelectrolytes (including but not limited to polyacrylic acid or polyvinyl phosphonic acid); amino groups (including derivatives thereof, such as, but not limited to alkylated amines, hydroxyalkylated amino group, guanidinium, and heterocylic groups containing an unaromatized nitrogen ring atom, such as, but not limited to morpholinyl or piperazinyl); carboxylic acids including but not limited to propiolic acid (which may provide a carboxylate anionic surface); phosphonic acids, including but not limited to ethynyl phosphonic acid (which may provide a phosphonate anionic surface); sulfonate anions; carboxybetaines; sulfobetaines; sulfamic acids; or amino acids.

In various embodiments, the covalently linked moiety configured to provide a layer of organic and/or hydrophilic molecules suitable for maintenance/expansion of biological micro-object(s) in the microfluidic device may include non-polymeric moieties such as an alkyl moiety, amino acid moiety, alcohol moiety, amino moiety, carboxylic acid moiety, phosphonic acid moiety, sulfonic acid moiety, sulfamic acid moiety, or saccharide moiety. Alternatively, the covalently linked moiety may include polymeric moieties, which may include any of these moieties.

In some embodiments, a microfluidic device may have a hydrophobic layer upon the inner surface of the base which includes a covalently linked alkyl moiety. The covalently linked alkyl moiety may comprise carbon atoms forming a linear chain (e.g., a linear chain of at least 10 carbons, or at least 14, 16, 18, 20, 22, or more carbons) and may be an unbranched alkyl moiety. In some embodiments, the alkyl group may include a substituted alkyl group (e.g., some of the carbons in the alkyl group can be fluorinated or perfluorinated). In some embodiments, the alkyl group may include a first segment, which may include a perfluoroalkyl group, joined to a second segment, which may include a non-substituted alkyl group, where the first and second segments may be joined directly or indirectly (e.g., by means of an ether linkage). The first segment of the alkyl group may be located distal to the linking group, and the second segment of the alkyl group may be located proximal to the linking group.

In other embodiments, the covalently linked moiety may include at least one amino acid, which may include more than one type of amino acid. Thus, the covalently linked moiety may include a peptide or a protein. In some embodiments, the covalently linked moiety may include an amino acid which may provide a zwitterionic surface to support cell growth, viability, portability, or any combination thereof.

In other embodiments, the covalently linked moiety may further include a streptavidin or biotin moiety. In some embodiments, a modified biological moiety such as, for example, a biotinylated protein or peptide may be introduced to the inner surface of a microfluidic device bearing covalently linked streptavidin, and couple via the covalently linked streptavidin to the surface, thereby providing a modified surface presenting the protein or peptide.

In other embodiments, the covalently linked moiety may include at least one alkylene oxide moiety and may include any alkylene oxide polymer as described above. One useful class of alkylene ether containing polymers is polyethylene glycol (PEG M_{w}<100,000 Da) or alternatively polyethylene oxide (PEO, M_{w}>100,000). In some embodiments, a PEG may have an M_{w} of about 1000 Da, 5000 Da, 10,000 Da or 20,000 Da. In some embodiments, the PEG polymer may further be substituted with a hydrophilic or charged moiety, such as but not limited to an alcohol functionality or a carboxylic acid moiety.

The covalently linked moiety may include one or more saccharides. The covalently linked saccharides may be mono-, di-, or polysaccharides. The covalently linked saccharides may be modified to introduce a reactive pairing moiety which permits coupling or elaboration for attachment to the surface. One exemplary covalently linked moiety may include a dextran polysaccharide, which may be coupled indirectly to a surface via an unbranched linker.

The coating material providing a conditioned surface may comprise only one kind of covalently linked moiety or may include more than one different kind of covalently linked moiety. For example, a polyethylene glycol conditioned surface may have covalently linked alkylene oxide moieties having a specified number of alkylene oxide units which are all the same, e.g., having the same linking group and covalent attachment to the surface, the same overall length, and the same number of alkylene oxide units. Alternatively, the coating material may have more than one kind of covalently linked moiety attached to the surface. For example, the coating material may include the molecules having covalently linked alkylene oxide moieties having a first specified number of alkylene oxide units and may further include a further set of molecules having bulky moieties such as a protein or peptide connected to a covalently attached alkylene oxide linking moiety having a greater number of alkylene oxide units. The different types of molecules may be varied in any suitable ratio to obtain the surface characteristics desired. For example, the conditioned surface having a mixture of first molecules having a chemical structure having a first specified number of alkylene oxide units and second molecules including peptide or protein moieties, which may be coupled via a biotin/streptavidin binding pair to the covalently attached alkylene linking moiety, may have a ratio of first molecules:second molecules of about 99:1; about 90:10; about 75:25; about 50:50; about 30:70; about 20:80; about 10:90; or any ratio selected to be between these values. In this instance, the first set of molecules having different, less sterically demanding termini and fewer backbone atoms can help to functionalize the entire substrate surface and thereby prevent undesired adhesion or contact with the silicon/silicon oxide, hafnium oxide or alumina making up the substrate itself. The selection of the ratio of mixture of first molecules to second molecules may also modulate the surface modification introduced by the second molecules bearing peptide or protein moieties.

Conditioned surface properties. Various factors can alter the physical thickness of the conditioned surface, such as the manner in which the conditioned surface is formed on the substrate (e.g., vapor deposition, liquid phase deposition, spin coating, flooding, and electrostatic coating). In some embodiments, the conditioned surface may have a thickness of about 1 nm to about 10 nm. In some embodiments, the covalently linked moieties of the conditioned surface may form a monolayer when covalently linked to the surface of the microfluidic device (which may include an electrode activation substrate having dielectrophoresis (DEP) or electrowetting (EW) electrodes) and may have a thickness of less than 10 nm (e.g., less than 5 nm, or about 1.5 to 3.0 nm). These values are in contrast to that of a surface prepared by spin coating, for example, which may typically have a thickness of about 30 nm. In some embodiments, the conditioned surface does not require a perfectly formed monolayer to be suitably functional for operation within a DEP-configured microfluidic device. In other embodiments, the conditioned surface formed by the covalently linked moieties may have a thickness of about 10 nm to about 50 nm.

Unitary or Multi-part conditioned surface. The covalently linked coating material may be formed by reaction of a molecule which already contains the moiety configured to provide a layer of organic and/or hydrophilic molecules suitable for maintenance/expansion of biological micro-object(s) in the microfluidic device, and may have a structure of Formula I, as shown below. Alternatively, the covalently linked coating material may be formed in a two-part sequence, having a structure of Formula II, by coupling the moiety configured to provide a layer of organic and/or hydrophilic molecules suitable for maintenance and/or expansion of biological micro-object(s) to a surface modifying ligand that itself has been covalently linked to the surface. In some embodiments, the surface may be formed in a two-part or three-part sequence, including a streptavidin/biotin binding pair, to introduce a protein, peptide, or mixed modified surface.

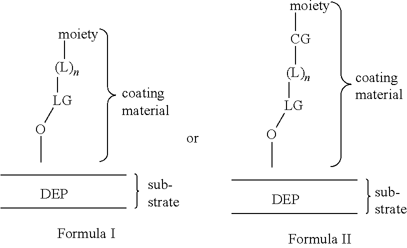

The coating material may be linked covalently to oxides of the surface of a DEP-configured or EW-configured substrate. The coating material may be attached to the oxides via a linking group (“LG”), which may be a siloxy or phosphonate ester group formed from the reaction of a siloxane or phosphonic acid group with the oxides. The moiety configured to provide a layer of organic and/or hydrophilic molecules suitable for maintenance/expansion of biological micro-object(s) in the microfluidic device can be any of the moieties described herein. The linking group LG may be directly or indirectly connected to the moiety configured to provide a layer of organic and/or hydrophilic molecules suitable for maintenance/expansion of biological micro-object(s) in the microfluidic device. When the linking group LG is directly connected to the moiety, optional linker (“L”) is not present and n is 0. When the linking group LG is indirectly connected to the moiety, linker L is present and n is 1. The linker L may have a linear portion where a backbone of the linear portion may include 1 to 200 non-hydrogen atoms selected from any combination of silicon, carbon, nitrogen, oxygen, sulfur and/or phosphorus atoms, subject to chemical bonding limitations as is known in the art. It may be interrupted with any combination of one or more moieties, which may be chosen from ether, amino, carbonyl, amido, and/or phosphonate groups, arylene, heteroarylene, or heterocyclic groups. In some embodiments, the coupling group CG represents the resultant group from reaction of a reactive moiety Rₓ and a reactive pairing moiety Rₚₓ (i.e., a moiety configured to react with the reactive moiety Rₓ). CG may be a carboxamidyl group, a triazolylene group, substituted triazolylene group, a carboxamidyl, thioamidyl, an oxime, a mercaptyl, a disulfide, an ether, or alkenyl group, or any other suitable group that may be formed upon reaction of a reactive moiety with its respective reactive pairing moiety. In some embodiments, CG may further represent a streptavidin/biotin binding pair.

Further details of suitable coating treatments and modifications, as well as methods of preparation, may be found at U.S. Patent Application Publication No. US2016/0312165 (Lowe, Jr., et al.), U.S. Patent Application Publication No US2017/0173580 (Lowe, Jr., et al), International Patent Application Publication WO2017/205830 (Lowe, Jr., et al.), and International Patent Application Publication WO2019/01880 (Beemiller et al.), each of which disclosures is herein incorporated by reference in its entirety.

Microfluidic device motive technologies. The microfluidic devices described herein can be used with any type of motive technology. As described herein, the control and monitoring equipment of the system can comprise a motive module for selecting and moving objects, such as micro-objects or droplets, in the microfluidic circuit of a microfluidic device. The motive technology(ies) may include, for example, dielectrophoresis (DEP), electrowetting (EW), and/or other motive technologies. The microfluidic device can have a variety of motive configurations, depending upon the type of object being moved and other considerations. Returning to FIG. 1A, for example, the support structure 104 and/or cover 110 of the microfluidic device 100 can comprise DEP electrode activation substrates for selectively inducing motive forces on micro-objects in the fluidic medium 180 in the microfluidic circuit 120 and thereby select, capture, and/or move individual micro-objects or groups of micro-objects.

In some embodiments, motive forces are applied across the fluidic medium 180 (e.g., in the flow path and/or in the sequestration pens) via one or more electrodes (not shown) to manipulate, transport, separate and sort micro-objects located therein. For example, in some embodiments, motive forces are applied to one or more portions of microfluidic circuit 120 in order to transfer a single micro-object from the flow path 106 into a desired microfluidic sequestration pen. In some embodiments, motive forces are used to prevent a micro-object within a sequestration pen from being displaced therefrom. Further, in some embodiments, motive forces are used to selectively remove a micro-object from a sequestration pen that was previously collected in accordance with the embodiments of the current disclosure.

In some embodiments, the microfluidic device is configured as an optically-actuated electrokinetic device, such as in optoelectronic tweezer (OET) and/or optoelectrowetting (OEW) configured device. Examples of suitable OET configured devices (e.g., containing optically actuated dielectrophoresis electrode activation substrates) can include those illustrated in U.S. Pat. No. RE 44,711 (Wu, et al.) (originally issued as U.S. Pat. No. 7,612,355), U.S. Pat. No. 7,956,339 (Ohta, et al.), U.S. Pat. No. 9,908,115 (Hobbs et al.), and U.S. Pat. No. 9,403,172 (Short et al), each of which is incorporated herein by reference in its entirety. Examples of suitable OEW configured devices can include those illustrated in U.S. Pat. No. 6,958,132 (Chiou, et al.), and U.S. Pat. No. 9,533,306 (Chiou, et al.), each of which is incorporated herein by reference in its entirety. Examples of suitable optically-actuated electrokinetic devices that include combined OET/OEW configured devices can include those illustrated in U.S. Patent Application Publication No. 2015/0306598 (Khandros, et al.), U.S. Patent Application Publication No 2015/0306599 (Khandros, et al.), and U.S. Patent Application Publication No. 2017/0173580 (Lowe, et al.), each of which is incorporated herein by reference in its entirety.

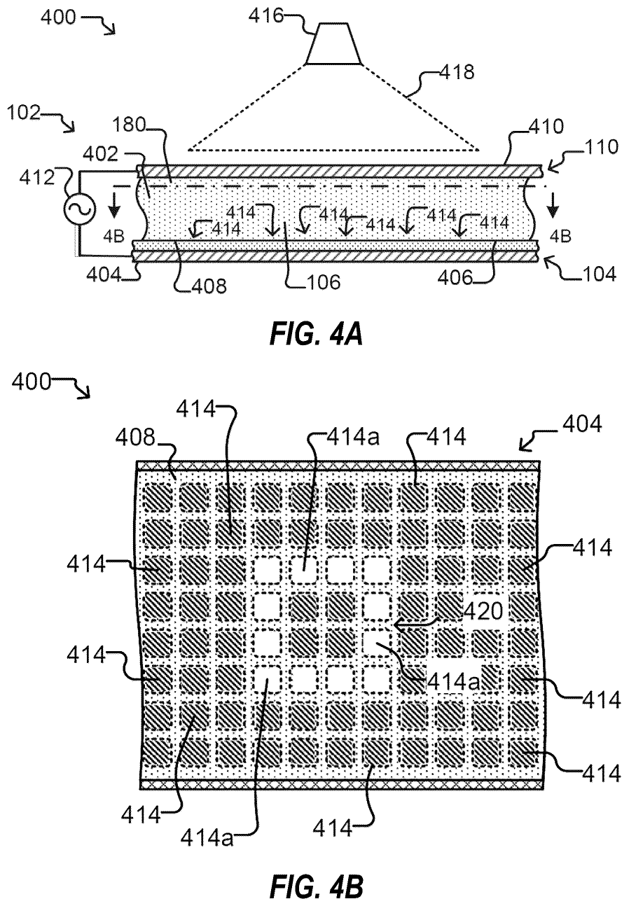

It should be understood that, for purposes of simplicity, the various examples of FIGS. 1-5B may illustrate portions of microfluidic devices while not depicting other portions. Further, FIGS. 1-5B may be part of, and implemented as, one or more microfluidic systems. In one non-limiting example, FIGS. 4A and 4B show a side cross-sectional view and a top cross-sectional view, respectively, of a portion of an enclosure 102 of the microfluidic device 400 having a region/chamber 402, which may be part of a fluidic circuit element having a more detailed structure, such as a growth chamber, a sequestration pen (which may be like any sequestration pen described herein), a flow region, or a flow channel. For instance, microfluidic device 400 may be similar to microfluidic devices 100, 175, 200, 300, 520 or any other microfluidic device as described herein. Furthermore, the microfluidic device 400 may include other fluidic circuit elements and may be part of a system including control and monitoring equipment 152, described above, having one or more of the media module 160, motive module 162, imaging module 164, optional tilting module 166, and other modules 168. Microfluidic devices 175, 200, 300, 520 and any other microfluidic devices described herein may similarly have any of the features described in detail for FIGS. 1A-1B and 4A-4B.

As shown in the example of FIG. 4A, the microfluidic device 400 includes a support structure 104 having a bottom electrode 404 and an electrode activation substrate 406 overlying the bottom electrode 404, and a cover 110 having a top electrode 410, with the top electrode 410 spaced apart from the bottom electrode 404. The top electrode 410 and the electrode activation substrate 406 define opposing surfaces of the region/chamber 402. A fluidic medium 180 contained in the region/chamber 402 thus provides a resistive connection between the top electrode 410 and the electrode activation substrate 406. A power source 412 configured to be connected to the bottom electrode 404 and the top electrode 410 and create a biasing voltage between the electrodes, as required for the generation of DEP forces in the region/chamber 402, is also shown. The power source 412 can be, for example, an alternating current (AC) power source.

In certain embodiments, the microfluidic device 200 illustrated in FIGS. 4A and 4B can have an optically-actuated DEP electrode activation substrate. Accordingly, changing patterns of light 418 from the light source 416, which may be controlled by the motive module 162, can selectively activate and deactivate changing patterns of DEP electrodes at regions 414 of the inner surface 408 of the electrode activation substrate 406. (Hereinafter the regions 414 of a microfluidic device having a DEP electrode activation substrate are referred to as “DEP electrode regions.”) As illustrated in FIG. 4B, a light pattern 418 directed onto the inner surface 408 of the electrode activation substrate 406 can illuminate select DEP electrode regions 414a (shown in white) in a pattern, such as a square. The non-illuminated DEP electrode regions 414 (cross-hatched) are hereinafter referred to as “dark” DEP electrode regions 414. The relative electrical impedance through the DEP electrode activation substrate 406 (i.e., from the bottom electrode 404 up to the inner surface 408 of the electrode activation substrate 406 which interfaces with the fluidic medium 180 in the flow region 106) is greater than the relative electrical impedance through the fluidic medium 180 in the region/chamber 402 (i.e., from the inner surface 408 of the electrode activation substrate 406 to the top electrode 410 of the cover 110) at each dark DEP electrode region 414. An illuminated DEP electrode region 414a, however, exhibits a reduced relative impedance through the electrode activation substrate 406 that is less than the relative impedance through the fluidic medium 180 in the region/chamber 402 at each illuminated DEP electrode region 414a.

With the power source 412 activated, the foregoing DEP configuration creates an electric field gradient in the fluidic medium 180 between illuminated DEP electrode regions 414a and adjacent dark DEP electrode regions 414, which in turn creates local DEP forces that attract or repel nearby micro-objects (not shown) in the fluidic medium 180. DEP electrodes that attract or repel micro-objects in the fluidic medium 180 can thus be selectively activated and deactivated at many different such DEP electrode regions 414 at the inner surface 408 of the region/chamber 402 by changing light patterns 418 projected from a light source 416 into the microfluidic device 400. Whether the DEP forces attract or repel nearby micro-objects can depend on such parameters as the frequency of the power source 412 and the dielectric properties of the fluidic medium 180 and/or micro-objects (not shown). Depending on the frequency of the power applied to the DEP configuration and selection of fluidic media (e.g., a highly conductive media such as PBS or other media appropriate for maintaining biological cells), negative DEP forces may be produced. Negative DEP forces may repel the micro-objects away from the location of the induced non-uniform electrical field. In some embodiments, a microfluidic device incorporating DEP technology may generate negative DEP forces.

The square pattern 420 of illuminated DEP electrode regions 414a illustrated in FIG. 4B is an example only. Any pattern of the DEP electrode regions 414 can be illuminated (and thereby activated) by the pattern of light 418 projected into the microfluidic device 400, and the pattern of illuminated/activated DEP electrode regions 414 can be repeatedly changed by changing or moving the light pattern 418.

In some embodiments, the electrode activation substrate 406 can comprise or consist of a photoconductive material. In such embodiments, the inner surface 408 of the electrode activation substrate 406 can be featureless. For example, the electrode activation substrate 406 can comprise or consist of a layer of hydrogenated amorphous silicon (a-Si:H). The a-Si:H can comprise, for example, about 8% to 40% hydrogen (calculated as 100*the number of hydrogen atoms/the total number of hydrogen and silicon atoms). The layer of a-Si:H can have a thickness of about 500 nm to about 2.0 μm. In such embodiments, the DEP electrode regions 414 can be created anywhere and in any pattern on the inner surface 408 of the electrode activation substrate 406, in accordance with the light pattern 418. The number and pattern of the DEP electrode regions 214 thus need not be fixed, but can correspond to the light pattern 418. Examples of microfluidic devices having a DEP configuration comprising a photoconductive layer such as discussed above have been described, for example, in U.S. Pat. No. RE 44,711 (Wu, et al.) (originally issued as U.S. Pat. No. 7,612,355), each of which is incorporated herein by reference in its entirety.

In other embodiments, the electrode activation substrate 406 can comprise a substrate comprising a plurality of doped layers, electrically insulating layers (or regions), and electrically conductive layers that form semiconductor integrated circuits, such as is known in semiconductor fields. For example, the electrode activation substrate 406 can comprise a plurality of phototransistors, including, for example, lateral bipolar phototransistors, with each phototransistor corresponding to a DEP electrode region 414. Alternatively, the electrode activation substrate 406 can comprise electrodes (e.g., conductive metal electrodes) controlled by phototransistor switches, with each such electrode corresponding to a DEP electrode region 414. The electrode activation substrate 406 can include a pattern of such phototransistors or phototransistor-controlled electrodes. The pattern, for example, can be an array of substantially square phototransistors or phototransistor-controlled electrodes arranged in rows and columns. Alternatively, the pattern can be an array of substantially hexagonal phototransistors or phototransistor-controlled electrodes that form a hexagonal lattice. Regardless of the pattern, electric circuit elements can form electrical connections between the DEP electrode regions 414 at the inner surface 408 of the electrode activation substrate 406 and the bottom electrode 404, and those electrical connections (i.e., phototransistors or electrodes) can be selectively activated and deactivated by the light pattern 418, as described above.

Examples of microfluidic devices having electrode activation substrates that comprise phototransistors have been described, for example, in U.S. Pat. No. 7,956,339 (Ohta et al.) and U.S. Pat. No. 9,908,115 (Hobbs et al.), the entire contents of each of which are incorporated herein by reference. Examples of microfluidic devices having electrode activation substrates that comprise electrodes controlled by phototransistor switches have been described, for example, in U.S. Pat. No. 9,403,172 (Short et al.), which is incorporated herein by reference in its entirety.

In some embodiments of a DEP configured microfluidic device, the top electrode 410 is part of a first wall (or cover 110) of the enclosure 402, and the electrode activation substrate 406 and bottom electrode 404 are part of a second wall (or support structure 104) of the enclosure 102. The region/chamber 402 can be between the first wall and the second wall. In other embodiments, the electrode 410 is part of the second wall (or support structure 104) and one or both of the electrode activation substrate 406 and/or the electrode 410 are part of the first wall (or cover 110). Moreover, the light source 416 can alternatively be used to illuminate the enclosure 102 from below.

With the microfluidic device 400 of FIGS. 4A-4B having a DEP electrode activation substrate, the motive module 162 of control and monitoring equipment 152, as described for FIG. 1A herein, can select a micro-object (not shown) in the fluidic medium 180 in the region/chamber 402 by projecting a light pattern 418 into the microfluidic device 400 to activate a first set of one or more DEP electrodes at DEP electrode regions 414a of the inner surface 408 of the electrode activation substrate 406 in a pattern (e.g., square pattern 420) that surrounds and captures the micro-object. The motive module 162 can then move the in situ-generated captured micro-object by moving the light pattern 418 relative to the microfluidic device 400 to activate a second set of one or more DEP electrodes at DEP electrode regions 414. Alternatively, the microfluidic device 400 can be moved relative to the light pattern 418.

In other embodiments, the microfluidic device 400 may be a DEP configured device that does not rely upon light activation of DEP electrodes at the inner surface 408 of the electrode activation substrate 406. For example, the electrode activation substrate 406 can comprise selectively addressable and energizable electrodes positioned opposite to a surface including at least one electrode (e.g., cover 110). Switches (e.g., transistor switches in a semiconductor substrate) may be selectively opened and closed to activate or inactivate DEP electrodes at DEP electrode regions 414, thereby creating a net DEP force on a micro-object (not shown) in region/chamber 402 in the vicinity of the activated DEP electrodes. Depending on such characteristics as the frequency of the power source 412 and the dielectric properties of the medium (not shown) and/or micro-objects in the region/chamber 402, the DEP force can attract or repel a nearby micro-object. By selectively activating and deactivating a set of DEP electrodes (e.g., at a set of DEP electrodes regions 414 that forms a square pattern 420), one or more micro-objects in region/chamber 402 can be selected and moved within the region/chamber 402. The motive module 162 in FIG. 1A can control such switches and thus activate and deactivate individual ones of the DEP electrodes to select, and move particular micro-objects (not shown) around the region/chamber 402. Microfluidic devices having a DEP electrode activation substrate that includes selectively addressable and energizable electrodes are known in the art and have been described, for example, in U.S. Pat. No. 6,294,063 (Becker, et al.) and U.S. Pat. No. 6,942,776 (Medoro), each of which is incorporated herein by reference in its entirety.

Regardless of whether the microfluidic device 400 has a dielectrophoretic electrode activation substrate, an electrowetting electrode activation substrate or a combination of both a dielectrophoretic and an electrowetting activation substrate, a power source 412 can be used to provide a potential (e.g., an AC voltage potential) that powers the electrical circuits of the microfluidic device 400. The power source 412 can be the same as, or a component of, the power source 192 referenced in FIG. 1A. Power source 412 can be configured to provide an AC voltage and/or current to the top electrode 410 and the bottom electrode 404. For an AC voltage, the power source 412 can provide a frequency range and an average or peak power (e.g., voltage or current) range sufficient to generate net DEP forces (or electrowetting forces) strong enough to select and move individual micro-objects (not shown) in the region/chamber 402, as discussed above, and/or to change the wetting properties of the inner surface 408 of the support structure 104 in the region/chamber 202, as also discussed above. Such frequency ranges and average or peak power ranges are known in the art. See, e.g., U.S. Pat. No. 6,958,132 (Chiou, et al.), U.S. Pat. No. RE44,711 (Wu, et al.) (originally issued as U.S. Pat. No. 7,612,355), and U.S. Patent Application Publication Nos. 2014/0124370 (Short, et al.), 2015/0306598 (Khandros, et al.), 2015/0306599 (Khandros, et al.), and 2017/0173580 (Lowe, Jr. et al.), each of which disclosures are herein incorporated by reference in its entirety.

Other forces may be utilized within the microfluidic devices, alone or in combination, to move selected micro-objects. Bulk fluidic flow within the microfluidic channel may move micro-objects within the flow region. Localized fluidic flow, which may be operated within the microfluidic channel, within a sequestration pen, or within another kind of chamber (e.g., a reservoir) can also be used to move selected micro-objects. Localized fluidic flow can be used to move selected micro-objects out of the flow region into a non-flow region such as a sequestration pen or the reverse, from a non-flow region into a flow region. The localized flow can be actuated by deforming a deformable wall of the microfluidic device, as described in U.S. Pat. No. 10,058,865 (Breinlinger, et al.), which is incorporated herein by reference in its entirety.

Gravity may be used to move micro-objects within the microfluidic channel, into a sequestration pen, and/or out of a sequestration pen or other chamber, as described in U.S. Pat. No. 9,744,533 (Breinlinger, et al.), which is incorporated herein by reference in its entirety. Use of gravity (e.g., by tilting the microfluidic device and/or the support to which the microfluidic device is attached) may be useful for bulk movement of cells into or out of the sequestration pens from/to the flow region. Magnetic forces may be employed to move micro-objects including paramagnetic materials, which can include magnetic micro-objects attached to or associated with a biological micro-object. Alternatively, or in additional, centripetal forces may be used to move micro-objects within the microfluidic channel, as well as into or out of sequestration pens or other chambers in the microfluidic device.

In another alternative mode of moving micro-objects, laser-generated dislodging forces may be used to export micro-objects or assist in exporting micro-objects from a sequestration pen or any other chamber in the microfluidic device, as described in International Patent Publication No. WO2017/117408 (Kurz, et al.), which is incorporated herein by reference in its entirety.

In some embodiments, DEP forces are combined with other forces, such as fluidic flow (e.g., bulk fluidic flow in a channel or localized fluidic flow actuated by deformation of a deformable surface of the microfluidic device, laser generated dislodging forces, and/or gravitational force), so as to manipulate, transport, separate and sort micro-objects and/or droplets within the microfluidic circuit 120. In some embodiments, the DEP forces can be applied prior to the other forces. In other embodiments, the DEP forces can be applied after the other forces. In still other instances, the DEP forces can be applied in an alternating manner with the other forces. For the microfluidic devices described herein, repositioning of micro-objects may not generally rely upon gravity or hydrodynamic forces to position or trap micro-objects at a selected position. Gravity may be chosen as one form of repositioning force, but the ability to reposition of micro-objects within the microfluidic device does not rely solely upon the use of gravity. While fluid flow in the microfluidic channels may be used to introduce micro-objects into the microfluidic channels (e.g., flow region), such regional flow is not relied upon to pen or unpen micro-objects, while localized flow (e.g., force derived from actuating a deformable surface) may, in some embodiments, be selected from amongst the other types of repositioning forces described herein to pen or unpen micro-objects or to export them from the microfluidic device.

When DEP is used to reposition micro-objects, bulk fluidic flow in a channel is generally stopped prior to applying DEP to micro-objects to reposition the micro-objects within the microfluidic circuit of the device, whether the micro-objects are being repositioned from the channel into a sequestration pen or from a sequestration pen into the channel. Bulk fluidic flow may be resumed thereafter.

System. Returning to FIG. 1A, a system 150 for operating and controlling microfluidic devices is shown, such as for controlling the microfluidic device 100. The electrical power source 192 can provide electric power to the microfluidic device 100, providing biasing voltages or currents as needed. The electrical power source 192 can, for example, comprise one or more alternating current (AC) and/or direct current (DC) voltage or current sources.

System 150 can further include a media source 178. The media source 178 (e.g., a container, reservoir, or the like) can comprise multiple sections or containers, each for holding a different fluidic medium 180. Thus, the media source 178 can be a device that is outside of and separate from the microfluidic device 100, as illustrated in FIG. 1A. Alternatively, the media source 178 can be located in whole or in part inside the enclosure 102 of the microfluidic device 100. For example, the media source 178 can comprise reservoirs that are part of the microfluidic device 100.

FIG. 1A also illustrates simplified block diagram depictions of examples of control and monitoring equipment 152 that constitute part of system 150 and can be utilized in conjunction with a microfluidic device 100. As shown, examples of such control and monitoring equipment 152 can include a master controller 154 comprising a media module 160 for controlling the media source 178, a motive module 162 for controlling movement and/or selection of micro-objects (not shown) and/or medium (e.g., droplets of medium) in the microfluidic circuit 120, an imaging module 164 for controlling an imaging device (e.g., a camera, microscope, light source or any combination thereof) for capturing images (e.g., digital images), and an optional tilting module 166 for controlling the tilting of the microfluidic device 100. The control equipment 152 can also include other modules 168 for controlling, monitoring, or performing other functions with respect to the microfluidic device 100. As shown, the monitoring equipment 152 can further include a display device 170 and an input/output device 172.

The master controller 154 can comprise a control module 156 and a digital memory 158. The control module 156 can comprise, for example, a digital processor configured to operate in accordance with machine executable instructions (e.g., software, firmware, source code, or the like) stored as non-transitory data or signals in the memory 158. Alternatively, or in addition, the control module 156 can comprise hardwired digital circuitry and/or analog circuitry. The media module 160, motive module 162, imaging module 164, optional tilting module 166, and/or other modules 168 can be similarly configured. Thus, functions, processes acts, actions, or steps of a process discussed herein as being performed with respect to the microfluidic device 100 or any other microfluidic apparatus can be performed by any one or more of the master controller 154, media module 160, motive module 162, imaging module 164, optional tilting module 166, and/or other modules 168 configured as discussed above. Similarly, the master controller 154, media module 160, motive module 162, imaging module 164, optional tilting module 166, and/or other modules 168 may be communicatively coupled to transmit and receive data used in any function, process, act, action or step discussed herein.

The media module 160 controls the media source 178. For example, the media module 160 can control the media source 178 to input a selected fluidic medium 180 into the enclosure 102 (e.g., through an inlet port 107). The media module 160 can also control removal of media from the enclosure 102 (e.g., through an outlet port (not shown)). One or more media can thus be selectively input into and removed from the microfluidic circuit 120. The media module 160 can also control the flow of fluidic medium 180 in the flow path 106 inside the microfluidic circuit 120. The media module 160 may also provide conditioning gaseous conditions to the media source 178, for example, providing an environment containing 5% CO₂ (or higher). The media module 160 may also control the temperature of an enclosure of the media source, for example, to provide feeder cells in the media source with proper temperature control.

Motive module. The motive module 162 can be configured to control selection and movement of micro-objects (not shown) in the microfluidic circuit 120. The enclosure 102 of the microfluidic device 100 can comprise one or more electrokinetic mechanisms including a dielectrophoresis (DEP) electrode activation substrate, optoelectronic tweezers (OET) electrode activation substrate, electrowetting (EW) electrode activation substrate, and/or an opto-electrowetting (OEW) electrode activation substrate, where the motive module 162 can control the activation of electrodes and/or transistors (e.g., phototransistors) to select and move micro-objects and/or droplets in the flow path 106 and/or within sequestration pens 124, 126, 128, and 130. The electrokinetic mechanism(s) may be any suitable single or combined mechanism as described within the paragraphs describing motive technologies for use within the microfluidic device. A DEP configured device may include one or more electrodes that apply a non-uniform electric field in the microfluidic circuit 120 sufficient to exert a dielectrophoretic force on micro-objects in the microfluidic circuit 120. An OET configured device may include photo-activatable electrodes to provide selective control of movement of micro-objects in the microfluidic circuit 120 via light-induced dielectrophoresis.

The imaging module 164 can control the imaging device. For example, the imaging module 164 can receive and process image data from the imaging device. Image data from the imaging device can comprise any type of information captured by the imaging device (e.g., the presence or absence of micro-objects, droplets of medium, accumulation of label, such as fluorescent label, etc.). Using the information captured by the imaging device, the imaging module 164 can further calculate the position of objects (e.g., micro-objects, droplets of medium) and/or the rate of motion of such objects within the microfluidic device 100.

The imaging device (part of imaging module 164, discussed below) can comprise a device, such as a digital camera, for capturing images inside microfluidic circuit 120. In some instances, the imaging device further comprises a detector having a fast frame rate and/or high sensitivity (e.g., for low light applications). The imaging device can also include a mechanism for directing stimulating radiation and/or light beams into the microfluidic circuit 120 and collecting radiation and/or light beams reflected or emitted from the microfluidic circuit 120 (or micro-objects contained therein). The emitted light beams may be in the visible spectrum and may, e.g., include fluorescent emissions. The reflected light beams may include reflected emissions originating from an LED or a wide spectrum lamp, such as a mercury lamp (e.g., a high-pressure mercury lamp) or a Xenon arc lamp. The imaging device may further include a microscope (or an optical train), which may or may not include an eyepiece.

Support Structure. System 150 may further comprise a support structure 190 configured to support and/or hold the enclosure 102 comprising the microfluidic circuit 120. In some embodiments, the optional tilting module 166 can be configured to activate the support structure 190 to rotate the microfluidic device 100 about one or more axes of rotation. The optional tilting module 166 can be configured to support and/or hold the microfluidic device 100 in a level orientation (i.e., at 0° relative to x- and y-axes), a vertical orientation (i.e., at 90° relative to the x-axis and/or the y-axis), or any orientation therebetween. The orientation of the microfluidic device 100 (and the microfluidic circuit 120) relative to an axis is referred to herein as the “tilt” of the microfluidic device 100 (and the microfluidic circuit 120). For example, support structure 190 can optionally be used to tilt the microfluidic device 100 (e.g., as controlled by optional tilting module 166) to 0.1°, 0.2°, 0.3°, 0.4°, 0.5°, 0.6°, 0.7°, 0.8°, 0.9°, 1°, 2°, 3°, 4°, 5°, 10°, 15°, 20°, 25°, 30°, 35°, 40°, 45°, 50°, 55°, 60°, 65°, 70°, 75°, 80°, 90° relative to the x-axis or any degree therebetween. When the microfluidic device is tilted at angles greater than about 15, tilting may be performed to create bulk movement of micro-objects into/out of sequestration pens from/into the flow region (e.g., microfluidic channel). In some embodiments, the support structure 190 can hold the microfluidic device 100 at a fixed angle of 0.1°, 0.2°, 0.3°, 0.4°, 0.5°, 0.6°, 0.7°, 0.8°, 0.9°, 1°, 2°, 3°, 4°, 5°, or 10° relative to the x-axis (horizontal), so long as DEP is an effective force to move micro-objects out of the sequestration pens into the microfluidic channel. Since the surface of the electrode activation substrate is substantially flat, DEP forces may be used even when the far end of the sequestration pen, opposite its opening to the microfluidic channel, is disposed at a position lower in a vertical direction than the microfluidic channel.

In some embodiments where the microfluidic device is tilted or held at a fixed angle relative to horizontal, the microfluidic device 100 may be disposed in an orientation such that the inner surface of the base of the flow path 106 is positioned at an angle above or below the inner surface of the base of the one or more sequestration pens opening laterally to the flow path. The term “above” as used herein denotes that the flow path 106 is positioned higher than the one or more sequestration pens on a vertical axis defined by the force of gravity (i.e., an object in a sequestration pen above a flow path 106 would have a higher gravitational potential energy than an object in the flow path), and inversely, for positioning of the flow path 106 below one or more sequestration pens. In some embodiments, the support structure 190 may be held at a fixed angle of less than about 5°, about 4°, about 3° or less than about 2° relative to the x-axis (horizontal), thereby placing the sequestration pens at a lower potential energy relative to the flow path. In some other embodiments, when long term culturing (e.g., for more than about 2, 3, 4, 5, 6, 7 or more days) is performed within the microfluidic device, the device may be supported on a culturing support and may be tilted at a greater angle of about 10°, 15°, 20°, 25°, 30°, or any angle therebetween to retain biological micro-objects within the sequestration pens during the long-term culturing period. At the end of the culturing period, the microfluidic device containing the cultured biological micro-objects may be returned to the support 190 within system 150, where the angle of tilting is decreased to values as described above, affording the use of DEP to move the biological micro-objects out of the sequestration pens. Further examples of the use of gravitational forces induced by tilting are described in U.S. Pat. No. 9,744,533 (Breinlinger et al.), the contents of which are herein incorporated by reference in its entirety.

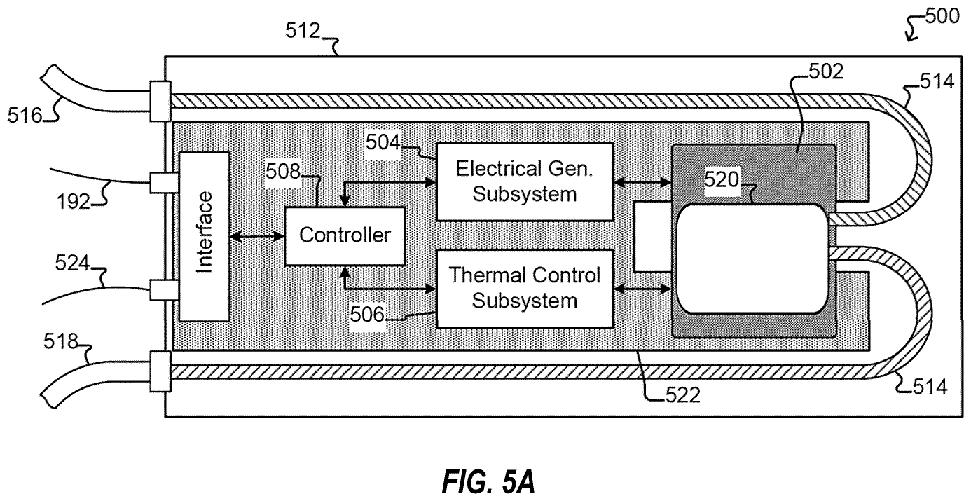

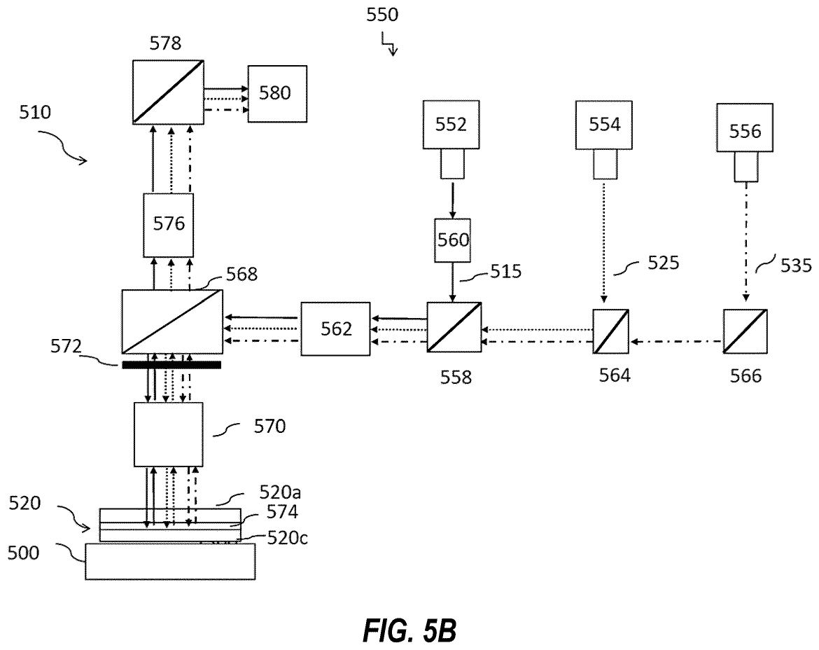

Nest. Turning now to FIG. 5A, the system 150 can include a structure (also referred to as a “nest”) 500 configured to hold a microfluidic device 520, which may be like microfluidic device 100, 200, or any other microfluidic device described herein. The nest 500 can include a socket 502 capable of interfacing with the microfluidic device 520 (e.g., an optically actuated electrokinetic device 100, 200, etc.) and providing electrical connections from power source 192 to microfluidic device 520. The nest 500 can further include an integrated electrical signal generation subsystem 504. The electrical signal generation subsystem 504 can be configured to supply a biasing voltage to socket 502 such that the biasing voltage is applied across a pair of electrodes in the microfluidic device 520 when it is being held by socket 502. Thus, the electrical signal generation subsystem 504 can be part of power source 192. The ability to apply a biasing voltage to microfluidic device 520 does not mean that a biasing voltage will be applied at all times when the microfluidic device 520 is held by the socket 502. Rather, in most cases, the biasing voltage will be applied intermittently, e.g., only as needed to facilitate the generation of electrokinetic forces, such as dielectrophoresis or electro-wetting, in the microfluidic device 520.

As illustrated in FIG. 5A, the nest 500 can include a printed circuit board assembly (PCBA) 522. The electrical signal generation subsystem 504 can be mounted on and electrically integrated into the PCBA 522. The exemplary support includes socket 502 mounted on PCBA 522, as well.

In some embodiments, the nest 500 can comprise an electrical signal generation subsystem 504 configured to measure the amplified voltage at the microfluidic device 520 and then adjust its own output voltage as needed such that the measured voltage at the microfluidic device 520 is the desired value. In some embodiments, the waveform amplification circuit can have a +6.5V to −6.5V power supply generated by a pair of DC-DC converters mounted on the PCBA 322, resulting in a signal of up to 13 Vpp at the microfluidic device 520.

In certain embodiments, the nest 500 further comprises a controller 508, such as a microprocessor used to sense and/or control the electrical signal generation subsystem 504. Examples of suitable microprocessors include the Arduino™ microprocessors, such as the Arduino Nano™. The controller 508 may be used to perform functions and analysis or may communicate with an external master controller 154 (shown in FIG. 1A) to perform functions and analysis. In the embodiment illustrated in FIG. 3A the controller 308 communicates with the master controller 154 (of FIG. 1A) through an interface (e.g., a plug or connector).

As illustrated in FIG. 5A, the support structure 500 (e.g., nest) can further include a thermal control subsystem 506. The thermal control subsystem 506 can be configured to regulate the temperature of microfluidic device 520 held by the support structure 500. For example, the thermal control subsystem 506 can include a Peltier thermoelectric device (not shown) and a cooling unit (not shown). In the embodiment illustrated in FIG. 5A, the support structure 500 comprises an inlet 516 and an outlet 518 to receive cooled fluid from an external reservoir (not shown) of the cooling unit, introduce the cooled fluid into the fluidic path 514 and through the cooling block, and then return the cooled fluid to the external reservoir. In some embodiments, the Peltier thermoelectric device, the cooling unit, and/or the fluidic path 514 can be mounted on a casing 512 of the support structure 500. In some embodiments, the thermal control subsystem 506 is configured to regulate the temperature of the Peltier thermoelectric device so as to achieve a target temperature for the microfluidic device 520. Temperature regulation of the Peltier thermoelectric device can be achieved, for example, by a thermoelectric power supply, such as a Pololu™ thermoelectric power supply (Pololu Robotics and Electronics Corp.). The thermal control subsystem 506 can include a feedback circuit, such as a temperature value provided by an analog circuit. Alternatively, the feedback circuit can be provided by a digital circuit.

The nest 500 can include a serial port 524 which allows the microprocessor of the controller 508 to communicate with an external master controller 154 via the interface. In addition, the microprocessor of the controller 508 can communicate (e.g., via a Plink tool (not shown)) with the electrical signal generation subsystem 504 and thermal control subsystem 506. Thus, via the combination of the controller 508, the interface, and the serial port 524, the electrical signal generation subsystem 504 and the thermal control subsystem 506 can communicate with the external master controller 154. In this manner, the master controller 154 can, among other things, assist the electrical signal generation subsystem 504 by performing scaling calculations for output voltage adjustments. A Graphical User Interface (GUI) (not shown) provided via a display device 170 coupled to the external master controller 154, can be configured to plot temperature and waveform data obtained from the thermal control subsystem 506 and the electrical signal generation subsystem 504, respectively. Alternatively, or in addition, the GUI can allow for updates to the controller 508, the thermal control subsystem 506, and the electrical signal generation subsystem 504.

Optical sub-system. FIG. 5B is a schematic of an optical sub-system 550 having an optical apparatus 510 for imaging and manipulating micro-objects in a microfluidic device 520, which can be any microfluidic device described herein. The optical apparatus 510 can be configured to perform imaging, analysis and manipulation of one or more micro-objects within the enclosure of the microfluidic device 520.

The optical apparatus 510 may have a first light source 552, a second light source 554, and a third light source 556. The first light source 552 can transmit light to a structured light modulator 560, which can include a digital mirror device (DMD) or a microshutter array system (MSA), either of which can be configured to receive light from the first light source 552 and selectively transmit a subset of the received light into the optical apparatus 510. Alternatively, the structured light modulator 560 can include a device that produces its own light (and thus dispenses with the need for a light source 552), such as an organic light emitting diode display (OLED), a liquid crystal on silicon (LCOS) device, a ferroelectric liquid crystal on silicon device (FLCOS), or a transmissive liquid crystal display (LCD). The structured light modulator 560 can be, for example, a projector. Thus, the structured light modulator 560 can be capable of emitting both structured and unstructured light. In certain embodiments, an imaging module and/or motive module of the system can control the structured light modulator 560.

In embodiments when the structured light modulator 560 includes a mirror, the modulator can have a plurality of mirrors. Each mirror of the plurality of mirrors can have a size of about 5 microns×5 microns to about 10 microns×10 microns, or any values therebetween. The structured light modulator 560 can include an array of mirrors (or pixels) that is 2000×1000, 2580×1600, 3000×2000, or any values therebetween. In some embodiments, only a portion of an illumination area of the structured light modulator 560 is used. The structured light modulator 560 can transmit the selected subset of light to a first dichroic beam splitter 558, which can reflect this light to a first tube lens 562.

The first tube lens 562 can have a large clear aperture, for example, a diameter larger than about 40 mm to about 50 mm, or more, providing a large field of view. Thus, the first tube lens 562 can have an aperture that is large enough to capture all (or substantially all) of the light beams emanating from the structured light modulator 560.

The structured light 515 having a wavelength of about 400 nm to about 710 nm, may alternatively or in addition, provide fluorescent excitation illumination to the microfluidic device.

The second light source 554 may provide unstructured brightfield illumination. The brightfield illumination light 525 may have any suitable wavelength, and in some embodiments, may have a wavelength of about 400 nm to about 760 nm. The second light source 554 can transmit light to a second dichroic beam splitter 564 (which also may receive illumination light 535 from the third light source 556), and the second light, brightfield illumination light 525, may be transmitted therefrom to the first dichroic beam splitter 558. The second light, brightfield illumination light 525, may then be transmitted from the first dichroic beam splitter 558 to the first tube lens 562.

The third light source 556 can transmit light through a matched pair relay lens (not shown) to a mirror 566. The third illumination light 535 may therefrom be reflected to the second dichroic beam splitter 5338 and be transmitted therefrom to the first beam splitter 5338, and onward to the first tube lens 5381. The third illumination light 535 may be a laser and may have any suitable wavelength. In some embodiments, the laser illumination 535 may have a wavelength of about 350 nm to about 900 nm. The laser illumination 535 may be configured to heat portions of one or more sequestration pens within the microfluidic device. The laser illumination 535 may be configured to heat fluidic medium, a micro-object, a wall or a portion of a wall of a sequestration pen, a metal target disposed within a microfluidic channel or sequestration pen of the microfluidic channel, or a photoreversible physical barrier within the microfluidic device, and described in more detail in U. S. Application Publication Nos. 2017/0165667 (Beaumont, et al.) and 2018/0298318 (Kurz, et al.), each of which disclosure is herein incorporated by reference in its entirety. In other embodiments, the laser illumination 535 may be configured to initiate photocleavage of surface modifying moieties of a modified surface of the microfluidic device or photocleavage of moieties providing adherent functionalities for micro-objects within a sequestration pen within the microfluidic device. Further details of photocleavage using a laser may be found in International Application Publication No. WO2017/205830 (Lowe, Jr. et al.), which disclosure is herein incorporated by reference in its entirety.

The light from the first, second, and third light sources (552, 554, 556) passes through the first tube lens 562 and is transmitted to a third dichroic beam splitter 568 and filter changer 572. The third dichroic beam splitter 568 can reflect a portion of the light and transmit the light through one or more filters in the filter changer 572 and to the objective 570, which may be an objective changer with a plurality of different objectives that can be switched on demand. Some of the light (515, 525, and/or 535) may pass through the third dichroic beam splitter 568 and be terminated or absorbed by a beam block (not shown). The light reflected from the third dichroic beam splitter 568 passes through the objective 570 to illuminate the sample plane 574, which can be a portion of a microfluidic device 520 such as the sequestration pens described herein.

The nest 500, as described in FIG. 5A, can be integrated with the optical apparatus 510 and be a part of the apparatus 510. The nest 500 can provide electrical connection to the enclosure and be further configured to provide fluidic connections to the enclosure. Users may load the microfluidic apparatus 520 into the nest 500. In some other embodiments, the nest 500 can be a separate component independent of the optical apparatus 510.

Light can be reflected off and/or emitted from the sample plane 574 to pass back through the objective 570, through the filter changer 572, and through the third dichroic beam splitter 568 to a second tube lens 576. The light can pass through the second tube lens 576 (or imaging tube lens 576) and be reflected from a mirror 578 to an imaging sensor 580. Stray light baffles (not shown) can be placed between the first tube lens 562 and the third dichroic beam splitter 568, between the third dichroic beam splitter 568 and the second tube lens 576, and between the second tube lens 576 and the imaging sensor 580.

Objective. The optical apparatus can comprise the objective lens 570 that is specifically designed and configured for viewing and manipulating of micro-objects in the microfluidic device 520. For example, conventional microscope objective lenses are designed to view micro-objects on a slide or through 5 mm of aqueous fluid, while micro-objects in the microfluidic device 520 are inside the plurality of sequestration pens within the viewing plane 574 which have a depth of 20, 30, 40, 50, 60 70, 80 microns or any values therebetween. In some embodiments, a transparent cover 520a, for example, glass or ITO cover with a thickness of about 750 microns, can be placed on top of the plurality of sequestration pens, which are disposed above a microfluidic substrate 520c. Thus, the images of the micro-objects obtained by using the conventional microscope objective lenses may have large aberrations such as spherical and chromatic aberrations, which can degrade the quality of the images. The objective lens 570 of the optical apparatus 510 can be configured to correct the spherical and chromatic aberrations in the optical apparatus 1350. The objective lens 570 can have one or more magnification levels available such as, 4X, 10X, 20X.

Modes of illumination. In some embodiments, the structured light modulator 560 can be configured to modulate light beams received from the first light source 552 and transmits a plurality of illumination light beams 515, which are structured light beams, into the enclosure of the microfluidic device, e.g., the region containing the sequestration pens. The structured light beams can comprise the plurality of illumination light beams. The plurality of illumination light beams can be selectively activated to generate a plurality of illuminations patterns. In some embodiments, the structured light modulator 560 can be configured to generate an illumination pattern, similarly as described for FIGS. 4A-4B, which can be moved and adjusted. The optical apparatus 560 can further comprise a control unit (not shown) which is configured to adjust the illumination pattern to selectively activate the one or more of the plurality of DEP electrodes of a substrate 520c and generate DEP forces to move the one or more micro-objects inside the plurality of sequestration pens within the microfluidic device 520. For example, the plurality of illuminations patterns can be adjusted over time in a controlled manner to manipulate the micro-objects in the microfluidic device 520. Each of the plurality of illumination patterns can be shifted to shift the location of the DEP force generated and to move the structured light for one position to another in order to move the micro-objects within the enclosure of the microfluidic apparatus 520.

In some embodiments, the optical apparatus 510 may be configured such that each of the plurality of sequestration pens in the sample plane 574 within the field of view is simultaneously in focus at the image sensor 580 and at the structured light modulator 560. In some embodiments, the structured light modulator 560 can be disposed at a conjugate plane of the image sensor 580. In various embodiments, the optical apparatus 510 can have a confocal configuration or confocal property. The optical apparatus 510 can be further configured such that only each interior area of the flow region and/or each of the plurality of sequestration pens in the sample plane 574 within the field of view is imaged onto the image sensor 580 in order to reduce overall noise to thereby increase the contrast and resolution of the image.

In some embodiments, the first tube lens 562 can be configured to generate collimated light beams and transmit the collimated light beams to the objective lens 570. The objective 570 can receive the collimated light beams from the first tube lens 562 and focus the collimated light beams into each interior area of the flow region and each of the plurality of sequestration pens in the sample plane 574 within the field of view of the image sensor 580 or the optical apparatus 510. In some embodiments, the first tube lens 562 can be configured to generate a plurality of collimated light beams and transmit the plurality of collimated light beams to the objective lens 570. The objective 570 can receive the plurality of collimated light beams from the first tube lens 562 and converge the plurality of collimated light beams into each of the plurality of sequestration pens in the sample plane 574 within the field of view of the image sensor 580 or the optical apparatus 510.

In some embodiments, the optical apparatus 510 can be configured to illuminate the at least a portion of sequestration pens with a plurality of illumination spots. The objective 570 can receive the plurality of collimated light beams from the first tube lens 562 and project the plurality of illumination spots, which may form an illumination pattern, into each of the plurality of sequestration pens in the sample plane 574 within the field of view. For example, each of the plurality of illumination spots can have a size of about 5 microns×5 microns; 10 microns×10 microns; 10 microns×30 microns, 30 microns×60 microns, 40 microns×40 microns, 40 microns×60 microns, 60 microns×120 microns, 80 microns×100 microns, 100 microns×140 microns and any values there between. The illumination spots may individually have a shape that is circular, square, or rectangular. Alternatively, the illumination spots may be grouped within a plurality of illumination spots (e.g., an illumination pattern) to form a larger polygonal shape such as a rectangle, square, or wedge shape. The illumination pattern may enclose (e.g., surround) an unilluminated space that may be square, rectangular or polygonal. For example, each of the plurality of illumination spots can have an area of about 150 to about 3000, about 4000 to about 10000, or 5000 to about 15000 square microns. An illumination pattern may have an area of about 1000 to about 8000, about 4000 to about 10000, 7000 to about 20000, 8000 to about 22000, 10000 to about 25000 square microns and any values there between.

The optical system 510 may be used to determine how to reposition micro-objects and into and out of the sequestration pens of the microfluidic device, as well as to count the number of micro-objects present within the microfluidic circuit of the device. Further details of repositioning and counting micro-objects are found in U. S. Application Publication No. 2016/0160259 (Du); U.S. Pat. No. 9,996,920 (Du et al.); and International Application Publication No. WO2017/102748 (Kim, et al.). The optical system 510 may also be employed in assay methods to determine concentrations of reagents/assay products, and further details are found in U.S. Pat. No. 8,921,055 (Chapman), 10,010,882 (White et al.), and 9,889,445 (Chapman et al.); International Application Publication No. WO2017/181135 (Lionberger, et al.); and International Application Serial No. PCT/US2018/055918 (Lionberger, et al.). Further details of the features of optical apparatuses suitable for use within a system for observing and manipulating micro-objects within a microfluidic device, as described herein, may be found in WO2018/102747 (Lundquist, et al), the disclosure of which is herein incorporated by reference in its entirety.

Additional system components for maintenance of viability of cells within the sequestration pens of the microfluidic device. In order to promote growth and/or expansion of cell populations, environmental conditions conducive to maintaining functional cells may be provided by additional components of the system. For example, such additional components can provide nutrients, cell growth signaling species, pH modulation, gas exchange, temperature control, and removal of waste products from cells.

### EXAMPLES

### General Materials and Methods

System and Microfluidic device: An OptoSelect chip, a microfluidic (or nanofluidic) device manufactured by Berkeley Lights, Inc. and controlled by an optical instrument which was also manufactured by Berkeley Lights, Inc. The instrument included: a mounting stage for the chip coupled to a temperature controller; a pump and fluid medium conditioning component; and an optical train including a camera and a structured light source suitable for activating phototransistors within the chip. The OptoSelect™ chip included a substrate configured with OptoElectroPositioning (OEP™) technology, which provides a phototransistor-activated OET force. The chip also included a plurality of microfluidic channels, each having a plurality of NanoPen™ chambers (orsequestration pens) fluidically connected thereto. The volume of each sequestration pen was around 1×10⁶ cubic microns.

Priming solution: Complete growth medium containing 0.1% Pluronic® F127 ((Life Technologies® Cat #P6866).

Preparation for culturing: The microfluidic device having a modified surface was loaded onto the system and purged with 100% carbon dioxide at 15 psi for 5 min. Immediately following the carbon dioxide purge, the priming solution was perfused through the microfluidic device at 5 microliters/sec for 8 min. Culture medium was then flowed through the microfluidic device at 5 microliters/sec for 5 min.

Priming regime. 250 microliters of 100% carbon dioxide was flowed in at a rate of 12 microliters/sec. This was followed by 250 microliters of PBS containing 0.1% Pluronic® F27 (Life Technologies® Cat #P6866), flowed in at 12 microliters/sec. The final step of priming included 250 microliters of PBS, flowed in at 12 microliters/sec. Introduction of the culture medium follows.

Perfusion regime. The perfusion method was either of the following two methods:
1. Perfuse at 0.01 microliters/sec for 2h; perfuse at 2 microliters/sec for 64 sec; and repeat.
2. Perfuse at 0.02 microliters/sec for 100 sec; stop flow 500 sec; perfuse at 2 microliters/sec for 64 sec; and repeat.

### Example 1. Preparation of a Functionalized Surface of an Unpatterned Silicon Wafer

A silicon wafer (780 microns thick, 1 cm by 1 cm) was treated in an oxygen plasma cleaner (Nordson Asymtek) for 5 min, using 100 W power, 240 mTorr pressure and 440 sccm oxygen flow rate. The plasma treated silicon wafer was treated in a vacuum reactor with (11-azidoundecyl) trimethoxy silane (300 microliters) in a foil boat in the bottom of the vacuum reactor in the presence of magnesium sulfate heptahydrate (0.5g, Acros Cat. #10034-99-8), as a water reactant source in a separate foil boat in the bottom of the vacuum reactor. The chamber was then pumped to 750 mTorr using a vacuum pump and then sealed. The vacuum reactor was placed within an oven heated at 110° C. for 24-48 h. This introduced a modified surface to the wafer, where the modified surface had a structure of Formula I:

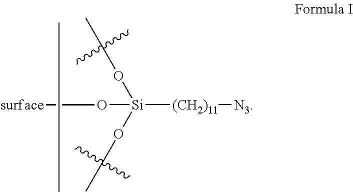

After cooling to room temperature and introducing argon to the evacuated chamber, the wafer was removed from the reactor. The wafer was rinsed with acetone, isopropanol, and dried under a stream of nitrogen. Confirmation of introduction of the modified surface was made by ellipsometry and contact angle goniometry.

Alternatively, the silicon wafer was cut to size to fit within the bottom of a flat bottomed wellplate before introducing the functionalized surface of Formula I upon it, and a plurality of the formatted silicon wafers were functionalized at the same time.

### Example 2. Preparation of a Planar Unpatterned Silicon Wafer Having a Streptavidin Functionalized Surface

The product silicon wafer from Example 1, having a surface of Formula I as described above, was treated with dibenzylcyclooctynyl (DBCO) Streptavidin (SAV), Nanocs, Cat. #SV1-DB-1, where there are 2-7 DBCO for each molecule of SAV) by contacting the silicon wafer with an aqueous solution containing a 2 micromolar solution of the commercially available DBCO-SAV. The reaction was allowed to proceed at room temperature for at least 1 h. The unpatterned silicon wafer having a modified surface of Formula II was then rinsed with 1×PBS.

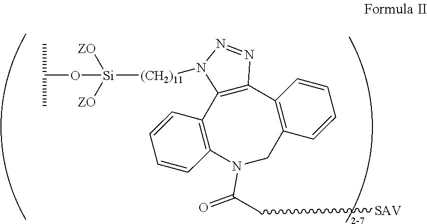

### Example 3. Synthesis of DBCO-Labeled Streptavidin (SAV) Compound 1

5 mg of lyophilized SAV (ThermoFisher PN #S888) was dissolved into 1 mL of 1×PBS (Gibco) and 1 mL of 2 mM Na₂CO₃ (Acros) in 1×PBS. 10 mg of neat DBCO-PEG13-NHS (Compound 2, Click Chemistry Tools PN #1015-10) was dissolved into 0.4 mL of dry DMSO. 16 uL of the DBCO-PEG13-NHS solution was added to the SAV solution and mixed at 400 RPM at 25° C. for 4 h on an Eppendorf ThermoMixer. The labeled SAV (Compound 1) was purified from the DBCO-PEG13-NHS by passing the reaction mixture through Zeba size exclusion chromatography spin columns (ThermoFisher PN #89882), and used without further purification.

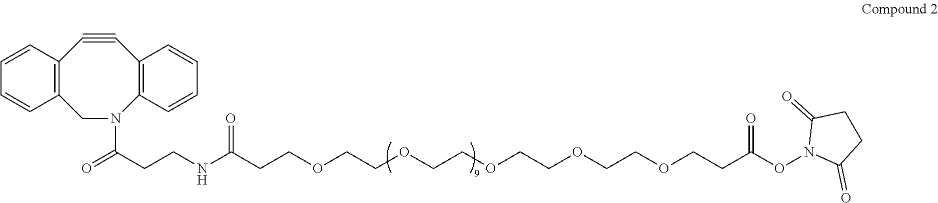

### Example 4. Preparation of a Planar Unpatterned Silicon Wafer Having a Streptavidin Functionalized Surface

The product silicon wafer from Example 1, having a surface of Formula I as described above, was treated with Compound 1 (DBCO linked Streptavidin (SAV), product of Example 3, having a PEG 13 linker, where there are 2-7 DBCO for each molecule of SAV) by contacting the silicon wafer with an aqueous solution containing 2 micromolar Compound 1. The reaction was allowed to proceed at room temperature for at least 1 h. The unpatterned silicon wafer having a streptavidin covalently functionalized surface including a PEG 13 linker was then rinsed with 1×PBS.

### Example 5. Comparison of Functionalization of Silicon Wafers with Commercially Available DBCO Linked SAV Compared with Functionalization Using Compound 1

Comparison of the SAV modified surfaces was made by ellipsometry and contact angle goniometry after each step of introduction of reactive azide moieties (Example 1); introduction of respective SAV layers; followed by introduction of biotinylated anti-CD28, where the concentrations of reagents and reaction conditions were the same. Sample 2, using the DBCO SAV reagent having a linker including a PEG13 moiety, clearly provided a more robust functionalization of SAV than that of Sample 1, and subsequently, more robust functionalization by biotinylated anti-CD2 binding to the SAV binding sites. See Table 2.

TABLE 2

| | | Overall | Overall | | Thickness of |
|---|---|---|---|---|---|
| | | Thickness | Thickness | Thickness of | anti-CD28 |
| | Thickness of | after DBCO | after anti-CD | SAV layer | layer |
| | azide layer | SAV coupling | 28 binding | introduced | introduced |
| Sample | (Angstroms) | (Angstroms) | (Angstroms) | (Angstroms) | (Angstroms) |
| Wafer 1, from | 13.31 | 25.22 | 28.54 | 11.91 | 3.32 |
| Ex. 2 | | | | | |
| Wafer 2, from | 13.43 | 45.08 | 62.37 | 31.65 | 17.29 |
| Ex. 4 | | | | | |

Without being bound by theory, it was shown that a linker having a length of at least 5 PEG repeat units up to about 20 PEG repeat units (alternatively, a linker having a length from about 20 Angstroms to about 100 Angstroms) may provide superior levels of coupling to the reactive moieties on this reactive surface of the silicon wafer. This is further demonstrated by the additional thickness of the layer of anti-CD28 introduced in Sample Wafer 2, as more SAV binding sites were available.

### Example 6. Preparation of Planar Patterned Surfaces, and Further Elaboration to Provide Antigen Presenting Surfaces within a Plurality of Regions Separated by a Differing Region Having No Activation Functionalization

Indium tin oxide (ITO) wafers were fabricated to have a patterned plurality of regions of amorphous silicon upon the ITO. The regions were a.) 1 micron diameter round amorphous silicon regions separated by three microns from each other or b.) 2 micron square amorphous silicon regions separated by 2 microns from each other.

The patterned wafers were cleaned prior to functionalization by sonication for 10 minutes in acetone, rinsed with deionized water, and dried (Step 1 of FIG. 8). The patterned wafers were then treated in an oxygen plasma cleaner (Nordson Asymtek) for 5 min, using 100 W power, 240 mTorr pressure and 440 sccm oxygen flow rate.

A schematic representation of the functionalization process is shown in FIG. 8.

Initial covalent modification of the ITO surface. The indium tin oxide base layer of the wafer was functionalized by reaction with 40 mM undecynyl phosphonic acid (Sikemia Catalog #SIK7110-10) in 50% N-methylpyrrolidine (NMP)/water solution (Step 2 of FIG. 8). The cleaned surface of the wafers was submerged in the solution within a vial and sealed. The vial was maintained in a 50° C. water bath overnight. The next day, the wafers were removed and washed with 50% isopropyl alcohol/water, followed by isopropyl alcohol.

A. Biotinylation of the ITO region of the patterned surface. The alkyne functionalized ITO region was further covalently modified by reaction with 1.5 mM biotin linked to an azido reactive moiety (azide-S—S-biotin, Broadpharm Catalog #BP-2877), 0.5 mM sodium ascorbate; and 1 mM Cu(II)SO₄/THPTA in water (Step 3 of FIG. 8). Care was taken to premix the copper ligand and sodium ascorbate prior to contact with the disulfide containing biotin reagent. The surfaces were allowed to remain in contact with the biotin reagent solution for one hour. The surfaces of the wafers were then washed with water, and dried, in preparation for the next step.

B. Covalent modification of the surfaces of the plurality of amorphous silicon regions of the patterned wafer. The patterned wafers having biotinylated surface within the ITO region of the wafer was treated in a vacuum reactor with (11-azidoundecyl) trimethoxy silane (Compound 3, 300 microliters) in a foil boat in the bottom of the vacuum reactor in the presence of magnesium sulfate heptahydrate (0.5g, Acros Cat. #10034-99-8), as a water reactant source in a separate foil boat in the bottom of the vacuum reactor (Step 4 of FIG. 8). The chamber was then pumped to 750 mTorr using a vacuum pump and then sealed. The vacuum reactor was placed within an oven heated at 110° C. overnight. This introduced a modified surface to the plurality of amorphous silicon regions on the wafer, where the modified surface had a structure of Formula I:

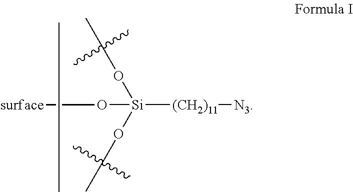

After cooling to room temperature and introducing argon to the evacuated chamber, the wafer was removed from the reactor. The wafer was rinsed with acetone, isopropanol, and dried under a stream of nitrogen. Metrology showed that the biotinylated ITO region of the patterned wafer did not have substantial amounts of contamination of the functionalizing ligands of Formula I; 10% or less contaminant was found.

C. Covalent modification of the biotin-modified ITO region of the patterned wafer to provide supportive moieties. The patterned wafers having a plurality of undecyl azido modified amorphous silicon regions separated by a biotin modified ITO region was reacted with a solution of streptavidin (SAV, 3.84 micromolar) in PBS containing 0.02% sodium azide and allowed to incubate for 30 min (Step 5 of FIG. 8). The wafer was then washed with PBS and dried.

The streptavidin modified surface of the ITO region of the patterned wafers is then modified by reaction with a 200 micromolar solution of biotin-RGD (Anaspec Catalog #AS-62347) in PBS containing 0.02% sodium azide, thereby providing adhesive moieties for general improvement in viability of the T lymphocytes when cultured upon these surfaces (Step 6 of FIG. 8). After incubating for 45 min, the wafers are rinsed with PBS and then dried.

Further generalization. The streptavidin modified surface of the ITO region of the patterned wafers may alternatively be modified by reaction with a 200 micromolar solution of biotin-PEG-5K (Jenkem Catalog #M-BIOTIN-5000) to provide hydrophilic moieties within this non-activating region of the patterned surface. Further the streptavidin surface may be modified by a mixture of the adhesive and hydrophilic moieties by reacting the streptavidin surface with a mixture of 200 micromolar stock solutions of the biotinylated moieties, in any ratio, e.g., 1:1:1:10; 10:1 or any ration therebetween.

D. Providing a secondary functionalized surface to the plurality of azido functionalized amorphous silicon regions of the patterned wafers. A solution of DBCO-SAV (Nanocs Catalog #SV1-DB-1, 2 micromolar) in PBS containing 0.02% sodium azide was contacted with the patterned wafer having a plurality of azido-functionalized amorphous silicon regions separated by a region of ITO having supportive moieties (e.g., adhesive motifs such as RGD, or hydrophilic moieties such as PEG-5K) covalently attached thereupon, for an incubation period of 30 min, providing a plurality of amorphous regions having a streptavidin functionalized surface separated by the region of supportively modified ITO surface (Step 7 of FIG. 8). The patterned wafers were maintained in PBS/0.02% sodium azide until final introduction of the antigen activating ligands.

E. Functionalization of streptavidin modified surfaces of the amorphous silicon regions of the patterned wafer (Step 8 of FIG. 8). Stepwise functionalization is performed similarly as in Example 11, first exposing the patterned surfaces to biotinylated Monomer MHC (HLA-A*02:01 MART-1 (MBL International Corp., Catalog No. MR01008, ELAGIGILTV (SEQ ID NO: 7)) in solution, and incubation for 45 minutes. After rinsing the patterned wafer having a plurality of MHC modified amorphous silicon regions with Wash Buffer, the patterned wafer is then contacted with a solution of biotinylated anti-CD28 (Miltenyi Biotec, Catalog #130-100-144) and incubated for 30-45 minutes to provide a plurality of pMHC/anti-CD28 regions separated by a supportively modified ITO region of the patterned wafer. Details of subsequent successful activation of CD8+ T lymphocytes using patterned surfaces containing the pMHC and anti-CD28 regions separated by the supportively modified regions are found in International Application Publication No. WO2019/018801, entitled “Antigenic-Presenting Synthetic Surfaces, Covalently Functionalized Surfaces, Activated T Cells, and Uses Thereof”, filed on Jul. 20, 2018, and International Application Publication No. WO2020/081875, entitled “Proto-Antigen-Presenting Synthetic Surfaces, Activated T Cells, and Uses Thereof”, filed on Oct. 17, 2019, of which each disclosure is herein incorporated by reference in its entirety.

### Example 7. Preparation of a Microfluidic Device Having Modified Interior Surfaces of Formula I

A microfluidic device (Berkeley Lights, Inc.) as described in the general experimental section above, having a first silicon electrode activation substrate and a second ITO substrate on the opposite wall, and photopatterned silicone microfluidic circuit material separating the two substrates, was treated in an oxygen plasma cleaner (Nordson Asymtek) for 1 min, using 100 W power, 240 mTorr pressure and 440 sccm oxygen flow rate. The plasma treated microfluidic device was treated in a vacuum reactor with 3-azidoundecyl) trimethoxy silane (300 microliters) in a foil boat in the bottom of the vacuum reactor in the presence of magnesium sulfate heptahydrate (0.5g, Acros), as a water reactant source in a separate foil boat in the bottom of the vacuum reactor. The chamber was then pumped to 750 mTorr using a vacuum pump and then sealed. The vacuum reactor was placed within an oven heated at 110° C. for 24-48 h. This introduced a modified surface to the microfluidic device, where the modified surface had a structure of Formula I:

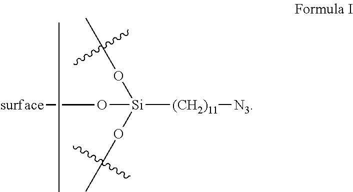

After cooling to room temperature and introducing argon to the evacuated chamber, the microfluidic device was removed from the reactor. The microfluidic device having the functionalized surface was rinsed with at least 250 microliters of deionized water, and was ready for further use.

### Example 8. Introduction of a T-Cell Activating Surface within a Microfluidic Device

A. The internal surfaces of an OptoSelect microfluidic device were covalently modified to include azido moieties as in Example 7 (Formula I). To functionalize the surface with streptavidin, the OptoSelect microfluidic device is first flushed repeatedly with 100% carbon dioxide, and then loaded with DBCO-streptavidin solution having a concentration from about 0.5 to about 2 micromolar, as produced in Example 4. After incubation for 15-30 minutes, during which the DBCO and azide groups coupled, the OptoSelect microfluidic device is washed repeatedly with 1×PBS to flush unbound DBCO-modified streptavidin.

This streptavidin surface is then further modified with biotinylated pMHC, and a selection of biotinylated anti CD28, biotinylated anti CD2 or any combination thereof. These molecules are suspended in PBS with 2% Bovine Serum Albumin at concentrations of about 1-10 micrograms/mL, in a ratio of pMHC molecules to antiCD28/antiCD2 from about 2:1 to about 1:2. This solution is perfused through the OptoSelect microfluidic device having streptavidin functionalized surfaces, facilitating conjugation to the surface. After one hour of incubation, the OptoSelect microfluidic device is flushed with PBS or media prior to loading cells.

B. Alternatively, biomolecules of interest are conjugated via biotin modification of the biomolecules to streptavidin prior to reaction with the azido-modified surfaces of the OptoSelect microfluidic device. DBCO-streptavidin and biotinylated biomolecule are prepared separately in PBS solution at concentrations in the range of 0.5-2 micromolar, then mixed at any desired ratio, as described below. After allowing the biotinylated biomolecules to conjugate to the streptavidin for at least 15 minutes, this complex is used to modify the surface of an azido-modified OptoSelect microfluidic device as described above.

Cells may be imported into the microfluidic device having at least one antigen-presenting inner surface and activated during periods of culturing similarly as described for activation of T cells with antigen-presenting beads of Example 18.

### Example 9. Covalent Modification and Functionalization of Silica Beads

9A. Silica beads having covalent PEG 3 disulfide biotin linked to streptavidin. Spherical silica beads (2.5 micron, G biosciences Catalog #786-915, having a substantially simple spherical volume, e.g., the surface area of the bead is within the range predicted by the relationship 4πr²+/−no more than 10%) were dispersed in isopropanol, and then dried. The dried beads were treated in an oxygen plasma cleaner (Nordson Asymtek) for 5 min, using 100 W power, 240 mTorr pressure and 440 sccm oxygen flow rate. The cleaned beads were treated in a vacuum reactor with (11-azidoundecyl) trimethoxy silane (300 microliters) in a foil boat in the bottom of the vacuum reactor in the presence of magnesium sulfate heptahydrate (0.5g, Acros Cat. #10034-99-8), as a water reactant source in a separate foil boat in the bottom of the vacuum reactor. The chamber was then pumped to 750 mTorr using a vacuum pump and then sealed. The vacuum reactor was placed within an oven heated at 110° C. for 24-48 h. This introduced a covalently modified surface to the beads, where the modified surface had an azide functionalized structure of Formula I:

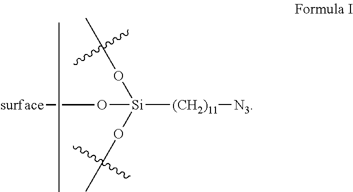

After cooling to room temperature and introducing argon to the evacuated chamber, the covalently modified beads were removed from the reactor. The beads having a covalently modified surface functionalized with azide reactive moieties were rinsed with acetone, isopropanol, and dried under a stream of nitrogen. The covalently modified azide functionalized beads were dispersed at a concentration of I mg/20 microliters in a 5.7 mM DMSO solution of dibenzylcyclooctynyl (DBCO)S—S biotin modified-PEG3 (Broadpharm, Cat. #BP-22453) then incubated at 90° C./2000 RPM in a thermomixer for 18 hours. The biotin modified beads were washed three times each in excess DMSO, then rinsed with PBS. The biotin modified beads in PBS were dispersed in PBS solution containing approximately 30 micromoles/700 microliter concentration streptavidin. The reaction mixture was shaken at 30° C./2000 RPM in a thermomixer for 30 minutes. At the completion of the reaction period, the covalently modified beads presenting streptavidin were washed three times in excess PBS. FTIR analysis determined that SAV was added to the surface (Data not shown). The disulfide containing linker may be particularly useful if cleavage from the surface may be desirable. The disulfide linker is susceptible to cleavage with dithiothreitol at concentrations that were found to be compatible with T lymphocyte viability (Data not shown).

9B. Silica beads having covalent PEG4 biotin linked to streptavidin diluted with PEG5-carboxylic acid surface-blocking molecular ligands. Beads having a covalently modified surface functionalized with azide reactive moieties of Formula 1, prepared as above in Example 9A, were rinsed with acetone, isopropanol, and dried under a stream of nitrogen. The covalently modified azide functionalized beads were dispersed at a concentration of 1 mg/10 microliters in a DMSO solution of 0.6 mM dibenzylcyclooctynyl (DBCO)-modified-PEG4-biotin (Broadpharm, Cat. #BP-22295), 5.4 mM dibenzylcyclooctynyl (DBCO)-modified-PEG5-carboxylic acid (Broadpharm, Cat. #BP-22449), and 100 mM sodium iodide then incubated at 30° C./1,000 RPM in a thermomixer for 18 hours. The biotin modified beads were washed three times each in excess DMSO, then rinsed with PBS. The biotin modified beads in PBS were dispersed in PBS solution containing approximately 10 nanomoles/1 milliliter concentration streptavidin. The reaction mixture was shaken at 30° C./1000 RPM in a thermomixer for 30 minutes. At the completion of the reaction period, the covalently modified beads presenting streptavidin were washed three times in excess PBS. FTIR analysis determined that SAV was added to the surface (Data not shown).

### Example 10. Preparation of an Antigen Presenting Surface of a Polymeric Bead

Streptavidin functionalized (covalently coupled) convoluted spherical polymeric beads (e.g., the actual surface area of the bead is greater than the relationship surface area=4πr²+/−no more than 10%, DynaBeads™ (ThermoFisher Catalog #11205D, bead stock at 6.67e8/mL)) were delivered (15 microliters; 1e7 beads) to a 1.5 mL microcentrifuge tube with 1 mL of Wash Buffer (DPBS (No Magnesium⁺², No Calcium⁺², 244 mL); EDTA (1 ml, final concentration 2 mM); and BSA (5 ml of 5%, final concentration 0.1%), and separated using a magnetic DynaBead rack. The wash/separation with 1 mL of the Wash Buffer was repeated, and a further 200 microliters of Wash Buffer was added with subsequent pulse centrifugation. Supernatant Wash Buffer was removed.

Wash Buffer (600 microliters) containing 1.5 micrograms biotinylated Monomer MHC (HLA-A*02:01 MART-1 (MBL International Corp., Catalog No. MR01008, ELAGIGILTV (SEQ ID NO: 7)) was dispensed into the microcentrifuge tube, and the beads were resuspended by pipetting up and down. The monomer was allowed to bind for 30 min at 4° C. After 15 minutes, the mixture was pipetted up and down again. The tube was pulse centrifuged and the supernatant liquid removed, and the tube was placed within the magnetic rack to remove more supernatant without removing beads.

A solution of biotinylated anti-CD28 (Miltenyi Biotec, Catalog #130-100-144, 22.5 microliters) in 600 microliters Wash Buffer was added to the microcentrifuge tube. The beads were resuspended by pipetting up and down. The beads were incubated at 4° C. for 30 min, resuspending after 15 min with another up and down pipetting. At the end of the incubation period, the tube was briefly pulse centrifuged. After placing back into the magnetic rack, and allowing separation for 1 min, the Buffer solution was aspirated away from the functionalized beads. The MHC monomer/anti-CD28 antigen presenting beads were resuspended in 100 microliters Buffer Wash, stored at 4° C., and used without further manipulation. The 1e7 2.80 micron diameter functionalized DynaBeads have a nominal (ideal predicted surface area of a sphere) surface area of about 24e6 square microns available for contact with T lymphocyte cells. However, the convolutions of this class of polymeric bead which are not necessarily accessible by T lymphocyte cells, are also functionalized in this method. Total ligand count may not reflect what is available to contact and activate T lymphocyte cells.

The MHC monomer/anti-CD28 antigen presenting beads were characterized by staining with Alexa Fluor 488-conjugated Rabbit anti Mouse IgG (H+L) Cross-adsorbed secondary antibody (Invitrogen Catalog #A-11059) and APC-conjugated anti-HLA-A2 antibody (Biolegend Catalog #343307), and characterized by flow cytometry. Further details of successful activation of CD8+ T lymphocytes by these antigen presenting beads, and their equivalent activity relative to activation by dendritic cells are found in International Application Publication No. WO2019/018801, entitled “Antigenic-Presenting Synthetic Surfaces, Covalently Functionalized Surfaces, Activated T Cells, and Uses Thereof”, filed on Jul. 20, 2018, and International Application Publication No. WO2020/081875, entitled “Proto-Antigen-Presenting Synthetic Surfaces, Activated T Cells, and Uses Thereof”, filed on Oct. 17, 2019, of which each disclosure is herein incorporated by reference in its entirety.

### Example 11. Preparation of Covalently Functionalized Glass Beads

Silica beads (2.5 micron, G biosciences Catalog #786-915, having a substantially simple spherical surface, e.g., the surface area of the bead is within the range predicted by the relationship 4πr²+/−no more than 10%) were dispersed in isopropanol, and then dried. The dried beads were treated in an oxygen plasma cleaner (Nordson Asymtek) for 5 min, using 100 W power, 240 mTorr pressure and 440 sccm oxygen flow rate. The cleaned beads were treated in a vacuum reactor with (11-azidoundecyl) trimethoxy silane (300 microliters) in a foil boat in the bottom of the vacuum reactor in the presence of magnesium sulfate heptahydrate (0.5g, Acros Cat. #10034-99-8), as a water reactant source in a separate foil boat in the bottom of the vacuum reactor. The chamber was then pumped to 750 mTorr using a vacuum pump and then sealed. The vacuum reactor was placed within an oven heated at 110° C. for 24-48 h. This introduced a covalently linked surface presenting reactive azide moieties to the beads, where the modified surface has a structure of Formula I.

After cooling to room temperature and introducing argon to the evacuated chamber, the intermediate reactive azide presenting beads were removed from the reactor and were rinsed with acetone, isopropanol, and dried under a stream of nitrogen. The azide presenting reactive beads (50 mg) were dispersed in 500 microliters DMSO with vigorous vortexing/brief sonication. The beads were pelleted, and 450 microliters of the DMSO were aspirated away from the beads. The pellet, in the remaining 50 microliters DMSO was vortexed vigorously to disperse. DBCO-SAV (52 microliters of 10 micromolar concentration, Compound 1) as synthesized in Example 3, having a PEG13 linker, was added. The beads were dispersed by tip mixing, followed by vortexing. 398 microliters of PBS with 0.02% sodium azide solution was added, followed by additional vortexing. The reaction mixture was incubated overnight on a thermomixer at 30° C., 1000 RPM.

After 16 hrs, 10 microliters of 83.7 mM DBCO-PEG5-acid were added to each sample and they were incubated an additional 30 minutes at 30° C./1,000 RPM. The beads were washed 3× in PBS/azide, then suspended in 500 microliters of the same.

These covalently functionalized beads are modified to introduce primary activating molecules and co-activating molecules as described below in Example 16.

### Example 12. Preparation of Covalently Functionalized Polystyrene Bead

Divinylbenzene-crosslinked polystyrene beads (14-20 micron, Cospheric Catalog #786-915) were dispersed in isopropanol, and then dried in a glass petri dish. The dried beads were treated in an oxygen plasma cleaner (Nordson Asymtek) for 40 seconds, using 100 W power, 240 mTorr pressure and 440 sccm oxygen flow rate. The cleaned beads were treated in a vacuum oven with (11-azidoundecyl) trimethoxy silane (Compound 5, 900 microliters) in a foil boat on the shelf of the oven in the presence of magnesium sulfate heptahydrate (1 g, Acros Cat. #10034-99-8), as a water reactant source in a separate foil boat on the same shelf of the oven. The oven was then pumped to 250 mTorr using a vacuum pump and then sealed. The oven was heated at 110° C. for 18-24 h. This introduced a covalently modified surface to the beads, where the modified surface had a structure of Formula I:

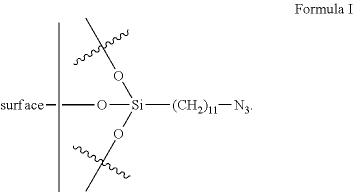

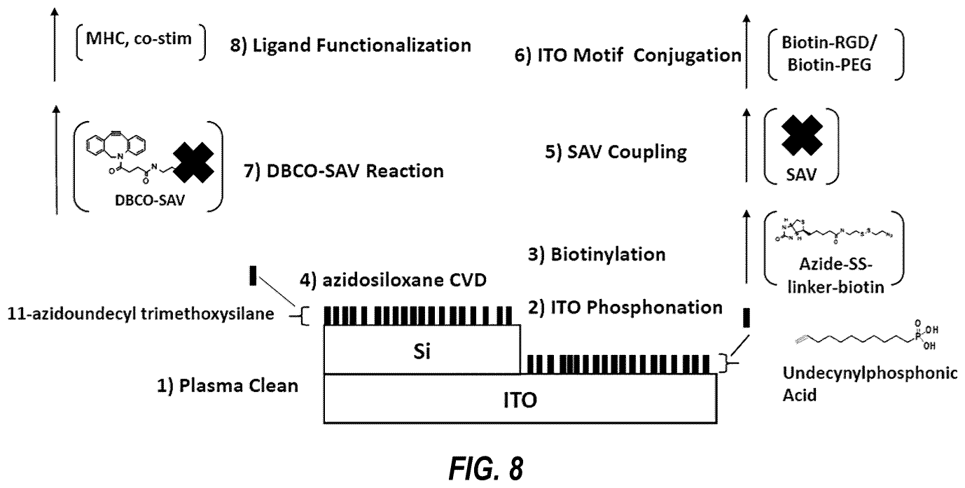

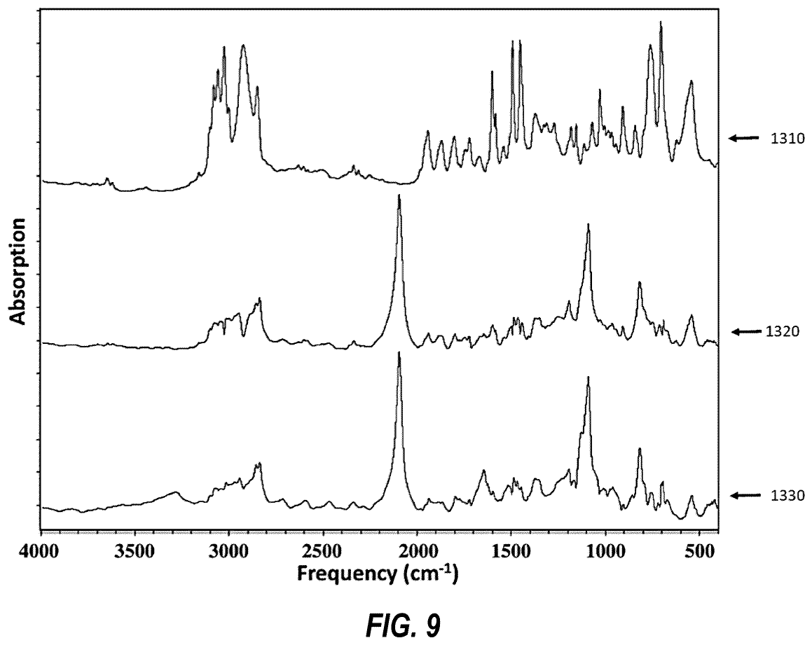

After pump-purging the oven three times, the covalently modified beads were removed from the oven and cooled. The covalently modified azide functionalized beads were dispersed at a concentration 15 mg/50 microliters in DMSO. To this, a 450 microliter solution of DBCO-labeled streptavidin (SAV) (Compound 1) at a concentration of 9.9 micromolar were added. The solution was then incubated at 30° C./1000 RPM in a thermomixer for 18 hours. The SAV modified beads were washed three times in PBS. FTIR analysis determined that SAV was added to the surface as shown in FIG. 9.

FIG. 9 shows superimposed FTIR traces of the functionalized bead as the covalently functionalized surface is built up. Trace 1310 showed the original unfunctionalized surface of the polystyrene bead. Trace 1320 showed the FTIR of the surface after introduction of the azide functionalized surface (having a structure of Formula I). Trace 1330 showed the FTIR of the surface after introduction of covalently linked PEG13-streptavidin surface to the polystyrene bead. Traces 1320 and 1330 showed introduction of FTIR absorption bands consistent with the introduction of each set of chemical species in the stepwise synthesis.

### Example 13. Preparation of an Antigen Presenting Surface of a Bead with Anti-CD28 and Anti-CD2

Streptavidin functionalized (covalently coupled) DynaBeads™ (ThermoFisher Catalog #11205D), bead stock at 6.67e8/mL, convoluted (as described above) polymeric beads) were delivered (15 microliters; 1e7 beads) to 1.5 mL microcentrifuges tube with 1 mL of Wash Buffer (DPBS (No Magnesium⁺², No Calcium⁺², 244 mL); EDTA (1 ml, final concentration 2 mM); and BSA (5 ml of 5%, final concentration 0.1%), and separated using a magnetic DynaBead rack. The wash/separation with 1 mL of the Wash Buffer was repeated, and a further 200 microliters of Wash Buffer was added with subsequent pulse centrifugation. Supernatant Wash Buffer was removed.

Wash Buffer (600 microliters) containing 0.5 micrograms biotinylated Monomer MHC (HLA-A*02:01 MART-1 (MBL International Corp., Catalog No. MR01008, ELAGIGILTV (SEQ ID NO: 7)) was dispensed into the microcentrifuge tubes, and the beads were resuspended by pipetting up and down. The monomer was allowed to bind for 30 min at 4° C. After 15 minutes, the mixtures were pipetted up and down again. The tubes were pulse-centrifuged and the supernatant liquid removed, and the tubes were placed within the magnetic rack to remove more supernatant without removing beads.

Solutions of biotinylated anti-CD28 (Biolegend, Catalog #302904) and biotinylated anti-CD2 (Biolegend, Catalog #300204) in 600 microliters Wash Buffer were added to the microcentrifuge tubes. Solutions contained a total of 3 micrograms of antibody. The solutions contained: 3 micrograms of anti-CD28 and 0 micrograms of anti-CD2, 2.25 micrograms of anti-CD28 and 0.75 micrograms of anti-CD2, 1.5 micrograms of anti-CD28 and 1.5 micrograms of anti-CD2, 0.75 micrograms of anti-CD28 and 2.25 micrograms of anti-CD2, or 0 micrograms of anti-CD28 and 3 micrograms of anti-CD2. The beads were resuspended by pipetting up and down. The beads were incubated at 4° C. for 30 min, then resuspended after 15 min with another up and down pipetting. At the end of the incubation period, the tube was briefly pulse centrifuged. After placing back into the magnetic rack, and allowing separation for 1 min, the Buffer solution was aspirated away from the functionalized beads. The MHC monomer/anti CD28 antigen presenting beads were resuspended in 100 microliters Buffer Wash, stored at 4° C., and used without further manipulation. The 1e7 2.80 micron diameter functionalized DynaBeads have a nominal surface area of about 24e6 square microns available for contact with T lymphocyte cells, but as described above, these convoluted spherical beads have a practical surface area of more than 10% above that of the nominal surface area.

### Example 14. Preparation of Covalently Functionalized Polymeric Beads. Preparation of an Intermediate Reactive Synthetic Surface

In the first step of the manufacturing process, M-450 Epoxy-functionalized paramagnetic convoluted polymeric beads (DynaBeads™, ThermoFisher Cat. #14011 (convoluted having the same meaning as above)) were reacted with Tetrabutylammonium Azide to prepare polymeric beads presenting azide reactive moieties capable of reacting with functionalizing reagents having Click chemistry compatible reactive groups.

### Example 15. Preparation of a Covalently Functionalized Synthetic Surface of a Bead

The azide-prepared convoluted beads from Example 14 were then reacted with dibenzocyclooctynyl (DBCO)-coupled Streptavidin to attach Streptavidin covalently to the polymeric beads. The DBCO-Streptavidin reagent was generated by reacting Streptavidin with amine-reactive DBCO-polyethylene glycol (PEG)13-NHS Ester, providing more than one attachment site per Streptavidin unit.

Further reaction with surface-blocking molecules. The resulting covalently functionalized polymeric beads presenting streptavidin functionalities from Example 15 may subsequently be treated with DBCO functionalized surface-blocking molecules to react with any remaining azide reactive moieties on the polymeric bead. In some instances, the DBCO functionalized surface-blocking molecule may include a PEG molecule. In some instances, the DBCO PEG molecule may be a DBCO PEG5-carboxylic acid. Streptavidin functionalized polymeric beads including additional PEG or PEG-carboxylic acid surface-blocking molecules provide superior physical behavior, demonstrating improved dispersal in aqueous environment. Additionally, the surface-blocking of remaining azide moieties prevents other unrelated/undesired components present in this or following preparation steps or activation steps from also covalently binding to the polymeric bead. Finally, introduction of the surface-blocking molecular ligands can prevent surface molecules present on the T lymphocytes from contacting reactive azide functionalities.

Further generalization. It may be desirable to modify the azide functionalized surface of Example 14 with a mixture of DBCO containing ligand molecules. For example, DBCO-polyethylene glycol (PEG)13-streptavidin (Compound 1, prepared as in Example 3) may be mixed with DBCO-PEG5-COOH (surface-blocking molecules) in various ratios, and then placed in contact with the azide functionalized beads. In some instances, the ratio of DBCO-streptavidin molecules to DBCO-PEG5-COOH may be about 1:9; about 1:6, about 1:4 or about 1:3. Without wishing to be bound by theory, surface-blocking molecular ligands prevent excessive loading of streptavidin molecules to the surface of the bead, and further provide enhanced physico-chemical behavior by providing additional hydrophilicity. The surface-blocking molecules are not limited to PEG5-COOH but may be any suitable surface-blocking molecule described herein.

### Example 16. Preparation of Covalently Modified Antigen Presenting Bead. Conjugation of Peptide-HLAs and Monoclonal Antibody Co-Activating Molecules

Materials: A. Antigen bearing major histocompatibility complex (MHC) I molecule. Biotinylated peptide-Human Leukocyte Antigen complexes (pMHC), were commercially available from MBL, Immunitrack or Biolegend. The biotinylated peptide-HLA complex included an antigenic peptide non-covalently bound to the peptide-binding groove of a Class I HLA molecule, which was produced and folded into the HLA complex at the manufacturer. The biotinylated peptide-HLA complex was also non-covalently bound to Beta2-Microglobulin. This complex was covalently biotinylated at the side chain amine of a lysine residue introduced by the BirA enzyme at a recognized location on the C-terminal peptide sequence of the HLA, also performed by the manufacturer.

B. Co-activating molecules. Biotinylated antibodies were used for costimulation and were produced from the supernatants of murine hybridoma cultures. The antibodies were conjugated to biotin through multiple amine functionalities of the side chains of lysines, randomly available at the surfaces of the antibodies. The biotinylated antibodies were commercially available (Biolegend, Miltenyi, or Thermo Fisher).

Biotinylated anti-CD28 useful in these experiments were produced from clone CD28.2, 15e8, or 9.3.

Biotinylated anti-CD2 useful in these experiments were produced from clone LT2 or RPA-2.10. Other clones may also be used in construction of covalently modified antigen presenting synthetic surfaces such as these polymeric beads.

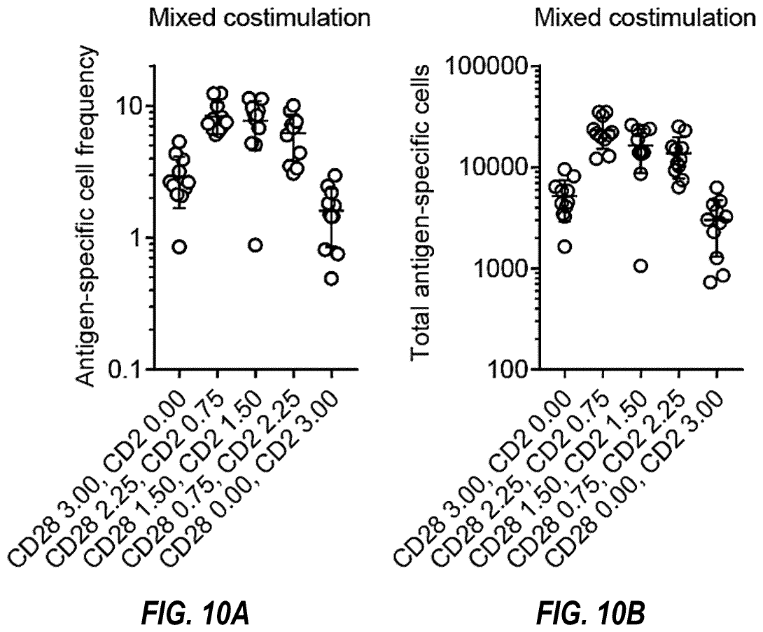

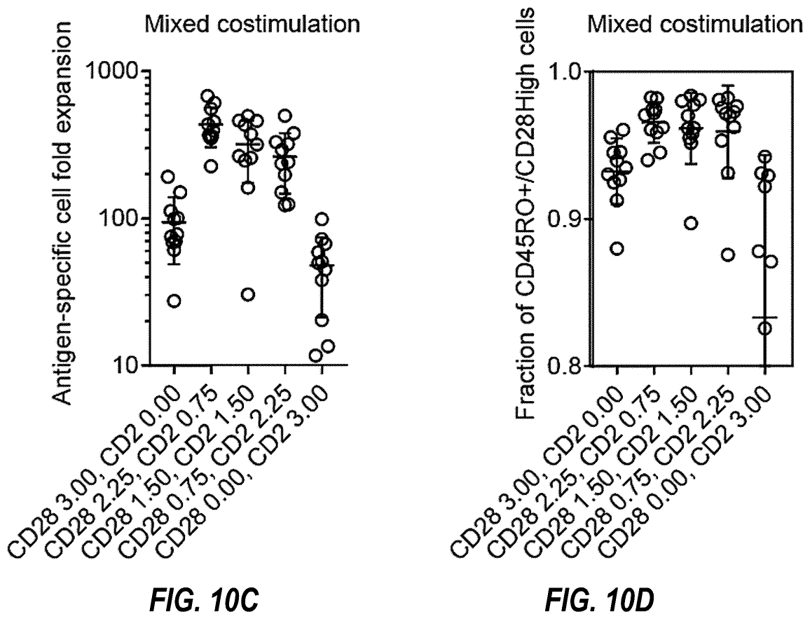

Conjugation of the primary activating and co-activating molecules to a covalently functionalized surface of a bead. The MHC molecule (containing the antigenic molecule) and the co-activating molecules were conjugated to beads produced in a two-step process. In various experiments, the ratio of the co-activating molecules—in this case, biotinylated anti-CD28 and biotinylated anti-CD2—may be varied in a range from about 100:1 to about 1:100; or from about 20:1 to about 1:20. In other experiments, the ratio of the co-activating molecules was from about 3:1 to about 1:3 or about 1:1. See FIGS. 10A-D.

pMHC loading. Streptavidin functionalized (covalently coupled) DynaBeads™ (ThermoFisher Catalog #11205D), bead stock at 6.67e8/mL, convoluted (as described above) polymeric beads) were washed with Wash Buffer (Dulbecco's Phosphate-Buffered Saline without Calcium or Magnesium; 0.1% Bovine Serum Albumin; 2 mM Ethylenediaminetetraacetic Acid). Wash buffer was pipetted into a tube, to which the Streptavidin beads were added. Typically, ˜1e7 beads were pipetted into 1 mL of Wash Buffer. The beads were collected against the wall of the tube using a magnet (e.g., DYNAL DynaMag-2, ThermoFisher Cat. #12-321-D). After the beads migrated to the wall of the tube, the Wash Buffer was removed via aspiration, avoiding the wall to which the beads were held. This wash process was repeated twice more. After the third wash, the beads were resuspended at 1.67e7 beads/mL in Wash Buffer.

The beads were then mixed with pMHC. The pMHC was added to the beads in Wash Buffer at a final concentration of 0.83 micrograms of pMHC/mL. The beads and pMHC were thoroughly mixed by vortexing, then incubated at 4° C. for 15 minutes. The beads were again vortexed, then incubated at 4° C. for an additional 15 minutes.

Co-activating molecule loading. The pMHC-functionalized beads were again captured via magnet, and the pMHC reagent mixture removed by aspiration. The beads were then brought to 1.67e7/mL in Wash Buffer. Biotinylated Anti-CD28 and biotinylated anti-CD2 (if used) were then added to the beads at a final concentration of 5 micrograms/mL of total antibody. If both anti-CD28 and anti-CD2 were used, then each antibody was added at 2.5 micrograms/mL.

The beads and pMHC were thoroughly mixed by vortexing, then incubated at 4° C. for 15 minutes. The beads are again vortexed, then incubated at 4° C. for an additional 15 minutes.

After modification by the biotinylated antibodies, the beads were captured via magnet, and the antibody mixture was removed by aspiration. The beads were resuspended in Wash Buffer at a final density of 1e8 beads/mL. The beads were used directly, without further washing.

Characterization. To assess the degree of loading and homogeneity of the resulting antigen-presenting beads, the beads were stained with antibodies that bind the pMHC and co-activating CD28/CD2 (if present) antibodies on the beads. The resulting amount of staining antibody was then quantified by flow cytometry. The number of pMHC and costimulatory antibodies on the beads was then determined using a molecular quantification kit (Quantum Simply Cellular, Bangs Labs) according to the manufacturer's instructions.

To analyze the beads, 2e5 beads were added to each of two microcentrifuge tubes with 1 mL of Wash Buffer. The pMHC quantification, and costimulation antibody quantification were performed in separate tubes. In each separate experiment, the beads were collected against the wall of the tube using a magnet, and the Wash Buffer removed. The beads were resuspended in the respective tubes in 0.1 mL of Wash Buffer, and each tube was briefly vortexed to separate the beads from the wall of the tube. To detect pMHC, 0.5 microliters of anti-HLA-A conjugated to APC (Clone BB7.2, Biolegend) was added to the first tube. The first tube was again vortexed briefly to mix the beads and detection antibody. To detect the costimulation antibodies, 0.5 microliters of anti-mouse IgG conjugated to APC was added to the second tube. Depending on the costimulation antibody clones used, different anti-mouse antibodies were used, e.g., RMG1-1 (Biolegend) is used to detect CD28.2 (anti-CD28) and RPA-2.10 (anti-CD2). The detection antibodies were incubated with the beads for 30 minutes in the dark at room temperature for each tube.

For each tube, the beads were then captured against the wall of the tube via magnet, and the staining solution was removed by aspiration. 1 mL of Wash Buffer was added to each tube, then aspirated to remove any residual staining antibody. The beads in each tube were resuspended in 0.2 mL of Wash Buffer and then the beads from each tube was transferred to a 5 mL Polystyrene tube, keeping the two sets of beads separate.

To quantify the loading of the different species, the beads were analyzed on a flow cytometer (FACS Aria or FACS Celesta, BD Biosciences). First, a sample of unstained product antigen-bearing beads is collected. A gate is drawn around the singlet and doublet beads. Doublet beads are discriminated from singlet beads based on their higher forward and side scatter amplitudes. Typically, approximately 10,000 bead events were recorded. The beads stained for pMHC and costimulation antibodies are then analyzed in separate experiments. Again, approximately 10,000 bead events were collected for each sample, and the APC median fluorescence intensity (MFI) and coefficient of variation of the APC MFI (100*[Standard Deviation of the MFI]/[MFI]) of the singlet bead events was recorded.

To determine the number of pMHC and costimulation antibodies per bead, a molecular quantification kit is used. The kit (Quantum Simply Cellular (Bangs Laboratories) included a set of beads with specified antibody binding capacities (determined by the manufacturer). These beads are used to capture the detection antibody. Briefly, the quantification beads are incubated with saturating amounts of the detection binding, then washed thoroughly to remove excess antibody. The beads with different binding capacities are mixed, along with negative control beads and resuspended in Wash Buffer. The mixed beads are then analyzed by Flow Cytometry. The APC MFI of each bead with specified binding capacity is recorded, and a linear fit of the MFI vs binding capacity is generated. The MFI of the aAPCs is then used to determine the number of detection antibodies bound per aAPC. This number is equal to the number of (pMHC or costimulation) antibodies on the bead. The following Table 3 shows results for:

A. Antigen-presenting convoluted polymeric beads produced in Examples 14-15 and functionalized above in this experiment.

B. Antigen-presenting substantially spherical silica beads as produced in Example 9B

TABLE 3

| | | Ligands/ | Ligands/ | Target antibodies |
|---|---|---|---|---|
| Condition | Labeling | Bead | sq um | (ug)/mg beads |
| A. Polymeric | HLA | 487209 | 19781 | 8.1 |
| bead | | | | |
| A. Polymeric | Costim | 426992 | 17336 | 7.1 |
| bead | | | | |
| B. 4 micron | HLA | 604471 | 12026 | 2.35 |
| monodisperse | | | | |
| silica (10B) | | | | |
| B. 4 micron | Costim | 760489 | 15129 | 2.96 |
| monodisperse | | | | |
| silica (10B) | | | | |

### Example 17. Stimulation by Antigen-Presenting Beads

Input cell populations. To increase the number of antigen-specific, CD8⁺ T cells plated per well, CD8+ T cells were first isolated from Peripheral Blood Mononuclear Cells using commercially available reagents. The cells can be isolated using negative selection, e.g., EasySep™ Human CD8+ T Cell Isolation Kit (StemCell Technologies) or by positive selection, e.g., CliniMACS CD8 Reagent (Miltenyi Biotec). The CD8⁺ T cells were isolated according to the manufacturers recommended protocol. Alternatively, different subsets of T cells can be isolated, e.g., Naïve CD8⁺ T cells only, or a less-stringent purification can be performed, e.g., depletion of Monocytes by CliniMACS CD14 Reagent (Miltenyi Biotec). Alternatively, if T cells specific for a Class II-restricted antigen are desired, CD4⁺ T cells can be isolated by corresponding methods.

First T cell stimulation period. The enriched CD8+ T cells were resuspended at 1e6/mL in media with IL-21 at 30 nanograms/mL (R&D Systems). The media used for T cells was Advanced RPMI 1640 Medium (Thermo Fisher) supplemented with 10% Human AB Serum (Corning CellGro) plus GlutaMax (Thermo Fisher) and 50 micromolar Beta-MercaptoEthanol (Thermo Fisher) or ImmunoCult™-XF T Cell Expansion Medium (StemCell Technologies).

Antigen-presenting beads were prepared as described in Example 16, where the convoluted polymeric beads were loaded at a final concentration of 0.83 micrograms of pMHC/mL. The resulting lot of beads was split into five portions, loading the costimulating ligands in the following proportions:
Set 1: CD28 at 3.00 micrograms/mL and CD2 at zero concentration.
Set 2: CD28 at 2.25 micrograms/mL and CD2 at 0.75 micrograms/mL.
Set 3: CD28 at 1.50 micrograms/mL and CD2 at 2.25 micrograms/mL.
Set 4: CD28 at 0.75 micrograms/mL and CD2 at 2.25 micrograms/mL.
Set 5: CD28 at 0.00 micrograms/mL and CD2 at 3.00 micrograms/mL.

To the T cells in media, an aliquot of each set of antigen-presenting beads were added to separate wells to a final concentration of 1 antigen-presenting bead per cell. The cells and antigen-presenting beads were mixed and seeded into tissue culture-treated, round-bottom, 96-well microplates. 0.2 mL (2e5 cells) was added to each well of the plate, which was then placed in a standard 5% CO₂, 37° C. incubator. Typically, 48-96 wells were used per plate. Two days later, IL-21 was diluted to 150 nanograms/mL in media. 50 microliters of IL-21 diluted in media was added to each well, and the plate was returned to the incubator.

After culturing the cells for an additional 5 days (seven days total), the cells were analyzed for antigen-specific T cell expansion. Alternatively, the cells were-stimulated in a second stimulation period as described in the following paragraphs to continue expanding antigen specific T cells.

Second T cell stimulation period. From each well of the above well plate at the conclusion of the first stimulation period, 50 microliters of media were removed, being careful not to disturb the cell pellet at the bottom of the well. IL-21 was diluted to 150 ng/mL in fresh media, and the antigen-presenting beads as produced above were added to the IL-21/media mixture at a final density of 4e6 antigen-presenting beads/mL. 50 microliters of this IL-21/antigen-presenting bead/media mixture was added to each well, resulting in an additional 2e5 antigen-presenting beads being added to each well. Optionally, the wellplate can be centrifuged for 5 minutes at 400×g to pellet the antigen-presenting beads onto the cells. The wellplate was returned to the incubator.

The next day (8 days from start of stimulation experiments), the wellplate was removed from the incubator, and 50 microliters of media was removed from each well. IL-2 (R&D Systems) was diluted into fresh media to 50 Units/mL. To this, media containing IL-2, IL-7 (R&D Systems) was added to a final concentration of 25 ng/mL. 50 microliters of this IL-2/IL-7/media mixture was added to each well, and the wellplate was returned to the incubator.

The following day (9 days from start of stimulation experiments), the wellplate was removed from the incubator, and 50 microliters of media was again removed from each well. IL-21 was diluted into fresh media to 150 nanograms/mL. 50 microliters of this IL-21/media mixture was added to each well, and the wellplate was returned to the incubator.

After culturing the cells for an additional 5 days (14 days from the start of stimulation experiments), the cells were typically analyzed for antigen-specific T cell expansion. However, the cells can be re-stimulated with more antigen-presenting beads for another period of culturing as above to continue expanding antigen specific T cells.

Analysis of antigen-specific T cell stimulation and expansion. Once the desired number of T cell stimulations were performed, the cells were analyzed for expansion of T cells specific for the pMHC complex used to prepare the antigen-presenting beads. Antigen-specific T cells are detected using Phycoerythrin (PE) conjugated Streptavidin, which is bound to 4 pMHC complexes. These complexes are referred to as tetramers. Typically, a tetramer manufactured with the same peptide used in the pMHC of the antigen-presenting beads was used to detect the antigen-specific T cells.

To detect and characterize the antigen-specific T cells, a mixture of PE-tetramer (MBL, Intl) and antibodies specific for various cell surface markers with various fluorophores, e.g., FITC-conjugated anti-CD28, PerCP-Cy5.5-conjugated anti-CD8, was prepared in FACS Buffer (Dulbecco's Phosphate-Buffered Saline without Calcium or Magnesium; 2% Fetal Bovine Serum; 5 mM Ethylenediaminetetraacetic Acid, 10 mM HEPES). The amount of antibody used was determined by titration against standard cell samples. The surface markers typically used for characterization used are: CD4, CD8, CD28, CD45RO, CD127 and CD197. Additionally, a Live/Dead cell discrimination dye, e.g., Zombie Near-IR (Biolegend) and Fc Receptor blocking reagent, e.g., Human TruStain FcX™ (Biolegend) were added to distinguish live cells and prevent non-specific antibody staining of any Fc-Receptor expressing cells in the culture, respectively.

Typically, the wells were mixed using a multi-channel micro-pipettor, and 50 microliters of cells from each well were transferred to a fresh, non-treated, round-bottom, 96-well microplate. The cells were washed by addition of 0.2 mL of FACS buffer to each well. Cells were centrifuged at 400×g for 5 minutes at room temperature, and the wash removed. To each well, 25 microliters of the Tetramer, Antibody, Live/Dead, Fc blocking reagent mixture was added. The cells were stained for 30 minutes under foil at room temperature. The cells were then washed again, and finally resuspended in FACS Buffer with CountBright Absolute Counting Beads (Thermo Fisher). The cells were then analyzed by Flow Cytometry (FACS Aria or FACS Celesta, BD Biosciences). The frequency of antigen-specific T cells was determined by gating first on Single/Live cells, then gating on CD8+/Tetramer⁺ cells. Appropriate gating conditions were determined from control stains, such as a negative control Tetramer with no known specificity (MBL, Intl) or antibody isotype controls. Within the antigen-specific T cell population, the frequency of CD45RO+/CD28^{High} cells was determined, as well as the number of cells expressing CD127. Activated T cells, which express CD45RO, that continue to express high levels of CD28 and CD127 have been shown to include memory precursor effector cells. Memory precursor cells have been shown to be less differentiated and have higher replicative potential than activated T cells that do not express these markers.

FIG. 10A: The frequency of MART1-specific T cells (percent of live cells) 7 days after stimulation with antigen-presenting beads prepared with the indicated amount (in micrograms) of anti-CD28 and/or anti-CD2. Each point represents a well of a 96-well microplate. Data is pooled from two independent experiments.

FIG. 10B: The total number of MART1-specific T cells 7 days after stimulation with antigen-presenting beads prepared with the indicated amount (in micrograms) of anti-CD28 and/or anti-CD2. Each point represents a well of a 96-well microplate. Data is pooled from two independent experiments.

FIG. 10C: The fold expansion of MART1-specific T cells 7 days after stimulation with aAPCs prepared with the indicated amount (in micrograms) of anti-CD28 and/or anti-CD2. Each dot represents a well of a 96-well microplate. Data was pooled from two independent experiments. Fold expansion is calculated by dividing the frequency of MART1 T cells in each well at day 7 by the frequency of MART1 T cells in the sample at day 0.

FIG. 10D: The fraction of MART1-specific T cells that were positive for CD45RO and expressing high levels of CD28 7 days after stimulation with aAPCs prepared with the indicated amount (in micrograms) of anti-CD28 and/or anti-CD2. Each dot represents a well of a 96-well microplate. Data was pooled from two independent experiments. Fold expansion is calculated by dividing the frequency of MART1 T cells in each well at day 7 by the frequency of MART1 T cells in the sample at day 0.

It was observed that production of antigen specific T cells was possible with a wide range of proportions of the costimulatory ligands anti-CD28 and anti-CD2. Production was possible using only one of the two costimulatory ligands. However, a combination of anti-CD28 and anti-CD2, including at ratios of anti-CD28:anti-CD2 from about 3:1 to about 1:3, provided increased measurements of each of the above characteristics.

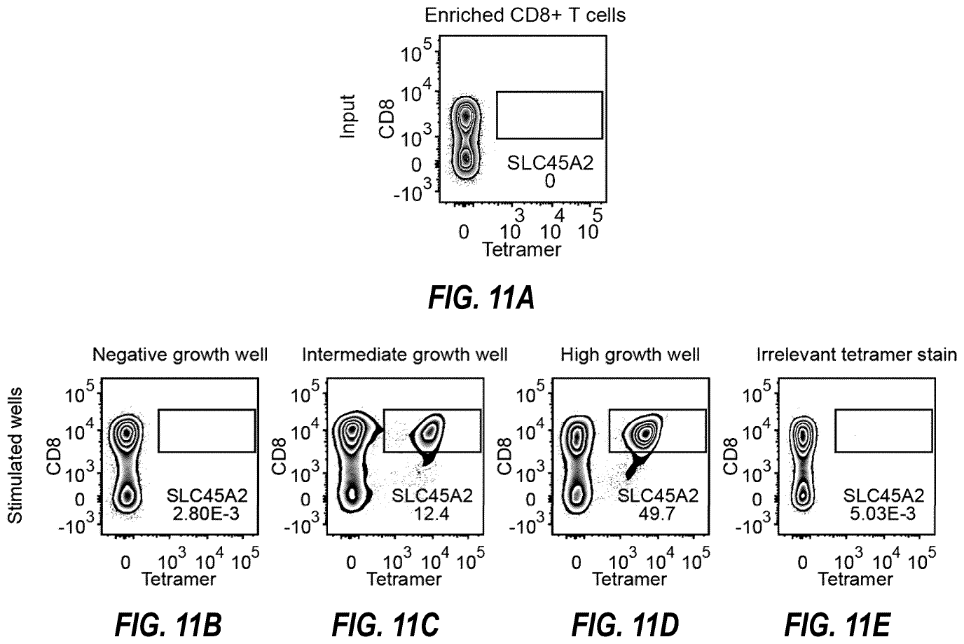

FIGS. 11A-11E: For T cells stimulated as described above, using the SLC45A2 antigen in the antigen-presenting beads produced as described above, exemplary Flow Cytometry graphs are shown. FIG. 11A showed the results of T cells, prior to stimulation (“Input”). Representative stimulated wells are shown in the lower panels: Negative growth well (FIG. 11B); intermediate growth well (FIG. 11C); High growth well (FIG. 11D); and Irrelevant Tetramer staining (FIG. 11E).

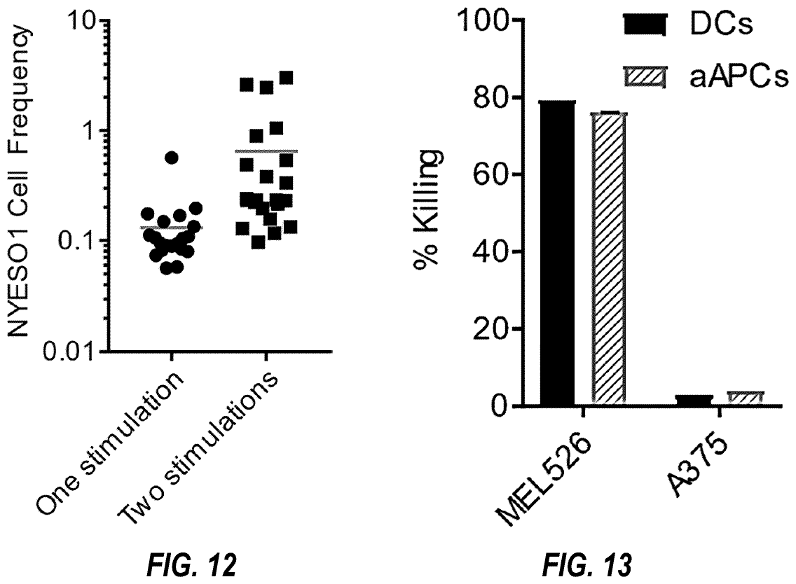

FIG. 12: For T cells stimulated as described above, using the NYESO1 antigen, the frequency of T cells positive for CD45RO and expressing high levels of CD28 are shown respectively after a single period of stimulation (7 days, left column) and after two periods of stimulation as described above (14 days, right column). Increased frequency of antigen specific activated T cells were observed.

Cytotoxicity: Killing of target tumor cells and non-target tumor cells by SLC45A2-specific T cells expanded using Dendritic cells pulsed with SLC45A2 antigen (DCs, Black bars) or antigen-presenting beads (presenting SLC45A2 antigen) produced as described above (gray hatched bars). See FIG. 13. Killing was measured by activation of Caspase-3 in target cells. MEL526 tumor cells express SLC45A2 and were killed by T cells expanded using both DCs and the antigen-presenting beads. A375 cells do not express SLC45A2 and were not killed by T cells expanded using DCs or the antigen-presenting beads. The antigen-presenting beads performed as well as the Dendritic cells.

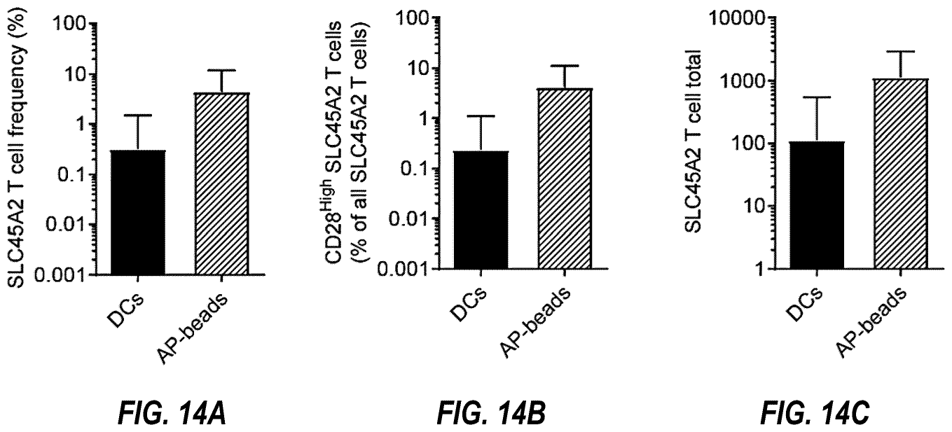

FIGS. 14A-14C show the comparison between the cell product of the dendritic cell stimulation and the antigen-presenting bead stimulated cell product. FIG. 14A showed that the percentage of Antigen Specific (AS) activated T cells is higher in the antigen-presenting bead stimulation experiment. FIG. 14B showed that the cell product of the antigen-presenting bead stimulation experiment has higher percentages of the desired CD45RO positive/highly CD28 positive phenotype, compared to that of the dendritic cell stimulated cell product. FIG. 14C showed that the actual numbers of antigen-specific T cells is higher in the cell product produced by the antigen-presenting bead stimulation experiment. Overall, antigen-presenting bead stimulation provides a more desirable cell product, and is a more controllable and cost effective method of activating T cells than the use of dendritic cell activation.

Further variations. The antigen-presenting beads may alternatively have protein fragment co-activating ligands instead of full antibody ligands as shown in this example. Details of preparation of protein fragment containing antigen-presenting beads, effective activation and expansion of antigen-specific CD8+ T cells, and demonstration of a memory precursor phenotype in those expanded cells is found in International Application Publication No. WO2019/018801, entitled “Antigenic-Presenting Synthetic Surfaces, Covalently Functionalized Surfaces, Activated T Cells, and Uses Thereof”, filed on Jul. 20, 2018, and International Application Publication No. WO2020/081875, entitled “Proto-Antigen-Presenting Synthetic Surfaces, Activated T Cells, and Uses Thereof”, filed on Oct. 17, 2019, of which each disclosure is herein incorporated by reference in its entirety.

### Example 18. Comparison Between Loading of and Activation with Convoluted Polymeric Beads Vs. Substantially Spherical Silica Beads

Example 18A. Comparison of activating species loading onto Polymer and Silica antigen presenting beads. Amounts of pMHC and costimulation antibodies that could be deposited onto Polymer and Silica beads was measured.

Streptavidin functionalized (covalently coupled) DynaBeads™ (ThermoFisher Catalog #11205D, bead stock at 6.67e8/mL) were delivered (15 microliters; 1e7 beads) to 1.5 mL microcentrifuges tube with 1 mL of Wash Buffer (DPBS (No Magnesium⁺², No Calcium⁺², 244 mL); EDTA (1 ml, final concentration 2 mM); and BSA (5 ml of 5%, final concentration 0.1%), and separated using a magnetic DynaBead rack. The wash/separation with 1 mL of the Wash Buffer was repeated, and a further 200 microliters of Wash Buffer was added with subsequent pulse centrifugation. Supernatant Wash Buffer was removed.

Biotin functionalized (covalently coupled) smooth silica beads were first coated with Streptavidin by storage in 100 micromolar Streptavidin. Approximately 5e6 beads were washed by dilution into 1 milliliter of Wash Buffer in a microcentrifuge tube, followed by centrifugation at 1,000×g for 1 minute. The supernatant was carefully removed by aspiration, and the wash process repeated twice more. Supernatant Wash Buffer was removed.

To prepare antigen presenting beads, Wash Buffer (600 microliters) containing 0.5 micrograms biotinylated Monomer MHC (HLA-A*02:01 SLC45A2 (Biolegend, Custom Product, SLYSYFQKV (SEQ ID NO: 5)) was dispensed into the tubes with the DynaBeads and Silica beads, and the beads were resuspended by pipetting up and down. The monomer was allowed to bind for 30 min at 4° C. After 15 minutes, the mixtures were pipetted up and down again. The tubes were centrifuged at 1,000×g for one minute, and the supernatant liquid removed.

Wash Buffer (600 microliters) with 1.5 micrograms of biotinylated anti-CD28 and 1.5 micrograms of biotinylated anti-CD2 was used to resuspend each bead sample, and the beads were resuspended by pipetting up and down. The antibodies were allowed to bind for 30 min at 4 C. After 15 minutes, the mixtures were pipetted up and down again. The tubes were centrifuged at 1,000×g for one minute, and the supernatant liquid removed. Finally, the beads were resuspended in 100 microliters of Wash Buffer.

Two samples of approximately 2e5 Polymer antigen presenting beads or 1e5 Silica antigen presenting beads were washed with Wash Buffer (1 milliliter). The bead samples were resuspended in 100 microliters of Wash Buffer and stained by addition of 1 microliter of APC-conjugated anti-HLA-A (Biolegend, Catalog Number 343308) or 1 microliter of APC-conjugated monoclonal anti-Mouse-IgG1 (Biolegend, Catalog Number 406610). The beads were mixed with the antibody and allowed to stain for 30 minutes in the dark. After staining, beads were washed, resuspended in Wash Buffer (200 microliters) and transferred to tubes for analysis by Flow Cytometry.

A set of Quantum Simply Cellular fluorescence quantitation beads (Bangs Labs, Catalog Number 815) was then prepared to determine the number of anti-HLA-A antibodies and anti-Mouse IgG1 antibodies bound to each antigen presenting bead sample. The quantitation beads have antibody binding capacities determined by the manufacturer. A drop of each bead with pre-determined binding capacity was placed in microcentrifuge tubes with 50 microliters of Wash Buffer. To the tubes, 5 microliters of APC-conjugated anti-HLA-A or APC-conjugated anti-Mouse IgG1 was added and mixed by vortexing. The beads were stained for 30 minutes in the dark, washed using the same method as above. The beads with different binding capacities were then pooled into one sample and transferred to a single tube. A drop of blank beads (no antibody binding capacity) was added and the beads were analyzed by Flow Cytometry.

The quantitation beads were analyzed by Flow Cytometry (BD FACSCelesta, Becton Dickinson and Company) by recording 5,000 events. The quantitation beads were identified by Forward Scatter and Side Scatter, and the median intensity in the APC channel of each bead recorded. This data was recorded in a proprietary Excel spreadsheet provided by the manufacturer (Bangs) that calculates a standard curve of APC intensity versus antibody binding capacity. After verifying that the calibration was linear, the antigen presenting bead samples were analyzed. The beads were identified by Forward and Side Scatter, and the median intensity in the APC channel recorded on the spreadsheet. The spreadsheet calculates the number of APC anti-HLA-A antibodies or APC anti-Mouse IgG1 on each antigen presenting bead. Assuming that 1 anti-HLA-A antibody binds to one pMHC on the antigen presenting bead, this value represents the number of pMHC molecules on each bead. Similarly, the number of costimulation antibodies can be determined.

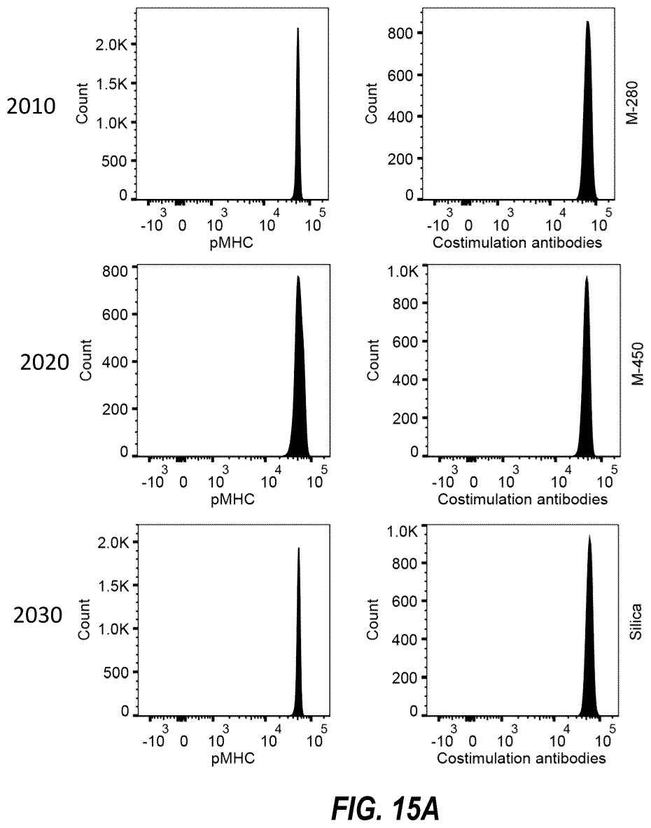

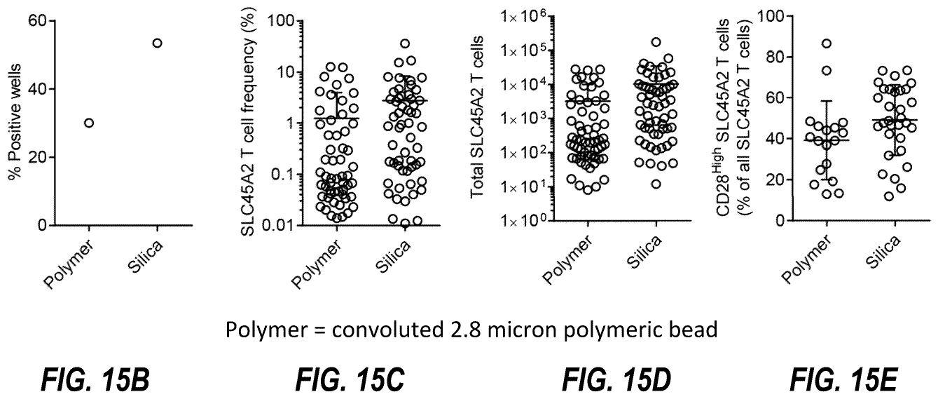

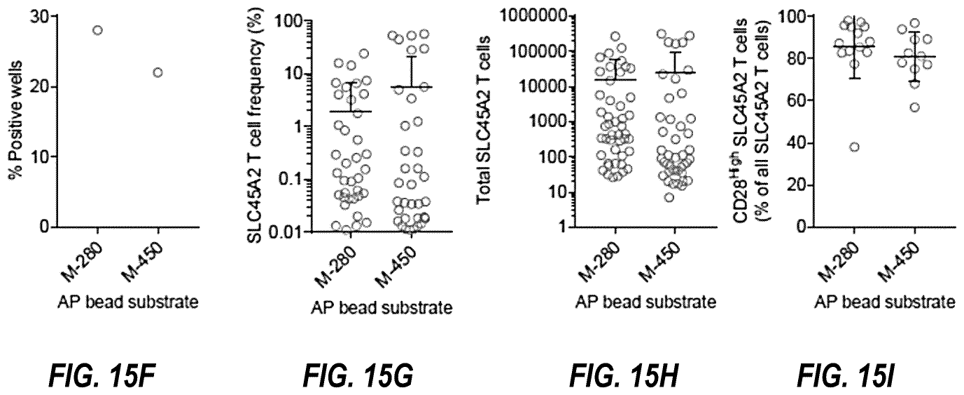

From the nominal surface area of each antigen presenting bead, the density (number of molecules/square micron of bead surface) of each species can be determined. The total number of pMHC on the Silica microspheres was determined to be approximately 800,000 pMHC/antigen presenting bead. The total number of costimulation antibodies was determined to be about 850,000 antibodies/bead. As there is no way to distinguish the anti-CD28 and anti-CD2 clones used to prepare the antigen presenting beads (they are the same isotype), it is assumed that the ratio of the two antibodies is 1:1. Due to the regularity of the Silica bead surfaces, the surface area can be reasonably modeled from a sphere. For a 4.08 micron diameter microsphere, this corresponds to a surface area of about 52.3 square microns. From this, it can be estimated that about 15,000 pMHC and 15,000 costimulation antibodies per square micron of bead surface are presented by the Silica antigen presenting beads as shown in Table 4. The distribution across each bead population for each ligand class is shown in FIG. 15A, where each row 2010, 2020, and 2030 shows the distribution of pMHC in the left hand graph, and the distribution of costimulation antibodies in the right hand graph for each type of bead. Row 2010 shows distribution of the ligands for 2.8 micron diameter convoluted polymer beads (Dynal). Row 2020 shows distribution of ligands for 4.5 micron diameter convoluted polymer beads (Dynal). Row 2030 shows distribution of ligands for a 2.5 micron diameter substantially spherical silica bead as produced in Example 9B. Tightly controlled populations of beads were produced, with the substantially spherical silica beads having even more tightly controlled distribution of ligands over the entire population, and slightly higher median distribution. Thus, the use of substantially spherical silica beads can lead to more reproducible and controllable production of these activating species. Additionally, since all of the ligands are accessible to T lymphocytes, unlike the convoluted polymer bead ligand distribution, more efficient use is made of precious biological ligands such as antibodies.

TABLE 4
Ligand quantification and density for convoluted polymer
beads and substantially spherical silica beads.

| | | | | Costimulation |
|---|---|---|---|---|
| | | pMHC | | Antibody |
| | | Density | Costimulation | Density |
| | pMHC/ | (molecules/ | antibodies/ | (molecules/ |
| Bead | bead | sq micron) | bead | sq micron) |
| M-280 | 487,209 | 19,781 | 426,992 | 17,336 |
| Polymer | | | | |
| Silica | 807,180 | 14,847 | 845,388 | 15,550 |

For Polymer beads, the convoluted surface makes the relationship between bead diameter and surface area less straightforward. From the quantitation, it was determined that Polymer antigen presenting beads based on M-280 DynaBeads had about 480,000 pMHC molecules and 425,000 costimulation antibodies on their surface. For a sphere of radius 1.4 microns (equal to the nominal radius of M-280 DynaBeads, this corresponds to about 20,000 pMHC and 17,000 costimulation antibodies per square micron, as shown in Table 4. However, due to the convoluted surface of the Polymer beads, the actual surface area is likely larger, and thus the actual density lower. From FIGS. 15E, 15F and 15G, though, it can be seen that these beads can be used as antigen presenting bead substrates to expand large numbers of antigen-specific T cells, where the expansion was performed in a similar manner as in Example 18B. In addition, from FIG. 15H, these antigen presenting beads generated large numbers of antigen-specific T cells with high expression of CD28, indicative of a memory precursor phenotype.

Antigen presenting beads were prepared in the same manner using M-450 Epoxy DynaBeads modified with Streptavidin. From flow cytometry, antigen presenting beads prepared from M-450 beads had approximately the same number of pMHC and costimulation antibody molecules as antigen presenting beads prepared with M-280 DynaBeads. As the M-450 DynaBeads are larger than the M-280 beads, this implies that the density of the activating species on the M-450 antigen presenting beads was about 2-3 times lower than on M-280 antigen presenting beads. However, as can be seen from FIG. 15F, M-450 antigen presenting beads generated positive wells (in which SLC45A2-specific T cells expanded to represent 0.5% or more of the live cells in the well) when used to expand SLC45A2 T cells. From FIGS. 15G and 15H, it can be seen that these wells generated SLC45A2 T cells at high frequencies, and the number of SLC45A2 T cells was comparable to the number obtained from M-280 antigen presenting beads. In addition, from FIG. 15I, the fraction of SLC45A2 T cells expressing high levels of CD28 was comparable when using M-280 or M-450 antigen presenting beads to expand SLC45A2 T cells.

Example 18B. Expansion of antigen-specific T cells with polymer vs Silica beads. Expansion of antigen-specific T cells using Silica antigen presenting beads was tested and compared to convoluted polymeric beads (polystyrene).

Streptavidin functionalized (covalently coupled, convoluted) DynaBeads™ (ThermoFisher Catalog #11205D, bead stock at 6.67e8/mL) were delivered (15 microliters; 1e7 beads) to 1.5 mL microcentrifuges tube with 1 mL of Wash Buffer (DPBS (No Magnesium⁺², No Calcium⁺² 244 mL); EDTA (1 ml, final concentration 2 mM); and BSA (5 ml of 5%, final concentration 0.1%), and separated using a magnetic DynaBead rack. The wash/separation with 1 mL of the Wash Buffer was repeated, and a further 200 microliters of Wash Buffer was added with subsequent pulse centrifugation. Supernatant Wash Buffer was removed.

Biotin functionalized (covalently coupled) smooth silica beads, prepared as in Example 9B, were first coated with Streptavidin by storage in 100 micromolar Streptavidin. Approximately 5e6 beads were washed by dilution into 1 milliliter of Wash Buffer in a microcentrifuge tube, followed by centrifugation at 1,000×g for 1 minute. The supernatant was carefully removed by aspiration, and the wash process repeated twice more. Supernatant Wash Buffer was removed.

To prepare antigen presenting beads, Wash Buffer (600 microliters) containing 0.5 micrograms biotinylated Monomer MHC (HLA-A*02:01 SLC45A2 (Biolegend, Custom Product, SLYSYFQKV (SEQ ID NO: 5)) was dispensed into the tubes with the DynaBeads and Silica beads, and the beads were resuspended by pipetting up and down. The monomer was allowed to bind for 30 min at 4° C. After 15 minutes, the mixtures were pipetted up and down again. The tubes were centrifuged at 1,000×g for one minute, and the supernatant liquid removed.

Wash Buffer (600 microliters) with 1.5 micrograms of biotinylated anti-CD28 and 1.5 micrograms of biotinylated anti-CD2 was used to resuspend each bead sample, and the beads were resuspended by pipetting up and down. The antibodies were allowed to bind for 30 min at 4° C. After 15 minutes, the mixtures were pipetted up and down again. The tubes were centrifuged at 1,000×g for one minute, and the supernatant liquid removed. Finally, the beads were resuspended in 100 microliters of Wash Buffer.

Cells: CD8+ T lymphocytes were enriched in a medium including RPMI plus 10% fetal bovine serum (FBS) from commercially available PBMCs following manufacturer's directions for EasySep™ Human CD8+ T Cell Isolation Kit, commercially available kit from StemCell Technologies Canada Inc. (Catalog #17953), by negative selection.

Culture medium and diluent for reagent additions: Advanced RPMI (ThermoFisher Catalog #12633020, 500 mL); 1× GlutaMAX (ThermoFisher Catalog #35050079, 5 mL); 10% Human AB serum (zen-bio, Catalog #HSER-ABP 100 mL, 50 mL); and 50 nM beta-mercaptoethanol (ThermoFisher Catalog #31350010, 50 nm stock, 0.5 mL, final conc 50 micromolar).

Experimental Setup: For each type of antigen presenting bead (Silica or Polymer, as prepared above in this example), a single 96 tissue-culture treated wellplate (VWR Catalog #10062-902) was used. Silica antigen presenting beads were mixed with CD8+ T lymphocytes at ˜1:2 beads:cell. CD8+ T lymphocytes (2e5) (80-90% pure) were added to each well with approximately 1e5 antigen presenting beads (wellplate 1). Polymer antigen presenting beads were mixed with CD8+ T lymphocytes at ˜1:1 beads:cell. CD8+ T lymphocytes (2e5) (80-90% pure) were added to each well with approximately 2e5 antigen presenting beads (wellplate 2).

Each wellplate was cultured at 37° C. On day 0, IL-21 (150 ng/milliliter) in CTL media, was added to each well of wellplates 1 and 2, providing a final concentration in each well of 30 ng/mL. On day 2, IL21 was added to each well of the wellplates, to a final concentration of 30 ng/mL. Culturing was continued to day 7.

Day 7. Restimulation. A second aliquot of antigen presenting beads was added to the corresponding wells in wellplate 1 and wellplate 2. For the Silica beads, approximately 1e5 beads (Silica beads as prepared above in this example) were added. For the Polymer beads, approximately 2e5 beads (convoluted polymer beads as prepared above in this example) were added. IL21 was added to each well of the wellplate to a final concentration of 30 ng/mL. Culturing was continued.

Day 8. Addition of 50 microliters of IL-2 (50 IU/mL) and IL-7 (25 ng/mL) was made to each well in wellplate 1 and wellplate 2 to provide a final concentration of 10 IU/mL and 5 ng/mL respectively. Culturing was continued.

Day 9. Addition of 50 microliters of IL-21 (150 ng/mL) was made to each occupied well of wellplate 1 and wellplate 2 to a final concentration of 30 ng/mL. Culturing was continued.

Day 14. The wells from each wellplate were individually stained for MHC tetramer (Tetramer PE, MBL Catalog #T02000, 1 microliter/well), CD4 (Biolegend Catalog #300530, 0.5 microliters/well); CD8 (Biolegend Catalog #301048, 0.5 microliters/well); CD28 (Biolegend Catalog #302906, 0.31 microliters/well); CD45RO (Biolegend Catalog #304210, 0.63 microliters/well); CCR7 (CD197, Biolegend Catalog #353208, 0.5 microliters/well); and viability (BD Catalog #565388, 0.125 microliters/well). Each well was resuspended with 150 microliters FACS buffer and 10 microliters of Countbright™ beads (ThermoFisher Catalog #C36950). FACS analysis was performed on a FACSCelesta™ flow cytometer (BD Biosciences).

FIG. 15B shows the percentage of positive wells (in which SLC45A2-specific T cells expanded to represent 0.5% or more of the live cells in the well) after expansion using the Polymer or Silica antigen presenting beads. FIG. 15C shows SLC45A2 T cell frequency (% of live cells in each well) after expansion with the Polymer or Silica antigen presenting beads. FIG. 15D shows the total number of SLC454A2 T cells in each of the wells. FIG. 15E shows the percentage of SLC45A2 T cells in the wells that expressed high levels of CD28, indicating the potential for differentiation into a memory T cell. From these plots, it can be seen that the Silica antigen presenting beads generate positive wells, and that the Silica antigen presenting beads expand SLC45A2 T cells as well or better than Polymer antigen presenting beads. In addition, the Silica antigen presenting beads produce cells with high expression of CD28, indicating that they support formation of memory precursor T cells, a desired phenotype for the cellular product.

For Polymer beads, the convoluted surface makes the relationship between bead diameter and surface area less straightforward. From the quantitation, it was determined that Polymer antigen presenting beads based on M-280 DynaBeads had about 480,000 pMHC molecules and 425,000 costimulation antibodies on their surface. For a sphere of radius 1.4 microns (equal to the nominal radius of M-280 DynaBeads, this corresponds to about 20,000 pMHC and 17,000 costimulation antibodies per square micron. However, due to the convoluted surface of the Polymer beads, the actual surface area is likely larger, and thus the actual density lower. However, from FIGS. 15F, 15G and 15H, it can be seen that these beads can be used as antigen presenting bead substrates to expand large numbers of antigen-specific T cells. In addition, from FIG. 15I, these antigen presenting beads generate large numbers of antigen-specific T cells with high expression of CD28, indicative of a memory precursor phenotype.

### Example 19A. Preparation of Antigen Presenting Beads with Defined Ligand Densities

Example 19A.1. Preparation of streptavidin presenting beads. Three-fold serial dilutions of pMHC (HLA-A*02:01 SLC45A2 (Biolegend, Custom Product, SLYSYFQKV (SEQ ID NO: 5)) in Wash Buffer were prepared. 20 microliters of Wash Buffer was added to a microcentrifuge tube for each serial dilution to be performed. Into the first serial dilution tube, 10 microliters of the pMHC were added. The diluted pMHC was mixed by vortexing. 10 uL of the diluted pMHC mixture was then used to prepare the subsequent serial dilution for a total of seven dilutions.

To determine the relationship between concentration of pMHC in solution and the density (molecules/unit area) deposited on the beads, Biotin functionalized (covalently coupled) smooth (e.g., substantially spherical as described above) 4 micron monodisperse silica beads (Cat. #SiO2MS-1.8 4.08 um-1g, Cospheric), prepared as in Example 9B, were first coated with Streptavidin by storage in 100 micromolar Streptavidin. Approximately 1e7 beads were washed by dilution into 1 milliliter of Wash Buffer in a microcentrifuge tube, followed by centrifugation at 1,000×g for 1 minute. The supernatant was carefully removed by aspiration, and the wash process repeated twice more. After washing, approximately 1e6 beads were delivered into eight microcentrifuge tubes, centrifuged again, and the supernatant carefully removed.

Example 19A.2. Preparation of beads having a range of MHC concentration. Wash Buffer (120 microliters) containing 4.5 micrograms biotinylated Monomer MHC (HLA-A*02:01 SLC45A2 (Biolegend, Custom Product, SLYSYFQKV (SEQ ID NO: 5)) was dispensed into one of the microcentrifuge tubes, and the beads were resuspended by pipetting up and down. The undiluted pMHC and serial dilutions of pMHC were further diluted into Wash Buffer (120 microliters) and used to resuspend beads, resulting in beads suspended in solutions with 4.5, 1.5, 0.5, 0.167, 0.056, 0.019, 0.006, or 0.002 micrograms of pMHC monomer per 5e6 beads. The monomer was allowed to bind for 30 min at 4° C. After 15 minutes, the mixtures were pipetted up and down again. The tubes were centrifuged, the supernatant liquid removed, and the beads resuspended at approximately 5e7/milliliter.

Approximately 1e5 beads prepared with each concentration of pMHC were washed with Wash Buffer (1 milliliter). The bead samples were resuspended in 100 microliters of Wash Buffer and stained by addition of 1 microliter of APC-conjugated anti-HLA-A (Biolegend, Catalog Number 343308). The beads were mixed with the antibody and allowed to stain for 30 minutes in the dark. After staining, beads were washed, resuspended in Wash Buffer (200 microliters) and transferred to tubes for analysis by Flow Cytometry.

A set of Quantum Simply Cellular fluorescence quantitation beads (Bangs Labs, Catalog Number 815) was then prepared to determine the number of anti-HLA-A antibodies bound to each antigen presenting bead sample. The quantitation beads have antibody binding capacities determined by the manufacturer. A drop of each bead with pre-determined binding capacity was placed in a microcentrifuge tube with 50 microliters of Wash Buffer. To the tube, 5 microliters of APC-conjugated anti-HLA-A was added and mixed by vortexing. The beads were stained for 30 minutes in the dark, washed using the same method as above. The beads with different binding capacities were then pooled into one sample and transferred to a single tube. A drop of blank beads (no antibody binding capacity) was added and the beads were analyzed by Flow Cytometry.

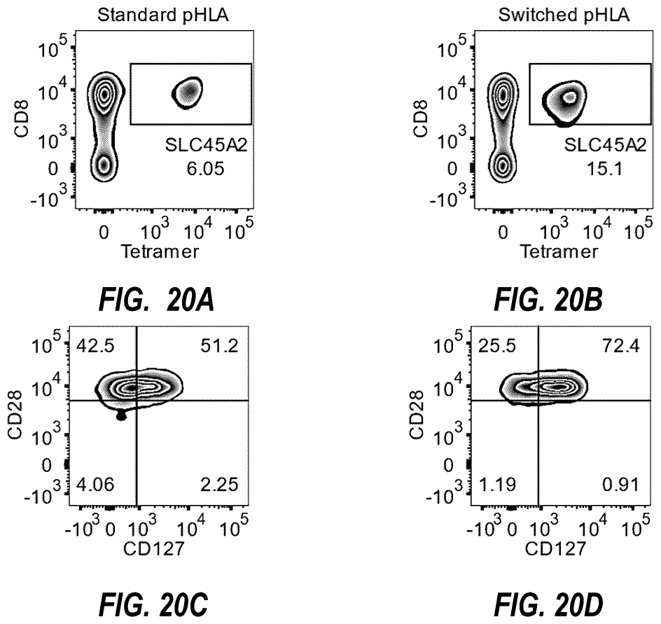

The quantitation beads were analyzed by Flow Cytometry (BD FACSCelesta, Becton Dickinson and Company) by recording 5,000 events. The quantitation beads were identified by Forward Scatter and Side Scatter, and the median intensity in the APC channel of each bead recorded. Quantitation was calculated as described in Example 18A, using the proprietary methodology provided by the quantitation bead manufacturer. From the antigen presenting bead standard, it is then possible to determine the concentration of pMHC in solution with beads that generates antigen presenting beads with a targeted density of pMHC, e.g., beads with approximately 10,000, 1,000, or 100 pMHC molecules per square micron, as seen in FIG. 20A.

Example 19A.3. Costimulation molecule concentration variation. Three-fold serial dilutions of biotinylated anti-CD28 and anti-CD2 in Wash Buffer were prepared. 20 microliters of anti-CD28 was mixed with 20 microliters of anti-CD2 in a microcentrifuge tube. Wash Buffer (20 microliters) was then added to a microcentrifuge tube for each serial dilution. The anti-CD28/anti-CD2 mixture (10 microliters) was then added to the first serial dilution tube. The solution was mixed using a vortexer, and 10 uL of the diluted anti-CD28/anti-CD2 mixture was then used to prepare the subsequent serial dilution for a total of seven dilutions.

To quantify the relationship between costimulation antibody in solution and the density (molecules/unit area) deposited on the beads, approximately 1e7 substantially spherical 4 micron silica beads, prepared as in Example 19A.1, having streptavidin binding moieties, were first washed by dilution into 1 milliliter of Wash Buffer in a microcentrifuge tube, followed by centrifugation at 1,000×g for 1 minute. The supernatant was carefully removed by aspiration, and the wash process repeated twice more. After washing, approximately 1e6 beads were delivered into eight microcentrifuge tubes, centrifuged again, and the supernatant carefully removed.

The beads were first functionalized with 1.0 micrograms of pMHC in Wash Buffer (1,200 microliters). After washing, the beads were resuspended in Wash Buffer (1,000 microliters). Into eight microcentrifuge tubes, 100 microliters of pMHC functionalized beads was dispended. The beads were centrifuged, and the supernatant carefully removed.

The undiluted mixed anti-CD28 and anti-CD2 and serial dilutions of anti-CD28/anti-CD2 were further diluted into Wash Buffer (120 microliters) and used to resuspend the beads, resulting in beads suspended in solutions with 4.5, 1.5, 0.5, 0.167, 0.056, 0.019, 0.006, or 0.002 micrograms of mixed costimulation antibodies monomer per 5e6 beads. The monomer was allowed to bind for 30 min at 4° C. After 15 minutes, the mixtures were pipetted up and down again. The tubes were centrifuged, the supernatant liquid removed, and the beads resuspended at approximately 5e7/milliliter.

Approximately 1e5 beads prepared with each concentration of costimulation antibodies was washed with Wash Buffer (1 milliliter). The bead samples were resuspended in 100 microliters of Wash Buffer and stained by addition of 1 microliter of APC-conjugated monoclonal anti-Mouse-IgG1 (Biolegend, Catalog Number 406610). The beads were mixed with the antibody and allowed to stain for 30 minutes in the dark. After staining, beads were washed, resuspended in Wash Buffer (200 microliters) and transferred to tubes for analysis by Flow Cytometry.

A set of Quantum Simply Cellular fluorescence quantitation beads (Bangs Labs, Catalog Number 815) was then prepared to determine the number of APC anti-Mouse IgG1 antibodies bound to each antigen presenting bead sample. A drop of each bead with pre-determined binding capacity was placed in a microcentrifuge tube with 50 microliters of Wash Buffer. To the tube, 5 microliters of APC-conjugated anti-Mouse IgG1 was added and mixed by vortexing. The beads were stained for 30 minutes in the dark, washed using the same method as above. The beads with different binding capacities were then pooled into one sample and transferred to a single tube. A drop of blank beads (no antibody binding capacity) was added and the beads were analyzed by Flow Cytometry.

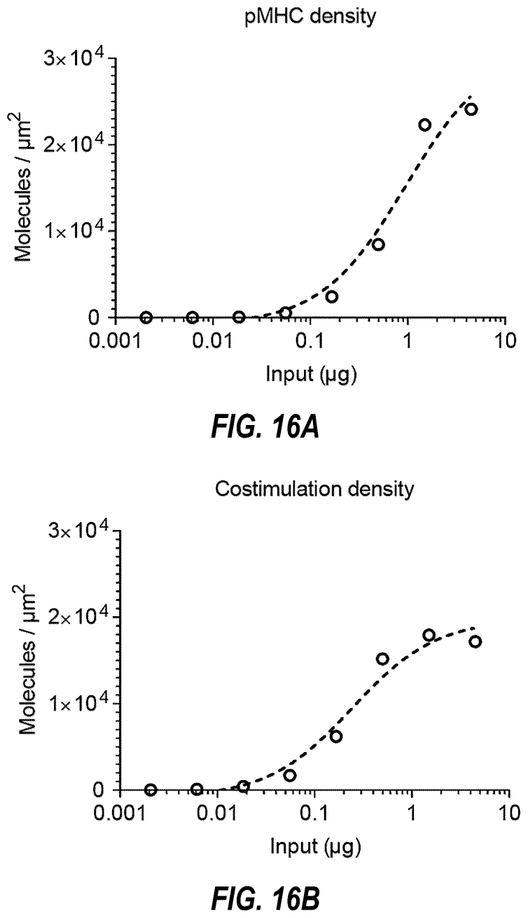

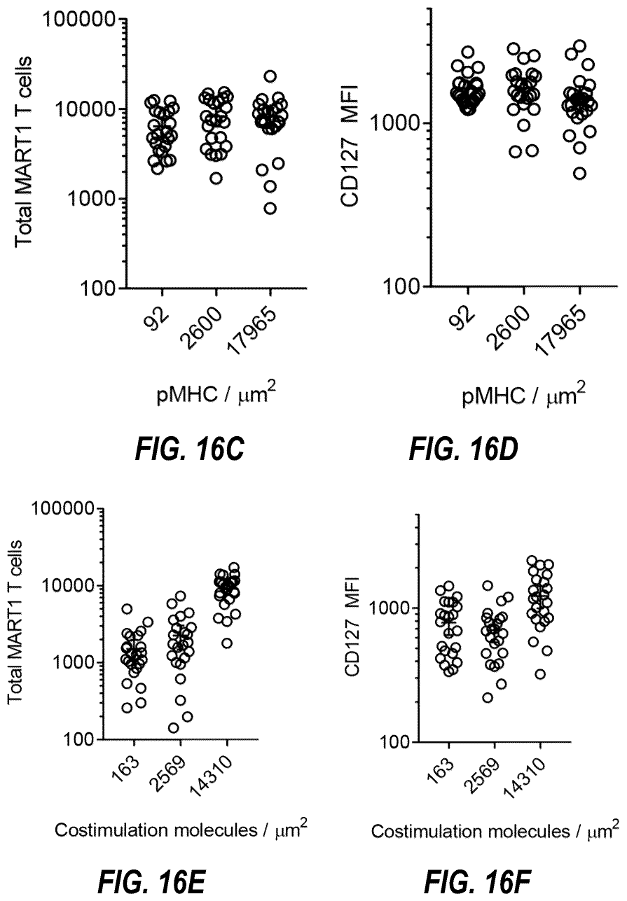

The quantitation beads were analyzed by Flow Cytometry (BD FACSCelesta, Becton Dickinson and Company) by recording 5,000 events. The quantitation beads were identified by Forward Scatter and Side Scatter, and the median intensity in the APC channel of each bead recorded. Quantitation was performed as described in Example 18A, using proprietary methods provided by the quantitation bead manufacturer. This quantitation method calculates the number of APC anti-Mouse IgG1 antibodies on each antigen presenting bead. Assuming that 1 anti-Mouse IgG1 antibody binds to one costimulation antibody on the antigen presenting bead, this value represents the number of costimulation antibodies on each bead. From the antigen presenting bead standards, it is then possible to determine the concentration of costimulation antibodies in solution with beads that generates antigen presenting beads with a targeted density of costimulation antibodies, e.g., beads with approximately 10,000, 1,000, or 100 costimulation molecules per square micron, as seen in FIG. 16B.

### Example 19B. Expansion of Antigen-Specific T Cells with Antigen Presenting Beads with Different Ligand Densities

Using the plots of pMHC and costimulation antibody concentration versus density on the resulting antigen presenting beads (FIG. 16A), it was determined what concentration of each pMHC should be used to prepare antigen presenting beads with ˜10,000, ˜1,000 or ˜100 pMHC per square micron of bead surface. This process was repeated to determine the concentration of anti-CD28 and anti-CD2 to be used to prepare antigen presenting beads with ˜10,000, ˜1,000 or ˜100 costimulation antibodies per square micron of bead surface.

Example 19B.1. Biotin functionalized (covalently coupled) smooth silica beads, prepared as in Example 19.A.1 were first coated with Streptavidin by storage in 100 micromolar Streptavidin. Approximately 5e7 beads were washed by dilution into 1 milliliter of Wash Buffer in a microcentrifuge tube, followed by centrifugation at 1,000×g for 1 minute. The supernatant was carefully removed by aspiration, and the wash process repeated twice more. After washing, approximately 5e6 beads were delivered into three microcentrifuge tubes, centrifuged again, and the supernatant carefully removed.

Example 19B.2. To prepare antigen presenting beads with titrated pMHC, Wash Buffer (600 microliters) containing 0.5, 0.056, or 0.006 micrograms biotinylated Monomer MHC (HLA-A*02:01 MART-1 (MBL International Corp., Catalog No. MR01008, ELAGIGILTV (SEQ ID NO: 7)) was dispensed into three microcentrifuge tubes, and the beads were resuspended by pipetting up and down. The monomer was allowed to bind for 30 min at 4° C. After 15 minutes, the mixtures were pipetted up and down again. The tubes were centrifuged at 1,000×g for one minute, and the supernatant liquid removed.

Wash Buffer (600 microliters) with 1.0 microgram of mixed biotinylated anti-CD28 and biotinylated anti-CD2 was used to resuspend each bead sample, and the beads were resuspended by pipetting up and down. The antibodies were allowed to bind for 30 min at 4° C. After 15 minutes, the mixtures were pipetted up and down again. The tubes were centrifuged at 1,000×g for one minute, and the supernatant liquid removed. Finally, the beads were resuspended in 100 microliters of Wash Buffer. The loading of the beads with the desired order of magnitude of pMHC and antibodies was verified by Flow Cytometry analysis and comparison to quantitation beads.

Wash Buffer (600 microliters) with 1.0 micrograms of anti-CD28 and anti-CD2 was used to resuspend each bead sample, and the beads were resuspended by pipetting up and down. The monomer was allowed to bind for 30 min at 4° C. After 15 minutes, the mixtures were pipetted up and down again. The tubes were centrifuged at 1,000×g for one minute, and the supernatant liquid removed. Finally, the beads were resuspended in 100 microliters of Wash Buffer. The loading of the beads with the desired order of magnitude of pMHC was verified by Flow Cytometry analysis and comparison to quantitation beads.

Example 19B.3. To prepare antigen presenting beads with titrated costimulation antibodies, Wash Buffer (1,200 microliters) containing 1.5 micrograms biotinylated Monomer MHC (HLA-A*02:01 MART-1 (MBL International Corp., Catalog No. MR01008, ELAGIGILTV (SEQ ID NO: 7)) was dispensed into a microcentrifuge tube containing 1.5e7 washed beads from Example 19.B.1, and the beads were resuspended by pipetting up and down. The monomer was allowed to bind for 30 min at 4° C. After 15 minutes, the mixtures were pipetted up and down again. The tubes were centrifuged at 1,000×g for one minute, and the supernatant liquid removed.

The beads were then resuspended in Wash Buffer (900 microliters), and 300 microliters of the beads transferred to 3 microcentrifuge tubes.

Wash Buffer (300 microliters) with 1.0 microgram of mixed anti-CD28 and anti-CD2, 0.111 micrograms of mixed antiCD28 and anti-CD2, or 0.012 micrograms of mixed anti-CD28 and anti-CD2 was mixed into the three bead samples, and the beads were thoroughly mixed by pipetting up and down. The antibodies were allowed to bind for 30 min at 4° C. After 15 minutes, the mixtures were pipetted up and down again. The tubes were centrifuged at 1,000×g for one minute, and the supernatant liquid removed. Finally, the beads were resuspended in 100 microliters of Wash Buffer. The loading of the beads with the desired order of magnitude of costimulation antibodies was verified by Flow Cytometry analysis and comparison to quantitation beads.

Example 19B.4. Stimulation. Cells: CD8+ T lymphocytes were enriched in a medium including RPMI plus 10% fetal bovine serum (FBS) from commercially available PBMCs following manufacturer's directions for EasySep™ Human CD8+ T Cell Isolation Kit, commercially available kit from StemCell Technologies Canada Inc. (Catalog #17953), by negative selection.

Culture medium and diluent for reagent additions: Advanced RPMI (ThermoFisher Catalog #12633020, 500 mL); 1× GlutaMAX (ThermoFisher Catalog #35050079, 5 mL); 10% Human AB serum (zen-bio, Catalog #HSER-ABP 100 mL, 50 mL); and 50 nM beta-mercaptoethanol (ThermoFisher Catalog #31350010, 50 nm stock, 0.5 mL, final conc 50 micromolar).

Experimental Setup: For each activation species titration (pMHC or costimulation antibodies), a single 96 tissue-culture treated wellplate (VWR Catalog #10062-902) was used. Antigen presenting beads with ˜10,000, ˜1,000 or ˜100 pMHC per square micron of bead surface and with ˜10,000 costimulation antibodies per square micron of bead surface (from Example 19.B.2) were mixed with CD8+ T lymphocytes at ˜1:2 beads:cell. CD8+ T lymphocytes (2e5) (80-90% pure) were added to each well with approximately 1e5 antigen presenting beads (wellplate 1). Antigen presenting beads with ˜10,000 pMHC per square micron of bead surface and with ˜10,000, ˜1,000 or ˜100 costimulation antibodies per square micron of bead surface (from Example 19.B.3) were mixed with CD8+ T lymphocytes at ˜1:2 beads:cell. CD8+ T lymphocytes (2e5) (80-90% pure) were added to each well with approximately 1e5 antigen presenting beads (wellplate 2).

Each wellplate was cultured at 37° C. On day 0, IL-21 (150 ng/milliliter) in CTL media, was added to each well of wellplates 1 and 2, providing a final concentration in each well of 30 ng/mL. On day 2, IL21 was added to each well of the wellplates, to a final concentration of 30 ng/mL. Culturing was continued to day 7.

Day 7. Restimulation. A second aliquot of antigen presenting beads with the targets density of pMHC or costimulation antibody was added to the corresponding wells in wellplate 1 and wellplate 2. IL21 was added to each well of the wellplate to a final concentration of 30 ng/mL. Culturing was continued.

Day 8. Addition of 50 microliters of IL-2 (50 IU/mL) and IL-7 (25 ng/mL) was made to each well in wellplate 1 and wellplate 2 to provide a final concentration of 10 IU/mL and 5 ng/mL respectively. Culturing was continued.

Day 9. Addition of 50 microliters of IL-21 (150 ng/mL) was made to each occupied well of wellplate 1 and wellplate 2 to a final concentration of 30 ng/mL. Culturing was continued.

Day 14. The wells from each wellplate were individually stained for MHC tetramer (Tetramer PE, MBL Catalog #T02000, 1 microliter/well), CD4 (Biolegend Catalog #300530, 0.5 microliters/well); CD8 (Biolegend Catalog #301048, 0.5 microliters/well); CD28 (Biolegend Catalog #302906, 0.31 microliters/well); CD45RO (Biolegend Catalog #304210, 0.63 microliters/well); CCR7 (CD197, Biolegend Catalog #353208, 0.5 microliters/well); and viability (BD Catalog #565388, 0.125 microliters/well). Each well was resuspended with 150 microliters FACS buffer and 10 microliters of Countbright™ beads (ThermoFisher Catalog #C36950). FACS analysis was performed on a FACSCelesta™ flow cytometer (BD Biosciences). FIG. 16C shows the number of MART1-specific T cells in each well expanded using antigen presenting beads with various densities of pMHC/square micron. FIG. 16D shows the expression level of CD127, a marker of memory precursor T cells, on the MART1-specific T cells from FIG. 16C. From these plots, it can be seen that the number of MART1-specific T cells and the expression of CD127 on these cells is insensitive to pMHC density when the density is ˜100 pMHC/square micron or higher.

FIG. 16E shows the number of MART1-specific T cells in each well expanded using antigen presenting beads with various densities of costimulation antibodies/square micron. FIG. 16F shows the expression level of CD127, a marker of memory precursor T cells, on the MART1-specific T cells from FIG. 16E. From these plots, it can be seen that the number of MART1-specific T cells and the expression of CD127 on these cells is sensitive to costimulation antibody density. Beads prepared with ˜10,000 costimulation antibodies per square micron, which nearly saturated the biotin binding sites of the bead (see FIG. 16B), generated the highest number of antigen-specific T cells, and those cells expressed the highest levels of CD127. As the number of costimulatory ligands was decreased to the lower end of the loading regime, primary stimulation by the pMHC was not as effectively co-stimulated, and the phenotype of the cell product is affected.

Cytotoxicity. Demonstration of antigen-specific T-cell cytotoxicity assays can be found in International Application Publication No. WO2019/018801, entitled “Antigenic-Presenting Synthetic Surfaces, Covalently Functionalized Surfaces, Activated T Cells, and Uses Thereof”, filed on Jul. 20, 2018, and International Application Publication No. WO2020/081875, entitled “Proto-Antigen-Presenting Synthetic Surfaces, Activated T Cells, and Uses Thereof”, filed on Oct. 17, 2019, of which each disclosure is herein incorporated by reference in its entirety.

### Example 20. Rapid Expansion of Antigen-Specific T Lymphocytes after Bead Stimulation and Characterization of Cellular Product

Typically after completion of antigen specific T lymphocyte activation as described in the preceding experiments, the antigen specific enriched T cells were sorted by FACS on an FACSAria Fusion System (Becton Dickinson, San Jose, CA) after staining 30 min RT in FACS buffer (1λDPBS w/o Ca²⁺Mg²⁺ (Cat. #4190250, ThermoFisher), 5 mM EDTA (Cat. #AM9260G, ThermoFisher), 10 mM HEPES (Cat. #15630080, ThermoFisher), 2% FBS) with anti-CD8-PerCPCy5.5 (Clone RPA-T8, 301032, Biolegend, San Diego, CA), Tetramer-PE (MBL International, Woburn, MA) specific to the antigen, and Zombie NIR (Cat. #423106, Biolegend, San Diego, CA) to exclude dead cells. Desired cells were purity sorted by gating: size, singles, live, CD8 positive, and Tetramer positive into CTL media (Advanced RPMI (Cat. #12633020, ThermoFisher), 1× Glutamax (Cat. #35050079, ThermoFisher), 10% Human Serum (Cat. #MT35060Cl, ThermoFisher), 50 uM b-Mercaptoethanol (Cat. #31350010, ThermoFisher) with 2 mM HEPES.

The sorted antigen-specific T cells were then expanded in at least one round of Rapid Expansion Protocol (REP), as described in Riddell, U.S. Pat. No. 5,827,642. Lymphoblastoid Cell Line cells (LCL, the LCL cell line was a gift from Cassian Yee, M.D. Anderson Cancer Center) were irradiated with 100 Gy and PBMC from 3 donors were irradiated with 50 Gy using an X-ray irradiator. Irradiated cells were washed in RPMI containing 10% FBS and mixed in a ratio of 1:5 (LCL:PBMC). These irradiated cells were added to either FACS-sorted T cells (for a first cycle of REP), or to the product of a first cycle of REP in 200 to 500-fold excess. Cultures were set up in T cell media (Advanced RPMI, 10% Human AB Serum, GlutaMax, 50 uM b-mercaptoethanol) supplemented with 50 U/mL IL-2 (Cat. #202-IL, R&D Systems) and 30 ng/mL anti-CD3 antibody (Cat. #16-0037-85, ThermoFisher). Cells were fed with fresh IL-2 on days 2, 5 and 10, and expanded according to their growth rates.

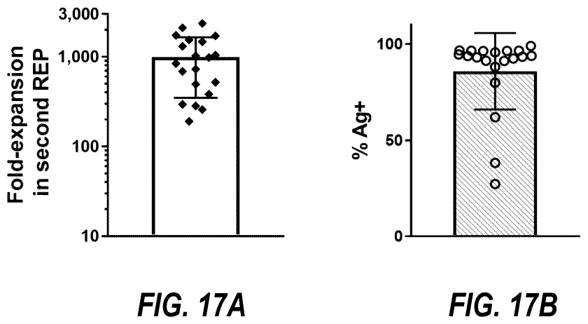

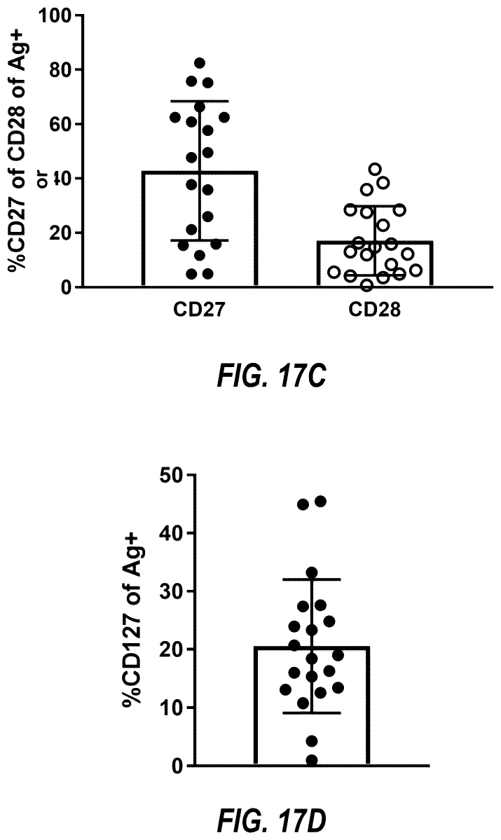

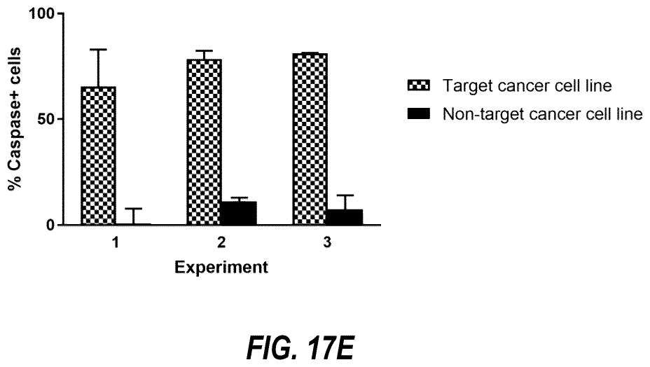

Expansion is typically 1,000-fold during a first REP cycle. Expansion during REP1 varied highly (316-7,800-fold, data not shown). Inaccurate quantification of low input cell numbers may have contributed to this variability. Shown here in FIG. 17A is fold-expansion obtained from a second REP protocol following the first cycle (n=20 experiments, 11 donors, 12 STIMs). Expansion ranged from about 200 up to about 2000 fold. However, there was no clear correlation between extent of expansion in REP1 and REP2 for a particular cell population in these experiments.

In FIG. 17B, the percentages of antigen-specific T cells in the REP populations are shown for the 20 experiments of the REP protocol. What was observed was that high percentages of antigen-specific T cells (% Ag+), typically ˜90%, were maintained during at least two REP cycles. In contrast, Low % Ag+ after REP1 led to low % Ag+ after REP2.

In FIG. 17C, the percentages of antigen-specific T cells also expressing co-stimulatory receptors CD27 and CD28, after the completion of REP2, are shown. In FIG. 17D, the percentages of antigen-specific T cells also expressing CD127, a marker for a central memory phenotype which can presage persistence in vivo, after the completion of REP2 is shown. While the distribution of expression of any of the markers was not tightly clustered, and some of the individual experiments showed low (e.g., a few percent) of cells that express the desired markers, the cellular products obtained in each of these experiments demonstrated sufficiently positive phenotype across all categories to render them candidates for in-vivo introduction. Some of the depressed values seen, such as expression of CD28, may be due to the extensive stimulation using CD28 ligands used during the activation cycles, leading to depressed expression of these surface markers.

In FIG. 17E, the results of antigen-specific cytotoxicity assay for each of three individual cellular populations, after two rounds of REP, are shown. Tumor cell lines obtained from melanoma cells, including Mel 526 cells and A375 cells, were tested. Each cell line was grown up in vitro according to standard procedures, then labeled with CellTrace™ Far Red dye (Cat. #C34572, ThermoFisher Scientific), which provides stable intracellular labelling. Each population of labeled tumor cells were suspended in T cell media (Adv. RPMI+10% Human AB serum (Cat. #35-060-Cl, Corning)+Gln+50 uM 2-mercaptoethanol (BME, Cat. #31350-010, Gibco, ThermoFisher Scientific) supplemented with 10 uM fluorogenic Caspase-3 substrate (DEVD, Green) (Nucview® 488, Cat. #10403, Biotium). A final concentration of the Caspase-3 substrate at 5 uM at Time=0 for the assay was provided. The Caspase-3 substrate provides no fluorescent signal until cleaved, so at Time=0, there was no fluorescent signal due to this reagent. T cells expanded against the SLC45A2 antigen, according to an endogenous T cell (ETC) protocol as described above, were placed in proximity, having a ratio of about 0-5 tumor cells per T cell. T cell media (Adv. RPMI+10% Human AB serum+Gln+50 uM BME) supplemented with 5 uM Caspase-3 substrate (Green) (Nucview 488 from Biotium) was then provided. The CellTrace Far Red label was cleaved, and now fluorescent Caspase-3 label was visualized. The Mel526 melanoma cell line expresses the SLC45A2 tumor-associated antigen and was expected to be targeted and killed by the SLC45A2-specific T cells. The A375 melanoma cell line does not express the SLC45A2 tumor-associated antigen and was not expected to be targeted or killed by the SLC45A2-specific T cells, and thus was used as a negative control for T cell cytotoxicity.

In each experiment, more than 50% of the targeted Mel526 cells exhibited Caspase 3 triggered fluorescent signal, while none to a few percent of the A375 non-targeted cells exhibited apoptotic behavior as signaled by the fluorogenic cleavage product of the Caspase-3 substrate. Therefore, the activated T cells still exhibited antigen-specific cell killing behavior after all of the rounds of activation and expansion.

Therefore, the processes of activation via antigen presentation on a synthetic surface as described herein can provide well controlled, reproducible and characterizable cellular products suitable for use in immunotherapy. The antigen-presenting surfaces described herein provide lower cost of manufacture for these individualized therapies compared to currently available experimental processes.

### Example 21. Binding of Immunogenic and Non-Immunogenic Peptides to MHC Class I Complexes

### Experimental Procedure

To test binding of Immunogenic and Non-Immunogenic peptides to an MHC Class I complex, a peptide-HLA-A*02:01 complex with an initial peptide LMYAKRAFV (SEQ ID NO: 4) in the peptide binding groove was purchased. The initial peptide included a dinitrophenyl (DNP) moiety conjugated to the lysine at position 5. Two peptide antigens were then tested for their ability to bind to the MHC Class I complex: SLYSYFQKV (SEQ ID NO: 5) derived from SLC45A2 and SLLPIMWQLY (SEQ ID NO: 6) derived from TCL1. The peptides were resuspended in DMSO to 5 mg/mL. The peptides were then further diluted ten-fold in PBS. To set up 0.05 mL peptide switching reactions, 20 micromolar SLYSYFQKV (SEQ ID NO: 5) or SLLPIMWQLY (SEQ ID NO: 6) peptide, 1 micromolar HLA-A*02:01 (with initial peptide) and 1 mM Glycl-Methionine (exchange factor) were mixed in Assay Buffer (PBS without Magnesium and Calcium, supplemented with 2 mM EDTA and 0.1% BSA). In addition, a control peptide switching reaction using the peptide ELAGIGILTV (SEQ ID NO: 7) was set up; this peptide is known to bind with high affinity to HLA-A*02:01 and is used to determine “complete” peptide exchange. The peptide switching reactions proceeded at room temperature for 4 hours, then the switched complexes were stored at 4° C. until further use.

Samples of unswitched peptide-MHC and switched peptide-MHC were then captured on Streptavidin-coated DynaBeads (ThermoFisher). About 107 DynaBeads per switching reaction were washed once with 1 mL of Assay Buffer and then captured on a magnetic rack. The peptide-MHC complexes were diluted to 0.83 micrograms/mL in Assay Buffer and used to resuspend the beads captured on the magnetic rack. The beads were mixed at 2,000 rpm for four minutes to capture the peptide-MHC complexes. The beads were again captured on a magnetic rack, washed once with 1 mL of Assay Buffer, and resuspended at about 10⁸ beads/mL. The peptide-MHC beads were then stored at 4° C. until they were analyzed for peptide exchange.

To quantify peptide exchange, a FITC-conjugated anti-DNP antibody specific for the DNP-conjugated initial peptide was added to a sample of each captured peptide-MHC. About 2×10⁵ beads of either unswitched peptide-MHC, control switched peptide-MHC, or the test switched peptide-MHCs were diluted into 0.1 mL of Assay Buffer in 1.5 mL microcentrifuge tubes. One microliter of FITC-conjugated anti-DNP antibody and one microliter of an APC-conjugated, conformationally sensitive antibody which only recognizes pMHCs in the folded, complex conformation (Clone W6/32, Biolegend) was then added to each tube. The samples were stained for 30 minutes in the dark. The beads were captured on a magnetic rack, the staining solution was removed, and then the beads were washed with 1 mL of Assay Buffer. Each bead sample was resuspended in Assay Buffer and transferred to a 5 mL Polystrene tube. The staining for pre-assembled peptide and intact pHLA complexes were detected by Flow Cytometry on a FACSCelesta with High-Throughput Sampler (BD Biosciences). The beads were identified by Forward Scatter- and Side Scatter-Amplitudes. Approximately 5,000 bead events were recorded for each sample. The Median Fluorescence Intensity (MFI) in the APC and FITC channels of each sample was then recorded.

To quantify peptide switching, the MFI of the unswitched sample was set as zero switching, and the MFI of the ELAGIGILTV (SEQ ID NO: 7) switched sample was set as 100%. The MFI of the test peptides (as determined using the FITC channel and the FITC-conjugated anti-DNP antibody) was then used to determine the percent switching according to the following formula:

100*[MFI(unswitched)−MFI(test peptide switching)]/[MFI(unswitched)−MFI(control switching)].

The MFI measurements determined using the APC channel and the APC-conjugated, conformationally sensitive antibody are not used in the formula (and, thus, are not required for the experimental measurement of peptide switching/binding). However, the APC signal can be useful in that it provides an indication that the MHC complexes on the beads remain properly folding following the peptide exchange reaction.

### Results

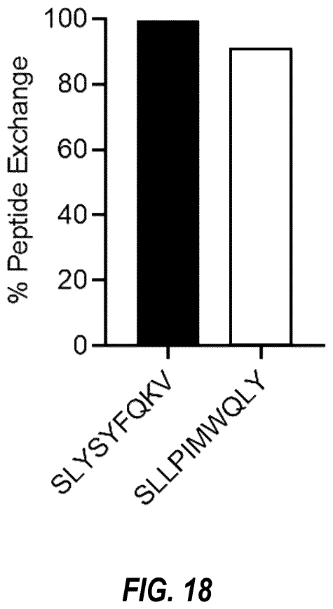

The quantitation of the peptide switching indicated that both the Immunogenic SLC45A2-derived and Non-Immunogenic TCL1-derived peptides were able to bind to HLA-A*02:01 (FIG. 18). SLC45A2-derived peptide switched nearly completely, relative to the control peptide (about 99% switching). The TCL1-derived peptide did not switch as efficiently, but still was able to generate about 90% peptide switching.

### Variations

The foregoing determination of peptide switching can be performed with any peptide antigen of interest, a different initial peptide (e.g., any initial peptide disclosed herein), and any of the exchange factors disclosed herein. Any form of initial peptide labeling could be employed, including direct conjugation with a fluorescent label; and use of the APC-conjugated, conformationally sensitive antibody (and the related MFI measurements) can be discarded. Furthermore, the experiment can be readily adapted to measurement of peptide switching on MHC Class II complexes.

### Example 22. Peptide Binding and Stability Under Culture Conditions

### Experimental Procedure

To assess the stability of the pMHC Class I-peptide antigen complexes under the conditions that are used to culture T Cells, pMHC Class I complexes were first bound to beads. Biotinylated HLA-A*02:01 complexes loaded with either SLYSYFQKV (SEQ ID NO: 5) or SLLPIMWQLY (SEQ ID NO: 6) peptides were diluted to 0.83 micrograms/mL in Assay Buffer (PBS with 2 mM EDTA and 0.1% Bovine Serum Albumin). To 0.6 mL of the pMHC solutions in microcentrifuge tubes, 107 Streptavidin-coated DynaBeads (M-280, ThermoFisher) were added. The beads were mixed in the pMHC solution for 4 minutes at 2,000 rpm on a ThermoMixer (Eppendorf). The pMHC-beads were then captured on a magnetic rack, and the solution containing unbound pMHC removed by aspiration. 1 mL of Assay Buffer was added to the tubes, and then aspirated. The beads were then resuspended in 0.1 mL of Assay Buffer. The beads were then stored at 4° C. until use.

To wells of a 96-well, round-bottom microplate, 0.2 mL of T Cell Culture Media (Advanced RPMI, 10% Human AB Serum, 1 mM GlutaMax) was added and equilibrated to 37° C. in a standard tissue culture incubator. To three wells each, four microliters of each pHLA-beads was added to a well. The process of adding beads to wells was repeated at time intervals resulting in beads that were held in the media at 37° C. for 48, 32, 24, 16, 8, 4, 2, and 1 hr. The plate was centrifuged at 400g for 5 minutes, and the media removed by flicking the plate. A sample of beads held at 4° C. for the duration of the time course was then added to three wells to create the 0 hr time point.

An APC-conjugated, conformationally sensitive antibody which only recognizes pMHC molecules in the folded, complex conformation (Clone W6/32, Biolegend) was then added to each well. The antibody was diluted 50-fold from the manufacturer stock into Assay Buffer, and 0.05 mL of antibody mixture was added to each well. The samples were stained for 30 minutes at room temperature under foil. The plate was centrifuged, and the staining solution removed by flicking the plate. 0.2 mL of Assay Buffer was added to each well of the plate, which was again centrifuged. The plate was flicked to remove the Assay Buffer, and each well resuspend in 0.15 mL of FACS buffer.

Antibody binding to the beads was then detected on a FACSCelesta with High-Throughput Sampler (BD Biosciences). Beads were identified by Forward Scatter- and Side Scatter-Amplitudes. Approximately 25,000 bead events were recorded for each sample. The Median Fluorescence Intensity (MFI) in the APC channel for the pHLA-beads in each sample was then recorded. The MFIs were then plotted against the time spent at 37° C. for each sample. The resulting decay curves were then fitted to an exponential decay curve using the curve_fit module in SciPy, a freely available Scientific Computing package for Python. The half-lives for the pHLA complexes were then calculated from the fitted decay constant.

### Results

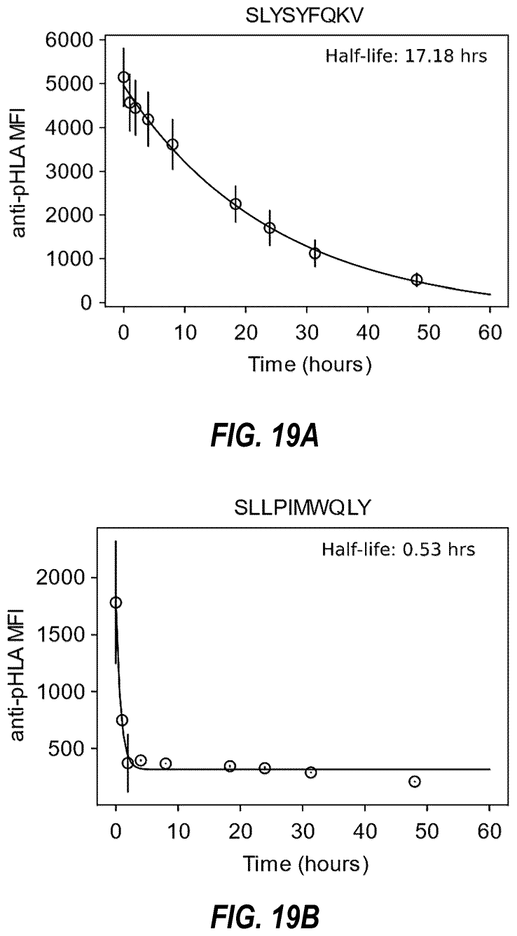

Results are shown in FIGS. 19A-B for SLYSYFQKV (SEQ ID NO: 5) and SLLPIMWQLY (SEQ ID NO: 6). The half-life of the SLYSYFQKV (SEQ ID NO: 5)-HLA-A*02:01 complex was estimated to be about 17 hours. The half life of the SLLPIMWQLY (SEQ ID NO: 6)-HLA-A*02:01 complex was estimated to be about 0.5 hours.

### Variations

The foregoing determination of MHC Class I-peptide antigen complex stability was performed with MHC Class I complexes that were folded with their respective peptide antigens. However, the same experimental measurement of stability could be performed with MHC complexes that undergo a peptide exchange reaction of the type described herein (e.g., as described in Example 21). Furthermore, the determination of complex stability can be performed with any peptide antigen of interest, and the experiment can be readily adapted to measurement of MHC Class II-peptide antigen complex stability.

### Example 23. Preparation and Use of Antigen-Presenting Beads

### Experimental Procedure

Antigen-presenting beads presenting the SLC45A2-derived peptide SLYSYFQKV (SEQ ID NO: 5) were prepared by two procedures. In the first procedure, pre-assembled Biotinylated peptide-HLA-A*02:01 complexes bearing the SLYSYFQKV (SEQ ID NO: 5) peptide antigen were purchased from a manufacturer (Biolegend, custom order). In the second procedure, Biotinylated peptide-HLA-A*02:01 complexes bearing the SLYSYFQKV (SEQ ID NO: 5) peptide antigen were prepared by first incubating SLYSYFQKV (SEQ ID NO: 5) peptide with an exchange factor and HLA-A*02:01 complexes pre-assembled with an initial peptide. Lyophilized SLYSYFQKV (SEQ ID NO: 5) peptide (GenScript) was dissolved in DMSO to 5 mg/mL. The peptide antigen was then further diluted ten-fold in PBS. To setup a 0.05 mL peptide switching reaction, 20 micromolar peptide, 1 micromolar HLA-A*02:01 and 1 mM Glycl-Methionine (exchange factor) were mixed in Assay Buffer. The peptide switching reaction proceeded at room temperature for 4 hours, then the switched MHC complexes were stored at 4 C until further use.

The pre-assembled peptide-MHC and peptide switched peptide-MHC complexes were then used to prepare APBs. Samples of about 1.2×10⁷ Streptavidin-coated DynaBeads (ThermoFisher) were washed once with 1 mL of Assay Buffer, then resuspended in Assay Buffer with either pre-assembled peptide-MHC or switched peptide-MHC at 0.83 micrograms/mL. The beads were mixed at 2,000 rpm for four minutes to capture peptide-MHC. The beads were captured on a magnetic rack, and the peptide-MHC functionalization mixture was then removed by aspiration. A mixture of Biotinylated anti-CD28 (Biolegend) and anti-CD2 (Biolegend) was then added to the beads (1:1 anti-CD28:CD2 at 5 micrograms/mL total antibody). The beads were again mixed at 2,000 rpm for four minutes. Beads were captured on a magnetic rack, and the antibody functionalization mixture was removed by aspiration. The beads were washed once with Assay Buffer, resuspended at a density of about 1e8 beads per mL, and then stored at 4° C. until use.

To expand antigen-specific T Cells with the APBs, CD8+ T Cells were isolated from PBMCs isolated from normal, healthy donors according to the manufacturer's recommended protocol (EasySep, StemCell Technologies). The CD8+ T Cells were split into two samples: one for the APBs prepared with pre-assembled peptide-MHC, and one for the APBs prepared with switched peptide-MHCs. The two types of APBs were mixed with the isolated CD8+ T Cells at a ratio of 1 Cell:1 APB in T Cell Culture Media with 30 ng/mL IL-21. After mixing the cells and beads in culture media, 0.2 mL per well was distributed into the wells of a tissue culture-treated, 96-well, round-bottom microplate. The plates were then incubated in a standard 5% C02, 37° C. incubator for two days. After two days in culture, IL-21 was diluted to 150 nanograms/mL in growth media. 50 microliters of IL-21 diluted in media is added to each well, and the plate was returned to the incubator additional culture.

After a total of seven days of culture, the cells were then restimulated with appropriate APBs. From each well of the plate, 0.05 mL of media were removed. IL-21 was diluted to 150 ng/mL in fresh media, and APBs were added to the IL-21/media mixture at a final density of 4×10⁶ APBs/mL. 50 microliters of this IL-21/APB/media mixture were added to each well, resulting in an additional 2×10⁵ APBs being added to each well. The plates were then returned to the incubator.

The next day, the plates were removed from the incubator, and 50 microliters of media again removed from each well. IL-2 (R&D Systems) was diluted into fresh media to 50 Units/mL. To this media containing IL-2, IL-7 (R&D Systems) was added at a final concentration of 12.5 ng/mL. 50 microliters of this IL-2/IL-7/media mixture was added to each well, and the well was returned to the incubator.

The next day, the plate was removed from the incubator, and 50 microliters of media again removed from each well. IL-21 was diluted into fresh media to 150 nanograms/mL. 50 microliters of this IL-21/media mixture was added to each well, and the well returned to the incubator.

After culturing the cells for an additional 5 days, the cells were analyzed for antigen-specific T Cell expansion and expression of memory precursor surface markers (CD45RO, CD28 and CD127).

### Results

Analysis of the stimulated wells of CD8+ T Cells indicated that the APBs generated using the switched peptide-MHCs successfully generated Antigen Specific T Cell colonies (FIGS. 20A-D). Analysis of the Antigen-Specific T Cells indicated that the cells expressed high levels of CD45RO, CD28 and CD127, which indicates the cells had taken on a Central Memory Precursor T Cell phenotype.

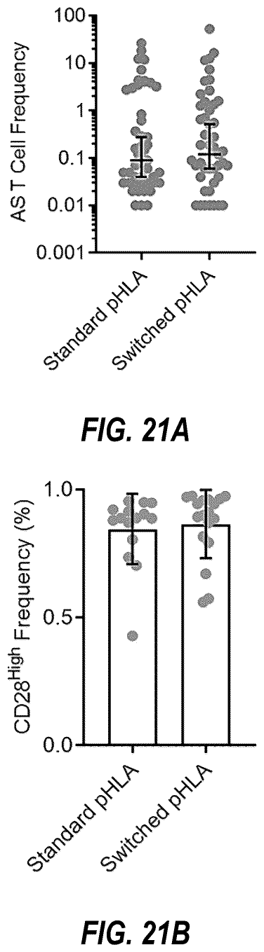

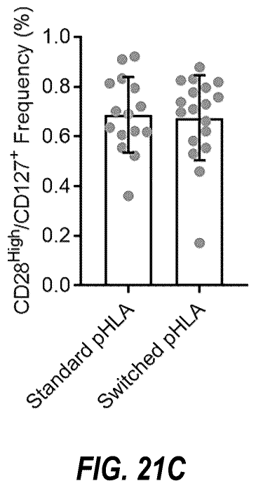

Comparing the APBs prepared from switched-peptide-MHCs vs conventional MHCs, we observed that the number of Antigen Specific (AS) T Cell colonies (wells in which the Antigen Specific T Cells expanded to greater than 0.5% of all cells in the well) (not shown) and the frequency of Antigen Specific T Cells in those colonies were similar using both types of APBs (FIG. 21A). In addition, the frequency of CD45RO+Antigen Specific T Cells expressing high levels of CD28 was consistently high, indicating that the switched peptide APBs supported IL-21 mediated support of CD28 expression (FIG. 21B). In addition, the number of CD1 27+Antigen Specific T Cells in the CD28High population was consistent between APB types (FIG. 21C).

This data indicates that peptide switching can be used to generate APBs that efficiently expand Antigen Specific T Cells with a Central Memory Precursor phenotype.

### Example 24. Testing Candidate Peptides as In-Place Peptides Stabilizing MHC Molecules

Candidate Peptide Identification. The list of published HLA-A*02:01 peptides was downloaded from the Immune Epitope Database (www.iedb.org). The list of peptides was first filtered for epitopes derived from Human proteins, then for 9-10mers, and finally for peptides with a Lysine at Position 4 or 5. This resulted in a list of 1,368 peptides. Epitopes from known Tumor-Associated Antigens, such as MART1, were not considered. A list of over 50 Epitopes, primarily from widely expressed, highly conserved proteins was selected from the larger list and are shown in Table 5.

TABLE 5

| | | | Predicted | Predicted |
|---|---|---|---|---|
| SEQ ID NO. | Peptide | Length | Affinity (nM) | Stability (hr) |
| SEQ ID NO: 31 | YTVKFALV | 8 | 82.32 | 1.5825 |
| SEQ ID NO: 32 | MTYKFPVT | 8 | 4946.5098 | 0.3193 |
| SEQ ID NO: 33 | QIPTKLAA | 8 | 15361.5996 | 0.3282 |
| SEQ ID NO: 34 | FLQEKSPAV | 9 | 3.07 | 4.8237 |
| SEQ ID NO: 35 | FLAIKLYGV | 9 | 3.19 | 9.0043 |
| SEQ ID NO: 36 | FLLDKKIGV | 9 | 3.73 | 6.6584 |
| SEQ ID NO: 37 | ALWAKSPGV | 9 | 4.99 | 5.8786 |
| SEQ ID NO: 38 | FLLEKSRVV | 9 | 7.71 | 4.5509 |
| SEQ ID NO: 39 | YLSEKVLAA | 9 | 8.02 | 6.1459 |
| SEQ ID NO: 40 | FLAVKPDGV | 9 | 13.71 | 1.5977 |
| SEQ ID NO: 41 | LLYFKVFGI | 9 | 13.76 | 5.5169 |
| SEQ ID NO: 4 | LMYAKRAFV | 9 | 21.17 | 2.7592 |
| SEQ ID NO: 42 | ILISKLLGA | 9 | 22.27 | 4.6864 |
| SEQ ID NO: 43 | YLLEKTRVA | 9 | 33.6 | 2.583 |
| SEQ ID NO: 44 | ALDFKLAAA | 9 | 77.81 | 1.2295 |
| SEQ ID NO: 3 | QLAKLMATL | 9 | 106.98 | 1.7018 |
| SEQ ID NO: 2 | RMQKEITAL | 9 | 142.28 | 1.2663 |
| SEQ ID NO: 45 | QLALKVEGV | 9 | 182.69 | 1.8798 |
| SEQ ID NO: 11 | IMALKQAGL | 9 | 264.42 | 0.5598 |
| SEQ ID NO: 1 | GMGQKDSYV | 9 | 267.75 | 1.447 |
| SEQ ID NO: 46 | FLKIKVTAA | 9 | 408.29 | 0.6906 |
| SEQ ID NO: 12 | FLAIKLYVG | 9 | 645.31 | 0.5 |
| SEQ ID NO: 47 | ALLAKRLGA | 9 | 2100.1101 | 0.6015 |
| SEQ ID NO: 13 | GAATKMAAV | 9 | 3260.8501 | 0.3735 |
| SEQ ID NO: 48 | GLTFKGPGA | 9 | 4459.0601 | 0.3391 |
| SEQ ID NO: 49 | YAVTKTDGI | 9 | 7557.0298 | 0.2862 |
| SEQ ID NO: 50 | IFINKLNGA | 9 | 13571.1602 | 0.445 |
| SEQ ID NO: 51 | ILKEKVHVG | 9 | 21064.4707 | 0.2595 |
| SEQ ID NO: 52 | KVLGKGSFG | 9 | 28109.5 | 0.1592 |
| SEQ ID NO: 53 | RTSQKGTVA | 9 | 29062.3496 | 0.2573 |
| SEQ ID NO: 54 | QSDPKPAVA | 9 | 31365.6191 | 0.1399 |
| SEQ ID NO: 55 | LFLPKLVVG | 9 | 34160.8594 | 0.1436 |
| SEQ ID NO: 56 | IDPYKEFGA | 9 | 35010.2617 | 0.1049 |
| SEQ ID NO: 57 | IDTYKEFGA | 9 | 36837.0898 | 0.1074 |
| SEQ ID NO: 58 | FLAIKKLYGV | 10 | 8.03 | 4.6103 |
| SEQ ID NO: 59 | NLLEKDYFGL | 10 | 10.97 | 1.9202 |
| SEQ ID NO: 60 | GLFGKTQVGV | 10 | 16.01 | 4.6994 |
| SEQ ID NO: 61 | SLLFKTSVGV | 10 | 26.25 | 2.7621 |
| SEQ ID NO: 62 | LLEGKVLPGV | 10 | 49.01 | 2.7821 |
| SEQ ID NO: 63 | LLYFKKVFGI | 10 | 52.6 | 3.1445 |
| SEQ ID NO: 64 | ALWAKSPGVL | 10 | 143.86 | 0.8368 |
| SEQ ID NO: 14 | QLALKKVEGV | 10 | 655.88 | 1.1942 |
| SEQ ID NO: 15 | IMALKKQAGL | 10 | 706.76 | 0.5612 |
| SEQ ID NO: 65 | FLKIKKVTAA | 10 | 1295.64 | 0.57 |
| SEQ ID NO: 16 | FLAIKKLYVG | 10 | 2071.94 | 0.41 |
| SEQ ID NO: 66 | ALLAKKRLGA | 10 | 3893.4299 | 0.5511 |
| SEQ ID NO: 67 | AALKKALAAA | 10 | 5693.2798 | 0.4598 |
| SEQ ID NO: 68 | YAVTKKTDGI | 10 | 13916.4697 | 0.2621 |
| SEQ ID NO: 69 | TTGLKPTVAL | 10 | 18363.5508 | 0.1841 |
| SEQ ID NO: 17 | TEIGKDVIGL | 10 | 21717.4805 | 0.2002 |
| SEQ ID NO: 70 | KVLGKGSFGK | 10 | 22916.7109 | 0.1779 |
| SEQ ID NO: 18 | ILKEKKVHVG | 10 | 28838.6406 | 0.2052 |

The predicted affinities of the peptides for HLA-A*02:01 and stabilities (half-lives) of the peptide-HLA complexes were calculated using NetMHCpan (a method using artificial neural networks described at Nielsen, et al., PLOS One, Aug. 29, 2007, https://doi.org/10.1371/journal.pone.0000796) and NetMHCstab (a method for predicting the stability of peptide binding to a number of different MHC molecules using artificial neural networks (ANNs) described at Jorgensen et al., Immunology 2014, 141(1): 18-26), respectively.

In addition, a few epitope sequences with high predicted affinities (Kd ˜5 nM) were modified to generate peptides having moderated, intermediate predicted affinities/stabilities, e.g., not as high of affinity. The sequence modifications were alteration of the P8-9 residues to less optimal anchors, e.g., changing GV to VG, and insertion of an additional Lysine after P4 residue to increase the peptide length to 10. For example, as shown in Table 6, EP10 ILKEKKVHVG (SEQ ID NO: 18) is derived from ILKEKVHVG (SEQ ID NO: 51); EP8 FLAIKKLYVG (SEQ ID NO: 16) is derived from FLAIKKLYGV (SEQ ID NO: 58) or from EP4 FLAIKLYVG (SEQ ID NO: 12) of Table 6; and EP6 QLALKKVEGV (SEQ ID NO: 14) is derived from QLALKVEGV (SEQ ID NO: 45).

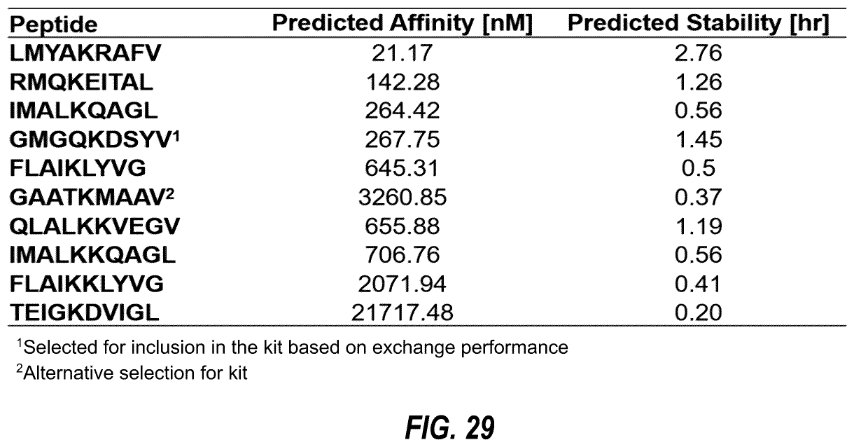

Peptides with predicted affinities ranging from ˜10 nM to ˜20,000 nM and predicted stabilities ranging from ˜2 hrs to 0.2 hrs (marked in bold font) were selected for further testing. The selected sequences are shown in Table 6 (also FIG. 29). Additionally, the structure of the labelled peptides sequence is also shown.

TABLE 6

| Candidate | Peptide | Labelled peptide | Predicted | Predicted | No. of AA |
|---|---|---|---|---|---|
| no. | sequence | structure | Affinity (nM) | Stability (hr) | residues |
| EP1 | RMQKEITAL | RMQ{Lys(FITC)} | 142.28 | 1.2663 | 9 |
| | (SEQ ID NO: 2) | EITAL | | | |
| | | (SEQ ID NO: 20) | | | |
| EP2 | IMALKQAGL | IMAL{Lys(FITC)} | 264.42 | 0.5598 | 9 |
| | (SEQ ID NO: 11) | QAGL | | | |
| | | (SEQ ID NO: 21) | | | |
| EP3 | GMGQKDSYV | GMGQ{Lys(FITC)} | 267.75 | 1.447 | 9 |
| | (SEQ ID NO: 1) | DSYV | | | |
| | | (SEQ ID NO: 19) | | | |
| EP4 | FLAIKLYVG | FLAK{Lys(FITC)} | 645.31 | 0.5 | 9 |
| | (SEQ ID NO: 12) | LYVG | | | |
| | | (SEQ ID NO: 22) | | | |
| EP5 | GAATKMAAV | GAAT{Lys(FITC)} | 3260.8501 | 0.3735 | 9 |
| | (SEQ ID NO: 13) | MAAV | | | |
| | | (SEQ ID NO: 23) | | | |
| EP6 | QLALKKVEGV | QLALK{Lys(FITC)} | 655.88 | 1.1942 | 10 |
| | (SEQ ID NO: 14) | VEGV | | | |
| | | (SEQ ID NO: 24) | | | |
| EP7 | IMALKKQAGL | IMALK{Lys(FITC)} | 706.76 | 0.5612 | 10 |
| | (SEQ ID NO: 15) | QAGL | | | |
| | | (SEQ ID NO: 25) | | | |
| EP8 | FLAIKKLYVG (SEQ | FLAIK{Lys(FITC)} | 2071.94 | 0.41 | 10 |
| | ID NO: 16) | LYVG | | | |
| | | (SEQ ID NO: 26) | | | |
| EP9 | TEIGKDVIGL (SEQ | TEIG{Lys(FITC)} | 21717.4805 | 0.2002 | 10 |
| | ID NO: 17) | DVIGL | | | |
| | | (SEQ ID NO: 27) | | | |
| EP10 | ILKEKKVHVG | ILKEK{Lys(FITC)} | 28838.6406 | 0.2052 | 10 |
| | (SEQ ID NO: 18) | VHVG | | | |
| | | (SEQ ID NO: 28) | | | |

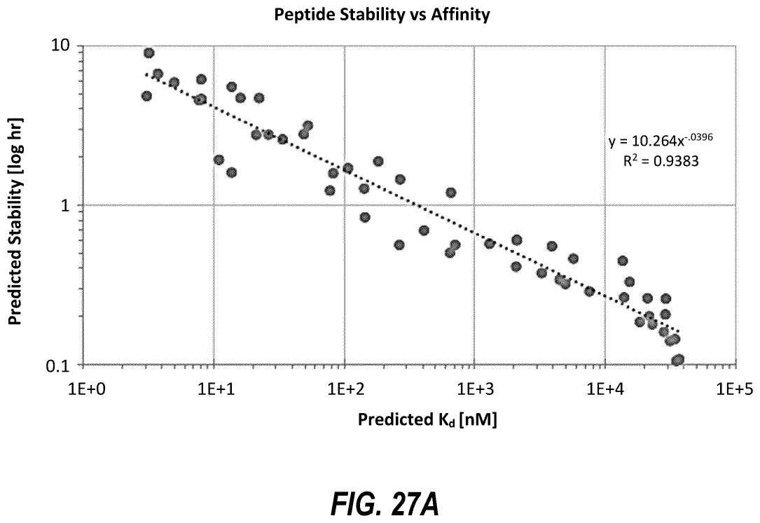

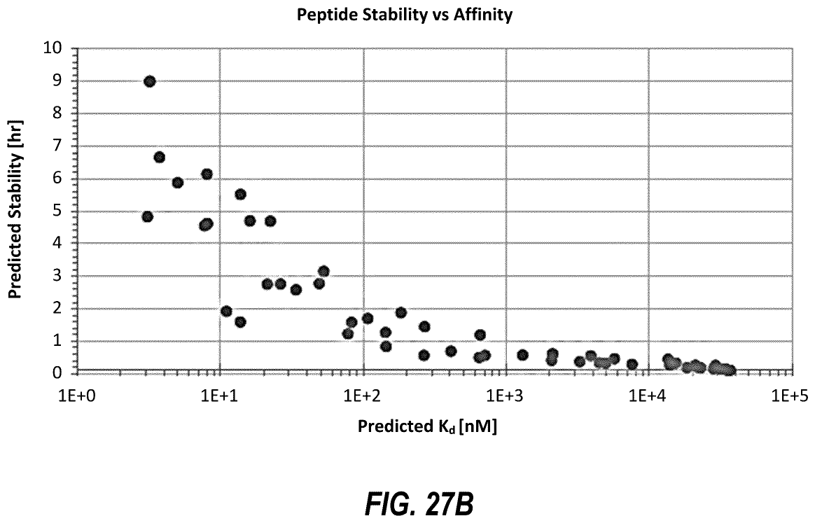

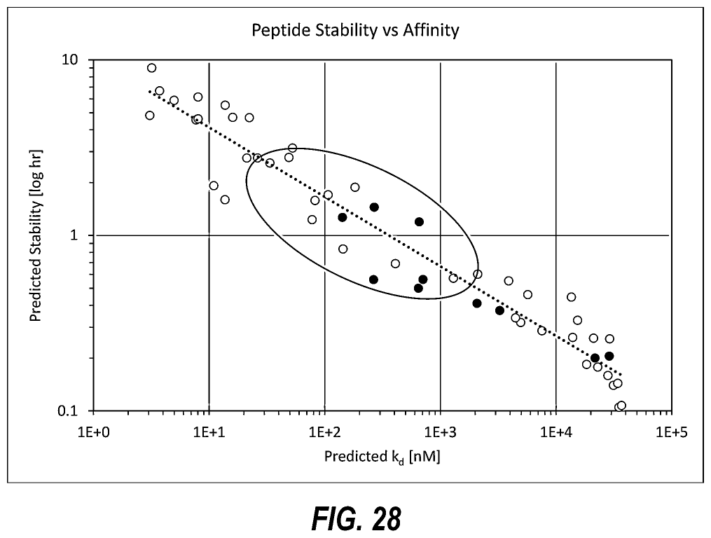

FIGS. 27A-27B show the predicted affinity (x-axis) of the peptides shown in Table 5 for HLA*A0201 against the predicted stability of the peptide within the complex. As shown in FIG. 28, the candidate peptide sequences of Table 6 (FIG. 29), are shown mapped (solid black data points) and are clustered in a mid-region of the affinity: stability graphed relationship. Suitable peptides for use as in-place (i.e., initial) peptides may be identified within the region roughly bounded by the oval. The ten candidate peptides are generally not the peptides with the highest affinity and/or highest stability.

As noted in FIG. 29, peptide sequences GMGQKDSYV (SEQ ID NO: 1) and GAATKMAAV (SEQ ID NO: 13) were found to have affinity and exchangeability lending each of them to be the most suitable in-place peptides, permitting stoichiometric loading of antigenic peptides and/or candidate antigenic peptides to be most efficiently loaded into the HLA, thus providing controllable manufacture of the initial peptide:MHC complex.

### Candidate In-Place Peptide Exchange Testing

Candidate peptides with a FITC-modified Lysine at Position 4 or 5 shown in Table 6 were purchased from a peptide manufacturer (Genscript). The fluorescent peptides were loaded into recombinant, biotinylated HLA-A*02:01 by a contract manufacturer (Biolegend). Successfully generated candidate pHLAs were then used in a catalyzed peptide exchange reaction to test loading of a peptide of interest (incoming peptide) under standard exchange conditions (room temperature, 10 mM Exchange Catalyst (GlyCha (glycine cyclohexylalanine)), assay buffer (e.g., PBS), 4 hour incubation). The peptide-HLA (pHLA) concentration in the reactions was 1 micromolar. The incoming peptide concentration was 20 micromolar.

For the incoming peptide, the peptide sequence LMYAKRAFV (SEQ ID NO: 4) (shown in FIG. 29) was selected as the peptide, based on its higher affinity and stability as predicted. This may be used as a model for candidate antigenic peptides, which need to displace an in-place peptide stabilizing the MHC (e.g., loaded into the MHC complex), in methods of discovering antigenic peptides and methods of activating lymphocytes as described throughout the specification.

After four hours, exchanged, unexchanged and non-fluorescent biotinylated HLA complexes were loaded onto Streptavidin coated capture beads. The HLA complex coated beads were analyzed for FITC Fluorescence on a Flow Cytometer. Highly exchangeable peptides result in loss of nearly all FITC signal due to displacement of the in-place peptide.

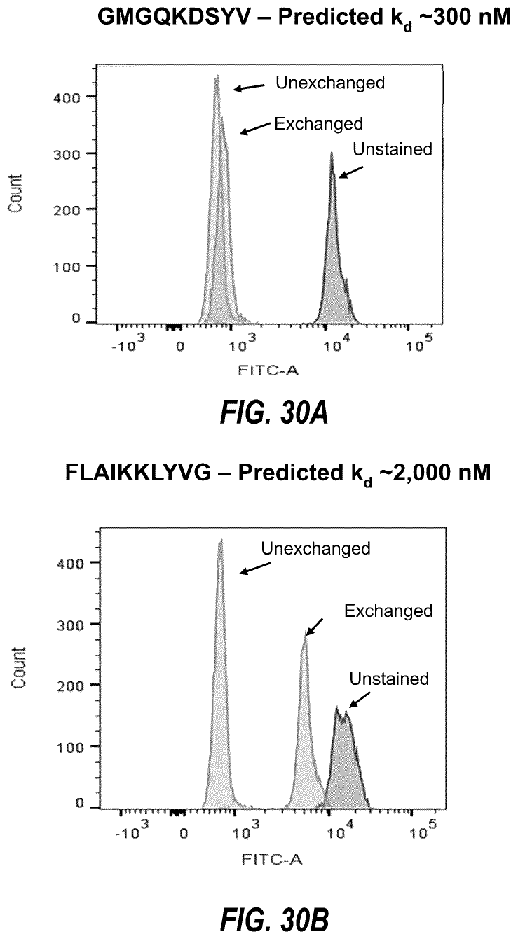

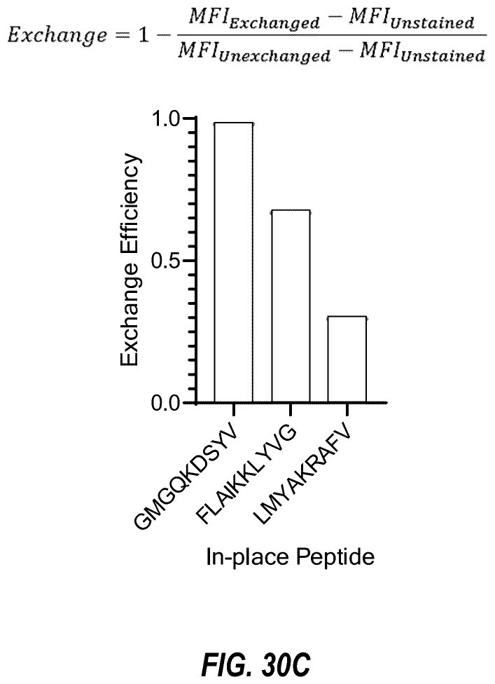

FIGS. 30A and 30B show the flow cytometry results showing counts of exchanged, unexchanged, and unstained HLA complexes with GMGQKDSYV (SEQ ID NO: 1) and FLAIKKLYVG (SEQ ID NO: 16), respectively, as the in-place peptide. FIG. 30C shows that the exchange efficiency can be calculated based on the flow cytometry results showing counts of exchanged, unexchanged, and unstained, and the exchange efficiencies for the three peptides. GMGQKDSYV (SEQ ID NO: 1) demonstrated exchange efficiency of about 1.0, indicating nearly complete exchange, while FLAIKKLYVG (SEQ ID NO: 16) had an exchange efficiency of about 0.65. LMYAKRAFV (SEQ ID NO: 4), having a higher affinity and stability, had an exchange efficiency of about 0.30.

### Additional Disclosed Items

Further additional embodiments are described below.

Embodiment 1 is a kit for generating an antigen-presenting surface, the kit includes: a covalently functionalized synthetic surface; a primary activating molecule including a major histocompatibility complex (MHC) molecule and a first reactive moiety, wherein the MHC molecule is configured to bind to a T cell receptor (TCR), and the first reactive moiety is configured to react with or bind to the covalently functionalized surface; and an initial peptide bound to the MHC molecule, wherein the initial peptide includes IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

Embodiment 2 is a kit for generating an antigen-presenting surface, the kit includes: a covalently functionalized synthetic surface; a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface; and an initial peptide bound to the MHC molecule, wherein the initial peptide includes GMGQKDSYV (SEQ ID NO: 1); RMQKEITAL (SEQ ID NO: 2); IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

Embodiment 3 is a kit for generating an antigen-presenting surface, the kit including: a covalently functionalized synthetic surface; a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface; and an initial peptide bound to the MHC molecule, wherein the initial peptide has an affinity for binding a binding groove of the MHC molecule including a predicted Kd from about 1E+1 nM to about 1E+5 nM, from about 1E+1 nM to about 2E+5 nM, from about 1E+2 nM to about 1E+5 nM, from about 1E+2 nM to about 2E+4 nM, from about 1E+2 nM to about 1E+3 nM, from about 1E+2 nm to about 1E+4 nM, from about 1E+1 nm to about 2E+4 nM, from about 1E+2 nM to about 1E+3 nM, from about 1.5E+2 nM to about 1E+5 nM.

Embodiment 4 is the kit of any one of embodiments 1 to 3, further including one or more of: at least one co-activating molecule that includes a second reactive moiety configured to react with or bind to the covalently functionalized surface, wherein each co-activating molecule is selected from a TCR co-activating molecule and an adjunct TCR activating molecule; a surface-blocking molecule capable of covalently binding to the covalently functionalized synthetic surface; a buffer suitable for performing an exchange reaction; and instructions for performing an exchange reaction wherein a peptide antigen displaces the initial peptide.

Embodiment 5 is the kit of any one of embodiments 1 to 4, further including an exchange factor, wherein the exchange factor is provided separately from the primary activating molecule and the initial peptide bound to the MHC molecule.

Embodiment 6 is the kit of embodiment 5, wherein the exchange factor includes Leu, Phe, Val, Arg, Met, Lys, Ile, homoleucine, cyclohexylalanine, or Norleucine as its C-terminal amino acid residue.

Embodiment 7 is the kit of embodiment 5 or embodiment 6, wherein the exchange factor includes Gly, Ala, Ser, or Cys as its penultimate C-terminal residue.

Embodiment 8 is the kit of any one of embodiments 5 to 7, wherein the exchange factor is 2 amino acid residues in length.

Embodiment 9 is the kit of any one of embodiments 1 to 8, further including a plurality of surface-blocking molecules, wherein the covalently functionalized surface further includes a first additional plurality of binding moieties configured for binding the surface-blocking molecule.

Embodiment 10 is the kit of any one of embodiments 1 to 9, wherein the initial peptide includes at least 4 or 5 amino acid residues; or has a length of about 4, 5, 6, 7, 8, 9, 10, 11, 12, 13, 14, 15, 16, 17, or 18 amino acid residues (e.g., ranging from 8 to 10 amino acid residues, 13 to 15 amino acid residues, or 13 to 18 amino acid residues).

Embodiment 11 is the kit of any one of embodiments 1 to 10, wherein the initial peptide includes a lysine as the fourth or fifth amino acid residue.

Embodiment 12 is the kit of any one of embodiments 1 to 11, wherein the initial peptide includes a label, optionally wherein the label is attached to a fourth or fifth amino acid residue.

Embodiment 13 is the kit of embodiment 12, wherein the label is a fluorescent label.

Embodiment 14 is the kit of any one of embodiments 1 to 13, wherein the initial peptide has a sequence including or consisting of a sequence from a naturally occurring (e.g., mammalian or human) polypeptide.

Embodiment 15 is the kit of any one of embodiments 1 to 14, wherein the sequence of the initial peptide consists of a sequence that appears in a wild-type (e.g., mammalian or human) polypeptide.

Embodiment 16 is the kit of any one of embodiments 1 to 15, wherein the initial peptide is non-immunogenic.

Embodiment 17 is the kit of any one of embodiments 1 to 16, wherein the sequence of the initial peptide includes or consists of a sequence from a highly conserved self peptide sequence (e.g., a peptide sequence with a below average mutation rate; optionally wherein the mutation rate is at least one or two standard deviations below the average amino acid mutation rate in the organism) and minimal immunogenicity.

Embodiment 18 is the kit of any one of embodiments 1 to 17, wherein the sequence of the initial peptide includes or consists of a sequence from a cytoskeletal polypeptide, e.g., an actin or tubulin polypeptide, or sequence from a ribosomal polypeptide, e.g., the RPSA, RPS2, RPL3, RPL4, RPL5, RPL6, RPL7A, or RPP0 polypeptides.

Embodiment 19 is the kit of any one of embodiments 1 to 18, wherein the initial peptide includes or consists of any one of SEQ ID NOs: 1-4 or any one of SEQ ID NOs: 1, 2, 11-18, or any one of SEQ ID NOs: 11-18.

Embodiment 20 is the kit of any one of embodiments 1 to 19, wherein the initial peptide includes GAATKMAAV (SEQ ID NO: 13).

Embodiment 21 is the kit of any one of embodiments 2 to 19, wherein the initial peptide includes GMGQKDSYV (SEQ ID NO: 1) or GAATKMAAV (SEQ ID NO: 13).

Embodiment 22 is the kit of any one of embodiments 1 to 21, wherein the affinity of the initial peptide to a binding groove of the MHC includes a predicted Kd from about 1E+2 nm to about 1E+5 nm.

Embodiment 23 is the kit of any one of embodiments 1 to 22, wherein the initial peptide binds the MHC molecule with high affinity, a low off-rate, and/or a long half-life.

Embodiment 24 is the kit of any one of embodiments 1 to 23, wherein the binding of the initial peptide to the MHC molecule has a half-life of at least about 0.1, 0.2, 0.4, 1.5, 2, 4, 6, 8, 10, 12, 14, 16, 18, 20, 24, 28, 32, 36, 48, or 72 hours, or the binding of the initial peptide to the MHC molecule has a half-life in the range of about 4-12, 8-16, 12-20, 20-28, 24-32, 28-36, 32-40, 36-48, or 48-72 hours.

Embodiment 25 is the kit of any one of embodiments 1 to 24, wherein the initial peptide binds the MHC molecule with a half-life of at least about 4 hours.

Embodiment 26 is the kit of any one of embodiments 1 to 25, wherein the covalently functionalized synthetic surface presents a plurality of azido groups.

Embodiment 27 is the kit of embodiment 26, wherein the first reactive moieties are configured to react with the azido groups of the covalently functionalized synthetic surface so as to form covalent bonds.

Embodiment 28 is the kit of any one of embodiments 1 to 27, wherein the covalently functionalized synthetic surface presents a plurality of biotin-binding agents, and wherein the first reactive moieties are configured to specifically bind to the biotin-binding agent.

Embodiment 29 is the kit of any one of embodiments 1 to 28, wherein the MHC molecule includes a human leukocyte antigen A (HLA-A) heavy chain.

Embodiment 30 is the kit of any one of embodiments 1 to 29, further including at least one co-activating molecule that includes a second reactive moiety configured to react with or bind to the covalently functionalized surface, wherein each co-activating molecule is selected from a TCR co-activating molecule and an adjunct TCR activating molecule; the TCR co-activating molecule includes a CD28 binding protein or a fragment thereof which retains binding ability with CD28; and the adjunct TCR activating molecule is configured to provide adhesion stimulation and/or the adjunct TCR activating molecular ligand includes a CD2 binding protein, anti-CD2 antibody, or a fragment thereof, wherein the fragment of the CD2 binding protein or anti-CD2 antibody retains binding ability with CD2.

Embodiment 31 is a method of identifying an initial peptide sequence for stabilizing a major histocompatibility complex (MHC) molecule, including: binding a first peptide sequence to a binding groove of the MHC molecule, wherein the first peptide sequence is a detectably labelled peptide sequence, thereby forming a detectably labelled peptide sequence:MHC molecule complex (LP:MHC complex) stabilizing the MHC molecule; performing an exchange reaction including contacting the LP:MHC complex with an exchange factor and a second peptide sequence for a first period of time, wherein the second peptide sequence is configured to stabilize the MHC molecule when bound to the binding groove; and detecting displacement of the detectably labelled peptide sequence from the binding groove of the MHC molecule.

Embodiment 32 is the method of embodiment 31, wherein the detectably labelled peptide sequence includes a highly conserved self peptide sequence (e.g., a peptide sequence with a below average mutation rate, optionally wherein the mutation rate is at least one or two standard deviations below the average amino acid mutation rate in the organism) and minimal immunogenicity.

Embodiment 33 is the method of embodiment 31 or embodiment 32, wherein the detectably labelled peptide sequence is labelled at an amino acid residue that does not interfere with forming the LP:MHC complex.

Embodiment 34 is the method of any one of embodiments 31 to 33, wherein an affinity of the detectably labelled peptide sequence for binding the binding groove includes a predicted Kd from about 1E+1 nM to about 1E+5 nM, from about 1E+1 nM to about 2E+5 nM, from about 1E+2 nM to about 1E+5 nM, from about 1E+2 nM to about 2E+4 nM, from about 1E+2 nM to about 1E+3 nM, from about 1E+2 nm to about 1E+4 nM, from about 1E+1 nm to about 2E+4 nM, from about 1E+2 nM to about 1E+3 nM, from about 1.5E+2 nM to about 1E+5.

Embodiment 35 is the method of any one of embodiments 31 to 34, wherein the detectably labelled peptide sequence binds the MHC molecule with high affinity, a low off-rate, and/or a long half-life.

Embodiment 36 is the method of any one of embodiments 31 to 35, wherein the exchange factor includes a Gly, Ala, Ser, or Cys as its penultimate C-terminal residue.

Embodiment 37 is the method of any one of embodiments 31 to 36, wherein the exchange factor includes Leu, Phe, Val, Arg, Met, Lys, Ile, homoleucine, cyclohexylalanine, or Norleucine as its C-terminal amino acid residue.

Embodiment 38 is the method of any one of embodiments 31 to 37, wherein the exchange factor is 2 amino acid residues in length.

Embodiment 39 is the method of any one of embodiments 32 to 38, wherein the first period of time is about 2 hr to about 6 hr or 1 hr to about 10 hr.

Embodiment 40 is the method of any one of embodiments 31 to 39, wherein detecting displacement of the detectably labelled peptide includes determining loss of fluorescence from the LP:MHC complex.

Embodiment 41 is the method of embodiment 40, wherein determining the loss of fluorescence from the LP:MHC complex includes capturing MHC molecule after the first period of time to capture objects; and determining fluorescence of the captured MHC molecule, thereby determining a proportion of displacement of the detectably labelled peptide from the LP:MHC complex.

Embodiment 42 is the method of any one of embodiments 31 to 41, wherein the detectably labelled peptide includes at least 4 or 5 amino acid residues; or has a length of about 4, 5, 6, 7, 8, 9, 10, 11, 12, 13, 14, 15, 16, 17, or 18 amino acid residues, optionally wherein the detectably labelled peptide has a length of 8 to 10 amino acid residues, 13 to 15 amino acid residues, or a length of 13 to 18 amino acid residues.

Embodiment 43 is the method of any one of embodiments 31 to 42, wherein the detectably labelled peptide sequence includes a lysine as the fourth or fifth amino acid residue of the detectably labelled peptide sequence.

Embodiment 44 is the method of embodiment 43, wherein the detectably labelled peptide is labelled at the lysine residue.

Embodiment 45 is the method of any one of embodiments 31 to 44, wherein the initial peptide binds the MHC molecule with high affinity, a low off-rate, and/or a long half-life, optionally wherein the binding of the initial peptide to the MHC molecule has a half-life of at least about 0.1, 0.2, 0.4, 1.5, 2, 4, 6, 8, 10, 12, 14, 16, 18, 20, 24, 28, 32, 36, 48, or 72 hours, or the binding of the initial peptide to the MHC molecule has a half-life in the range of about 4-10, 4-12, 8-16, 10-15, 12-20, 15-20, 20-25, 20-28, 24-32, 25-30, 28-36, 30-35, 32-40, 35-40, 36-48, or 48-72 hours.

Embodiment 46 is the method of any one of embodiments 31 to 45, wherein the detectably labelled peptide has an amino acid sequence selected from: IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

Embodiment 47 is the method of any one of embodiment 31 to 46, wherein the detectably labelled peptide includes or consists of any one of SEQ ID NOs: 1-4 or any one of SEQ ID NOs: 1, 2, 11-18, or any one of SEQ ID NOs: 11-18.

Embodiment 48 is the method of any one of embodiments 31 to 47, wherein the detectably labelled peptide has an amino acid sequence selected from: GMGQKDSYV (SEQ ID NO: 1); RMQKEITAL (SEQ ID NO: 2); IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

Embodiment 49 is the method of any one of embodiments 31 to 48, wherein the detectably labelled peptide has an amino acid sequence of GAATKMAAV (SEQ ID NO: 13).

Embodiment 50 is the method of any one of embodiments 31 to 48, wherein the detectably labelled peptide has an amino acid sequence of GMGQKDSYV (SEQ ID NO: 1) or GAATKMAAV (SEQ ID NO: 13).

Embodiment 51 is the method of any one of embodiments 31 to 50, wherein the detectably labelled peptide sequence has a sequence including or consisting of a sequence from a naturally occurring (e.g., mammalian or human) polypeptide.

Embodiment 52 is the method of any one of embodiments 31 to 51, wherein the sequence of the detectably labelled peptide sequence consists of a sequence that appears in a wild-type (e.g., mammalian or human) polypeptide.

Embodiment 53 is the method of any one of embodiments 31 to 52, wherein the detectably labelled peptide sequence is non-immunogenic.

Embodiment 54 is the method of any one of embodiments 31 to 53, wherein the detectably labelled peptide sequence includes or consists of a sequence from a cytoskeletal polypeptide, e.g., an actin or tubulin polypeptide, or sequence from a ribosomal polypeptide, e.g., the RPSA, RPS2, RPL3, RPL4, RPL5, RPL6, RPL7A, or RPP0 polypeptides.

Embodiment 55 is the method of any one of embodiments 31 to 54, wherein the detectably labelled peptide sequence includes C-terminal amino acid residues selected to modulate release kinetics from a binding pocket of the binding groove of the MHC.

Embodiment 56 is the method of embodiment 55, wherein the binding pocket is a F binding pocket of the binding groove.

Embodiment 57 is the method of any one of embodiments 31 to 56, wherein the second peptide sequence displaces at least 60% (65%, 70%, 75%, 80%, 85%, 90%, 95%, 99%) of the detectably labelled peptide bound to the MHC molecules, thereby identifying the second peptide as a suitable initial peptide sequence.

Embodiment 58 is the method of any one of embodiments 31 to 57, wherein when the MHC molecule includes an HLA-A molecule, the second peptide sequence is LMYAKRAFV (SEQ ID NO: 4).

Embodiment 59 is the method of any one of embodiments 31 to 58, wherein the MHC molecule includes HLA-A*01, HLA-A*02, HLA-A*03, HLA-A*11, or HLA-A*24.

Embodiment 60 is the method of any one of embodiments 31 to 59, wherein the MHC molecule includes HLA-B*07, HLA-B*27, HLA-B*40, HLA-B*44, or HLA-B*58.

Embodiment 61 is a kit for generating an antigen-presenting surface, the kit including: (a) a covalently functionalized synthetic surface; (b) a primary activating molecule that includes a major histocompatibility complex (MHC) molecule configured to bind to a T cell receptor (TCR), and a first reactive moiety configured to react with or bind to the covalently functionalized surface; and (c) an initial peptide bound to the MHC molecule, wherein the initial peptide is a peptide sequence configured to stabilize the MHC molecule identified by the method of any one of embodiments 31 to 60.

Embodiment 62 is the kit of embodiment 61 further including one or more of: at least one co-activating molecule that includes a second reactive moiety configured to react with or bind to the covalently functionalized surface, wherein each co-activating molecule is selected from a TCR co-activating molecule and an adjunct TCR activating molecule; a surface-blocking molecule capable of covalently binding to the covalently functionalized synthetic surface; a buffer suitable for performing an exchange reaction; and instructions for performing an exchange reaction wherein a peptide antigen displaces the initial peptide.

Embodiment 63 is the kit of embodiment 61 or embodiment 62, further including an exchange factor, wherein the exchange factor is provided separately from the primary activating molecule and the initial peptide bound to the MHC molecule.

Embodiment 64 is the kit of embodiment 63, wherein the exchange factor includes Leu, Phe, Val, Arg, Met, Lys, Ile, homoleucine, cyclohexylalanine, or Norleucine as its C-terminal amino acid residue.

Embodiment 65 is the kit of embodiment 63 or embodiment 64, wherein the exchange factor includes Gly, Ala, Ser, or Cys as its penultimate C-terminal residue.

Embodiment 66 is the kit of any one of embodiments 63 to 65, wherein the exchange factor is 2 amino acid residues in length.

Embodiment 67 is the kit of any one of embodiments 61 to 66, further including a plurality of surface-blocking molecules, wherein the covalently functionalized surface further includes a first additional plurality of binding moieties configured for binding the surface-blocking molecule.

Embodiment 68 is the kit of any one of embodiments 61 to 67, wherein the initial peptide includes a label.

Embodiment 69 is the kit of embodiment 68, wherein the label is attached to the fourth or fifth amino acid residue (e.g., lysine).

Embodiment 70 is the kit of embodiment 68 or 69, wherein the label is a fluorescent label.

Embodiment 71 is the kit of any one of embodiments 61 to 70, wherein the affinity of the initial peptide to a binding groove of the MHC includes a predicted Kd from about 1E+2 nm to about 1E+5 nm.

Embodiment 72 is the kit of any one of embodiments 61 to 71, wherein the initial peptide binds the MHC molecule with high affinity, a low off-rate, and/or a long half-life.

Embodiment 73 is the kit of any one of embodiments 61 to 72, wherein the binding of the initial peptide to the MHC molecule has a half-life of at least about 0.1, 0.2, 0.4, 1.5, 2, 4, 6, 8, 10, 12, 14, 16, 18, 20, 24, 28, 32, 36, 48, or 72 hours, or the binding of the initial peptide to the MHC molecule has a half-life in the range of about 4-10, 4-12, 8-16, 10-15, 12-20, 15-20, 20-25, 20-28, 24-32, 25-30, 28-36, 30-35, 32-40, 35-40, 36-48, or 48-72 hours.

Embodiment 74 is the kit of any one of embodiments 61 to 73, wherein the initial peptide binds the MHC molecule with a half-life of at least about 4 hours.

Embodiment 75 is the kit of any one of embodiments 61 to 74, wherein the covalently functionalized synthetic surface presents a plurality of azido groups.

Embodiment 75A is the method of embodiment 75, wherein the first reactive moieties are configured to react with the azido groups of the covalently functionalized synthetic surface so as to form covalent bonds.

Embodiment 76 is the kit of any one of embodiments 61 to 75, wherein the covalently functionalized synthetic surface presents a plurality of biotin-binding agents, and wherein the first reactive moieties are configured to specifically bind to the biotin-binding agent.

Embodiment 77 is the kit of any one of embodiments 61 to 76, wherein the MHC molecule includes a human leukocyte antigen A (HLA-A) heavy chain.

Embodiment 78 is the kit of any one of embodiments 61 to 77, further including at least one co-activating molecule that includes a second reactive moiety configured to react with or bind to the covalently functionalized surface, wherein each co-activating molecule is selected from a TCR co-activating molecule and an adjunct TCR activating molecule; wherein the TCR co-activating molecule includes a CD28 binding protein or a fragment thereof which retains binding ability with CD28; and wherein the adjunct TCR activating molecule is configured to provide adhesion stimulation and/or the adjunct TCR activating molecular ligand includes a CD2 binding protein, anti-CD2 antibody, or a fragment thereof, wherein the fragment of the CD2 binding protein or anti-CD2 antibody retains binding ability with CD2.

Embodiment 79 is a method of forming a proto-antigen-presenting surface, the method including: synthesizing a plurality of primary activating molecules, wherein synthesizing each of the plurality of primary activating molecules includes reacting a major histocompatibility complex (MHC) molecule with an initial peptide (e.g. in the presence of an initial peptide), thereby forming a primary activating molecule, including the MHC molecule complexed with the initial peptide; wherein the initial peptide is the peptide sequence configured to stabilize the MHC molecule identified by the method of any one of embodiments 31 to 60; and reacting the plurality of primary activating molecules with a first plurality of binding moieties disposed on a covalently functionalized synthetic surface, thereby forming the proto-antigen-presenting surface.

Embodiment 80 is the method of embodiment 79, wherein each of the primary activating molecules includes a first reactive moiety; reacting the plurality of primary activating molecules with the first plurality of binding moieties disposed on the covalently functionalized synthetic surface includes reacting the first reactive moiety of each of the primary activating molecules with a corresponding one of the first plurality of binding moieties.

Embodiment 81 is the method embodiment 79 or embodiment 80, wherein reacting the plurality of primary activating molecules with the first plurality of binding moieties disposed on the covalently functionalized synthetic surface further includes adding a first reactive moiety to the MHC molecule of the plurality of primary activating molecules prior to reacting the plurality of primary activating molecules with the first plurality of binding moieties.

Embodiment 82 is the method of any one of embodiments 79 to 81, further including reacting the plurality of primary activating molecules with an exchange factor and, optionally in the presence of a peptide antigen, thereby forming an antigen-presenting surface.

Embodiment 83 is the method of embodiment 82, wherein the exchange factor includes Leu, Phe, Val, Arg, Met, Lys, Ile, homoleucine, cyclohexylalanine, or Norleucine as its C-terminal amino acid residue.

Embodiment 84 is the method of embodiment 82 or 83, wherein the exchange factor includes Gly, Ala, Ser, or Cys as its penultimate C-terminal residue.

Embodiment 85 is the method of any one of embodiments 82 to 84, wherein the exchange factor is 2 amino acid residues in length.

Embodiment 86 is the method of any one of embodiments 79 to 85, wherein the initial peptide binds the MHC molecule with a half-life of at least about 4 hours.

Embodiment 87 is the method of any one of embodiments 79 to 86, wherein the covalently functionalized synthetic surface presents a plurality of azido groups.

Embodiment 88 is the method of embodiment 87, wherein the first reactive moieties are configured to react with the azido groups of the covalently functionalized synthetic surface so as to form covalent bonds.

Embodiment 89 is the method of any one of embodiments 79 to 88, wherein a plurality of co-activating molecular ligands, each including a second reactive moiety and a TCR co-activating molecule or an adjunct TCR activating molecule, are present on the covalently functionalized synthetic surface or are added to the covalently functionalized synthetic surface by reacting the plurality of co-activating molecules with a second plurality of binding moieties of the covalently functionalized synthetic surface configured for binding the second reactive moiety.

Embodiment 90 is the method of embodiments 79 to 89, wherein the covalently functionalized synthetic surface presents a plurality of biotin-binding agents, and wherein the first reactive moieties are configured to specifically bind to the biotin-binding agent.

Embodiment 91 is the method of embodiment 90, wherein the covalently functionalized surface includes a portion configured to exclude biotin-binding agent or biotin functionalities which is disposed at at least one surface of the microfluidic channel of the microfluidic device.

Embodiment 92 is the method of any one of embodiments 79 to 91, wherein the MHC molecule includes a human leukocyte antigen A (HLA-A) heavy chain.

Embodiment 93 is the method of embodiment 89, wherein the TCR co-activating molecule includes a CD28 binding protein or a fragment thereof which retains binding ability with CD28.

Embodiment 94 is the method of embodiment 89 or embodiment 93, wherein the adjunct TCR activating molecule is configured to provide adhesion stimulation and/or the adjunct TCR activating molecular ligand includes a CD2 binding protein, anti-CD2 antibody, or a fragment thereof, wherein the fragment of the CD2 binding protein or anti-CD2 antibody retains binding ability with CD2.

Embodiment 95 is a method of analyzing stability of a complex including a major histocompatibility complex (MHC) molecule and a peptide antigen, wherein the MHC molecule is: configured to bind to a T cell receptor (TCR); and stabilized by complexation with an initial peptide which is identified by the method of any one of embodiments 31-60, wherein the method includes: contacting a plurality of the MHC molecules with the peptide antigen and an exchange factor, thereby forming peptide antigen-bound MHC molecules; and measuring total binding and/or an extent of dissociation of the peptide antigen from the MHC molecule.

Embodiment 96 is the method of embodiment 95, wherein the initial peptide is bound to the MHC molecule before contacting the plurality of the MHC molecules with the peptide antigen and the exchange factor.

Embodiment 97 is the method of embodiment 95 or embodiment 96, wherein measuring total binding and/or the extent of dissociation includes measuring binding of an agent to the MHC molecule, wherein the agent specifically binds to (i) the initial peptide, and/or (ii) a peptide-bound conformation of the MHC molecule.

Embodiment 98 is the method of embodiment 97, wherein the agent includes an antibody, optionally wherein the antibody is produced by clone W6/32.

Embodiment 99 is the method of any one of embodiments 95 to 98, wherein measuring total binding and/or the extent of dissociation includes performing flow cytometry.

Embodiment 100 is the method of any one of embodiments 95 to 99, wherein the MHC molecules are disposed on a covalently functionalized synthetic surface.

Embodiment 101 is the method of embodiment 100 wherein the MHC molecules are disposed on the covalently functionalized synthetic surface via reaction of a reactive moiety of each of the MHC molecules with a binding moiety of the covalently functionalized surface.

Embodiment 102 is the method of any one of embodiments 97 to 99, wherein the agent does not recognize (or bind to) a peptide-unbound conformation of the MHC molecule.

Embodiment 103 is the method of any one of embodiments 95 to 102, wherein the method further includes determining one or more kinetic parameters of the peptide antigen-bound MHC molecules, optionally wherein the one or more kinetic parameters include a half-life and/or an off-rate.

Embodiment 104 is the method of any one of embodiments 95 to 103, wherein the method results in identification of a peptide with a half-life of at least about 0.1, 0.2, 0.4, 1.5, 2, 4, 6, 8, 10, 12, 14, 16, 18, 20, 24, 28, 32, 36, 48, or 72 hours (e.g., at least about 4, 6, 8, 10, 12, 14, 16, or 18 hours); or results in identification of a peptide with a half-life in the range of about 4 to about 40 hours (e.g., about 4 to about 10 hours, about 10 to about 15 hours, about 15 to about 20 hours, about 20 to about 25 hours, about 25 to about 30 hours, about 30 to about 35, or about 35 to about 40 hours).

Embodiment 105 is the method of any one of embodiments 95 to 104, wherein the exchange factor includes Leu, Phe, Val, Arg, Met, Lys, Ile, homoleucine, cyclohexylalanine, or Norleucine as its C-terminal amino acid residue.

Embodiment 106 is the method of any one of embodiments 95 to 105, wherein the exchange factor includes Gly, Ala, Ser, or Cys as its penultimate C-terminal residue.

Embodiment 107 is the method of any one of embodiments 95 to 106, wherein the exchange factor is 2 amino acid residues in length.

Embodiment 108 is the method of any one of embodiments 95 to 107, wherein the initial peptide binds the MHC molecule with a half-life of at least about 0.1, 0.2, 0.4, 1.5, 2, 4, 6, 8, 10, 12, 14, 16, 18, 20, 24, 28, 32, 36, 48, or 72 hours, or in the range of about 4-10, 4-12, 8-16, 10-15, 12-20, 15-20, 20-25, 20-28, 24-32, 25-30, 28-36, 30-35, 32-40, 35-40, 36-48, or 48-72 hours.

Embodiment 109 is the method of any one of embodiments 100 to 108, wherein the covalently functionalized synthetic surface presents a plurality of azido groups.

Embodiment 110 is the method of any one of embodiments 100 to 109, wherein a plurality of co-activating molecular ligands, each including a second reactive moiety and a TCR co-activating molecule or an adjunct TCR activating molecule, are present on the covalently functionalized synthetic surface or are added to the covalently functionalized synthetic surface by reacting the plurality of co-activating molecules with a second plurality of binding moieties of the covalently functionalized synthetic surface configured for binding the second reactive moiety.

Embodiment 111 is the method of any one of embodiments 95 to 110, wherein the MHC molecule includes a human leukocyte antigen A (HLA-A) heavy chain.

Embodiment 112 is the method of embodiment 110, wherein the TCR co-activating molecule includes a CD28 binding protein or a fragment thereof which retains binding ability with CD28.

Embodiment 113 is the method of embodiment 110 or embodiment 112, wherein the adjunct TCR activating molecule is configured to provide adhesion stimulation and/or the adjunct TCR activating molecular ligand includes a CD2 binding protein, anti-CD2 antibody, or a fragment thereof, wherein the fragment of the CD2 binding protein or anti-CD2 antibody retains binding ability with CD2.

Embodiment 114 is a method of analyzing stability of a plurality of complexes each including a histocompatibility complex (MHC) molecule and a peptide antigen, including performing the method of any one of embodiments 95 to 113 with each of a plurality of different peptide antigens.

Embodiment 115 is the method of embodiment 114, wherein the initial peptide includes at least 4 or 5 amino acid residues; or has a length of about 4, 5, 6, 7, 8, 9, 10, 11, 12, 13, 14, 15, 16, 17, 18 or amino acid residues (e.g., ranging from 8 to 10 amino acid residues, 13 to 15 amino acid residues, or 13 to 18 amino acid residues).

Embodiment 116 is the method of embodiment 114 or 115, wherein the initial peptide includes a lysine as the fourth or fifth amino acid residue.

Embodiment 117 is a proto-antigen-presenting surface, the surface including: a plurality of primary activating molecular ligands, wherein each primary activating molecular ligand includes a major histocompatibility complex (MHC) molecule configured to bind to a T cell receptor (TCR) of a T cell; and wherein an initial peptide is bound to the MHC molecule, wherein the initial peptide is the peptide sequence configured to stabilize the MHC molecule identified by the method of any one of embodiments 31 to 60; and a plurality of co-activating molecular ligands each including a TCR co-activating molecule or an adjunct TCR activating molecule.

Embodiment 118 is the proto-antigen-presenting surface of embodiment 117, wherein each of the plurality of primary activating molecular ligands and/or each of the plurality of co-activating molecular ligands is specifically bound to the proto-antigen presenting surface.

Embodiment 119 is the proto-antigen-presenting surface of embodiment 117 or embodiment 118, wherein the plurality of co-activating molecular ligands includes TCR co-activating molecules and adjunct TCR activating molecules, and a ratio of the TCR co-activating molecules to the adjunct TCR activating molecules of the plurality of co-activating molecular ligands is about 100:1 to about 1:100 mol:mol (or about 100:1 to about 90:1, about 90:1 to about 80:1, about 80:1 to about 70:1, about 70:1 to about 60:1, about 60:1 to about about 50:1, about 50:1 to about 40:1, about 40:1 to about 30:1, about 30:1 to about 20:1, about 20:1 to about 10:1, about 10:1 to about 1:1, about 1:1 to about 1:10, about 1:10 to about 1:20, about 1:20 to about 1:30, about 1:30 to about 1:40, about 1:40 to about 1:50, about 1:50 to about 1:60, about 1:60 to about 1:70, about 1:70 to about 1:80, about 1:80 to about 1:90, or about 1:90 to about 1:100; or from about 3:1 to about 1:3; or from about 1:2 to about 2:1; or about 1:1).

Embodiment 120 is the proto-antigen-presenting surface of any one of embodiments 117 to 119, wherein a ratio of the primary activating molecular ligands to the co-activating molecular ligands present on the proto-antigen-presenting surface is about 1:10 to about 2:1, about 1:5 to about 2:1, about 1:2 to about 2:1, about 1:10 to about 1:1, about 1:5 to about 1:1, about 1:1 to about 2:1, or about 1:2 to about 1:1 mol:mol.

Embodiment 121 is the proto-antigen-presenting surface of any one of embodiments 117 to 120, further including a plurality of adhesion stimulatory molecular ligands, optionally wherein each adhesive molecular ligand includes a ligand for a cell adhesion receptor including an ICAM protein sequence or fragment thereof which retains activity of the parent molecule.

Embodiment 122 is the proto-antigen-presenting surface of any one of embodiments 117 to 121, further including a plurality of growth-stimulatory molecular ligands, wherein each of the growth-stimulatory molecular ligands includes a growth factor receptor ligand.

Embodiment 123 is a method of preparing an antigen-presenting surface including a peptide antigen, the method including: reacting the peptide antigen with a proto-antigen-presenting surface according to any one of embodiments 117 to 122, wherein the initial peptide is substantially displaced and the peptide antigen becomes associated with the MHC molecules.

Embodiment 124 is the method of embodiment 123, wherein the peptide antigen includes a tumor-associated antigen.

Embodiment 125 is the method of embodiment 123 or embodiment 124, wherein reacting the peptide antigen with the proto-antigen-presenting surface is performed in the presence of an exchange factor.

Embodiment 126 is the method of any one of embodiments 123 to 125, further including contacting a T lymphocyte with the antigen-presenting surface including the peptide antigen.

Embodiment 127 is the method of embodiment 126, wherein the T lymphocyte is from a subject in need of treatment for cancer.

Embodiment 128 is method of embodiment 127, wherein the T lymphocyte is CD8⁺.

Embodiment 129 is a population of T cells including activated T cells produced by the method of any one of embodiments 126 to 128.

Embodiment 130 is a microfluidic device or pharmaceutical composition including the population of embodiment 129.

Embodiment 131 is a method of screening a plurality of peptide antigens for T-cell activation, the method including: reacting a plurality of different peptide antigens with a plurality of proto-antigen-presenting surfaces according to any one of embodiments 117 to 122, thereby substantially displacing exchange factors or initial peptides and forming a plurality of antigen-presenting surfaces; contacting a plurality of T cells with the antigen-presenting surfaces; and monitoring the T cells for activation, wherein activation of a T cell indicates that a peptide antigen associated with the surface with which the T cell was contacted is able to contribute to T cell activation.

Embodiment 132 is the method of embodiment 131, wherein the proto-antigen-presenting surfaces are reacted separately with the plurality of different peptide antigens, thereby generating a plurality of different antigen-presenting surfaces, or wherein the proto-antigen-presenting surfaces are reacted separately with pools of members of the plurality of different peptide antigens, thereby generating a plurality of different antigen-presenting surfaces.

Embodiment 133 is the kit any one of embodiments 1 to 30, 61 to 78, or the method of any one of embodiments 79 to 94, or 100 to 110, or the proto-antigen-presenting surface of any one of embodiments 117 to 122, wherein the covalently functionalized synthetic surface further includes a plurality of surface-blocking molecular ligands: each of the plurality of surface-blocking molecular ligands includes a hydrophilic moiety, an amphiphilic moiety, a zwitterionic moiety, and/or a negatively charged moiety; each of the plurality of surface-blocking molecular ligands includes a linker and a terminal surface-blocking group, optionally wherein the linkers of the plurality of surface-blocking molecular ligands are of the same length; each of the plurality of surface-blocking molecular ligands includes a linker and a terminal surface-blocking group, wherein the terminal surface-blocking group includes a hydrophilic moiety, amphiphilic moiety, zwitterionic moiety, and/or negatively charged moiety, optionally wherein the linkers of the plurality of surface-blocking molecular ligands are of the same length; each of the plurality of surface-blocking molecular ligands is covalently bound to the covalently functionalized synthetic surface or the proto-antigen-presenting surface; or each of the plurality of surface-blocking molecular ligands includes a linker and a terminal surface-blocking group, wherein the plurality of the surface-blocking molecular ligands includes 2, 3, or 4 different surface-blocking groups and/or 2, 3, 4, or more different lengths of linkers, chosen in any combination.

Embodiment 134 is the kit or method of any one of embodiments 1 to 30, 61 to 78, 79 to 94, or 100 to 110, wherein the covalently functionalized synthetic surface is a wafer, an inner surface of a tube, an inner surface of a microfluidic device, or a bead.

Embodiment 135 is the kit or method of embodiment 134, wherein the inner surface of the microfluidic device is within a chamber of the microfluidic device.

Embodiment 136 is the kit or method of embodiment 135, wherein the chamber is a sequestration pen and the microfluidic device further includes a flow region for containing a flow of a first fluidic medium; and the sequestration pen includes an isolation region for containing a second fluidic medium, the isolation region having a single opening, wherein the isolation region of the sequestration pen is an unswept region of the microfluidic device; and a connection region fluidically connecting the isolation region to the flow region; optionally wherein the microfluidic device includes a microfluidic channel including at least a portion of the flow region.

Embodiment 137 is the kit or the method of any one of embodiments 1 to 30, 61 to 78, or 80 to 94, wherein the first reactive moieties include or consist essentially of biotin.

Embodiment 138 is the kit or the method of any one of embodiments 28 to 30, 76 to 78, 90 to 94, or 133 to 137, wherein the biotin-binding agent is covalently attached to the covalently functionalized synthetic surface.

Embodiment 139 is the kit or the method of any one of embodiments 28 to 30, 76 to 78, 90 to 94, or 133 to 137, wherein the biotin-binding agent is noncovalently attached to the covalently functionalized synthetic surface through biotin functionalities.

Embodiment 140 is the kit or the method of any one of embodiments 28 to 30, 76 to 78, 90 to 94, or 133 to 139, wherein the biotin-binding agent is streptavidin.

Embodiment 141 is the kit or the method of any one of embodiments 4 to 18, 20 to 30, 62 to 78, 89 to 94, 110 to 113, or 133 to 140, wherein the covalently functionalized synthetic surface or the proto-antigen-presenting surface presents a plurality of biotin-binding agents, and wherein the second reactive moieties are configured to specifically bind to the biotin-binding agent.

Embodiment 142 is the kit, the method, or the surface of any one of embodiments 5 to 18, 20 to 30, 31 to 60, 63 to 78, 82 to 94, 95 to 116, 117 to 128, 131 to 132, or 133 to 141, wherein the exchange factor includes a free N-terminal amine.

Embodiment 143 is the kit, the method, or the surface of any one of embodiments 5 to 18, 20 to 30, 31 to 60, 63 to 78, 82 to 94, 95 to 116, 117 to 128, 131 to 132, or 133 to 142, wherein the exchange factor includes Gly as its penultimate C-terminal residue.

Embodiment 144 is the kit, the method, or the surface of any one of embodiments 5 to 18, 20 to 30, 31 to 60, 63 to 78, 82 to 94, 95 to 116, 117 to 128, 131 to 132, or 133 to 143, wherein the exchange factor is 2, 3, 4, or 5 amino acid residues in length.

Embodiment 145 is the kit, the method, or the surface of any one of embodiments 5 to 18, 20 to 30, 31 to 60, 63 to 78, 82 to 94, 95 to 116, 117 to 128, 131 to 132, or 133 to 144, wherein the exchange factor includes a linkage between its C-terminal and penultimate C-terminal residues which is a peptide bond, lactam, or piperazinone.

Embodiment 146 is the kit, the method, or the surface of any one of the embodiments 9 to 18, 20 to 30, 67 to 78, 90 to 92, 123 to 128, or 133 to 145, wherein: the plurality of surface-blocking molecular ligands all have the same terminal surface-blocking group; or the plurality of surface-blocking molecular ligands have a mixture of terminal surface-blocking groups; optionally wherein each of the plurality of surface-blocking molecular ligands includes a polyethylene glycol (PEG) moiety, a carboxylic acid moiety, or a combination thereof.

Embodiment 147 is the kit, the method, or the surface of any one of embodiments 20 to 30, 76 to 78, 90 to 94, or 133 to 146, wherein the covalently functionalized synthetic surface or the proto-antigen-presenting surface further includes a first portion and a second portion, wherein the distribution of the at least one plurality of biotin-binding agent or biotin functionalities is located in the first portion of the covalently modified synthetic surface, and the distribution of the at least one plurality of the surface-blocking molecular ligands is located in the second portion.

Embodiment 148 is the kit, the method, or the surface of embodiment 147, wherein a second plurality of surface-blocking molecular ligands is disposed in the first portion of the covalently functionalized synthetic surface or the proto-antigen-presenting surface.

Embodiment 149 is the kit, the method, or the surface of embodiment 148, wherein the first region including at least the subset of the plurality of the streptavidin or biotin functionalities has an area of about 0.10 square microns to about 4.0 square microns or about 0.8 square microns to about 4.0 square microns.

Embodiment 150 is the kit, the method, the surface of any one of embodiments 1 to 30, 61 to 78, 79 to 94, 100, 101, 109 to 110, 117 to 122, 123 to 130, or 131-149, wherein the covalently functionalized synthetic surface or the proto-antigen-presenting surface includes glass, polymer, metal, ceramic, and/or a metal oxide.

Embodiment 151 is the kit, the method, or the surface of any one of embodiments 1 to 30, 61 to 78, 79 to 94, 100, 101, 109 to 110, 117 to 122, 123 to 130, or 131-149, wherein the covalently functionalized synthetic surface or the proto-antigen-presenting surface is a bead.

Embodiment 152 is the kit or the method of any one of embodiments 4 to 30, 62 to 75, 76 to 78, 89 to 94, 110 to 116, 119 to 128, or 131 to 151, wherein a ratio of the TCR co-activating molecules to the adjunct TCR activating molecules of the plurality of co-activating molecular ligands is about 100:1 to about 1:100 (or about 100:1 to about 90:1, about 90:1 to about 80:1, about 80:1 to about 70:1, about 70:1 to about 60:1, about 60:1 to about about 50:1, about 50:1 to about 40:1, about 40:1 to about 30:1, about 30:1 to about 20:1, about 20:1 to about 10:1, about 10:1 to about 1:1, about 1:1 to about 1:10, about 1:10 to about 1:20, about 1:20 to about 1:30, about 1:30 to about 1:40, about 1:40 to about 1:50, about 1:50 to about 1:60, about 1:60 to about 1:70, about 1:70 to about 1:80, about 1:80 to about 1:90, or about 1:90 to about 1:100).

Embodiment 153 is the kit or the method of any one of embodiments 4 to 30, 62 to 75, 76 to 78, 89 to 94, 110 to 116, 119 to 128, or 131 to 152, wherein the ratio of the TCR co-activating molecules to the adjunct TCR activating molecules of the plurality of co-activating molecular ligands is about 10:1 to about 1:20.

Embodiment 154 is the kit, the method, or the surface of embodiment 153, wherein the ratio of the TCR co-activating molecules to the adjunct TCR activating molecules of the plurality of co-activating molecular ligands is about 10:1 to about 1:10.

Embodiment 155 is the kit, the method, or the surface of any one of embodiments 1-154, wherein the MHC molecule includes a human leukocyte antigen A (HLA-A) heavy chain.

Embodiment 156 is the kit, the method, or the surface of embodiment 155, wherein the HLA-A heavy chain is an HLA-A*01, HLA-A*02, HLA-A*03, HLA-A*11, HLA-A*23, HLA-A*24, HLA-A*25, HLA-A*26, HLA-A*29, HLA-A*30, HLA-A*31, HLA-A*32, HLA-A*33, HLA-A*34, HLA-A*43, HLA-A*66, HLA-A*68, HLA-A*69, HLA-A*74, or HLA-A*80 heavy chain.

Embodiment 157 is the kit, the method, or the surface of any one of embodiments 1 to 153, wherein the MHC molecule includes a human leukocyte antigen B (HLA-B) heavy chain.

Embodiment 158 is the kit, the method, or the surface of embodiment 157, wherein the HLA-B heavy chain is an HLA-B*07, HLA-B*08, HLA-B*13, HLA-B*14, HLA-B*15, HLA-B*18, HLA-B*27, HLA-B*35, HLA-B*37, HLA-B*38, HLA-B*39, HLA-B*40, HLA-B*41, HLA-B*42, HLA-B*44, HLA-B*45, HLA-B*46, HLA-B*47, HLA-B*48, HLA-B*49, HLA-B*50, HLA-B*51, HLA-B*52, HLA-B*53, HLA-B*54, HLA-B*55, HLA-B*56, HLA-B*57, HLA-B*58, HLA-B*59, HLA-B*67, HLA-B*73, HLA-B*78, HLA-B*81, HLA-B*82, or HLA-B*83 heavy chain.

Embodiment 159 is the kit, the method, or the surface of any one of embodiments 1 to 154, wherein the MHC molecule includes a human leukocyte antigen C (HLA-C) heavy chain.

Embodiment 160 is the kit, the method, or the surface of embodiment 159, wherein the HLA-C heavy chain is an HLA-C*01, HLA-C*02, HLA-C*03, HLA-C*04, HLA-C*05, HLA-C*06, HLA-C*07, HLA-C*08, HLA-C*12, HLA-C*14, HLA-C*15, HLA-C*16, HLA-C*17, or HLA-C*18 heavy chain.

Embodiment 161 is the kit, or the method, or the surface of any one of embodiments 4 to 30, 62 to 75, 76 to 78, 89 to 94, 110 to 116, 119 to 128, or 131 to 160, wherein the TCR co-activating molecule includes a protein.

Embodiment 162 is the kit, or the method, or the surface of embodiment 161, wherein the TCR co-activating molecule further includes a site-specific C-terminal biotin moiety.

Embodiment 163 is the kit, or the method, or the surface of embodiment 161 or 162, wherein the TCR co-activating molecule includes a CD28 binding protein or a fragment thereof which retains binding ability with CD28.

Embodiment 164 is the kit, or the method, or the surface of embodiment 163, wherein the CD28 binding protein includes a CD80 molecule or a fragment thereof, wherein the fragment retains binding ability to CD28.

Embodiment 165 is the kit, or the method, or the surface of embodiment 161 or 162, wherein the TCR co-activating molecule includes an anti-CD28 antibody or fragment thereof, wherein the fragment retains binding activity with CD28.

Embodiment 166 is the kit, or the method, or the surface of any one of embodiments 4 to 30, 62 to 75, 76 to 78, 89 to 94, 110 to 116, 119 to 128, or 131 to 165, wherein the adjunct TCR activating molecule is configured to provide adhesion stimulation.

Embodiment 167 is the kit, or the method, or the surface of any one of embodiments 4 to 30, 62 to 75, 76 to 78, 89 to 94, 110 to 116, 119 to 128, or 131 to 166, wherein the adjunct TCR activating molecular ligand includes a CD2 binding protein or a fragment thereof, wherein the fragment retains binding ability with CD2.

Embodiment 168 is the kit, or the method, or the surface of embodiment 167, wherein the CD2 binding protein further includes a site-specific C-terminal biotin moiety.

Embodiment 169 is the kit, or the method, or the surface of embodiment 167 or 168, wherein the adjunct TCR activating molecular ligand includes a CD58 molecule or fragment thereof, wherein the fragment retains binding activity with CD2.

Embodiment 170 is the kit, or the method, or the surface of embodiment 167 or 168, wherein the adjunct TCR activating molecule includes an anti-CD2 antibody or a fragment thereof, wherein the fragment retains binding activity with CD2.

Embodiment 171 is the method or the surface of any one of embodiments 79 to 94, 117 to 122, or 131 to 170, wherein the plurality of primary activating molecular ligands is disposed upon at least a portion of the antigen-presenting surface at a density from about 4×10² to about 3×10⁴ molecules per square micron, in each portion or sub-region where it is attached.

Embodiment 172 is the method or the surface of embodiment 171, wherein the plurality of primary activating molecular ligands is disposed upon at least a portion of the antigen-presenting surface at a density from about 4×10² to about 2×10³ molecules per square micron.

Embodiment 173 is the method or the surface of embodiment 171, wherein the plurality of primary activating molecular ligands is disposed upon at least a portion of the antigen-presenting surface at a density from about 2×10³ to about 5×10³ molecules per square micron.

Embodiment 174 is the method or the surface of embodiment 171, wherein the plurality of primary activating molecular ligands is disposed upon at least a portion of a surface of the antigen-presenting surface at a density from about 5×10³ to about 2×10⁴ molecules per square micron, about 1×10⁴ to about 2×10⁴ molecules per square micron, or about 1.25×10⁴ to about 1.75×10⁴ molecules per square micron.

Embodiment 175 is the method or the surface of any one of embodiments 171 to 174, wherein the plurality of primary activating molecular ligands is disposed upon substantially all of the antigen-presenting surface at the stated density.

Embodiment 176 is the method or the surface of any one of embodiments 79 to 94, 117 to 122, or 131 to 175, wherein the plurality of co-activating molecular ligands is disposed upon at least a portion the antigen-presenting surface at a density from about 5×10² to about 2×10⁴ molecules per square micron or about 5×10² to about 1.5×10⁴ molecules per square micron.

Embodiment 177 is the method or the surface of embodiment 176, wherein the plurality of co-activating molecular ligands is disposed upon at least a portion of the antigen-presenting surface at a density from about 5×10³ to about 2×10⁴ molecules per square micron, about 5×10³ to about 1.5×10⁴ molecules per square micron, about 1×10⁴ to about 2×10⁴ molecules per square micron, about 1×10⁴ to about 1.5×10⁴ molecules per square micron, about 1.25×10⁴ to about 1.75×10⁴ molecules per square micron, about 1.25×10⁴ to about 1.5×10⁴ molecules per square micron, about 2×10³ to about 5×10³ molecules per square micron, or about 5×10² to about 2×10³ molecules per square micron.

Embodiment 178 is the method or the surface of embodiment 176 or 177, wherein the plurality of co-activating molecular ligands is disposed upon substantially all of the antigen-presenting surface at the stated density.

Embodiment 179 is the method or the surface of any one of embodiments 79 to 94, 117 to 122, or 131 to 178, wherein a ratio of the primary activating molecular ligands to the co-activating molecular ligands present on the antigen-presenting surface is about 1:10 to about 2:1, about 1:5 to about 2:1, about 1:2 to about 2:1, about 1:10 to about 1:1, about 1:5 to about 1:1, about 1:1 to about 2:1, or about 1:2 to about 1:1.

Embodiment 180 is the method or the surface of any one of embodiments 79 to 94, 117 to 122, or 131 to 179, wherein each of the plurality of primary activating molecular ligands is noncovalently bound to a binding moiety, and further wherein the binding moiety is covalently bound to the antigen-presenting surface.

Embodiment 182 is the method or the surface of embodiment 180, wherein each of the plurality of primary activating molecular ligands includes a biotin and is noncovalently bound to a biotin-binding agent, and further wherein the biotin-binding agent is covalently bound to the antigen-presenting surface.

Embodiment 183 is the method or the surface of any one of embodiments 79 to 94, 117 to 122, or 131 to 179, wherein each of the plurality of primary activating molecular ligands is noncovalently bound to a binding moiety, and further wherein the binding moiety is noncovalently bound to the antigen-presenting surface.

Embodiment 184 is the method or the surface of embodiment 183, wherein each of the plurality of primary activating molecular ligands includes a biotin moiety, the binding moiety includes a biotin-binding agent, and the biotin-binding agent is noncovalently bound to a second biotin moiety covalently attached to the antigen-presenting surface.

Embodiment 185 is the method or the surface of embodiment 183 or 184, wherein the biotin-binding agent is streptavidin.

Embodiment 186 is the method or the surface of any one of embodiments 79 to 94, 117 to 122, or 131 to 185, wherein each of the plurality of co-activating molecular ligands is non-covalently attached to a streptavidin and the streptavidin is non-covalently attached to a streptavidin binding molecule, further wherein the streptavidin binding molecule is covalently attached via a linker to the proto-antigen-presenting surface, optionally wherein the streptavidin binding molecule includes biotin.

Embodiment 187 is the method or the surface of any one of embodiments 79 to 94, 117 to 122, or 131 to 185, wherein each of the plurality of co-activating molecular ligands is covalently connected to the surface via a linker.

Embodiment 188 is the method or the surface of any one of embodiments 79 to 94, 117 to 122, or 131 to 185, wherein each of the plurality of co-activating molecular ligands is non-covalently attached to a streptavidin moiety; and the streptavidin moiety is covalently attached to the antigen-presenting surface.

Embodiment 189 is the surface or the method of any one of embodiments 117 to 122, or 131 to 188, wherein further including a plurality of adhesion stimulatory molecular ligands, optionally wherein each adhesive molecular ligand includes a ligand for a cell adhesion receptor including an ICAM protein sequence or fragment thereof which retains activity of the parent molecule.

Embodiment 190 is the surface or the method of embodiment 189, wherein the adhesion stimulatory molecular ligand is covalently connected to the antigen-presenting surface via a linker.

Embodiment 191 is the surface or the method of embodiment 190, wherein the adhesion stimulatory molecular ligand is non-covalently attached to a streptavidin moiety, wherein the streptavidin moiety is covalently attached via a linker to the antigen-presenting surface.

Embodiment 192 is the surface or the method of embodiment 190, wherein the adhesion stimulatory molecular ligand is non-covalently attached to a streptavidin, wherein the streptavidin is noncovalently attached to a biotin and the biotin is covalently attached via a linker to the antigen-presenting surface.

Embodiment 193 is the surface or the method of any one of embodiments 117 to 122, or 131 to 192, further including a plurality of growth-stimulatory molecular ligands, wherein each of the growth-stimulatory molecular ligands includes a growth factor receptor ligand.

Embodiment 194 is the surface or the method of embodiment 193, wherein the growth factor receptor ligand includes a cytokine or fragment thereof, wherein the fragment retains receptor binding ability, optionally wherein the cytokine includes IL-21.

Embodiment 195 is the surface or the method of any one of embodiments 117 to 122, or 131 to 194, further including a first portion and a second portion, wherein the distribution of the plurality of primary activating molecular ligands and the distribution of the plurality of co-activating molecular ligands are located in the first portion of the antigen-presenting surface, and the second portion is configured to substantially exclude the primary activating molecular ligands.

Embodiment 196 is the surface or the method of embodiment 195, wherein at least one plurality of surface-blocking molecular ligands is located in the second portion of the at least one inner surface of the antigen-presenting surface.

Embodiment 197 is the surface or the method of embodiment 195 or 196, wherein the first portion of the antigen-presenting surface further includes a plurality of first regions, each first region including at least a subset of the plurality of the primary activating molecular ligands, wherein each of the plurality of first regions is separated from another of the plurality of first region by the second portion configured to substantially exclude primary activating molecular ligands.

Embodiment 198 is the surface or the method of embodiment 197, wherein each of the plurality of first regions including the at least a subset of the plurality of the primary activating molecular ligands further includes a subset of the plurality of the co-activating molecular ligands.

Embodiment 199 is the surface or the method of embodiment 197 or 198, wherein each of the plurality of first regions including at least the subset of the plurality of the primary activating molecular ligands has an area of about 0.10 square microns to about 4.0 square microns.

Embodiment 200 is the surface or the method of any one of embodiments 197 to 199, wherein the area of each of the plurality of first regions including at least the subset of the plurality of the primary activating molecular ligands is about 0.8 square microns to about 4.0 square microns.

Embodiment 201 is the surface or the method of any one of embodiments 197 to 200, wherein each of the plurality of first regions further includes at least a subset of a plurality of adhesion stimulatory molecular ligands, and optionally wherein each of the adhesion stimulatory molecular ligands includes a ligand for a cell adhesion receptor including an ICAM protein sequence or fragment thereof which retains activity of the parent molecule.

Embodiment 202 is the surface or the method of any one of embodiments 197 to 201, wherein the second portion configured to substantially exclude the primary activating molecular ligands is also configured to substantially exclude co-activating molecular ligands.

Embodiment 203 is the surface or the method of any one of embodiments 197 to 202, wherein the second portion configured to substantially exclude the primary activating molecular ligands is further configured to include a plurality of growth stimulatory molecular ligands, wherein each of the growth stimulatory molecular ligands includes a growth factor receptor ligand.

Embodiment 204 is the surface or the method of any one of embodiments 197 to 203, wherein the second portion configured to substantially exclude the primary activating molecular ligands includes a plurality of adhesion stimulatory molecular ligands, wherein each of the adhesion stimulatory molecular ligands includes a ligand for a cell adhesion receptor including an ICAM protein sequence or fragment thereof which retains activity of the parent molecule.

Embodiment 205 is the surface or the method of any one of embodiments 197 to 204, wherein the antigen-presenting surface is a surface of a microfluidic device and each of the plurality of first regions including at least a subset of the plurality of primary activating molecular ligands is disposed at least one surface within a chamber of the microfluidic device.

Embodiment 206 is the surface or the method of embodiment 148, wherein the second plurality of surface-blocking molecular ligands limits the density of functionalizing moieties of an antigen-presenting synthetic surface formed from the covalently functionalized synthetic surface.

Embodiment 207 is the method of any one of embodiments 117 to 122, or 131 to 206, wherein further including reacting a plurality of surface-blocking molecules with a first additional plurality of binding moieties of the covalently functionalized surface, wherein each of the binding moieties of the first additional plurality is configured for binding the surface-blocking molecule.

Embodiment 208 is the method of any one of embodiments 117 to 122, or 131 to 207, further including reacting a plurality of adhesion stimulatory molecular ligands, wherein each adhesion stimulatory molecular ligand includes a ligand for a cell adhesion receptor including an ICAM protein sequence or fragment thereof which retains activity of the parent molecule, with a second additional plurality of binding moieties of the covalently functionalized surface, wherein each of the binding moieties of the second additional plurality is configured for binding with the cell adhesion receptor ligand molecule.

Embodiment 209 is the kit, the method, or the surface of any one of embodiments 1 to 3, 19, 61 to 78, or 131 to 208, including a peptide antigen.

Embodiment 210 is the kit, the method, or the surface of any one of embodiments 4 to 30, 82 to 94, 95 to 113, 114 to 116, 117 to 122, 123 to 132, or 209, wherein the peptide antigen includes a tumor-associated antigen.

1. (canceled)

2. (canceled)

3. A kit for generating an antigen-presenting surface, the kit comprising:
a covalently functionalized synthetic surface;
a primary activating molecule comprising a major histocompatibility complex (MHC) molecule and a first reactive moiety, wherein the MHC molecule is configured to bind to a T cell receptor (TCR), and the first reactive moiety is configured to react with or bind to the covalently functionalized surface;
an initial peptide bound to the MHC molecule, wherein the initial peptide has an affinity for binding a binding groove of the MHC molecule comprising a predicted Kd from 1E+1 nM to 1E+5 nM, from 1E+1 nM to 2E+5 nM, from 1E+2 nM to 1E+5 nM, from 1E+2 nM to 2E+4 nM, from 1E+2 nM to 1E+3 nM, from 1E+2 nm to 1E+4 nM, from 1E+1 nm to 2E+4 nM, from 1E+2 nM to 1E+3 nM, from 1.5E+2 nM to 1E+5 nM;
at least one co-activating molecule that comprises a second reactive moiety configured to react with or bind to the covalently functionalized surface, wherein each co-activating molecule is selected from a TCR co-activating molecule and an adjunct TCR activating molecule;
a buffer suitable for performing an exchange reaction; and
optionally, instructions for performing an exchange reaction wherein a peptide antigen displaces the initial peptide.

4. (canceled)

5. The kit of claim 3, further comprising an exchange factor, wherein the exchange factor is provided separately from the primary activating molecule and the initial peptide bound to the MHC molecule, optionally, wherein:
the exchange factor comprises Leu, Phe, Val, Arg, Met, Lys, Ile, homoleucine, cyclohexylalanine, or norleucine as its C-terminal amino acid residue; and/or
the exchange factor comprises Gly, Ala, Ser, or Cys as its penultimate C-terminal residue; and/or
the exchange factor is 2 amino acid residues in length.

6. (canceled)

7. (canceled)

8. (canceled)

9. The kit of claim 3, further comprising a plurality of surface-blocking molecules, wherein the covalently functionalized surface further comprises a first additional plurality of binding moieties configured for binding the surface-blocking molecule.

10. The kit of claim 3, wherein the covalently functionalized synthetic surface further comprises a plurality of surface-blocking molecular ligands:
each of the plurality of surface-blocking molecular ligands comprises a hydrophilic moiety, an amphiphilic moiety, a zwitterionic moiety, and/or a negatively charged moiety;
each of the plurality of surface-blocking molecular ligands comprises a linker and a terminal surface-blocking group;
each of the plurality of surface-blocking molecular ligands comprises a linker and a terminal surface-blocking group, wherein the terminal surface-blocking group comprises a hydrophilic moiety, amphiphilic moiety, zwitterionic moiety, and/or negatively charged moiety;
each of the plurality of surface-blocking molecular ligands is covalently bound to the covalently functionalized synthetic surface or the proto-antigen-presenting surface; or
each of the plurality of surface-blocking molecular ligands comprises a linker and a terminal surface-blocking group, wherein the plurality of the surface-blocking molecular ligands comprises 2, 3, or 4 different surface-blocking groups and/or 2, 3, 4, or more different lengths of linkers, chosen in any combination.

11. The kit of claim 3, wherein:
the initial peptide has a length of 4, 5, 6, 7, 8, 9, 10, 11, 12, 13, 14, 15, 16, 17, or 18 amino acid residues; and/or
the initial peptide comprises a lysine as the fourth or fifth amino acid residue counting from the N-terminus thereof; and/or
the initial peptide comprises a label attached to a fourth or fifth amino acid residue; and/or
the initial peptide comprises GMGQKDSYV (SEQ ID NO: 1) or GAATKMAAV (SEQ ID NO:13).

12. (canceled)

13. (canceled)

14. (canceled)

15. (canceled)

16. The kit of claim 3, wherein the initial peptide binds the MHC molecule with a half-life of at least hours.

17. The kit of claim 3, wherein the covalently functionalized synthetic surface is a bead.

18. (canceled)

19. (canceled)

20. The kit of claim 3, wherein the MHC molecule comprises a human leukocyte antigen A (HLA-A) heavy chain.

21. The kit of claim 3, further comprising at least one co-activating molecule that comprises a second reactive moiety configured to react with or bind to the covalently functionalized surface, wherein
each co-activating molecule is selected from a TCR co-activating molecule and an adjunct TCR activating molecule;
the TCR co-activating molecule comprises a CD28 binding protein or a fragment thereof which retains binding ability with CD28; and
the adjunct TCR activating molecule is configured to provide adhesion stimulation and/or the adjunct TCR activating molecular ligand comprises a CD2 binding protein, anti-CD2 antibody, or a fragment thereof, wherein the fragment of the CD2 binding protein or anti-CD2 antibody retains binding ability with CD2.

22. A method of identifying an initial peptide sequence for stabilizing a major histocompatibility complex (MHC) molecule, comprising:
binding a first peptide sequence to a binding groove of the MHC molecule, wherein the first peptide sequence is a detectably labelled peptide sequence, thereby forming a detectably labelled peptide sequence:MHC molecule complex (LP:MHC complex) stabilizing the MHC molecule;
performing an exchange reaction comprising contacting the LP:MHC complex with an exchange factor and a second peptide sequence for a first period of time, wherein the second peptide sequence is configured to stabilize the MHC molecule when bound to the binding groove; and
detecting displacement of the detectably labelled peptide sequence from the binding groove of the MHC molecule;
wherein the second peptide sequence displaces at least equal to or more than 60% of the detectably labelled peptide bound to the MHC molecules, thereby identifying the second peptide sequence as a suitable initial peptide sequence.

23. (canceled)

24. The method of claim 22, wherein the detectably labelled peptide sequence is labelled at an amino acid residue that does not interfere with forming the LP:MHC complex, and optionally, wherein detecting displacement of the detectably labelled peptide comprises determining loss of fluorescence from the LP:MHC complex.

25. The method of claim 22, wherein an affinity of the detectably labelled peptide sequence for binding the binding groove comprises a predicted Kd from 1E+1 nM to 2E+5 nM, or wherein the detectably labelled peptide sequence binds the MHC molecule with a half-life of at least 0.1, 0.2, 0.4, 1.5, 2, 4, 6, 8, 10, 12, 14, 16, 18, 20, 24, 28, 32, 36, 48, or 72 hours.

26. (canceled)

27. The method of claim 22, wherein:
the exchange factor comprises a Gly, Ala, Ser, or Cys as its penultimate C-terminal residue; and/or
the exchange factor comprises Leu, Phe, Val, Arg, Met, Lys, Ile, homoleucine, cyclohexylalanine, or norleucine as its C-terminal amino acid residue; and/or
the exchange factor is 2 amino acid residues in length.

28. (canceled)

29. (canceled)

30. The method of claim 22, wherein the first period of time is 2 hr to 6 hr or 1 hr to 10 hr.

31. (canceled)

32. (canceled)

33. The method of claim 22, wherein the detectably labelled peptide sequence:
has a length of 4, 5, 6, 7, 8, 9, 10, 11, 12, 13, 14, 15, 16, 17, or 18 amino acid residues; and/or
comprises a lysine as the fourth or fifth amino acid residue of the detectably labelled peptide sequence; and/or
has an amino acid sequence selected from: IMALKQAGL (SEQ ID NO: 11); FLAIKLYVG (SEQ ID NO: 12); GAATKMAAV (SEQ ID NO: 13); QLALKKVEGV (SEQ ID NO: 14); IMALKKQAGL (SEQ ID NO: 15); FLAIKKLYVG (SEQ ID NO: 16); TEIGKDVIGL (SEQ ID NO: 17); or ILKEKKVHVG (SEQ ID NO: 18).

34. (canceled)

35. (canceled)

36. (canceled)

37. (canceled)

38. (canceled)

39. The method of claim 22, wherein the MHC molecule comprises HLA-A*01, HLA-A*02, HLA-A*03, HLA-A*11, or HLA-A*24 or wherein the MHC molecule comprises HLA-B*07, HLA-B*27, HLA-B*40, HLA-B*44, or HLA-B*58.

40. (canceled)

41. A method of analyzing stability of a complex comprising a major histocompatibility complex (MHC) molecule and a peptide antigen,
wherein the MHC molecule is:
configured to bind to a T cell receptor (TCR); and
stabilized by complexation with an initial peptide which is identified by the method of claim 22;
wherein the method comprises:
contacting a plurality of the MHC molecules with the peptide antigen and an exchange factor, thereby forming peptide antigen-bound MHC molecules; and
measuring total binding and/or an extent of dissociation of the peptide antigen from the MHC molecule, optionally, further comprises determining one or more kinetic parameters of the peptide antigen-bound MHC molecules.

42. The method of claim 41, wherein measuring total binding and/or the extent of dissociation comprises measuring binding of an agent to the MHC molecule, wherein the agent specifically binds to (i) the initial peptide, and/or (ii) a peptide-bound conformation of the MHC molecule, and wherein the agent does not recognize a peptide-unbound conformation of the MHC molecule, optionally, wherein the initial peptide binds the MHC molecule with a half-life of at least 4 hours.

43. The method of claim 41, wherein the MHC molecules are disposed on a covalently functionalized synthetic surface via reaction of a reactive moiety of each of the MHC molecules with a binding moiety of the covalently functionalized surface.

44. (canceled)

45. (canceled)

46. (canceled)

47. The method of claim 41, wherein the exchange factor comprises Leu, Phe, Val, Arg, Met, Lys, Ile, homoleucine, cyclohexylalanine, or norleucine as its C-terminal amino acid residue or wherein the exchange factor comprises Gly, Ala, Ser, or Cys as its penultimate C-terminal residue.

48. (canceled)

49. (canceled)

50. (canceled)
